(19) United States
(12) Patent Application Publication
(10) Pub. No.: US 2026/0108521 A1
(43) Pub. Date: Apr. 23, 2026

(54) GSPT1 DEGRADERS, COMPOSITIONS COMPRISING THE DEGRADER, AND METHODS OF USING THE SAME

(71) Applicant: BIOFRONT LTD, Grand Cayman (KY)

(72) Inventors: Lichao FANG, Beijing (CN); Miao LIU, Beijing (CN); Tianwei MA, Beijing (CN)

(21) Appl. No.: 19/117,970

(22) PCT Filed: Oct. 4, 2023
(86) PCT No.: PCT/CN2023/123087

§ 371 Date: Apr. 3, 2025

(30) Foreign Application Priority Data
Oct. 4, 2022 (WO) PCT/CN2022/123708

(51) Int. Cl. A61K 31/513; A61K 31/454; A61K 31/4545; C07D 401/14; C07D 417/14; C07D 471/04; C07D 498/04

(52) U.S. Cl. CPC A61K 31/513; A61K 31/454; A61K 31/4545; C07D 401/14; C07D 417/14; C07D 471/04; C07D 498/04

(57) ABSTRACT

Provided compounds of Formula (I), a tautomer thereof, a deuterated derivative of the compound or the tautomer, and a pharmaceutically acceptable salt of the foregoing, compositions comprising the compounds of Formula (I), a tautomer thereof, a deuterated derivative of the compound or the tautomer, and/or a pharmaceutically acceptable salt of the foregoing, and methods of using the same, in treating, for example, the diseases, disorders, or conditions mediated by the degradation of G1 to S phase transition protein 1 (GSPT1).

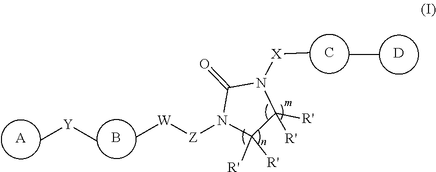

### FIELD OF THE INVENTION

This disclosure provides compounds of Formula I, a tautomer thereof, a deuterated derivative of the compound or the tautomer, and a pharmaceutically acceptable salt of the foregoing, compositions comprising the compounds of Formula I, a tautomer thereof, a deuterated derivative of the compound or the tautomer, and/or a pharmaceutically acceptable salt of the foregoing, and methods of using the same, in treating, for example, the diseases, disorders, or conditions mediated by the degradation of G1 to S phase transition 1 (GSPT1) protein.

### BACKGROUND OF THE INVENTION

Aberrant protein function and mis-regulation of protein synthesis may contribute to uncontrolled cell growth, proliferation, and migration, leading to cancer. Termination of translation is a GTP-dependent process that is regulated by two key protein eukaryotic release factors eRF1 and eRF3. The translation termination factor eRF3, also known as GSPT1 (G1 to S phase transition 1) protein, is a GTPase that interacts with eRF1 to promote stop codon recognition and release of nascent peptide from ribosome (Chauvin et al., Involvement of Human Release Factors eRF3a and eRF3b in Translation Termination and Regulation of the Termination Complex Formation, Mol Cell Biol., 2005, 25(14): 5801-5811). GSPT1 protein activates eRF1 in a GTP-dependent manner and its GTPase activity requires complexing with eRF1 and ribosomes to form the functional translation termination complexes (Zhouravleva et al., Termination of translation in eukaryotes is governed by two interacting polypeptide chain release factors, eRF1 and eRF3, EMBO J., 1995, 14, 4065-4072; Frolova et al., Eukaryotic polypeptide chain release factor eRF3 is an eRF1- and ribosome-dependent guanosine triphosphatase, RNA, 1996, 2, 334-341). In addition to its role in translation termination in response to the termination codons (Hoshino et al., A human homologue of the yeast GST1 gene codes for a GTP-binding protein and is expressed in a proliferation-dependent manner in mammalian cells, EMBO J., 1989, 8, 3807-3814; Aliouat et al., Divergent effects of translation termination factor eRF3A and nonsense-mediated mRNA decay factor UPF1 on the expression of uORF carrying mRNAs and ribosome protein genes, RNA Biol., 2020, 17(2):227-239), GSPT1 protein is involved in cell cycle regulation, cytoskeleton organization, and apoptosis. Accordingly, decreased levels of GSPT1 may impair control of cell proliferation and facilitate cell migration and scar formation. Indeed, increased expression of GSPT1 protein has been reported in human malignancies, including acute myeloid leukemia, multiple myeloma, breast cancer, hepatocellular carcinoma, prostate cancer, lung cancer and gastric cancer (Brito et al., Polyglycine expansions in eRF3/GSPT1 are associated with gastric cancer susceptibility, Carcinogenesis, 2005, 26, 2046-2049; Wright and Lange, Newer Potential Biomarkers in Prostate Cancer, Rev. Urol, 2007, 9(4), 207-213; Malta-Vacas, et al., eRF3a/GSPT1 12-GGC allele increases the susceptibility for breast cancer development, Oncol. Rep., 2009, 21(6):1551-1558; Miri et al., GGCn polymorphism of eRF3a/GSPT1 gene and breast cancer susceptibility, Med. Oncol., 2012, 29(3):1581-1585; Hashimoto, et al., Translation termination factor eRF3 is targeted for caspase-mediated proteolytic cleavage and degradation during DNA damage-induced apoptosis, Apoptosis, 2012, 17(12):1287-1299; Tian, The role of miR-144/GSPT1 axis in gastric cancer, Eur. Rev. Med. Pharmacol. Sci., 2018, 22(13):4138-4145; Sun, et al., LncRNA DLX6-AS1 promotes the proliferation, invasion, and migration of non-small cell lung cancer cells by targeting the miR-27b-3p/GSPT1 axis, Onco. Targets Ther., 2019; 12: 3945-3954; Zhang, et al., Downregulation of microRNA-27b-3p via aberrant DNA methylation contributes to malignant behavior of gastric cancer cells by targeting GSPT1, Biomed Pharmacother., 2019, 119:109417; Powell, et al., Selective Degradation of GSPT1 by Cereblon Modulators Identified via a Focused Combinatorial Library, ACS Chem. Biol., 2020, 15(10):2722-2730; Nishiguchi, et al., Identification of Potent, Selective, and Orally Bioavailable Small-Molecule GSPT1/2 Degraders from a Focused Library of Cereblon Modulators, J. Med. Chem., 2021, 64(11):7296-7311; Surka, et al., CC-90009, a novel cereblon E3 ligase modulator, targets acute myeloid leukemia blasts and leukemia stem cells, Blood, 2021,137(5): 661-677). Thus, GSPT1 protein has been identified as an oncogenic driver and a novel cancer target through which one may compromise active translation that contributes to malignant phenotypes of cancer cells. One mechanism to disrupt protein drivers of a disease is to decrease the cellular concentrations of these proteins by protein degradation. Cereblon is a protein that forms an E3 ubiquitin ligase complex, which ubiquinates various other proteins for further degradation.

Despite different GSPT1 degraders have been tested in clinical trials and in preclinical, dose-limiting toxicities and limited efficacy were observed. A novel GSPT1 degrader has the potential to improve clinical outcome.

### SUMMARY OF THE INVENTION

One aspect of the present disclosure provides a compound selected from compounds of Formula I, a tautomer thereof, a deuterated derivative of the compound or the tautomer, or a pharmaceutically acceptable salt of the foregoing, which can be employed in the treatment of diseases mediated by the degradation of GSPT1 protein. For example, disclosed herein is a compound of the following structural Formula I:

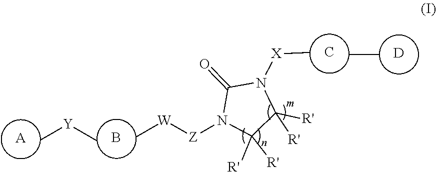

a tautomer thereof, a deuterated derivative of the compound or the tautomer, or a pharmaceutically acceptable salt of the foregoing, wherein:
(i) each R′ is independently chosen from hydrogen, halogen groups, linear, branched, and cyclic alkyl groups;
(ii) m and n are independently chosen from 0, 1, and 2;
(iii) X and Z are independently absent or is chosen from linear, branched, cyclic alkylene groups, linear, branched, and cyclic heteroalkylene groups, and linear, branched, cyclic alkyl groups;
(iv) Y and W are independently absent or chosen from —O—, —C(O)—, —C(O)R^{x}—, —C(S)—, —C(S)R^{x}—, —[C(R^{x}R^{y})]ₚ—, —S—, —S(O)₂—, —S(O)₂R^{x}—, NR^{x}—, and —NR^{x}C(O)—; further wherein p is chosen from 1, 2, 3, 4, 5, and 6; and R^{x} is chosen from hydrogen, linear, branched, and cyclic alkyl groups, carbocyclic groups, heterocyclic groups, aryl groups, and heteroaryl groups;
(v) ring A is chosen from

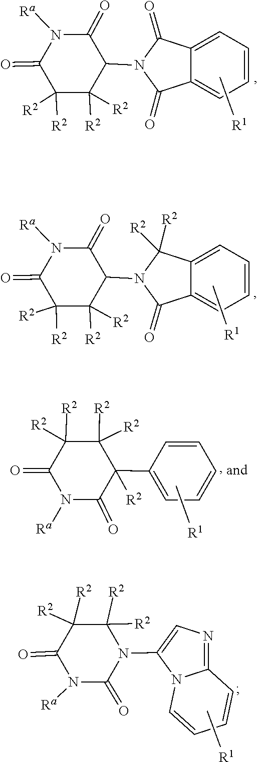

wherein R^{a} is chosen from hydrogen, linear, branched, and cyclic alkyl groups, and pro-drug groups; each R¹ and each R² are independently chosen from hydrogen, halogen groups, OR^{z}, and linear, branched, and cyclic alkyl groups; further wherein R^{z} is chosen from hydrogen, linear, branched, and cyclic alkyl groups, carbocyclic groups, heterocyclic groups, aryl groups, and heteroaryl groups;
(vi) ring B is absent or is chosen from optionally substituted cycloalkyl groups and heterocycloalkyl groups;
(vii) ring C is absent or is chosen from optionally substituted aryl groups and heteroaryl groups,
(viii) ring D is absent or is chosen from optionally substituted cycloalkyl groups, heterocycloalkyl groups, and heteroaryl groups;
   wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—C(O)N(C₁-C₆)₂ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
—S(O)₂C₁-C₆ linear, branched, and cyclic alkyl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

In one aspect of the present disclosure, the compounds of Formula I are selected from Compounds 1 to 24 shown below, a tautomer thereof, a deuterated derivative of the compound or the tautomer, and a pharmaceutically acceptable salt of the foregoing.

In some embodiments, the present disclosure provides pharmaceutical compositions comprising a compound of Formula I, a tautomer thereof, a deuterated derivative of the compound or the tautomer, and/or a pharmaceutically acceptable salt of the foregoing, and a pharmaceutically acceptable carrier. In some embodiments, the pharmaceutical compositions may comprise a compound selected from Compounds 1 to 24 shown below, a tautomer thereof, a deuterated derivative of the compound or the tautomer, and/or a pharmaceutically acceptable salt of the foregoing. These compositions may further comprise an additional active pharmaceutical agent.

Another aspect of the present disclosure provides methods of treating a disease, a disorder, or a condition mediated by the degradation of the GSPT1 protein in a subject, comprising administering a therapeutically effective amount of a compound of Formula I, a tautomer thereof, a deuterated derivative of the compound or the tautomer, and/or a pharmaceutically acceptable salt of the foregoing, or a pharmaceutical composition comprising any of the foregoing. In some embodiments, the methods of treatment comprise administering to a subject, a therapeutically effective amount of a compound selected from Compounds 1 to 24 shown below, a tautomer thereof, a deuterated derivative of the compound or the tautomer, and/or a pharmaceutically acceptable salt of the foregoing, or a pharmaceutical composition comprising any of the foregoing. In some embodiments, the methods of treatment comprise administering to a subject, a therapeutically effective amount of a compound selected from Compounds A to F shown below, a tautomer thereof, a deuterated derivative of the compound or the tautomer, and/or a pharmaceutically acceptable salt of the foregoing, or a pharmaceutical composition comprising any of the foregoing.

In some embodiments disclosed herein, the methods of treatment comprise administration of an additional active pharmaceutical agent to the subject in need thereof, either in the same pharmaceutical composition as a compound of Formula I, a tautomer thereof, a deuterated derivative of the compound or the tautomer, and/or a pharmaceutically acceptable salt of the foregoing, or in a separate composition. In some embodiments disclosed herein, the methods of treatment comprise administering a compound selected from Compounds 1 to 24 shown below, a tautomer thereof, a deuterated derivative of the compound or the tautomer, and/or a pharmaceutically acceptable salt of the foregoing with an additional active pharmaceutical agent either in the same composition or in a separate composition. In some embodiments disclosed herein, the methods of treatment comprise administering a compound selected from Compounds A to F shown below, a tautomer thereof, a deuterated derivative of the compound or the tautomer, and/or a pharmaceutically acceptable salt of the foregoing with an additional active pharmaceutical agent either in the same composition or in a separate composition.

Also disclosed herein are methods of decreasing GSPT1 protein activity, comprising administering to a subject a therapeutically effective amount of a compound of Formula I, a tautomer thereof, a deuterated derivative of the compound or the tautomer, and/or a pharmaceutically acceptable salt of the foregoing, or a pharmaceutical composition comprising any of the foregoing. In some embodiments disclosed herein, the methods of degrading a GSPT1 protein comprise administering to a subject, a compound selected from Compounds 1 to 24 shown below, a tautomer thereof, a deuterated derivative of the compound or the tautomer, and/or a pharmaceutically acceptable salt of the foregoing, or a pharmaceutical composition comprising any of the foregoing. In some embodiments disclosed herein, the methods of degrading a GSPT1 protein comprise administering to a subject, a compound selected from Compounds A to F shown below, a tautomer thereof, a deuterated derivative of the compound or the tautomer, and/or a pharmaceutically acceptable salt of the foregoing, or a pharmaceutical composition comprising any of the foregoing.

### BRIEF DESCRIPTION OF THE FIGURES

The foregoing summary, as well as the following detailed description of the disclosure, will be better understood when read in conjunction with the appended drawings. For the purpose of illustrating the present disclosure, the appended drawings illustrate some, but not all, alternative embodiments. It should be understood, however, that the disclosure is not limited to the precise arrangements and instrumentalities shown. These drawings, which are incorporated into and constitute part of the specification, assist in explaining the principles of the disclosure.

FIG. 1 shows a Western blot of the degradation of GSPT1 in HL-60 cells by compound A of the present disclosure.

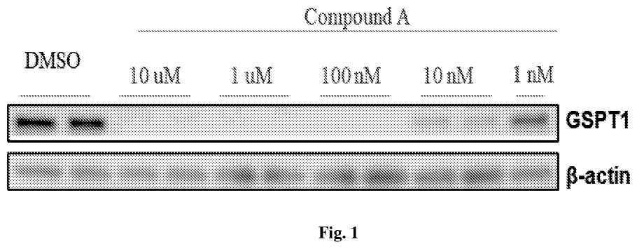

FIG. 2 shows a Western blot of the degradation of GSPT1 in HL-60 cells by compounds A and 8 of the present disclosure.

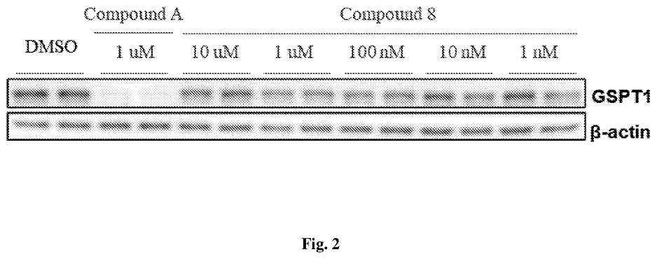

FIG. 3 shows a Western blot of the degradation of GSPT1 in HL-60 cells by compound 28 of the present disclosure.

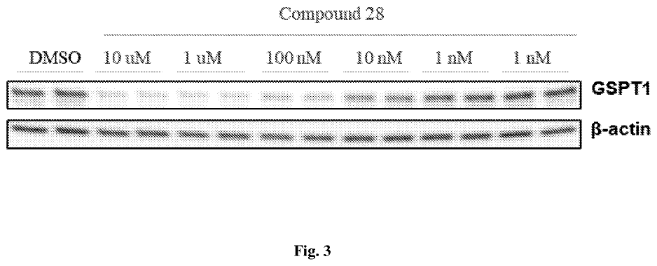

FIG. 4 shows a Western blot of the degradation of GSPT1 in HL-60 cells by compound F of the present disclosure.

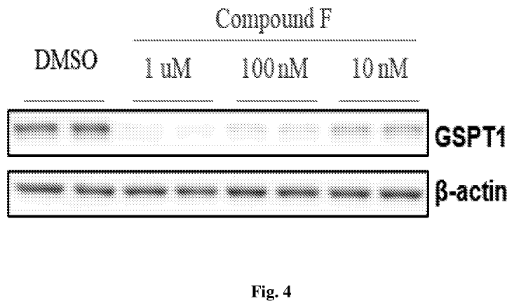

### DETAILED DESCRIPTION

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “GSPT1” or “GSPT1 protein” as used herein interchangeably, is also known as the translation termination factor eRF3. The G1 to S phase transition 1 (GSPT1) protein is a GTPase that interacts with eRF1 to promote stop codon recognition and release of nascent peptide from ribosome. It is involved in cell cycle regulation, cytoskeleton organization and apoptosis.

The term “degrader” as used herein, refers to a molecule agent that binds to a protein kinase, such as hematopoietic progenitor kinase 1 and subsequently lowers the steady state protein levels of the kinase. In some embodiments, a degrader as disclosed herein lowers steady state protein kinase levels by at least 10%, at least 20%, at least 30%, at least 40%, at least 50%, at least 60%, at least 65%, at least 70%, at least 75%, at least 80%, at least 85%, at least 90%, at least 95%, at least 96%, at least 97%, at least 98%, or at least 99%. In some embodiments, a degrader as disclosed herein lowers steady state protein kinase levels by at least 65%. In some embodiments, a degrader as disclosed herein lowers steady state protein kinase levels by at least 85%.

The term “compound,” when referring to a compound of the present disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of the present disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by the present disclosure are those that result in the formation of stable or chemically feasible compounds.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of the present disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the present disclosure. Unless otherwise stated, all tautomeric forms of the compounds of the present disclosure are within the scope of the present disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives disclosed herein. Thus, unless otherwise stated, when a reference is made to a “deuterated derivative” of a compound of the present disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 30 alkyl carbon atoms. In some embodiments, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched.

The term “cycloalkyl” refers to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentyl, and cyclohexyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄ hydrocarbon that is completely saturated, or is partially saturated as it contains one or more units of unsaturation but is not aromatic, wherein any individual ring in said bicyclic ring system has 3 to 7 members. Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl. Non-limiting examples of monocyclic carbocyclyls include cyclopropyl, cyclobutyl, cyclopentanyl, cyclohexyl, cyclopentenyl, cyclohexenyl, etc.

The term “alkylene” as used herein, refers to a divalent alkyl radical. Representative examples of C₁₋₁₀ alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched.

The term “alkynyl” as used herein, refers to an unsaturated straight or branched hydrocarbon having at least one carbon-carbon triple bond, such as a straight or branched group of 2 to 8 carbon atoms, referred to herein as C₂₋₈alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom. In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-membered heterocyclyl. In some embodiments, the heterocyclyl is a 4- to 10-memberedheterocyclyl. In some embodiments, the heterocyclyl is a 3- to 8-membered heterocyclyl. In some embodiments, the heterocyclyl is a 5- to 10-memberedheterocyclyl. In some embodiments, the heterocyclyl is a 5- to 8-membered heterocyclyl. In some embodiments, the heterocyclyl is a 5- or 6-memberedheterocyclyl. In some embodiments, the heterocyclyl is a 6-membered heterocyclyl. Non-limiting examples of monocyclic heterocyclyls include piperidinyl, piperazinyl, morpholinyl, tetrahydropyranyl, azetidinyl, oxetanyl, tetrahydrothiophenyl, dihyropyranyl, tetrahydropyridinyl, etc.

The term “heteroatom” means one or more of oxygen, sulfur, and nitrogen, including, any oxidized form of nitrogen or sulfur, or silicon; the quaternized form of any basic nitrogen or a substitutable nitrogen of a heterocyclic ring, for example, N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refer to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bicyclic heteroaryls include, for example, the following combinations of monocyclic rings: a monocyclic heteroaryl fused to another monocyclic heteroaryl; and a monocyclic heteroaryl fused to a phenyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

A “spirocyclic ring system” refers to a ring system having two or more cyclic rings, where every two rings share only one common atom.

The term “pro-drug group” refers to a group that is covalently attached to a compound and results in a compound with improved oral bioavailability and/or tumor targeting and/or that is more active in vivo. Certain compounds of Formula I may include a pro-drug group, as described in Hydrolysis in Drug and Prodrug Metabolism: Chemistry, Biochemistry, and Enzymology (see Testa, Bernard and Mayer, Joachim M. Wiley-VHCA, Zurich, Switzerland 2003). Pro-drugs of the compounds described herein are structurally modified forms of the compound that readily undergo chemical changes under physiological conditions to provide the active compound. Pro-drugs are often useful because, in some situations, they may be easier to administer than the parent drug. They may, for instance, be bioavailable by oral administration whereas the parent drug is not. A wide variety of pro-drug derivatives are known in the art, such as those that rely on hydrolytic cleavage or oxidative activation of the pro-drug. An example, without limitation, of a pro-drug group would be a portion of a compound such as an ester, but then is metabolically hydrolyzed to the carboxylic acid to release the active entity. Additional examples of pro-drug groups include peptidyl derivatives of a compound.

Non-limiting examples of suitable solvents that may be used in the present disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in the present disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of the present disclosure. Suitable pharmaceutically acceptable salts are, for example, those disclosed in S. M. Berge, et al. J. Pharmaceutical Sciences, 1977, 66, pp. 1-19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. The present disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal, including but not limited to, a human.

The term “therapeutically effective amount” refers to that amount of a compound that produces the desired effect for which it is administered (e.g., improvement in symptoms of diseases, disorders, and conditions mediated by the degradation of GSPT1, lessening the severity of diseases, disorders, and conditions mediated by the degradation of GSPT1 or a symptom thereof, and/or reducing progression of diseases, disorders, and conditions mediated by the degradation of GSPT1 or a symptom thereof). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, lower risk of diseases, disorders, and conditions mediated by the degradation of GSPT1, and disease-related complications. Improvements in or lessening the severity of any of these symptoms can be readily assessed according to methods and techniques known in the art or subsequently developed.

The term “cancer” includes, but is not limited to, the following cancers: epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary′ thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.

Compounds and compositions of the application can be administered in therapeutically effective amounts in a combinational therapy with one or more therapeutic agents (pharmaceutical combinations) or modalities, e.g., conventional chemotherapeutic agents or any other anti-proliferative, anti-cancer, and/or non-drug therapies, etc. For example, additive or synergistic effects can occur with anti-proliferative or anti-cancer substances. Where the compounds of the application are administered in conjunction with other therapies, dosages of the co-administered compounds will of course vary depending on the type of co-drug employed, on the specific drug employed, on the condition being treated and so forth. Combination therapy includes the administration of the subject compounds in further combination with one or more other biologically active ingredients (such as, but not limited to, conventional chemotherapeutic agents, a kinase inhibitor, a second and different antineoplastic agent, and non-drug therapies (such as, but not limited to, surgery or radiation treatment). For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

### II. Compounds and Compositions

In a first embodiment, a compound of the present disclosure is a compound of the following structural formula I:

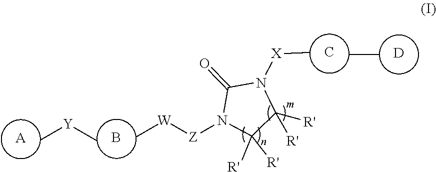

a tautomer thereof, a deuterated derivative of the compound or the tautomer, or a pharmaceutically acceptable salt the foregoing, wherein:
(i) each R′ is independently chosen from hydrogen, halogen groups, linear, branched, and cyclic alkyl groups;
(ii) m and n are independently chosen from 0, 1, and 2;
(iii) X and Z are independently absent or is chosen from linear, branched, cyclic alkylene groups, linear, branched, and cyclic heteroalkylene groups, and linear, branched, cyclic alkyl groups;
(iv) Y and W are independently absent or chosen from —O—, —C(O)—, —C(O)R^{x}—, —C(S)—, —C(S)R^{x}—, [C(R^{x}R^{y})]ₚ—, —S—, —S(O)₂—, —S(O)₂R^{x}—, NR^{x}—, and —NR^{x}C(O)—; further wherein p is chosen from 1, 2, 3, 4, 5, and 6; and R^{x} is chosen from hydrogen, linear, branched, and cyclic alkyl groups, carbocyclic groups, heterocyclic groups, aryl groups, and heteroaryl groups;
(v) ring A is chosen from

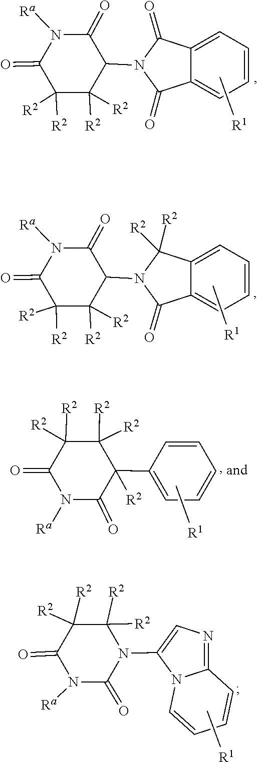

wherein R^{a} is chosen from hydrogen, linear, branched, an cyclic alkyl groups, an pro-drug groups; each R¹ and each R² are independently chosen from hydrogen, halogen groups, OR^{z}, and linear, branched, and cyclic alkyl groups; further wherein R^{z} is chosen from hydrogen, linear, branched, and cyclic alkyl groups, carbocyclic groups, heterocyclic groups, aryl groups, and heteroaryl groups;
(vi) ring B is absent or is chosen from optionally substituted cycloalkyl groups and heterocycloalkyl groups;
(vii) ring C is absent or is chosen from optionally substituted aryl groups and heteroaryl groups,
(viii) ring D is absent or is chosen from optionally substituted cycloalkyl groups, heterocycloalkyl groups, and heteroaryl groups;
   wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—C(O)N(C₁-C₆)₂ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
—S(O)₂C₁-C₆ linear, branched, and cyclic alkyl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

In a second embodiment, in a compound, tautomer, deuterated derivative, or pharmaceutically acceptable salt of the present disclosure, X is absent; and all other variables not specifically defined herein are as defined in the first embodiment.

In a third embodiment, in a compound, tautomer, deuterated derivative, or pharmaceutically acceptable salt of the present disclosure, X is a linear alkylene group; and all other variables not specifically defined herein are as defined in the first embodiment.

In a fourth embodiment, in a compound, tautomer, deuterated derivative, or pharmaceutically acceptable salt of the present disclosure, X is a methylene group; and all other variables not specifically defined herein are as defined in the first embodiment.

In a fifth embodiment, in a compound, tautomer, deuterated derivative, or pharmaceutically acceptable salt of the present disclosure, X is an ethylene group; and all other variables not specifically defined herein are as defined in the first embodiment.

In a sixth embodiment, in a compound, tautomer, deuterated derivative, or pharmaceutically acceptable salt of the present disclosure, Z is absent; and all other variables not specifically defined herein are as defined in the first embodiment.

In a seventh embodiment, in a compound, tautomer, deuterated derivative, or pharmaceutically acceptable salt of the present disclosure, Z is a linear alkylene group; and all other variables not specifically defined herein are as defined in the first embodiment.

In an eighth embodiment, in a compound, tautomer, deuterated derivative, or pharmaceutically acceptable salt of the present disclosure, Z is a methylene group; and all other variables not specifically defined herein are as defined in the first embodiment.

In a ninth embodiment, in a compound, tautomer, deuterated derivative, or pharmaceutically acceptable salt of the present disclosure, Z is an ethylene group; and all other variables not specifically defined herein are as defined in the first embodiment.

In a tenth embodiment, in a compound, tautomer, deuterated derivative, or pharmaceutically acceptable salt of the present disclosure, ring B is chosen from heterocycloalkyl groups; and all other variables not specifically defined herein are as defined in the first embodiment.

In a eleventh embodiment, in a compound, tautomer, deuterated derivative, or pharmaceutically acceptable salt of the present disclosure, ring B is chosen from

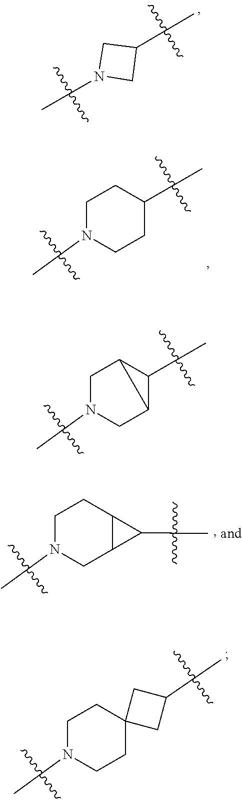 [SMILES: *C1CN(*)C1 ; *C1CCN(*)CC1 ; *C1C2CN(*)CC12 ; *C1C2CCN(*)CC12 ; *C1CC2(CCN(*)CC2)C1]

and; and all other variables not specifically defined herein are as defined in the first embodiment.

In a twelfth embodiment, in a compound, tautomer, deuterated derivative, or pharmaceutically acceptable salt of the present disclosure, ring C is an optionally substituted aryl group; and all other variables not specifically defined herein are as defined in the first embodiment.

In a thirteenth embodiment, in a compound, tautomer, deuterated derivative, or pharmaceutically acceptable salt of the present disclosure, ring C is phenyl; and all other variables not specifically defined herein are as defined in the first embodiment.

In a fourteenth embodiment, in a compound, tautomer, deuterated derivative, or pharmaceutically acceptable salt of the present disclosure, ring C is phenyl substituted with a halo group; and all other variables not specifically defined herein are as defined in the first embodiment.

In a fifteenth embodiment, in a compound, tautomer, deuterated derivative, or pharmaceutically acceptable salt of the present disclosure, ring C is phenyl substituted with a fluorine; and all other variables not specifically defined herein are as defined in the first embodiment.

In a sixteenth embodiment, in a compound, tautomer, deuterated derivative, or pharmaceutically acceptable salt of the present disclosure, ring C is phenyl substituted with a chlorine; and all other variables not specifically defined herein are as defined in the first embodiment.

In a seventeenth embodiment, in a compound, tautomer, deuterated derivative, or pharmaceutically acceptable salt of the present disclosure, ring C is phenyl substituted with a bromine; and all other variables not specifically defined herein are as defined in the first embodiment.

In an eighteenth embodiment, in a compound, tautomer, deuterated derivative, or pharmaceutically acceptable salt of the present disclosure, ring C is phenyl substituted with an alkyl group; and all other variables not specifically defined herein are as defined in the first embodiment.

In a nineteenth embodiment, in a compound, tautomer, deuterated derivative, or pharmaceutically acceptable salt of the present disclosure, ring C is phenyl substituted with a cycloalkyl group; and all other variables not specifically defined herein are as defined in the first embodiment.

In a twentieth embodiment, in a compound, tautomer, deuterated derivative, or pharmaceutically acceptable salt of the present disclosure, ring C is phenyl substituted with a cyclopropyl group; and all other variables not specifically defined herein are as defined in the first embodiment.

In a twenty-first embodiment, in a compound, tautomer, deuterated derivative, or pharmaceutically acceptable salt of the present disclosure, ring C is an optionally substituted heteroaryl group; and all other variables not specifically defined herein are as defined in the first embodiment.

In a twenty-second embodiment, in a compound, tautomer, deuterated derivative, or pharmaceutically acceptable salt of the present disclosure, ring C is pyridinyl; and all other variables not specifically defined herein are as defined in the first embodiment.

In a twenty-third embodiment, in a compound, tautomer, deuterated derivative, or pharmaceutically acceptable salt of the present disclosure, ring C is pyridinyl substituted with a halo group; and all other variables not specifically defined herein are as defined in the first embodiment.

In a twenty-fourth embodiment, in a compound, tautomer, deuterated derivative, or pharmaceutically acceptable salt of the present disclosure, ring C is pyridinyl substituted with a fluorine; and all other variables not specifically defined herein are as defined in the first embodiment.

In a twenty-fifth embodiment, in a compound, tautomer, deuterated derivative, or pharmaceutically acceptable salt of the present disclosure, ring C is pyridinyl substituted with a chlorine; and all other variables not specifically defined herein are as defined in the first embodiment.

In a twenty-sixth embodiment, in a compound, tautomer, deuterated derivative, or pharmaceutically acceptable salt of the present disclosure, ring C is pyridinyl substituted with a bromine; and all other variables not specifically defined herein are as defined in the first embodiment.

In a twenty-seventh embodiment, in a compound, tautomer, deuterated derivative, or pharmaceutically acceptable salt of the present disclosure, ring C is quinolyl.

In a twenty-eighth embodiment, in a compound, tautomer, deuterated derivative, or pharmaceutically acceptable salt of the present disclosure, ring D is an optionally substituted heteroaryl group; and all other variables not specifically defined herein are as defined in the first embodiment.

In a twenty-ninth embodiment, in a compound, tautomer, deuterated derivative, or pharmaceutically acceptable salt of the present disclosure, ring D is pyridinyl; and all other variables not specifically defined herein are as defined in the first embodiment.

In a thirtieth embodiment, in a compound, tautomer, deuterated derivative, or pharmaceutically acceptable salt of the present disclosure, ring D is pyridinyl substituted with a halo group; and all other variables not specifically defined herein are as defined in the first embodiment.

In a thirty-first embodiment, in a compound, tautomer, deuterated derivative, or pharmaceutically acceptable salt of the present disclosure, ring D is pyridinyl substituted with a fluorine; and all other variables not specifically defined herein are as defined in the first embodiment.

In a thirty-second embodiment, in a compound, tautomer, deuterated derivative, or pharmaceutically acceptable salt of the present disclosure, ring D is pyridinyl substituted with a chlorine; and all other variables not specifically defined herein are as defined in the first embodiment.

In a thirty-third embodiment, in a compound, tautomer, deuterated derivative, or pharmaceutically acceptable salt of the present disclosure, ring D is pyridinyl substituted with a bromine; and all other variables not specifically defined herein are as defined in the first embodiment.

In a thirty-fourth embodiment, in a compound, tautomer, deuterated derivative, or pharmaceutically acceptable salt of the present disclosure, ring D is thiazolyl; and all other variables not specifically defined herein are as defined in the first embodiment.

In a thirty-fifth embodiment, in a compound, tautomer, deuterated derivative, or pharmaceutically acceptable salt of the present disclosure, ring D is thiazolyl substituted with an alkyl group; and all other variables not specifically defined herein are as defined in the first embodiment.

In a thirty-sixth embodiment, in a compound, tautomer, deuterated derivative, or pharmaceutically acceptable salt of the present disclosure, ring D is thiazolyl substituted with a methyl; and all other variables not specifically defined herein are as defined in the first embodiment.

In a thirty-seventh embodiment, in a compound, tautomer, deuterated derivative, or pharmaceutically acceptable salt of the present disclosure, if ring C and ring D are absent, X is a linear alkyl group; and all other variables not specifically defined herein are as defined in the first embodiment.

In a thirty-eighth embodiment, in a compound, tautomer, deuterated derivative, or pharmaceutically acceptable salt of the present disclosure, X is a methyl group; and all other variables not specifically defined herein are as defined in the first embodiment.

In a thirty-ninth embodiment, in a compound, tautomer, deuterated derivative, or pharmaceutically acceptable salt of the present disclosure, if ring C and ring D are absent, X is a branched alkyl group; and all other variables not specifically defined herein are as defined in the first embodiment.

In a fortieth embodiment, in a compound, tautomer, deuterated derivative, or pharmaceutically acceptable salt of the present disclosure, X is a tert-butyl group.

In a forty-first embodiment, in a compound, tautomer, deuterated derivative, or pharmaceutically acceptable salt of the present disclosure, if ring C and ring D are absent, X is a cyclic alkyl group; and all other variables not specifically defined herein are as defined in the first embodiment.

In a forty-second embodiment, in a compound, tautomer, deuterated derivative, or pharmaceutically acceptable salt of the present disclosure, X is a cyclohexyl group; and all other variables not specifically defined herein are as defined in the first embodiment.

In a forty-third embodiment, in a compound, tautomer, deuterated derivative, or pharmaceutically acceptable salt of the present disclosure, m is 1 and n is 1; and all other variables not specifically defined herein are as defined in the first embodiment.

In a forty-fourth embodiment, in a compound, tautomer, deuterated derivative, or pharmaceutically acceptable salt of the present disclosure, each R′ is hydrogen; and all other variables not specifically defined herein are as defined in the first embodiment.

In a forty-fifth embodiment, in a compound, tautomer, deuterated derivative, or pharmaceutically acceptable salt of the present disclosure, m is 2 and n is 1; and all other variables not specifically defined herein are as defined in the first embodiment.

In a forty-sixth embodiment, in a compound, tautomer, deuterated derivative, or pharmaceutically acceptable salt of the present disclosure, each R′ is hydrogen; and all other variables not specifically defined herein are as defined in the first embodiment.

In a forty-seventh embodiment, in a compound, tautomer, deuterated derivative, or pharmaceutically acceptable salt of the present disclosure, ring A is

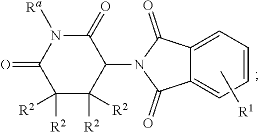

and all other variables not specifically defined herein are as defined in the first embodiment.

In a forty-eighth embodiment, in a compound, tautomer, deuterated derivative, or pharmaceutically acceptable salt of the present disclosure, R^{a} is chosen from hydrogen, linear, branched, and cyclic alkyl groups, and pro-drug groups; and all other variables not specifically defined herein are as defined in the first embodiment.

In a forty-ninth embodiment, in a compound, tautomer, deuterated derivative, or pharmaceutically acceptable salt of the present disclosure, ring A is

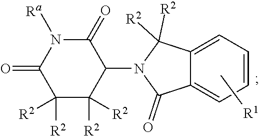

and all other variables not specifically defined herein are as defined in the first embodiment.

In a fiftieth embodiment, in a compound, tautomer, deuterated derivative, or pharmaceutically acceptable salt of the present disclosure, R^{a} is chosen from hydrogen, linear, branched, and cyclic alkyl groups, and pro-drug groups; and all other variables not specifically defined herein are as defined in the first embodiment.

In a fifty-first embodiment, in a compound, tautomer, deuterated derivative, or pharmaceutically acceptable salt of the present disclosure, ring A is

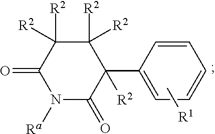

and all other variables not specifically defined herein are as defined in the first embodiment.

In a fifty-second embodiment, in a compound, tautomer, deuterated derivative, or pharmaceutically acceptable salt of the present disclosure, R^{a} is chosen from hydrogen, linear, branched, and cyclic alkyl groups, and pro-drug groups; and all other variables not specifically defined herein are as defined in the first embodiment.

In a fifty-third embodiment, in a compound, tautomer, deuterated derivative, or pharmaceutically acceptable salt of the present disclosure, ring A is

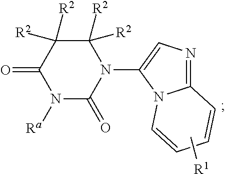

and all other variables not specifically defined herein are as defined in the first embodiment.

In a fifty-fourth embodiment, in a compound, tautomer, deuterated derivative, or pharmaceutically acceptable salt of the present disclosure, R^{a} is chosen from hydrogen, linear, branched, and cyclic alkyl groups, and pro-drug groups; and all other variables not specifically defined herein are as defined in the first embodiment.

In certain embodiments, at least one compound of the present disclosure is selected from Compounds 1 to 35 shown in Table 1 below, a tautomer thereof, a deuterated derivative of the compound or the tautomer, or a pharmaceutically acceptable salt of the foregoing.

TABLE 1

| | |
|---|---|
| 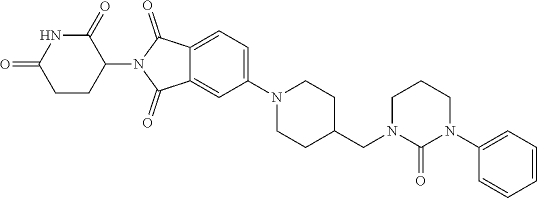 [SMILES: O=C1CCC(N2C(=O)c3ccc(N4CCC(CN5CCCN(c6ccccc6)C5=O)CC4)cc3C2=O)C(=O)N1] | 1 |
| 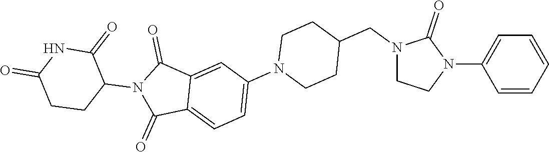 [SMILES: O=C1CCC(N2C(=O)c3ccc(N4CCC(CN5CCN(c6ccccc6)C5=O)CC4)cc3C2=O)C(=O)N1] | 2 |
| 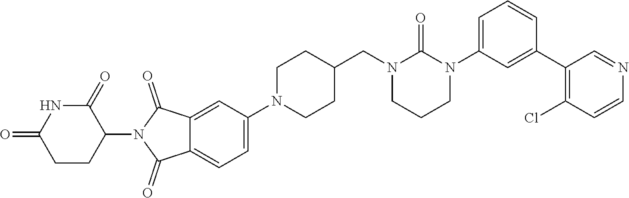 [SMILES: O=C1CCC(N2C(=O)c3ccc(N4CCC(CN5CCCN(c6cccc(-c7cnccc7Cl)c6)C5=O)CC4)cc3C2=O)C(=O)N1] | 3 |
| 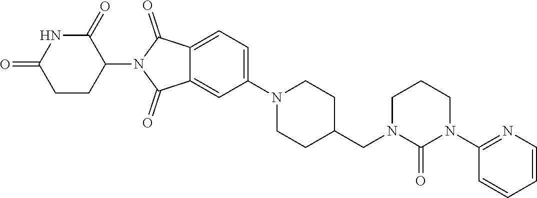 [SMILES: O=C1CCC(N2C(=O)c3ccc(N4CCC(CN5CCCN(c6ccccn6)C5=O)CC4)cc3C2=O)C(=O)N1] | 4 |
| 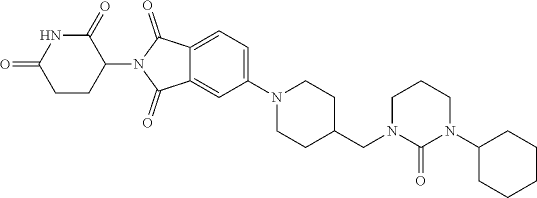 [SMILES: O=C1CCC(N2C(=O)c3ccc(N4CCC(CN5CCCN(C6CCCCC6)C5=O)CC4)cc3C2=O)C(=O)N1] | 5 |
| 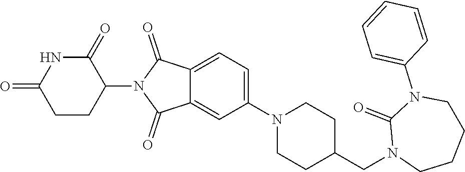 [SMILES: O=C1CCC(N2C(=O)c3ccc(N4CCC(CN5CCCCN(c6ccccc6)C5=O)CC4)cc3C2=O)C(=O)N1] | 6 |
| 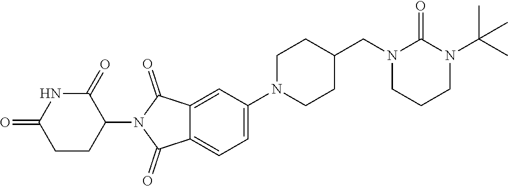 [SMILES: CC(C)(C)N1CCCN(CC2CCN(c3ccc4c(c3)C(=O)N(C3CCC(=O)NC3=O)C4=O)CC2)C1=O] | 7 |
| 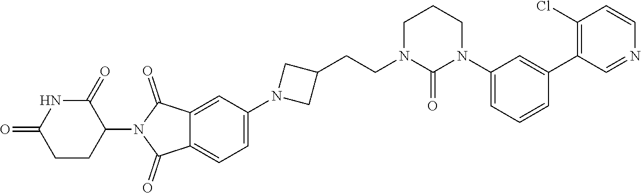 [SMILES: O=C1CCC(N2C(=O)c3ccc(N4CC(CCN5CCCN(c6cccc(-c7cnccc7Cl)c6)C5=O)C4)cc3C2=O)C(=O)N1] | 8 |
| 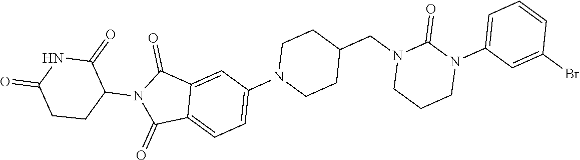 [SMILES: O=C1CCC(N2C(=O)c3ccc(N4CCC(CN5CCCN(c6cccc(Br)c6)C5=O)CC4)cc3C2=O)C(=O)N1] | 9 |
| 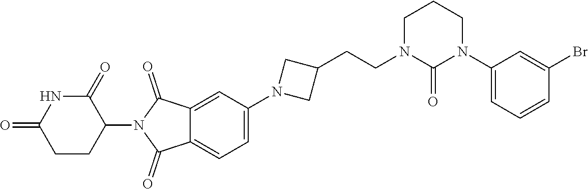 [SMILES: O=C1CCC(N2C(=O)c3ccc(N4CC(CCN5CCCN(c6cccc(Br)c6)C5=O)C4)cc3C2=O)C(=O)N1] | 10 |
| 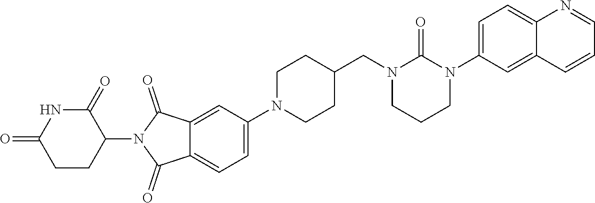 [SMILES: O=C1CCC(N2C(=O)c3ccc(N4CCC(CN5CCCN(c6ccc7ncccc7c6)C5=O)CC4)cc3C2=O)C(=O)N1] | 11 |
| 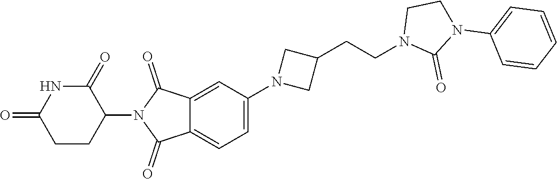 [SMILES: O=C1CCC(N2C(=O)c3ccc(N4CC(CCN5CCN(c6ccccc6)C5=O)C4)cc3C2=O)C(=O)N1] | 12 |
| 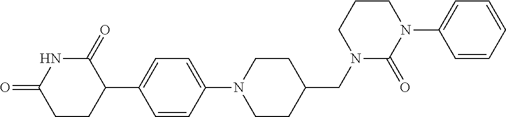 [SMILES: O=C1CCC(c2ccc(N3CCC(CN4CCCN(c5ccccc5)C4=O)CC3)cc2)C(=O)N1] | 13 |
| 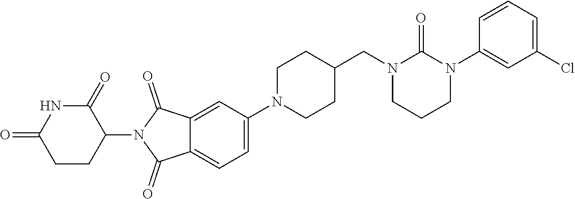 [SMILES: O=C1CCC(N2C(=O)c3ccc(N4CCC(CN5CCCN(c6cccc(Cl)c6)C5=O)CC4)cc3C2=O)C(=O)N1] | 14 |
| 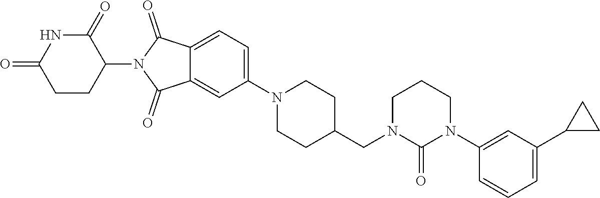 [SMILES: O=C1CCC(N2C(=O)c3ccc(N4CCC(CN5CCCN(c6cccc(C7CC7)c6)C5=O)CC4)cc3C2=O)C(=O)N1] | 15 |
| 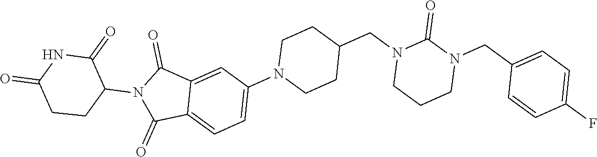 [SMILES: O=C1CCC(N2C(=O)c3ccc(N4CCC(CN5CCCN(Cc6ccc(F)cc6)C5=O)CC4)cc3C2=O)C(=O)N1] | 16 |
| 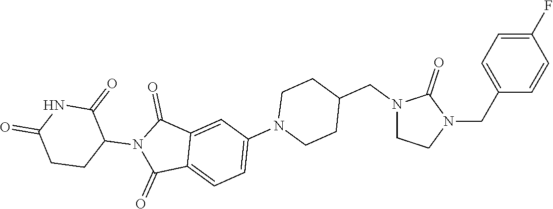 [SMILES: O=C1CCC(N2C(=O)c3ccc(N4CCC(CN5CCN(Cc6ccc(F)cc6)C5=O)CC4)cc3C2=O)C(=O)N1] | 17 |
| 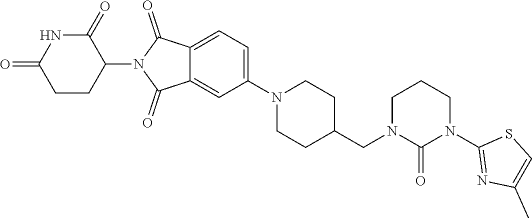 [SMILES: Cc1csc(N2CCCN(CC3CCN(c4ccc5c(c4)C(=O)N(C4CCC(=O)NC4=O)C5=O)CC3)C2=O)n1] | 18 |
| 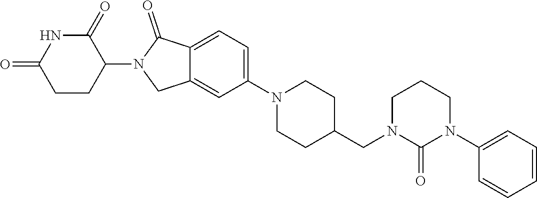 [SMILES: O=C1CCC(N2Cc3cc(N4CCC(CN5CCCN(c6ccccc6)C5=O)CC4)ccc3C2=O)C(=O)N1] | 19 |
| 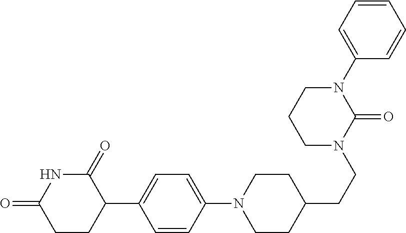 [SMILES: O=C1CCC(c2ccc(N3CCC(CCN4CCCN(c5ccccc5)C4=O)CC3)cc2)C(=O)N1] | 20 |
| 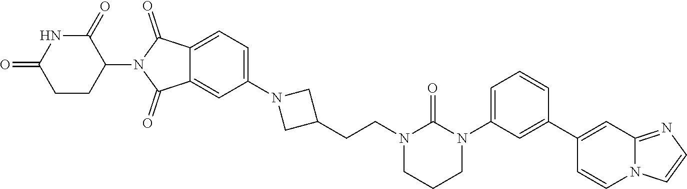 [SMILES: O=C1CCC(N2C(=O)c3ccc(N4CC(CCN5CCCN(c6cccc(-c7ccn8ccnc8c7)c6)C5=O)C4)cc3C2=O)C(=O)N1] | 21 |
| 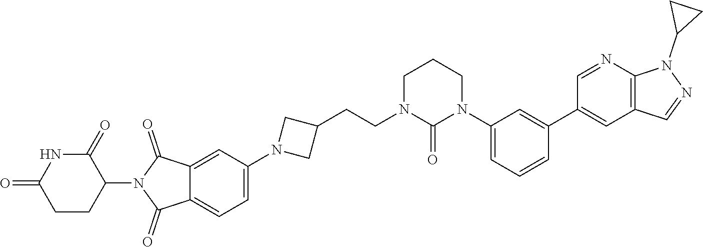 [SMILES: O=C1CCC(N2C(=O)c3ccc(N4CC(CCN5CCCN(c6cccc(-c7cnc8c(cnn8C8CC8)c7)c6)C5=O)C4)cc3C2=O)C(=O)N1] | 22 |
| 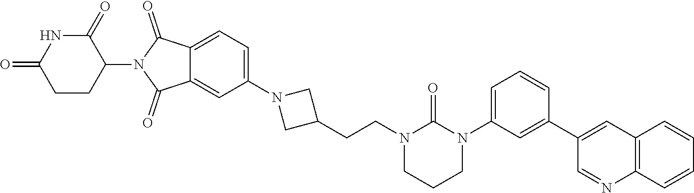 [SMILES: O=C1CCC(N2C(=O)c3ccc(N4CC(CCN5CCCN(c6cccc(-c7cnc8ccccc8c7)c6)C5=O)C4)cc3C2=O)C(=O)N1] | 23 |
| 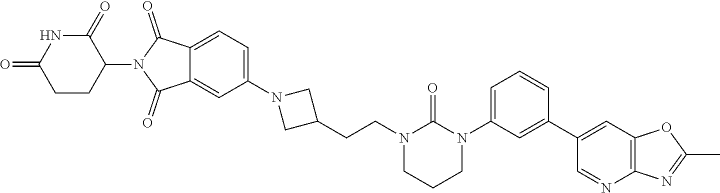 [SMILES: Cc1nc2ncc(-c3cccc(N4CCCN(CCC5CN(c6ccc7c(c6)C(=O)N(C6CCC(=O)NC6=O)C7=O)C5)C4=O)c3)cc2o1] | 24 |
| 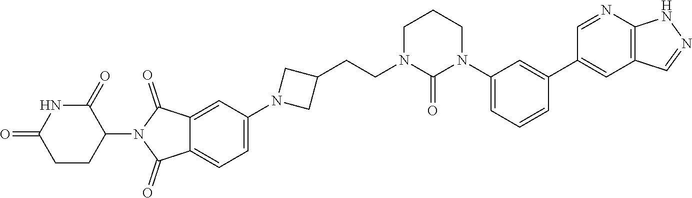 [SMILES: O=C1CCC(N2C(=O)c3ccc(N4CC(CCN5CCCN(c6cccc(-c7cnc8[nH]ncc8c7)c6)C5=O)C4)cc3C2=O)C(=O)N1] | 25 |
| 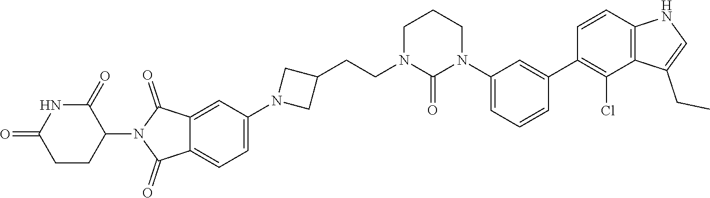 [SMILES: CCc1c[nH]c2ccc(-c3cccc(N4CCCN(CCC5CN(c6ccc7c(c6)C(=O)N(C6CCC(=O)NC6=O)C7=O)C5)C4=O)c3)c(Cl)c12] | 26 |
| 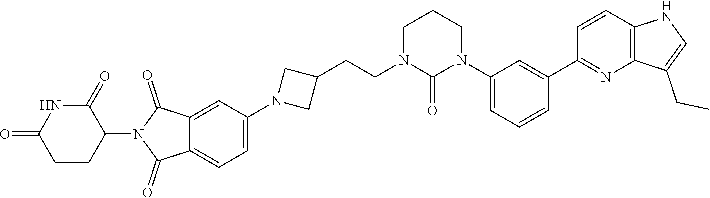 [SMILES: CCc1c[nH]c2ccc(-c3cccc(N4CCCN(CCC5CN(c6ccc7c(c6)C(=O)N(C6CCC(=O)NC6=O)C7=O)C5)C4=O)c3)nc12] | 27 |
| 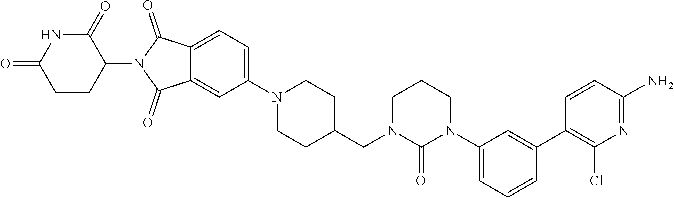 [SMILES: Nc1ccc(-c2cccc(N3CCCN(CC4CCN(c5ccc6c(c5)C(=O)N(C5CCC(=O)NC5=O)C6=O)CC4)C3=O)c2)c(Cl)n1] | 28 |
| 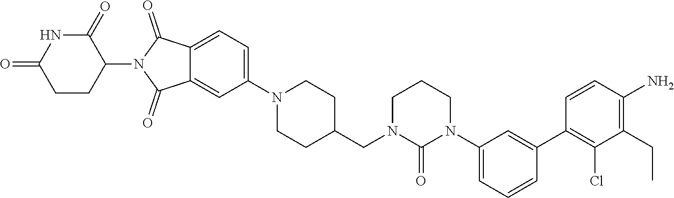 [SMILES: CCc1c(N)ccc(-c2cccc(N3CCCN(CC4CCN(c5ccc6c(c5)C(=O)N(C5CCC(=O)NC5=O)C6=O)CC4)C3=O)c2)c1Cl] | 29 |
| 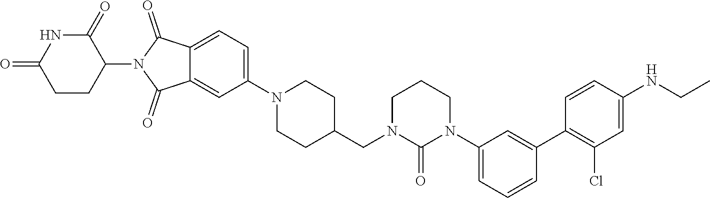 [SMILES: CCNc1ccc(-c2cccc(N3CCCN(CC4CCN(c5ccc6c(c5)C(=O)N(C5CCC(=O)NC5=O)C6=O)CC4)C3=O)c2)c(Cl)c1] | 30 |
| 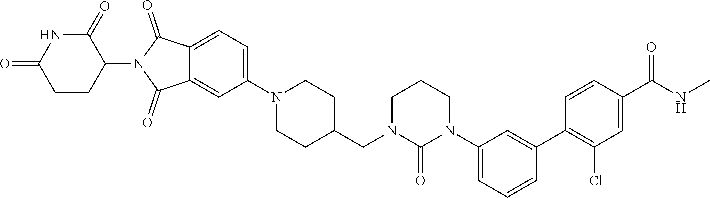 [SMILES: CNC(=O)c1ccc(-c2cccc(N3CCCN(CC4CCN(c5ccc6c(c5)C(=O)N(C5CCC(=O)NC5=O)C6=O)CC4)C3=O)c2)c(Cl)c1] | 31 |
| 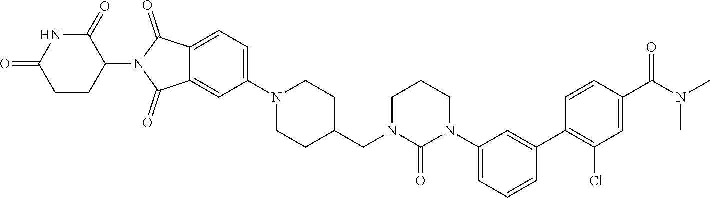 [SMILES: CN(C)C(=O)c1ccc(-c2cccc(N3CCCN(CC4CCN(c5ccc6c(c5)C(=O)N(C5CCC(=O)NC5=O)C6=O)CC4)C3=O)c2)c(Cl)c1] | 32 |
| 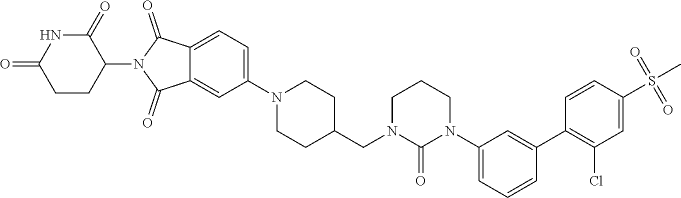 [SMILES: CS(=O)(=O)c1ccc(-c2cccc(N3CCCN(CC4CCN(c5ccc6c(c5)C(=O)N(C5CCC(=O)NC5=O)C6=O)CC4)C3=O)c2)c(Cl)c1] | 33 |
| 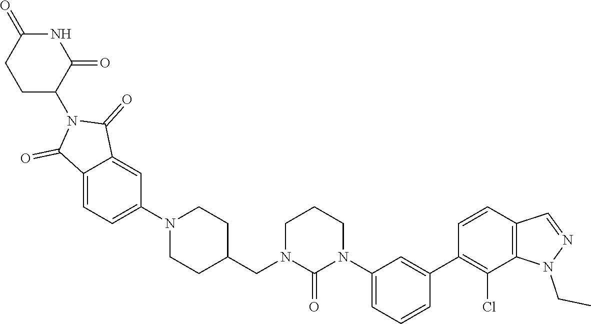 [SMILES: CCn1ncc2ccc(-c3cccc(N4CCCN(CC5CCN(c6ccc7c(c6)C(=O)N(C6CCC(=O)NC6=O)C7=O)CC5)C4=O)c3)c(Cl)c21] | 34 |
| 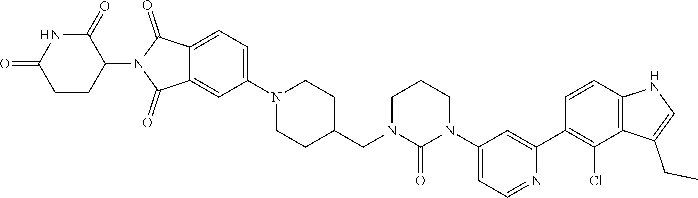 [SMILES: CCc1c[nH]c2ccc(-c3cc(N4CCCN(CC5CCN(c6ccc7c(c6)C(=O)N(C6CCC(=O)NC6=O)C7=O)CC5)C4=O)ccn3)c(Cl)c12] | 35 |

Another aspect of the present disclosure provides pharmaceutical compositions comprising at least one compound selected from a compound of Formula I, Compounds 1 to 35, a tautomer thereof, a deuterated derivative of the compound or the tautomer, or a pharmaceutically acceptable salt of the foregoing, or a pharmaceutical composition comprising any of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of the present disclosure can be employed in combination therapies; that is, the pharmaceutical compositions disclosed herein can further include an additional active pharmaceutical agent. Alternatively, a pharmaceutical composition comprising a compound selected from a compound of Formula I, Compounds 1 to 35, a tautomer thereof, a deuterated derivative of the compound or the tautomer, or a pharmaceutically acceptable salt of the foregoing, or a pharmaceutical composition comprising any of the foregoing can be administered as a separate composition concurrently with, prior to, or subsequent to, a composition comprising an additional active pharmaceutical agent.

As discussed above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of the present disclosure, such as by producing any undesirable biological effect or otherwise interacting in a deleterious manner with any other component(s) of the pharmaceutical composition, its use is contemplated to be within the scope of the present disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### III. Methods of Treatment and Uses

In another aspect of the present disclosure, a compound, tautomer, deuterated derivative, or pharmaceutically acceptable salt as disclosed herein, including a compound of Formula I, Compounds 1 to 35, a tautomer thereof, a deuterated derivative of the compound or the tautomer, Compounds A to F, or a pharmaceutically acceptable salt of the foregoing, or the pharmaceutical composition thereof, is for use in treating a disease, a disorder, or a condition mediated by the degradation of the GSPT1 protein. In another aspect, disclosed herein is use of the compound, tautomer, deuterated derivative, and/or the pharmaceutically acceptable salt thereof as disclosed herein, including a compound of Formula I, Compounds 1 to 24, a tautomer thereof, a deuterated derivative of the compound or the tautomer, and/or a pharmaceutically acceptable salt of the foregoing, Compounds A to F, or the pharmaceutical composition thereof, for the manufacture of a medicament for treating a disease, a disorder, or a condition mediated by the degradation of the GSPT1 protein. In yet another aspect, disclosed herein is a method of treating a disease, a disorder, or a condition mediated by the degradation of the GSPT1 protein in a subject, comprising administering a therapeutically effective amount of a compound, tautomer, deuterated derivative, and/or pharmaceutically acceptable salt as disclosed herein, including a compound of Formula I, Compounds 1 to 35, a tautomer thereof, a deuterated derivative of the compound or the tautomer, and/or a pharmaceutically acceptable salt of the foregoing, Compounds A to F, or the pharmaceutical composition thereof.

In some embodiments, the disease, the disorder, or the condition is cancer. In some embodiments, the cancer is a solid tumor. In some embodiments, the solid tumor is chosen from brain cancer, breast cancer, gastric cancer, renal cancer, prostate cancer, testis cancer, colorectal cancer, lung cancer, bladder cancer, urothelial cancer, cervical cancer, head and neck cancer, esophageal and gastric cancer, osteosarcoma, cervical cancer, endometrial cancer, ovarian cancer, squamous cell cancer, peritoneal cancer, neuroendocrine cancer, hepatocellular carcinoma, pancreatic cancer, genitourinary tract cancer, larynx cancer, skin cancer, nervous system cancer, thyroid cancer, and rhabdosarcoma. In some embodiments, the cancer is a hematologic cancer. In some embodiments, the hematologic cancer is chosen from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, and non-Hodgkin lymphoma.

In some embodiments, the cancer is chosen from cancers of epidermoid oral such as buccal cavity, lip, tongue, mouth, pharynx; cardiac cancers such as sarcoma (angiosarcoma, fibrosarcoma, rhabdomyosarcoma, liposarcoma), myxoma, rhabdomyoma, fibroma, lipoma, and teratoma; lung cancers such as bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatosis hamartoma, mesothelioma; gastrointestinal cancers such as esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Kaposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; genitourinary tract cancers including kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver cancers such as hepatoma (hepatocellular carcinoma), cholangiocarcinoma, hepatoblastoma, angiosarcoma, hepatocellular adenoma, hemangioma, biliary passages; bone cancers such as osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; cancers of the nervous system, including skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); gynecological cancers including uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosathecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; hematologic cancers such as blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma) hairy cell; lymphoid disorders; skin cancers including malignant melanoma, basal cell carcinoma, squamous cell carcinoma, Kaposi's sarcoma, keratoacanthoma, moles dysplastic nevi, lipoma, angioma, dermatofibroma, keloids, psoriasis; cancers of the thyroid gland such as papillary thyroid carcinoma, follicular thyroid carcinoma; medullary′ thyroid carcinoma, undifferentiated thyroid cancer, multiple endocrine neoplasia type 2A, multiple endocrine neoplasia type 2B, familial medullary thyroid cancer, pheochromocytoma, paraganglioma; and cancers of the adrenal glands like neuroblastoma.

In another aspect of the present disclosure, a compound, tautomer, deuterated derivative, and/or pharmaceutically acceptable salt as disclosed herein, including a compound of Formula I, Compounds 1 to 24, a tautomer thereof, a deuterated derivative of the compound or the tautomer, and/or a pharmaceutically acceptable salt of the foregoing, Compounds A to F, or the pharmaceutical composition thereof, is for use in decreasing GSPT1 activity. In another aspect, disclosed herein is use of a compound, tautomer, deuterated derivative, and/or pharmaceutically acceptable salt as disclosed herein, including a compound of Formula I, Compounds 1 to 24, a tautomer thereof, a deuterated derivative of the compound or the tautomer, and/or a pharmaceutically acceptable salt of the foregoing, Compounds A to F, or the pharmaceutical composition thereof, for the manufacture of a medicament for decreasing protein kinase activity. In yet another aspect, disclosed herein is a method of decreasing GSPT1 activity, comprising administering a therapeutically effective amount of a compound, tautomer, deuterated derivative, and/or pharmaceutically acceptable salt as disclosed herein to a subject, including a compound of Formula I, Compounds 1 to 35, a tautomer thereof, a deuterated derivative of the compound or the tautomer, and/or a pharmaceutically acceptable salt of the foregoing, Compounds A to F, or the pharmaceutical composition thereof. In yet another aspect, disclosed herein is a method of decreasing GSPT1 activity, comprising contacting said protein kinase with a compound, tautomer, deuterated derivative, and/or pharmaceutically acceptable salt as disclosed herein to a subject, including a compound of Formula I, Compounds 1 to 24, a tautomer thereof, a deuterated derivative of the compound or the tautomer, and/or a pharmaceutically acceptable salt of the foregoing, Compounds A to F, or the pharmaceutical composition thereof.

A compound of Formula I, Compounds 1 to 35, a tautomer thereof, a deuterated derivative of the compound or the tautomer, Compounds A to F, and/or a pharmaceutically acceptable salt of the foregoing, or the pharmaceutical composition thereof may be administered once daily, twice daily, or three times daily, for example, for the treatment of a disease, a disorder, or a condition mediated by the degradation of GSPT1.

In some embodiments, 2 mg to 1500 mg or 5 mg to 1000 mg of a compound of Formula I, Compounds 1 to 35, a tautomer thereof, a deuterated derivative of the compound or the tautomer, and/or a pharmaceutically acceptable salt of the foregoing, Compounds A to F, or the pharmaceutical composition thereof are administered once daily, twice daily, or three times daily.

A compound of Formula I, Compounds 1 to 35, a tautomer thereof, a deuterated derivative of the compound or the tautomer, and/or a pharmaceutically acceptable salt of the foregoing, Compounds A to F, or the pharmaceutical composition thereof may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes, for example, intravenous, intraperitoneal, subcutaneous, intramuscular, transepithelial, nasal, intrapulmonary, intrathecal, rectal and topical modes of administration. Parenteral administration can, for example, be by continuous infusion over a selected period of time. Other forms of administration contemplated in the present disclosure are as described in International Patent Application Nos. WO 2013/075083, WO 2013/075084, WO 2013/078320, WO 2013/120104, WO 2014/124418, WO 2014/151142, and WO 2015/023915.

Useful dosages or a therapeutically effective amount of a compound or pharmaceutically acceptable salt thereof as disclosed herein can be determined by comparing their in vitro activity and in vivo activity in animal models. Methods for the extrapolation of effective dosages in mice and other animals, to humans are known to the art; for example, see U.S. Pat. No. 4,938,949.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, tautomers, pharmaceutically acceptable salts, and deuterated derivatives disclosed herein are based upon the free base form of the reference compound. For example, “1000 mg of at least one compound chosen from compounds of Formula I and pharmaceutically acceptable salts thereof” includes 1000 mg of compound of Formula I and a concentration of a pharmaceutically acceptable salt of compounds of Formula I equivalent to 1000 mg of compounds of Formula I.

In another aspect of the present disclosure, the compounds and the compositions disclosed herein can be administered in therapeutically effective amounts in a combinational therapy with one or more therapeutic agents (pharmaceutical combinations) or modalities, e.g., anti-proliferative, anti-cancer, immunomodulatory or anti-inflammatory agent, and/or non-drug therapies, etc. For example, synergistic effects can occur with anti-proliferative, anti-cancer, immunomodulatory or anti-inflammatory substances. Where the compounds disclosed herein are administered in conjunction with other therapies, dosages of the co-administered compounds will of course vary depending on the type of co-drug employed, on the specific drug employed, on the condition being treated and so forth. Combination therapy includes the administration of the subject compounds in further combination with one or more other biologically active ingredients (such as a second kinase inhibitor, a second and different antineoplastic agent, and non-drug therapies (such as surgery or radiation treatment). For instance, the compounds disclosed herein can be used in combination with other pharmaceutically active compounds, preferably compounds that are able to enhance the effect of the compounds disclosed herein. The compounds disclosed herein can be administered simultaneously (as a single preparation or separate preparation) or sequentially to the other drug therapy or treatment modality. In general, a combination therapy envisions administration of two or more drugs during a single cycle or course of therapy. In another aspect of the disclosure, the compounds may be administered in combination with one or more separate pharmaceutical agents, e.g., a chemotherapeutic agent, an immunotherapeutic agent, or an adjunctive therapeutic agent. In an embodiment, the separate pharmaceutical agent is selected from an anti-PD1 antibody (e.g. pembrolizumab), an HDAC inhibitor r (e.g. panobinostat, romidepsin, vorinostat, or citarinostat), a BCL-2 inhibitor (e.g. venetoclax), a BTK inhibitor (e.g. ibrutinib or acalabrutinib), an mTOR inhibitor (e.g. everolimus), a PI3K inhibitor r (e.g. idelalisib), a PKCβinhibitor (e.g. enzastaurin), a SYK inhibitor (e.g. fostamatinib), a JAK2 inhibitor (e.g. fedratinib, pacritinib, ruxolitinib, baricitinib, gandotinib, lestaurtinib, or momelotinib), an Aurora kinase inhibitor (e.g. alisertib), an EZF12 inhibitor (e.g. tazemetostat, GSK126, CPI-1205, 3-deazaneplanocin A, EPZ005687, Ell, UNC1999, or sinefungin), a BET inhibitor (e.g. birabresib), a hypomethylating agent (e.g. 5-azacytidine or decitabine), a DOTIL inhibitor (e.g. pinometostat), a FIAT inhibitor (e.g. C646), a WDR5 inhibitor (e.g. OICR-9429), a DNMT1 inhibitor (e.g. GSK3484862), an LSD-1 inhibitor (e.g. Compound C or seclidemstat), a G9A inhibitor (e.g. UNC0631), a PRMT5 inhibitor (e.g. GSK3326595), a BRD inhibitor (e.g. LP99), a SUV420FU/F12 inhibitor (e.g. A-196), a CARMI inhibitor (e.g. EZM2302), a PLKI inhibitor (e.g. BI2536), an NEK2 inhibitor (e.g. JF1295), an MEK inhibitor (e.g. trametinib, binimetinib, cobimetinib, selumetinib), a PF1F19 inhibitor, a PIM inhibitor (e.g. LGF1-447), an IGF-IR inhibitor (e.g. linsitinib), an XPO1 inhibitor (e.g. selinexor), a BIRC5 inhibitor (e.g. YM1 55), a PARP inhibitor (e.g. Olaparib), an EGFR inhibitor (e.g. Osimertinib), a HER2/NEU inhibitor (i.e. tucatinib), an SRC inhibitor (i.e. dasatinib), an AKT inhibitor (i.e. Ipatasertib), platinum, or a chemotherapy (e.g, bendamustine, bleomycin, doxorubicin, etoposide, methotrexate, cytarabine, vincristine, ifosfamide, melphalan, oxaliplatin, cisplatin, taxanes or dexamethasone).

### Non-Limiting Exemplary Embodiments

1. A compound of Formula (I):

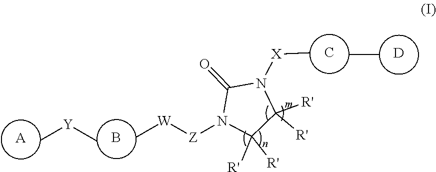

a tautomer thereof, a deuterated derivative of the compound or the tautomer, or a pharmaceutically acceptable salt of the foregoing, wherein:
   (ix) each R′ is independently chosen from hydrogen, halogen groups, linear, branched, and cyclic alkyl groups;
   (x) m and n are independently chosen from 0, 1, and 2;
   (xi) X and Z are independently absent or is chosen from linear, branched, cyclic alkylene groups, linear, branched, and cyclic heteroalkylene groups, and linear, branched, cyclic alkyl groups;
   (xii) Y and W are independently absent or chosen from —O—, —C(O)—, —C(O)R^{x}—, —C(S)—, —C(S)R^{x}—, —[C(R^{x}R^{y})]ₚ—, —S—, —S(O)₂—, —S(O)₂R^{x}—, NR^{x}—, and —NR^{x}C(O)—; further wherein p is chosen from 1, 2, 3, 4, 5, and 6; and R^{x} is chosen from hydrogen, linear, branched, and cyclic alkyl groups, carbocyclic groups, heterocyclic groups, aryl groups, and heteroaryl groups;
   (xiii) ring A is chosen from

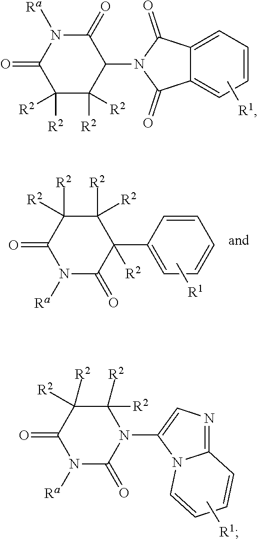

wherein R^{a} is chosen from hydrogen, linear, branched, and cyclic alkyl groups, and pro-drug groups; each R¹ and each R² are independently chosen from hydrogen, halogen groups, OR^{z}, and linear, branched, and cyclic alkyl groups; further wherein R^{z} is chosen from hydrogen, linear, branched, and cyclic alkyl groups, carbocyclic groups, heterocyclic groups, aryl groups, and heteroaryl groups;
(xiv) ring B is absent or is chosen from optionally substituted cycloalkyl groups and heterocycloalkyl groups;
(xv) ring C is absent or is chosen from optionally substituted aryl groups and heteroaryl groups,
(xvi) ring D is absent or is chosen from optionally substituted cycloalkyl groups, heterocycloalkyl groups, and heteroaryl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
   halogen groups,
   hydroxy,
   thiol,
   amino,
   cyano,
   —OC(O)C₁-C₆ linear, branched, and cyclic alkyl groups,
   —C(O)OC₁-C₆ linear, branched, and cyclic alkyl groups,
   —NHC₁-C₆ linear, branched, and cyclic alkyl groups,
   —N(C₁-C₆ linear, branched, and cyclic alkyl groups)₂,
   —NHC(O)C₁-C₆ linear, branched, and cyclic alkyl groups,
   —C(O)NHC₁-C₆ linear, branched, and cyclic alkyl groups,
   —C(O)N(C₁-C₆)₂ linear, branched, and cyclic alkyl groups,
   —NHaryl groups,
   —N(aryl groups)₂,
   —NHC(O)aryl groups,
   —C(O)NHaryl groups,
   —NHheteroaryl groups,
   —N(heteroaryl groups)₂,
   —NHC(O)heteroaryl groups,
   —C(O)NHheteroaryl groups,
   —S(O)₂C₁-C₆ linear, branched, and cyclic alkyl groups,
   C₁-C₆ linear, branched, and cyclic alkyl groups,
   C₂-C₆ linear, branched, and cyclic alkenyl groups,
   C₁-C₆ linear, branched, and cyclic hydroxyalkyl groups,
   C₁-C₆ linear, branched, and cyclic aminoalkyl groups,
   C₁-C₆ linear, branched, and cyclic alkoxy groups,
   C₁-C₆ linear, branched, and cyclic thioalkyl groups,
   C₁-C₆ linear, branched, and cyclic haloalkyl groups,
   C₁-C₆ linear, branched, and cyclic haloaminoalkyl groups,
   C₁-C₆ linear, branched, and cyclic halothioalkyl groups,
   C₁-C₆ linear, branched, and cyclic haloalkoxy groups,
   benzyloxy, benzylamino, and benzylthio groups,
   3 to 6-membered heterocycloalkenyl groups,
   3 to 6-membered heterocyclic groups, and
   5 and 6-membered heteroaryl groups.
2. The compound, tautomer, deuterated derivative, or pharmaceutically acceptable salt of embodiment 1, wherein X is absent.
3. The compound, tautomer, deuterated derivative, or pharmaceutically acceptable salt of embodiment 1, wherein X is a linear alkylene group.
4. The compound, tautomer, deuterated derivative, or pharmaceutically acceptable salt of embodiment 3, wherein X is a methylene group.
5. The compound, tautomer, deuterated derivative, or pharmaceutically acceptable salt of embodiment 3, wherein X is an ethylene group.
6. The compound, tautomer, deuterated derivative, or pharmaceutically acceptable salt of any of embodiments 1-5, wherein Z is absent.
7. The compound, tautomer, deuterated derivative, or pharmaceutically acceptable salt of any of embodiments 1-5, wherein Z is a linear alkylene group.
8. The compound, tautomer, deuterated derivative, or pharmaceutically acceptable salt of embodiment 7, wherein Z is a methylene group.
9. The compound, tautomer, deuterated derivative, or pharmaceutically acceptable salt of embodiment 7, wherein Z is an ethylene group.
10. The compound, tautomer, deuterated derivative, or pharmaceutically acceptable salt of any of embodiments 1-9, wherein ring B is chosen from heterocycloalkyl groups.
11. The compound, tautomer, deuterated derivative, or pharmaceutically acceptable salt of embodiment 10, wherein ring B is chosen from

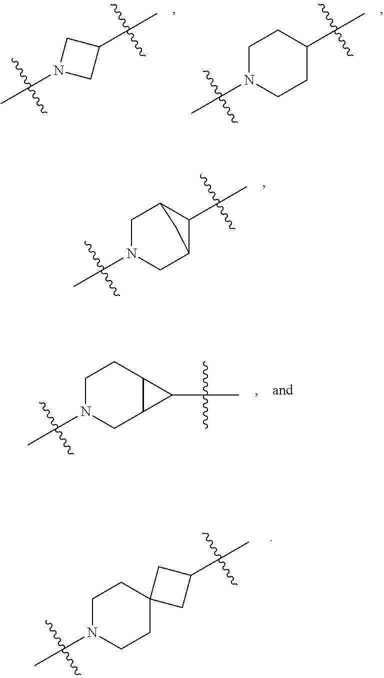 [SMILES: *C1CN(*)C1 ; *C1CCN(*)CC1 ; *C1C2CC1CN(*)C2 ; *C1C2CCN(*)CC12 ; *C1CC2(CCN(*)CC2)C1]

12. The compound, tautomer, deuterated derivative, or pharmaceutically acceptable salt of any of embodiments 1-11, wherein ring C is an optionally substituted aryl group.
13. The compound, tautomer, deuterated derivative, or pharmaceutically acceptable salt of embodiment 12, wherein ring C is phenyl.
14. The compound, tautomer, deuterated derivative, or pharmaceutically acceptable salt of embodiment 12, wherein ring C is phenyl substituted with a halo group.
15. The compound, tautomer, deuterated derivative, or pharmaceutically acceptable salt of embodiment 14, wherein ring C is phenyl substituted with a fluorine.
16. The compound, tautomer, deuterated derivative, or pharmaceutically acceptable salt of embodiment 14, wherein ring C is phenyl substituted with a chlorine.
17. The compound, tautomer, deuterated derivative, or pharmaceutically acceptable salt of embodiment 14, wherein ring C is phenyl substituted with a bromine.
18. The compound, tautomer, deuterated derivative, or pharmaceutically acceptable salt of embodiment 12, wherein ring C is phenyl substituted with an alkyl group.
19. The compound, tautomer, deuterated derivative, or pharmaceutically acceptable salt of embodiment 12, wherein ring C is phenyl substituted with a cycloalkyl group.
20. The compound, tautomer, deuterated derivative, or pharmaceutically acceptable salt of embodiment 19, wherein ring C is phenyl substituted with a cyclopropyl group.
21. The compound, tautomer, deuterated derivative, or pharmaceutically acceptable salt of any of embodiments 1-11, wherein ring C is an optionally substituted heteroaryl group.
22. The compound, tautomer, deuterated derivative, or pharmaceutically acceptable salt of embodiment 21, wherein ring C is pyridinyl.
23. The compound, tautomer, deuterated derivative, or pharmaceutically acceptable salt of embodiment 21, wherein ring C is pyridinyl substituted with a halo group.
24. The compound, tautomer, deuterated derivative, or pharmaceutically acceptable salt of embodiment 23, wherein ring C is pyridinyl substituted with a fluorine.
25. The compound, tautomer, deuterated derivative, or pharmaceutically acceptable salt of embodiment 23, wherein ring C is pyridinyl substituted with a chlorine.
26. The compound, tautomer, deuterated derivative, or pharmaceutically acceptable salt of embodiment 23, wherein ring C is pyridinyl substituted with a bromine.
27. The compound, tautomer, deuterated derivative, or pharmaceutically acceptable salt of embodiment 21, wherein ring C is quinolyl.
28. The compound, tautomer, deuterated derivative, or pharmaceutically acceptable salt of any of embodiments 1-27, wherein ring D is an optionally substituted heteroaryl group.
29. The compound, tautomer, deuterated derivative, or pharmaceutically acceptable salt of embodiment 28, wherein ring D is pyridinyl.
30. The compound, tautomer, deuterated derivative, or pharmaceutically acceptable salt of embodiment 28, wherein ring D is pyridinyl substituted with a halo group.
31. The compound, tautomer, deuterated derivative, or pharmaceutically acceptable salt of embodiment 30, wherein ring D is pyridinyl substituted with a fluorine.
32. The compound, tautomer, deuterated derivative, or pharmaceutically acceptable salt of embodiment 30, wherein ring D is pyridinyl substituted with a chlorine.
33. The compound, tautomer, deuterated derivative, or pharmaceutically acceptable salt of embodiment 30, wherein ring D is pyridinyl substituted with a bromine.
34. The compound, tautomer, deuterated derivative, or pharmaceutically acceptable salt of embodiment 28, wherein ring D is thiazolyl.
35. The compound, tautomer, deuterated derivative, or pharmaceutically acceptable salt of embodiment 28, wherein ring D is thiazolyl substituted with an alkyl group.
36. The compound, tautomer, deuterated derivative, or pharmaceutically acceptable salt of embodiment 30, wherein ring D is thiazolyl substituted with a methyl.
37. The compound, tautomer, deuterated derivative, or pharmaceutically acceptable salt of embodiment 1, wherein if ring C and ring D are absent, X is a linear alkyl group.
38. The compound, tautomer, deuterated derivative, or pharmaceutically acceptable salt of embodiment 37, wherein X is a methyl group.
39. The compound, tautomer, deuterated derivative, or pharmaceutically acceptable salt of embodiment 1, wherein if ring C and ring D are absent, X is a branched alkyl group.
40. The compound, tautomer, deuterated derivative, or pharmaceutically acceptable salt of embodiment 39, wherein X is a tert-butyl group.
41. The compound, tautomer, deuterated derivative, or pharmaceutically acceptable salt of embodiment 1, wherein if ring C and ring D are absent, X is a cyclic alkyl group.
42. The compound, tautomer, deuterated derivative, or pharmaceutically acceptable salt of embodiment 41, wherein X is a cyclohexyl group.
43. The compound, tautomer, deuterated derivative, or pharmaceutically acceptable salt of any of embodiments 1-42, wherein m is 1 and n is 1.
44. The compound, tautomer, deuterated derivative, or pharmaceutically acceptable salt of embodiment 43, wherein each R′ is hydrogen.
45. The compound, tautomer, deuterated derivative, or pharmaceutically acceptable salt of any of embodiments 1-42, wherein m is 2 and n is 1.
46. The compound, tautomer, deuterated derivative, or pharmaceutically acceptable salt of embodiment 45, wherein each R′ is hydrogen.
47. The compound, tautomer, deuterated derivative, or pharmaceutically acceptable salt of any of embodiments 1-46, wherein ring A is

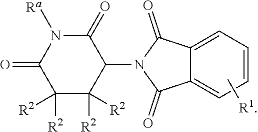

48. The compound, tautomer, deuterated derivative, or pharmaceutically acceptable salt of embodiment 47, wherein R^{a} is chosen from hydrogen, linear, branched, and cyclic alkyl groups, and pro-drug groups.
49. The compound, tautomer, deuterated derivative, or pharmaceutically acceptable salt of any of embodiments 1-46, wherein ring A is

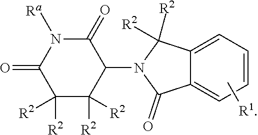

50. The compound, tautomer, deuterated derivative, or pharmaceutically acceptable salt of embodiment 49, wherein R^{a} is chosen from hydrogen, linear, branched, and cyclic alkyl groups, and pro-drug groups.
51. The compound, tautomer, deuterated derivative, or pharmaceutically acceptable salt of any of embodiments 1-46, wherein ring A is

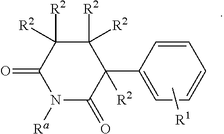

52. The compound, tautomer, deuterated derivative, or pharmaceutically acceptable salt of embodiment 51, wherein R^{a} is chosen from hydrogen, linear, branched, and cyclic alkyl groups, and pro-drug groups.
53. The compound, tautomer, deuterated derivative, or pharmaceutically acceptable salt of any of embodiments 1-46, wherein ring A is

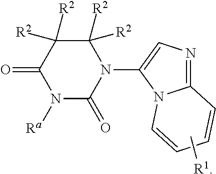

54. The compound, tautomer, deuterated derivative, or pharmaceutically acceptable salt of embodiment 53, wherein R^{a} is chosen from hydrogen, linear, branched, and cyclic alkyl groups, and pro-drug groups.
55. A compound chosen from

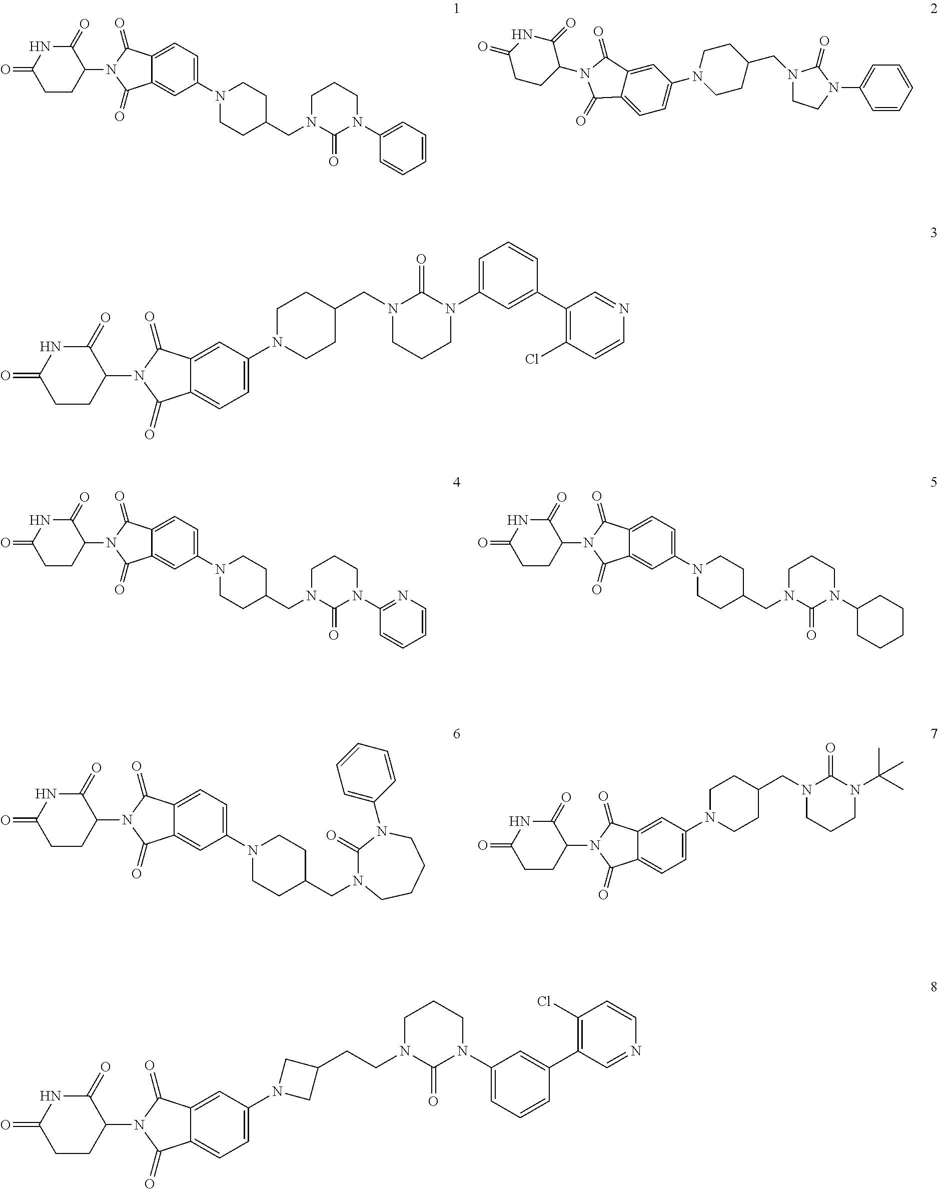

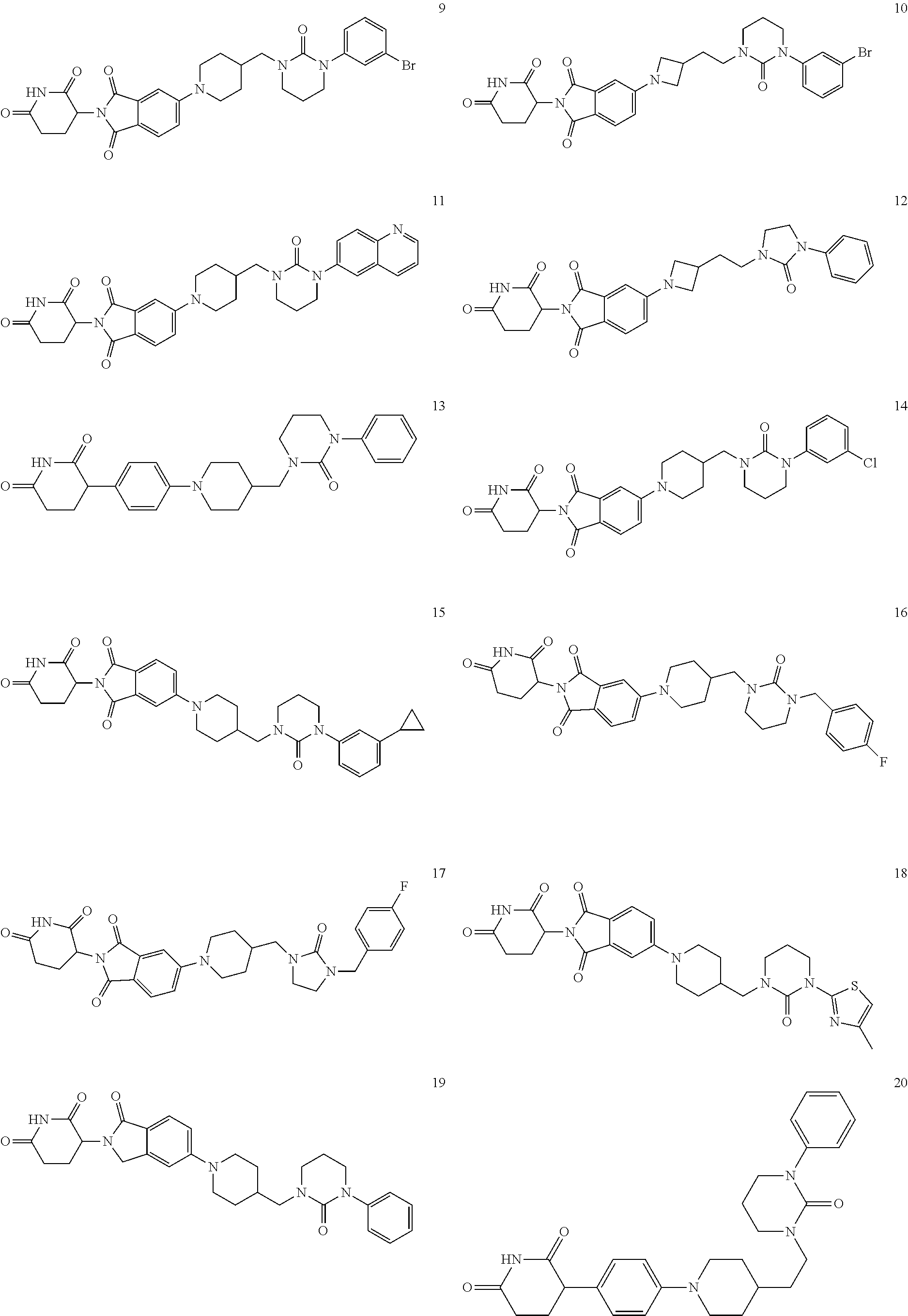

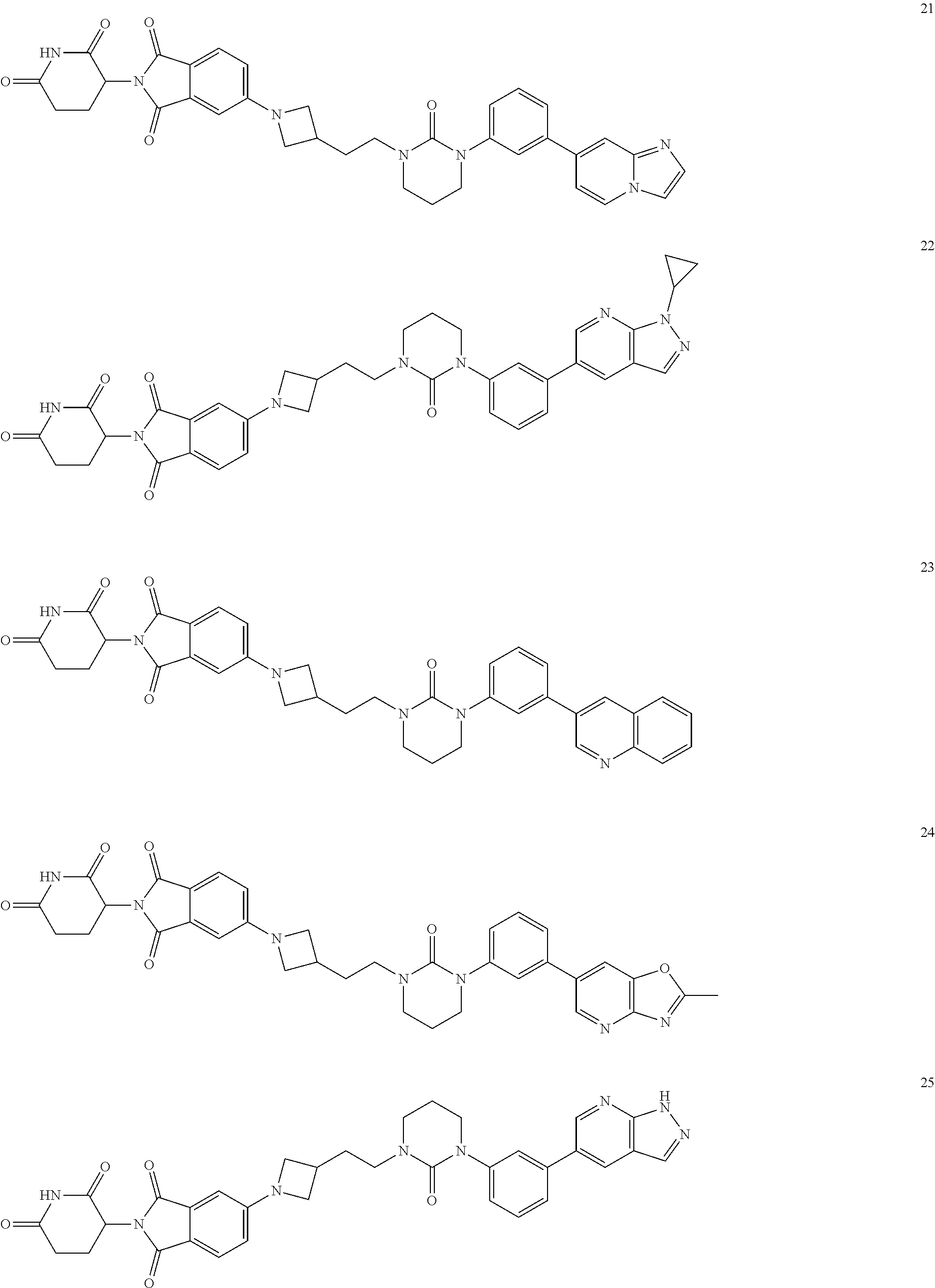 [SMILES: O=C1CCC(N2C(=O)c3ccc(N4CC(CCN5CCCN(c6cccc(-c7ccn8ccnc8c7)c6)C5=O)C4)cc3C2=O)C(=O)N1 ; O=C1CCC(N2C(=O)c3ccc(N4CC(CCN5CCCN(c6cccc(-c7cnc8c(cnn8C8CC8)c7)c6)C5=O)C4)cc3C2=O)C(=O)N1 ; O=C1CCC(N2C(=O)c3ccc(N4CC(CCN5CCCN(c6cccc(-c7cnc8ccccc8c7)c6)C5=O)C4)cc3C2=O)C(=O)N1 ; Cc1nc2ncc(-c3cccc(N4CCCN(CCC5CN(c6ccc7c(c6)C(=O)N(C6CCC(=O)NC6=O)C7=O)C5)C4=O)c3)cc2o1 ; O=C1CCC(N2C(=O)c3ccc(N4CC(CCN5CCCN(c6cccc(-c7cnc8[nH]ncc8c7)c6)C5=O)C4)cc3C2=O)C(=O)N1]

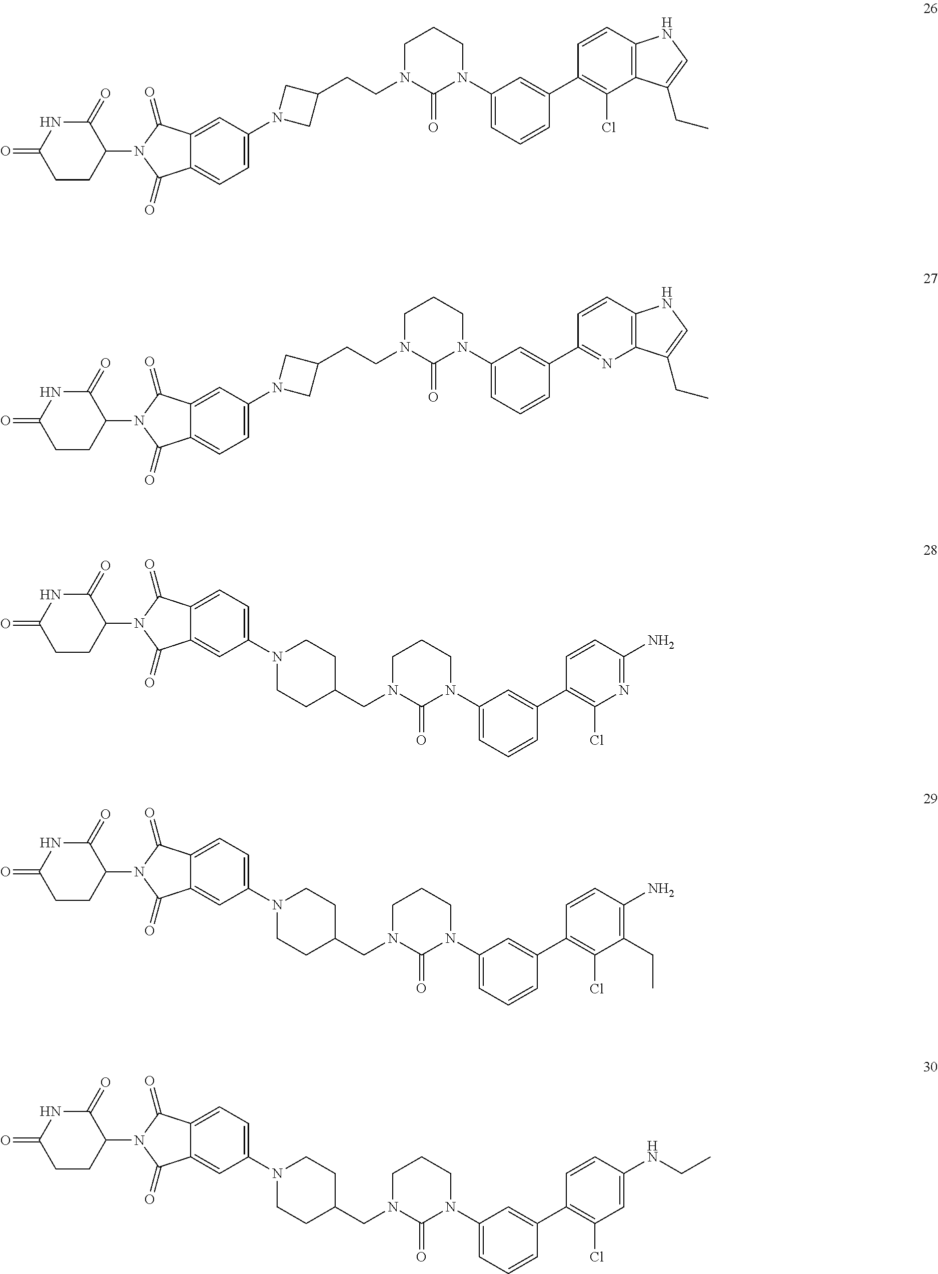 [SMILES: CCc1c[nH]c2ccc(-c3cccc(N4CCCN(CCC5CN(c6ccc7c(c6)C(=O)N(C6CCC(=O)NC6=O)C7=O)C5)C4=O)c3)c(Cl)c12 ; CCc1c[nH]c2ccc(-c3cccc(N4CCCN(CCC5CN(c6ccc7c(c6)C(=O)N(C6CCC(=O)NC6=O)C7=O)C5)C4=O)c3)nc12 ; Nc1ccc(-c2cccc(N3CCCN(CC4CCN(c5ccc6c(c5)C(=O)N(C5CCC(=O)NC5=O)C6=O)CC4)C3=O)c2)c(Cl)n1 ; CCc1c(N)ccc(-c2cccc(N3CCCN(CC4CCN(c5ccc6c(c5)C(=O)N(C5CCC(=O)NC5=O)C6=O)CC4)C3=O)c2)c1Cl ; CCNc1ccc(-c2cccc(N3CCCN(CC4CCN(c5ccc6c(c5)C(=O)N(C5CCC(=O)NC5=O)C6=O)CC4)C3=O)c2)c(Cl)c1]

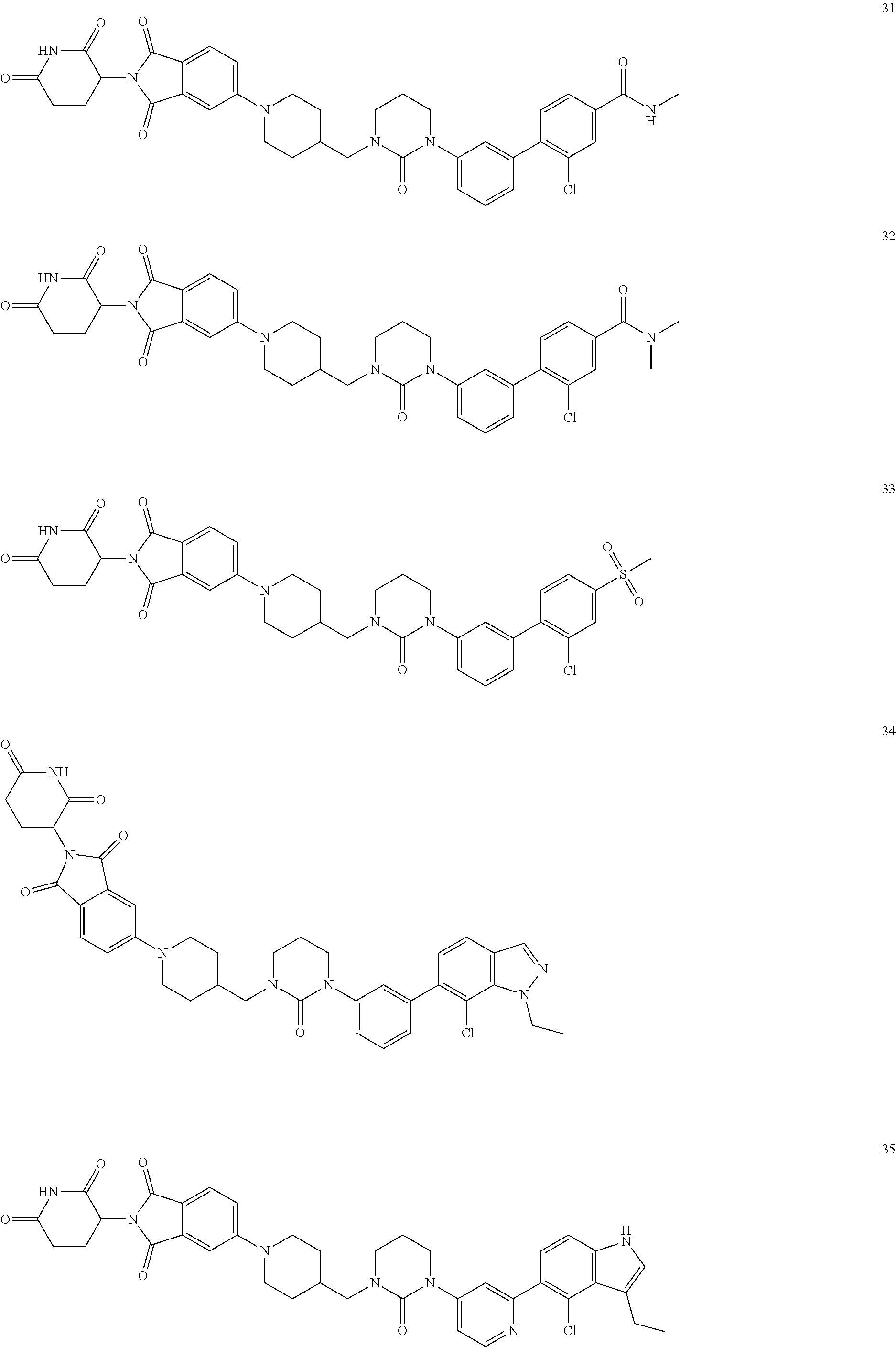 [SMILES: CNC(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CN(C)C(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CS(=O)(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CCn1ncc2ccc(-c3cccc(N4CCCN(CC5CCN(c6ccc7c(c6)C(=O)N(C6CCC(=O)NC6=O)C7=O)CC5)C4=O)c3)c(Cl)c21 ; CCc1c[nH]c2ccc(-c3cc(N4CCCN(CC5CCN(c6ccc7c(c6)C(=O)N(C6CCC(=O)NC6=O)C7=O)CC5)C4=O)ccn3)c(Cl)c12]

a tautomer thereof, a deuterate derivative of the compound or the tautomer, or a pharmaceutically acceptable salt of the foregoing.
56. A pharmaceutical composition comprising a compound, tautomer, deuterated derivative, and/or pharmaceutically acceptable salt according to any one of embodiments 1-53 and at least one pharmaceutically acceptable carrier.
57. A method for treating or alleviating a disease, a disorder or a condition mediated by the degradation of the GSPT1 protein, comprising administering to a subject in need thereof a therapeutically effective amount of a compound, tautomer, deuterated derivative, and/or pharmaceutically acceptable salt according to any one of the embodiments 1-53 or the pharmaceutical composition according to embodiment 54.
58. A method for decreasing GSPT1 protein activity in a disease, a disorder or a condition, comprising administering to a subject in need thereof a therapeutically effective amount of a compound, tautomer, deuterated derivative, and/or pharmaceutically acceptable salt according to any one of the embodiments 1-53 or the pharmaceutical composition according to embodiment 54.
59. A method for treating or alleviating a disease, a disorder or a condition mediated by the degradation of the GSPT1 protein, comprising administering to a subject in need thereof a therapeutically effective amount of a compound chosen from

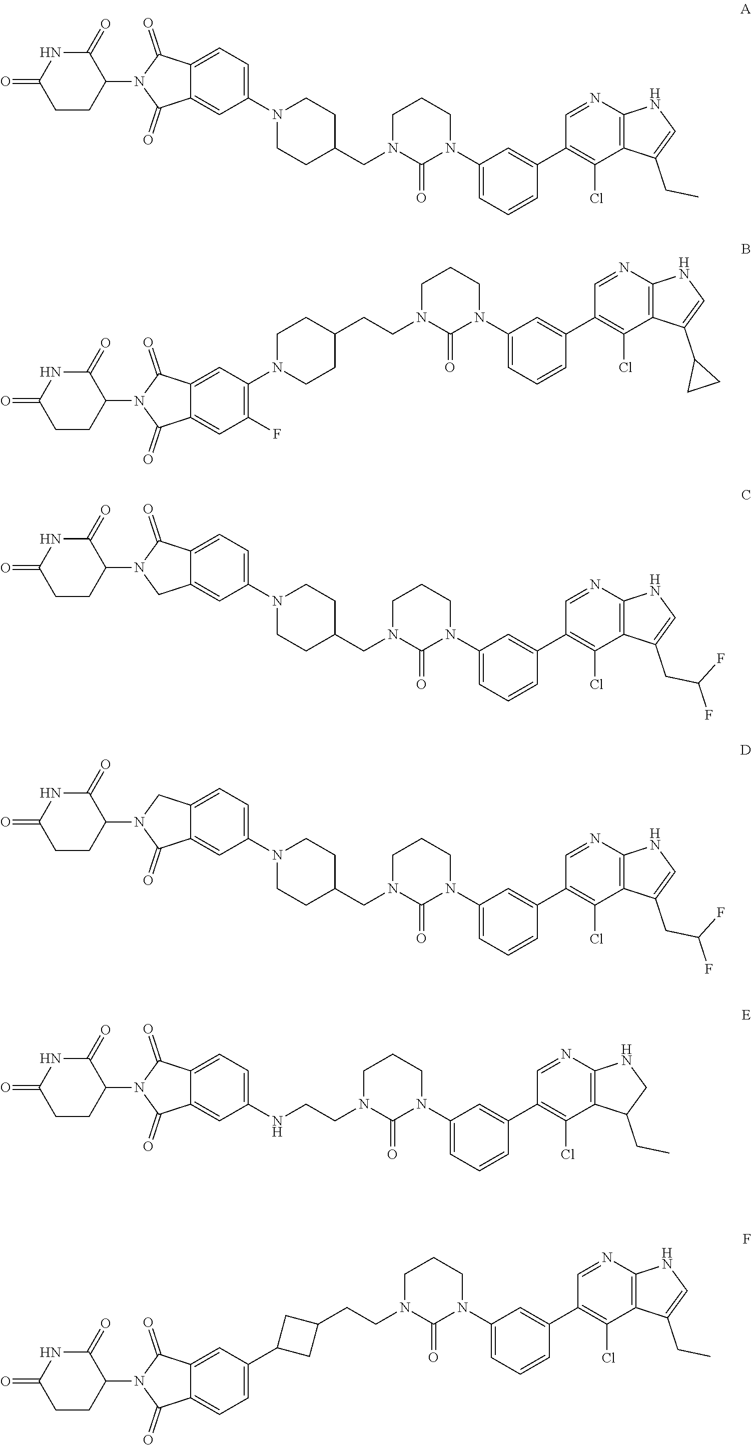

a tautomer thereof, a deuterated derivative of the compound or the tautomer, or a pharmaceutically acceptable salt of the foregoing.
60. A method for decreasing GSPT1 protein activity in a disease, a disorder or a condition, comprising administering to a subject in need thereof a therapeutically effective amount of a compound chosen from

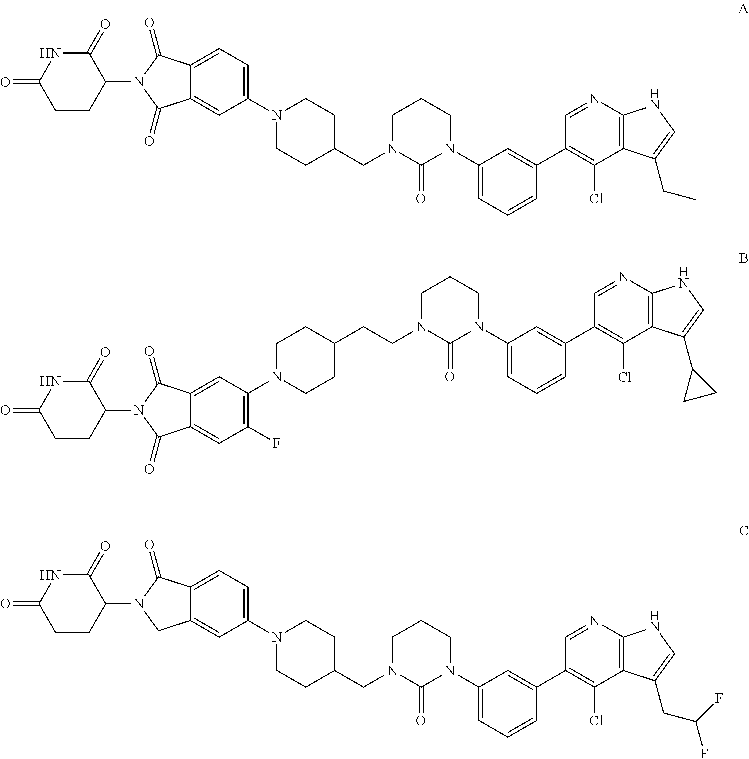 [SMILES: CCc1c[nH]c2ncc(-c3cccc(N4CCCN(CC5CCN(c6ccc7c(c6)C(=O)N(C6CCC(=O)NC6=O)C7=O)CC5)C4=O)c3)c(Cl)c12 ; O=C1CCC(N2C(=O)c3cc(F)c(N4CCC(CCN5CCCN(c6cccc(-c7cnc8[nH]cc(C9CC9)c8c7Cl)c6)C5=O)CC4)cc3C2=O)C(=O)N1 ; O=C1CCC(N2Cc3cc(N4CCC(CN5CCCN(c6cccc(-c7cnc8[nH]cc(CC(F)F)c8c7Cl)c6)C5=O)CC4)ccc3C2=O)C(=O)N1]

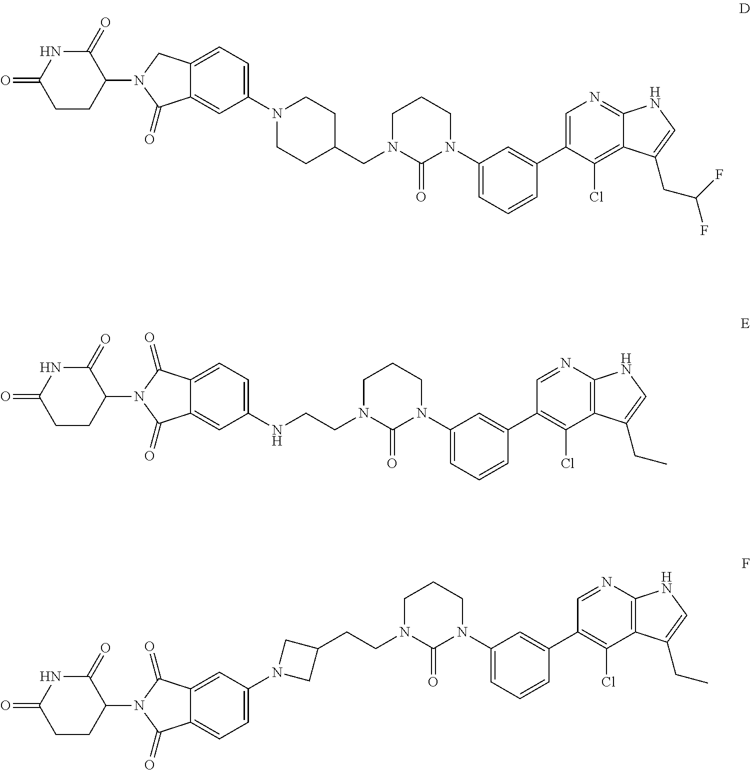 [SMILES: O=C1CCC(N2Cc3ccc(N4CCC(CN5CCCN(c6cccc(-c7cnc8[nH]cc(CC(F)F)c8c7Cl)c6)C5=O)CC4)cc3C2=O)C(=O)N1 ; CCc1c[nH]c2ncc(-c3cccc(N4CCCN(CCNc5ccc6c(c5)C(=O)N(C5CCC(=O)NC5=O)C6=O)C4=O)c3)c(Cl)c12 ; CCc1c[nH]c2ncc(-c3cccc(N4CCCN(CCC5CN(c6ccc7c(c6)C(=O)N(C6CCC(=O)NC6=O)C7=O)C5)C4=O)c3)c(Cl)c12]

a tautomer thereof, a deuterated derivative of the compound or the tautomer, or a pharmaceutically acceptable salt of the foregoing.
61. The method of any of embodiment 57-60, wherein the disease, the disorder, or the condition is cancer.
62. The method of embodiment 61, wherein the cancer is a solid tumor.
63. The method of embodiment 62, wherein the solid tumor is chosen from brain cancer, breast cancer, gastric cancer, renal cancer, prostate cancer, testis cancer, colorectal cancer, lung cancer, bladder cancer, urothelial cancer, cervical cancer, head and neck cancer, esophageal and gastric cancer, osteosarcoma, cervical cancer, endometrial cancer, ovarian cancer, squamous cell cancer, peritoneal cancer, neuroendocrine cancer, hepatocellular carcinoma, pancreatic cancer, genitourinary tract cancer, larynx cancer, skin cancer, nervous system cancer, thyroid cancer, and rhabdosarcoma.
64. The method of embodiment 61, wherein the cancer is a hematologic cancer.
65. The method of embodiment 64, wherein the hematologic cancer is chosen from chronic myeloid leukemia (CML), acute myeloid leukemia (AML), chronic lymphoid leukemia (CLL), acute lymphoid leukemia (ALL), hairy cell leukemia, chronic myelomonocytic leukemia (CMML), juvenile myelomonocyte leukemia (JMML), large granular lymphocytic leukemia (LGL), acute lymphocytic leukemia, acute lymphoblastic leukemia, B-cell lymphoma, T-cell-lymphoma, Hodgkin's lymphoma, non-Hodgkin's lymphoma, hairy cell lymphoma, Burkett's lymphoma, Hodgkin lymphoma, and non-Hodgkin lymphoma.
66. The method of any of embodiments 57-65, further comprising administering to the subject an existing standard treatment or an FDA-approved therapy.
67. The method of any of embodiments 57-65, further comprising administering to the subject at least one additional pharmaceutical agent.
68. The method of embodiment 67, wherein the at least one additional pharmaceutical agent is chosen from a chemotherapeutic agent, an immunotherapeutic agent, and an adjunctive therapeutic agent.

### Examples

### Synthesis of Compounds

To fully understand the present disclosure, the following examples are disclosed. It should be understood that these examples are for illustrative purposes only and are not to be construed as limiting the present disclosure in any manner.

All the specific and generic compounds, and the intermediates disclosed for making those compounds, are considered to be part of the present disclosure.

The compounds of the present disclosure may be made according to standard chemical practices or as disclosed herein. Throughout the following synthetic schemes and in the descriptions for preparing compounds of Formula I, Compounds 1 to 35, pharmaceutically acceptable salts of any of those compounds, solvates of any of the foregoing, and deuterated derivatives of any of the foregoing, the following abbreviations are used:

### Abbreviations

Å=angstrom
Ac=acetyl
Ac₂O=acetic anhydride
Boc₂O=di-tert-butyl dicarbonate
DCM=dichloromethane
DIEA=N,N-Diisopropylethylamine or N-ethyl-N-isopropyl-propan-2-amine
DMAP=dimethylamino pyridine
DMA=dimethyl acetamide
DME=dimethoxyethane
DMF=dimethylformamide
DMSO=dimethyl sulfoxide
EtOAc/EA=Ethyl Acetate
EtOH=ethanol
HOAc=acetic acid
KOAc=potassium acetate
LiHMDS=lithium bis(trimethylsilyl)amide
MeMgBr=methylmagnesium bromide
MeOH=methanol
NaOAc=sodium acetate
NBS=N-bromosuccinimide
Pd(dppf)₂Cl₂=[1,1′-Bis(diphenylphosphino)ferrocene]dichloropalladium(II)
PTSA=p-Toluenesulfonic acid monohydrate
rt=room (ambient) temperature
T3P=2,4,6-Tripropyl-1,3,5,2,4,6-trioxatriphosphorinane-2,4,6-trioxide
TEA=triethylamine
TFA=trifluoroacetic acid
THF=tetrahydrofuran
TsCl=p-toluene sulfonyl chloride
UV=ultra-violet
X-Phos=2-dicyclohexylphosphino-2′,4′,6′-triisopropylbiphenyl

### General Synthesis Procedure I

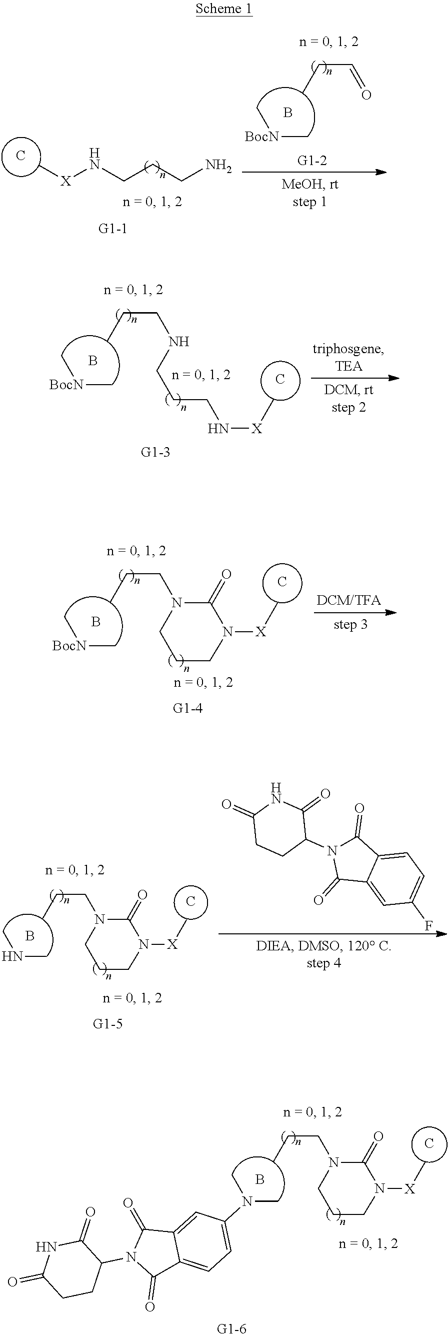

Step 1. Preparation of G1-3: To a solution of primary amine substrate G1-1 (46.96 mmol) in MeOH (200 mL) was added aldehyde G1-2 (46.96 mmol) and AcOH (0.5 mL) and NaCNBH₃ (8.85 g, 140.88 mmol) at 0° C. The reaction mixture was stirred at rt for 2 hrs. The solvent was removed under reduced pressure and the residue was purified by Combi-flash column [DCM/MeOH (10% NH3)=0˜10%] to give the product.

Step 2. Preparation of G1-4: To a solution of G1-3 (42.19 mmol) in DCM (200 mL) was added TEA (8.5 g, 84.37 mmol) and triphosgene (6.25 g, 21.09 mmol) at 0° C. The reaction mixture was stirred at it for 2 hrs. After the reaction completed, H₂O (500 mL) was added to the reaction mixture, and then extracted with DCM (500 mL×3). The combined organic layer was washed with brine (300 mL×2), then dried over anhydrous Na₂SO₄. After filtration, the solution was concentration under vacuum, and the residue was purified by Combi-flash (DCM/MeOH=0˜10%) to give the product.

Step 3. Preparation of G1-5: the solution of G1-4 (0.53 mmol) in DCM/TFA (1:1, 4 mL) was stirred at 25° C. for 1 hour. The resulting mixture was concentrated to give the product, which was used directly for next step.

Step 4. Preparation of G1-6: To a solution of G1-5 (0.5 mmol) in DMSO (5 mL) was added 2-(2,6-dioxopiperidin-3-yl)-5-fluoroisoindole-1,3-dione (131 mg, 0.5 mmol) and DIEA (184 mg, 1.42 mmol). The reaction mixture was stirred at 120° C. under N₂ for 2 hrs. After the reaction completed, H₂O (30 mL) was added to the reaction mixture, and then extracted with EA (50 mL×3). The combined organic layer was washed with brine (50 mL×3), then dried over anhydrous Na₂SO₄. After filtration, the solution was concentrated under vacuum. The residue was purified by prep-HPLC [Gemini-C18 150×21.2 mm, 5 um; ACN—H₂O (0.1% TFA), 50-70] to give the desired product.

### Synthesis of Intermediates

### Intermediate A1: 5-bromo-4-chloro-3-ethyl-1H-pyrrolo[2,3-b]pyridine

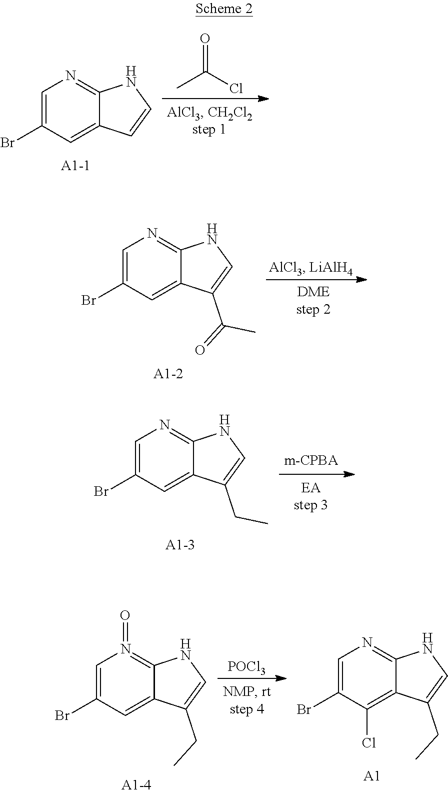

Step 1. Preparation of 1-(5-bromo-1H-pyrrolo[2,3-b]pyridin-3-yl)ethan-1-one: To a solution of 5-bromo-1H-pyrrolo[2,3-b]pyridine (50 g, 0.25 mol) in DCM (550 mL) was added AlCl₃ (101.27 g, 0.76 mol) and acetyl chloride (21.92 g, 0.28 mol) at 0° C. under N₂. The reaction mixture was stirred at rt under N₂ for 7 hrs. MeOH (300 mL) was added to the reaction mixture and the solvent was removed under reduced pressure. The reaction solution was adjusted to pH 6-7 with 3 N aqueous NaOH and extracted with EA (500 mL×3). The combined organic layer was washed with brine (300 mL×3), then dried over with anhydrous Na₂SO₄. After filtration, the solution was concentrated under vacuum, and the crude product was purified by Combi-flash (PE/EtOAc=2:1) to give the product 1-(5-bromo-1H-pyrrolo[2,3-b]pyridin-3-yl)ethan-1-one as yellow solid (43.24 g, 71%). Mass (m/z): 241.0 [M+H]⁺.

Step 2. Preparation of 5-bromo-3-ethyl-1H-pyrrolo[2,3-b]pyridine: To a solution of AlCl₃ (27.8 g, 0.20 mol) in DME (200 mL) was added LiAlH₄ (4.39 g, 0.1 mol) and 1-(5-bromo-1H-pyrrolo[2,3-b]pyridin-3-yl)ethan-1-one (10 g, 0.04 mol) at 0° C. The reaction mixture was stirred at rt under N₂ for 3 hs. After the reaction completed, H₂O (500 mL) was added to the reaction mixture, and then extracted with EA (200 mL×3). The combined organic layer was washed with brine (100 mL×2), then dried over with anhydrous Na₂SO₄. The reaction mixture was filtered, the filtrate was concentrated under vacuum to afford compound product 5-bromo-3-ethyl-1H-pyrrolo[2,3-b]pyridine as yellow solid (11.5 g, 74%). Mass (m/z): 225.0 [M+H]⁺.

Step 3. Preparation of 5-bromo-3-ethyl-1H-pyrrolo[2,3-b]pyridine 7-oxide: To a solution of 5-bromo-3-ethyl-1H-pyrrolo[2,3-b]pyridine (25 g, 0.11 mol) in EA (100 mL) was added 3-Chloroperoxybenzoic acid (26.84 g, 0.155 mol). The reaction mixture was stirred at RT for 3 hrs. The solution was washed with sat. Na₂CO₃ (20 mL) and brine (20 mL), then dried over with anhydrous Na₂SO₄. The reaction mixture was filtered, the filtrate was concentrated to dryness to give the desired product as a white solid (17.4 g, yield: 64.6%). Mass (m/z): 240.7 [M+H]⁺.

Step 4. Preparation of 5-bromo-4-chloro-3-ethyl-1H-pyrrolo[2,3-b]pyridine: To a solution of 5-bromo-3-ethyl-1H-pyrrolo[2,3-b]pyridine 7-oxide (17.3 g, 71.8 mmol) in NMP (15 mL) was added phosphoryl trichloride (55.05 g, 35.9 mmol) at 0° C. The reaction mixture was stirred at rt for 16 hrs. The mixture was quenched with water (50 mL), extracted with EA (30 mL×3), washed with sat. brine, filtrated, concentrated, the residue was purified by flash column (PE/EA=5:1) to give the desired product as a white solid (4.1 g, yield: 22%). Mass (m/z): 258.7 [M+H]⁺.

### Intermediate A2: 5-bromo-4-chloro-3-cyclopropyl-1H-pyrrolo[2,3-b]pyridine

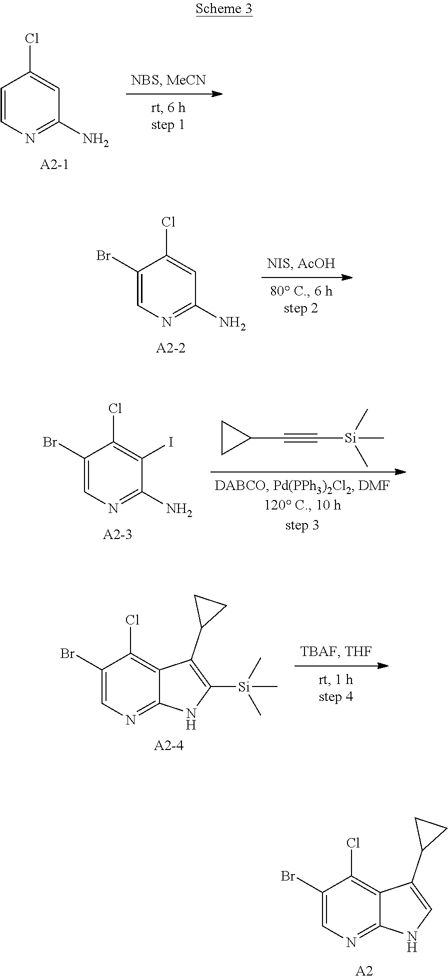

Step 1. Preparation of 5-bromo-4-chloropyridin-2-amine: To a solution of compound 4-chloropyridin-2-amine (300 g, 2.34 mol, 1.0 eq) in acetonitrile (3000 mL) was added NBS (458 g, 2.57 mol, 1.1 eq) in several portions. The reaction mixture was stirred at room temperature for 6 hrs. Then the reaction was poured into water, filtered. The filter cake was washed with PE and dried to afford compound 5-bromo-4-chloropyridin-2-amine (407 g, 83.9% yield) as a yellow solid. Mass (m/z): 207[M+H]⁺. ¹HNMR (400 MHz, DMSO-d₆) δ 8.10 (s, 1H), 6.67 (s, 1H), 6.45 (s, 2H).

Step 2. Preparation of 5-bromo-4-chloro-3-iodopyridin-2-amine: To a solution of compound 5-bromo-4-chloropyridin-2-amine (407 g, 1.97 mol, 1.0 eq) in AcOH (2000 mL) was added NIS (666 g, 2.96 mol, 1.5 eq) in several portions. The reaction mixture was stirred at 80° C. for 4 hrs. The reaction was cooled to room temperature, poured into ice water (5000 mL), adjusted PH>7 with K₂CO₃, extracted with EA (5000 mL×3), washed with a solution of Na₂SO₃ (5000 mL) and brine (5000 mL). The organic phase was concentrated in vacuo to afford compound 5-bromo-4-chloro-3-iodopyridin-2-amine (500 g, 76.3% yield) as a yellow solid. Mass (m/z): 332.7[M+H]⁺. ¹HNMR (400 MHz, DMSO-d₆) δ 8.10 (s, 1H), 6.62 (s, 2H).

Step 3. Preparation of 5-bromo-4-chloro-3-cyclopropyl-2-(trimethylsilyl)-1H-pyrrolo[2,3-b]pyridine: To a solution of compound 5-bromo-4-chloro-3-iodopyridin-2-amine (100 g, 0.300 mol, 1.0 eq), DABCO (101 g, 0.900 mol, 3.0 eq) in DMF (2000 mL) under N₂ was added Pd(PPh₃)₂Cl₂ (21.1 g, 0.03 mol, 0.1 eq). Then compound (cyclopropylethynyl)trimethylsilane (166 g, 1.20 mol, 4.0 eq) was added. The reaction was degassed for 3 times under N₂. The reaction mixture was stirred at 120° C. for 10 hrs. The reaction was filtered, quenched with water (2000 mL), extracted with EA (2000 mL×3), washed with brine (2000 mL), dried over Na₂SO₄, filtered, concentrated in vacuo. The crude was purified by chromatography on sili-gel with THF/PE (1:15) to afford compound 5-bromo-4-chloro-3-cyclopropyl-2-(trimethylsilyl)-1H-pyrrolo[2,3-b]pyridine (27 g, 26.2% yield) as a yellow solid.

Mass (m/z): 344.9[M+H]⁺.

Step 4. Preparation of 5-bromo-4-chloro-3-cyclopropyl-1H-pyrrolo[2,3-b]pyridine: To a mixture of compound 5-bromo-4-chloro-3-cyclopropyl-2-(trimethylsilyl)-1H-pyrrolo[2,3-b]pyridine (27 g, 79.0 mmol, 1.0 eq) in THF (237 mL) was added TBAF in THF (1.0 M, 237 mL, 3.0 eq) and H₂O (4.27 g, 237 mmol, 3.0 eq). The reaction mixture was stirred at room temperature for 1 hrs. The reaction was quenched with water (1000 mL), extracted with EA (1000 mL×3), washed with brine (1000 mL), dried over Na₂SO₄, filtered, concentrated in vacuo. The crude was purified by chromatography on sili-gel with THF/PE (1:4) to afford the product compound 5-bromo-4-chloro-3-cyclopropyl-1H-pyrrolo[2,3-b]pyridine (15 g, 70.4% yield) as a faint yellow solid. Mass (m/z): 272.9[M+H]⁺. ¹HNMR (400 MHz, DMSO-d₆) δ 11.92 (s, 1H), 8.36 (s, 1H), 7.33-7.34 (d, J=4.0 Hz, 1H), 2.11-2.16 (m, 1H), 0.84-0.86 (m, 2H), 0.62-0.64 (m, 2H).

### Intermediate A3: 5-bromo-4-chloro-3-(2,2-difluoroethyl)-1H-pyrrolo[2,3-b]pyridine

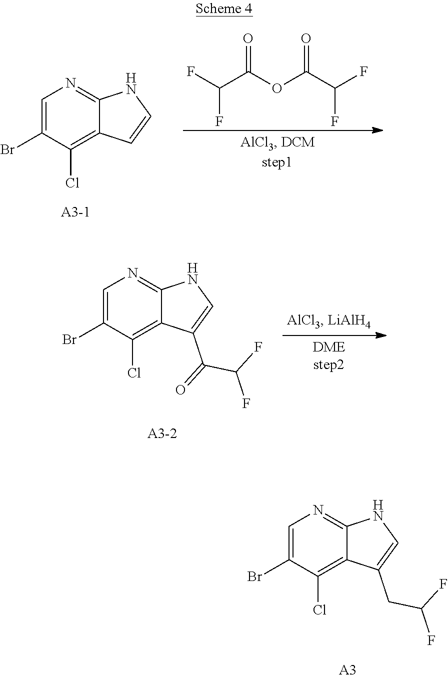 [SMILES: Clc1c(Br)cnc2[nH]ccc12 ; O=C(OC(=O)C(F)F)C(F)F ; O=C(c1c[nH]c2ncc(Br)c(Cl)c12)C(F)F ; FC(F)Cc1c[nH]c2ncc(Br)c(Cl)c12 ; "A3"]

Step 1. Preparation of 1-{5-bromo-4-chloro-1H-pyrrolo[2,3-b]pyridin-3-yl}-2,2-difluoroethanone: To a solution of 3-bromo-4-chloro-7H-pyrrolo[2,3-b]pyridine (500 mg, 2.16 mol) in DCM (10 mL) was added AlCl₃ (863.78 mg, 6.48 mmol) and 2,2-difluoroacetyl 2,2-difluoroacetate (751.9 mg, 4.32 mol) at 0° C. The reaction mixture was stirred at 25° C. under N₂ for 7 hrs. MeOH (30 mL) was added to the reaction mixture and the solvent was removed under reduced pressure. The residue was adjusted to pH 6-7 with 3 N aqueous NaOH and extracted with EA (100 mL×3). The combined organic layers were washed with brine (30 mL×3), then dried over Na₂SO₄. After filtration, the filtrate was concentrated under vacuum, the residue was purified by Combi-flash (eluting with PE/=2:1) to give the product as yellow solid (200 mg, 11.67%). Mass (m/z): 308.7 [M+H]⁺.

Step 2. Preparation of 3-bromo-4-chloro-5-(2,2-difluoroethyl)-7H-pyrrolo[2,3-b]pyridine: To a solution of AlCl₃ (200 mg, 0.65 mmol) in DME (10 mL) was added LiAlH₄ (64.62 mg, 1.62 mmol) and 1-{5-bromo-4-chloro-1H-pyrrolo[2,3-b]pyridin-3-yl}-2,2-difluoroethanone (430.69 g, 3.23 mmol) at 0° C. The reaction mixture was stirred at 25° C. under N₂ for 3 hrs. After the reaction completed, H₂O (100 mL) was added, then extracted with EA (20 mL×3). The combined organic layer was washed with brine (10 mL×2), then dried over Na₂SO₄. The reaction mixture was filtered, the filtrate was concentrated under vacuum and purified by combi-flash, eluting with PE/EA (1:1) to afford compound product 3-bromo-4-chloro-5-(2,2-difluoroethyl)-7H-pyrrolo[2,3-b]pyridine (50 mg, 13.09%) as a brown solid compound. Mass (m/z): 295.0 [M+H]⁺.

### Intermediate A4: tert-butyl 4-((3-(3-bromophenyl)-2-oxotetrahydropyrimidin-1(2H)-yl)methyl)piperidine-1-carboxylate

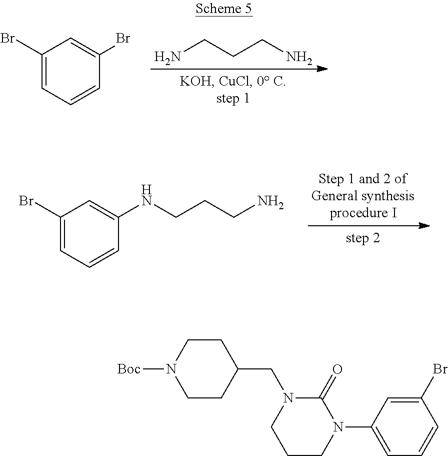 [SMILES: Brc1cccc(Br)c1 ; NCCCN ; NCCCNc1cccc(Br)c1 ; CC(C)(C)OC(=O)N1CCC(CN2CCCN(c3cccc(Br)c3)C2=O)CC1]

Step 1. Preparation of N¹-(3-bromophenyl)propane-1,3-diamine: To a solution of 1,3-dibromobenzene (18 g, 63.63 mmol) and propane-1,3-diamine (14.1 g, 190.87 mmol) and KOH (7.14 g, 127.25 mmol) and CuCl (630 mg, 6.36 mmol), the resulting mixture was stirred at 0° C. under N₂ for 16 hrs. After the reaction completed, H₂O (500 mL) was added to the reaction mixture, and then extracted with DCM (500 mL×3). The combined organic layer was washed with brine (300 mL×2), then dried over anhydrous Na₂SO₄. After filtration, the solution was concentration under vacuum, and the residue was purified by Combi-flash [DCM/MeOH (10% NH₃)=0˜10%] to give the product as brown oil (10.7 g, 73%). Mass (m/z): 231.1 [M+H]⁺.

Step 2. Preparation of tert-butyl 4-((3-(3-bromophenyl)-2-oxotetrahydropyrimidin-1(2H)-yl)methyl)piperidine-1-carboxylate: Following step 1 and 2 of general synthesis procedure I, from N¹-(3-bromophenyl)propane-1,3-diamine and tert-butyl 4-formylpiperidine-1-carboxylate, compound tert-butyl 4-((3-(3-bromophenyl)-2-oxotetrahydropyrimidin-1(2H)-yl)methyl)piperidine-1-carboxylate was obtained as yellow oil (11.1 g, 58%). Mass (m/z): 473.9 [M+H]⁺.

### Intermediate A5: tert-butyl 3-(2-(3-(3-bromophenyl)-2-oxotetrahydropyrimidin-1(2H)-yl)ethyl)azetidine-1-carboxylate

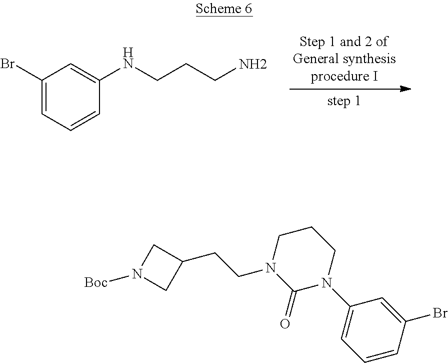 [SMILES: NCCCNc1cccc(Br)c1 ; CC(C)(C)OC(=O)N1CC(CCN2CCCN(c3cccc(Br)c3)C2=O)C1]

Step 1. Preparation of tert-butyl 3-(2-(3-(3-bromophenyl)-2-oxotetrahydropyrimidin-1(2H)-yl)ethyl)azetidine-1-carboxylate: Following step 1 and 2 of general synthesis procedure I, from N¹-(3-bromophenyl)propane-1,3-diamine and tert-butyl 3-(2-oxoethyl)azetidine-1-carboxylate, compound tert-butyl 3-(2-(3-(3-bromophenyl)-2-oxotetrahydropyrimidin-1(2H)-yl)ethyl)azetidine-1-carboxylate was obtained as yellow oil (880 mg, 39%). Mass (m/z): 462.2 [M+H]⁺.

### Intermediate A6 and A7: 3-{1-oxo-6-[4-({2-oxo-3-[3-(4,4,5,5-tetramethyl-1,3,2-dioxaborolan-2-yl)phenyl]-1,3-diazinan-1-yl}methyl)piperidin-1-yl]-3H-isoindol-2-yl}piperidine-2,6-dione and 3-(1-oxo-5-(4-((2-oxo-3-(3-(4,4,5,5-tetramethyl-1,3,2-dioxaborolan-2-yl)phenyl)tetrahydropyrimidin-1(2H)-yl)methyl)piperidin-1-yl)isoindolin-2-yl)piperidine-2,6-dione

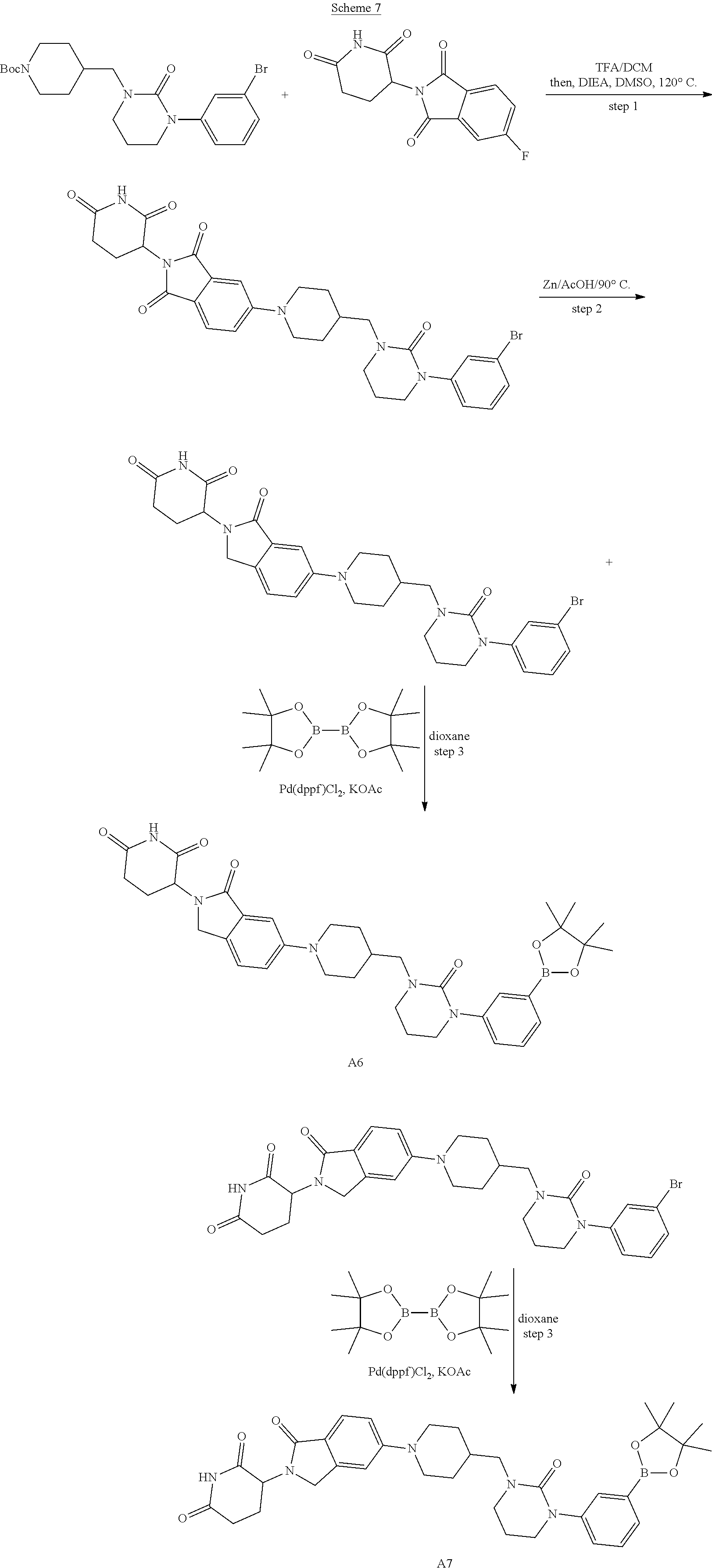

Step 1. Preparation of 5-(4-{[3-(3-bromophenyl)-2-oxo-1,3-diazinan-1-yl]methyl} piperidin-1-yl)-2-(2,6-dioxopiperidin-3-yl)isoindole-1,3-dione: To a solution of tert-butyl 4-((3-(3-bromophenyl)-2-oxotetrahydropyrimidin-1(2H)-yl)methyl)piperidine-1-carboxylate (1.3 g, 2.8 mmol) in DCM (40 mL) was added TFA (2 mL) and H₂O (2 mL). The reaction mixture was stirred at 25° C. for 16 hrs. Water (30 mL) was added and the mixture was extracted with DCM (40 mL×2). The combined DCM layers were washed with saturated NaHCO₃ (30 mL>2) and brine (40 mL), dried over Na₂SO₄ and concentrated to give crude product. A solution of this crude product, 1-(3-bromophenyl)-3-(piperidin-4-ylmethyl)-1,3-diazinan-2-one (1 g, 2.8 mmol) and DIEA (1.09 g, 8.4 mmol) in DMSO (20 mL) was stirred under nitrogen at 120° C. for 2 hrs. Water (50 mL) was added and the mixture was extracted with EA (30 mL×3). The combined organic layers were washed with brine (50 mL×3), dried over Na₂SO₄. Then by filtration, the filtrate was concentrated. The residue was purified by flash Chromatography (PE/EA=1:1) to give the product as a brown solid (1.1 g, 57%). Mass (m/z): 607.7 [M+H]⁺.

Step 2. Preparation of 3-[5-(4-{[3-(3-bromophenyl)-2-oxo-1,3-diazinan-1-yl]methyl}piperidin-1-yl)-1-oxo-3H-isoindol-2-yl]piperidine-2,6-dione and 3-(5-(4-((3-(3-bromophenyl)-2-oxotetrahydropyrimidin-1(2H)-yl)methyl)piperidin-1-yl)-1-oxoisoindolin-2-yl)piperidine-2,6-dione: To a solution of 5-(4-{[3-(3-bromophenyl)-2-oxo-1,3-diazinan-1-yl]methyl}piperidin-1-yl)-2-(2,6-dioxopiperidin-3-yl)isoindole-1,3-dione (1.4 g, 2.3 mmol) in AcOH (20 mL) was added Zn powder (1.5 g, 2.3 mmol), the reaction mixture was stirred at 90° C. for 16 hrs. The reaction mixture was filtered and the filtrate was concentrated. Water (30 mL) was added and the mixture was extracted with DCM (30 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, 5 um; ACN—H₂O (0.1% TFA), 55-60] to give the products: 3-[5-(4-{[3-(3-bromophenyl)-2-oxo-1,3-diazinan-1-yl]methyl}piperidin-1-yl)-1-oxo-3H-isoindol-2-yl]piperidine-2,6-dione (430 mg, 27%). Mass (m/z): 680.3 [M+H]⁺.

3-(5-(4-((3-(3-bromophenyl)-2-oxotetrahydropyrimidin-1(2H)-yl)methyl)piperidin-1-yl)-1-oxoisoindolin-2-yl)piperidine-2,6-dione (180 mg, 11%). Mass (m/z): 680.3 [M+H]⁺.

Step 3. Preparation of 3-{1-oxo-6-[4-({2-oxo-3-[3-(4,4,5,5-tetramethyl-1,3,2-dioxaborolan-2-yl)phenyl]-1,3-diazinan-1-yl}methyl)piperidin-1-yl]-3H-isoindol-2-yl}piperidine-2,6-dione and 3-(1-oxo-5-(4-((2-oxo-3-(3-(4,4,5,5-tetramethyl-1,3,2-dioxaborolan-2-yl)phenyl)tetrahydropyrimidin-1(2H)-yl)methyl)piperidin-1-yl)isoindolin-2-yl)piperidine-2,6-dione: To a solution of 3-[6-(4-{[3-(3-bromophenyl)-2-oxo-1,3-diazinan-1-yl]methyl}piperidin-1-yl)-1-oxo-3H-isoindol-2-yl]piperidine-2,6-dione (430 mg, 0.72 mmol), 4,4,5,5-tetramethyl-2-(4,4,5,5-tetramethyl-1,3,2-dioxaborolan-2-yl)-1,3,2-dioxaborolane (275 mg, 1.08 mmol) and KOAc (213 mg, 2.17 mmol) in 1.4-dioxane (10 mL) was added Pd(dppf)Cl₂ (53 mg, 0.072 mmol). The mixture was stirred under nitrogen at 110° C. for 2 hrs. Water (15 mL) was added and the mixture was extracted with EA (10 mL×3). The combined organic layers were washed with brine (20 mL×2), dried over Na₂SO₄. Then by filtration, the filtrate was concentrated. The residue was purified by flash Chromatography (DCM/MeOH=20:1) to give the product as a brown solid (300 mg, 58%). Mass (m/z): 642.4 [M+H]⁺.

Following the same procedure from 3-(5-(4-((3-(3-bromophenyl)-2-oxotetrahydropyrimidin-1(2H)-yl)methyl)piperidin-1-yl)-1-oxoisoindolin-2-yl)piperidine-2,6-dione, product 3-(1-oxo-5-(4-((2-oxo-3-(3-(4,4,5,5-tetramethyl-1,3,2-dioxaborolan-2-yl)phenyl)tetrahydropyrimidin-1(2H)-yl)methyl)piperidin-1-yl)isoindolin-2-yl)piperidine-2,6-dione was obtained as a brown solid (140 mg, 64%). Mass (m/z): 642.3 [M+H]⁺.

### Synthesis of Exemplary Compounds

### Compound 1: 2-(2,6-dioxopiperidin-3-yl)-5-{4-[(2-oxo-3-phenyl-1,3-diazinan-1-yl)methyl]piperidin-1-yl}isoindole-1, 3-dione

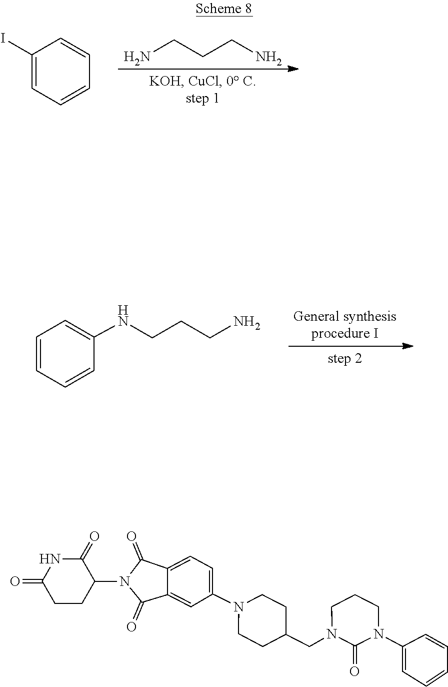 [SMILES: Ic1ccccc1 ; NCCCN ; NCCCNc1ccccc1 ; O=C1CCC(N2C(=O)c3ccc(N4CCC(CN5CCCN(c6ccccc6)C5=O)CC4)cc3C2=O)C(=O)N1]

Step 1. Preparation of N-(3-aminopropyl)aniline: To a solution of iodobenzene (3 g, 0.015 mol) was added propane-1,3-diamine (3.27 g, 0.04 mol), CuCl (0.15 g, 1.4 mmol) and KOH (1.65 g, 0.03 mol). The reaction mixture was stirred at 0° C. under N₂ for 2 hrs. After the reaction completed, H₂O (50 mL) was added to the reaction mixture, and then extracted with EA (50 mL×3). The combined organic layer was washed with brine (30 mL×2), then dried over anhydrous Na₂SO₄. After filtration, the solution was concentrated under vacuum. The residue was purified by Combi-flash (DCM/MeOH=0˜5%) to give the desired product (1.97 g, 80%) as yellow oil. Mass (m/z): 151.2 [M+H]⁺.

Step 2. Preparation of 2-(2,6-dioxopiperidin-3-yl)-5-{4-[(2-oxo-3-phenyl-1,3-diazinan-1-yl)methyl]piperidin-1-yl}isoindole-1, 3-dione: Following general synthesis procedure I, from N-(3-aminopropyl)aniline and tert-butyl 4-formylpiperidine-1-carboxylate, compound 2-(2,6-dioxopiperidin-3-yl)-5-{4-[(2-oxo-3-phenyl-1,3-diazinan-1-yl)methyl]piperidin-1-yl}isoindole-1, 3-dione was obtained

(60 mg, 24%) as a yellow solid. Mass (m/z): 530.3 [M+H]⁺. ¹H NMR (400 MHz, DMSO-d₆) δ 11.07 (s, 1H), 7.64 (d, J=8.4 Hz, 1H), 7.26-7.22 (m, 6H), 7.10 (t, J=7.0 Hz, 1H), 5.06 (dd, J=12.8, 5.2 Hz, 1H), 4.07 (d, J=13.0 Hz, 2H), 3.65 (t, J=5.6 Hz, 2H), 3.38 (t, J=5.8 Hz, 2H), 3.19 (d, J=7.2 Hz, 2H), 3.00-2.88 (m, 3H), 2.56 (dd, J=17.6, 10.8 Hz, 2H), 2.07-1.94 (m, 4H), 1.71 (d, J=11.2 Hz, 2H), 1.20 (d, J=10.2 Hz, 2H).

### Compound 2: 2-(2,6-dioxopiperidin-3-yl)-5-(4-((2-oxo-3-phenylimidazolidin-1-yl)methyl) piperidin-1-yl)isoindoline-1,3-dione

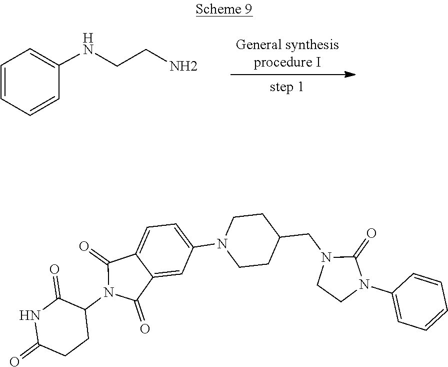 [SMILES: NCCNc1ccccc1 ; O=C1CCC(N2C(=O)c3ccc(N4CCC(CN5CCN(c6ccccc6)C5=O)CC4)cc3C2=O)C(=O)N1]

Step 1. Preparation of 2-(2,6-dioxopiperidin-3-yl)-5-(4-((2-oxo-3-phenylimidazolidin-1-yl)methyl)piperidin-1-yl)isoindoline-1,3-dione: Following general synthesis procedure I, from N¹-phenylethane-1,2-diamine and tert-butyl 4-formylpiperidine-1-carboxylate, compound 2-(2,6-dioxopiperidin-3-yl)-5-(4-((2-oxo-3-phenylimidazolidin-1-yl)methyl)piperidin-1-yl)isoindoline-1,3-dione was obtained as a yellow solid (130 mg, 21%). Mass (m/z): 516.2 [M+H]⁺. ¹H NMR (400 MHz, DMSO-d₆) δ 11.08 (s, 1H), 7.66 (d, J=8.6 Hz, 1H), 7.56 (d, J=7.8 Hz, 2H), 7.36-7.20 (m, 4H), 6.98 (t, J=7.2 Hz, 1H), 5.07 (dd, J=12.8, 5.2 Hz, 1H), 4.06 (s, 2H), 3.85-3.77 (m, 2H), 3.54-3.45 (m, 2H), 3.09 (d, J=7.2 Hz, 2H), 2.98 (t, J=11.8 Hz, 2H), 2.87 (d, J=11.8 Hz, 1H), 2.56 (dd, J=18.8, 12.4 Hz, 2H), 1.99 (dd, J=20.0, 14.8 Hz, 2H), 1.74 (d, J=13.6 Hz, 2H), 1.22 (d, J=11.8 Hz, 2H).

### Compound 3: 5-(4-((3-(3-(4-chloropyridin-3-yl)phenyl)-2-oxotetrahydropyrimidin-1(2H)-yl)methyl)piperidin-1-yl)-2-(2,6-dioxopiperidin-3-yl)isoindoline-1,3-dione

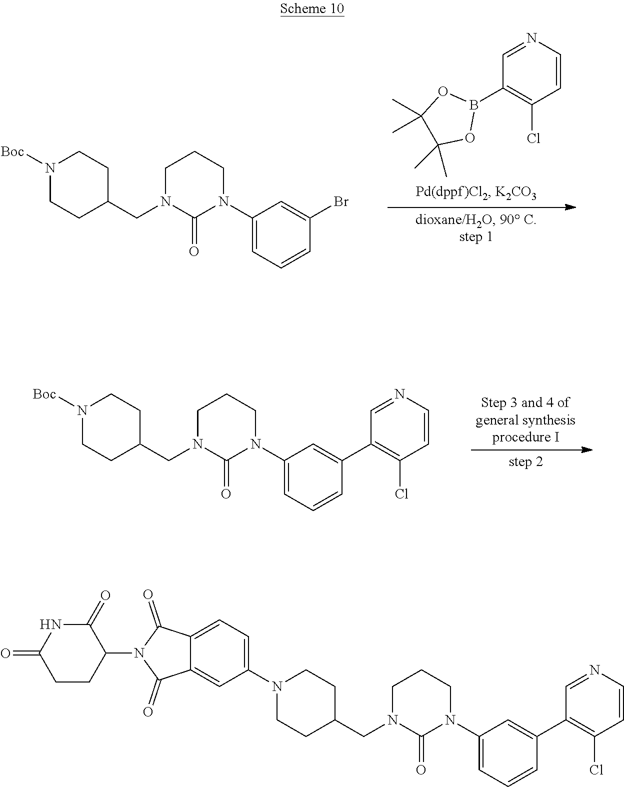 [SMILES: CC(C)(C)OC(=O)N1CCC(CN2CCCN(c3cccc(Br)c3)C2=O)CC1 ; CC1(C)OB(c2cnccc2Cl)OC1(C)C ; CC(C)(C)OC(=O)N1CCC(CN2CCCN(c3cccc(-c4cnccc4Cl)c3)C2=O)CC1 ; O=C1CCC(N2C(=O)c3ccc(N4CCC(CN5CCCN(c6cccc(-c7cnccc7Cl)c6)C5=O)CC4)cc3C2=O)C(=O)N1]

Step 1. Preparation of tert-butyl 4-((3-(3-(4-chloropyridin-3-yl)phenyl)-2-oxotetrahydropyrimidin-1(2H)-yl)methyl)piperidine-1-carboxylate: To a solution of tert-butyl 4-((3-(3-bromophenyl)-2-oxotetrahydropyrimidin-1(2H)-yl)methyl)piperidine-1-carboxylate (200 mg, 0.44 mmol) in dioxane/H₂O (10:1, 10 mL) was added 4-chloro-3-(4,4,5,5-tetramethyl-1,3,2-dioxaborolan-2-yl)pyridine (124 mg, 0.52 mmol), K₂CO₃ (130 mg, 0.94 mmol) and Pd(dppf)Cl₂ (34 mg, 0.05 mmol). The reaction mixture was stirred at 90° C. under N₂ for 16 hrs. The solvent was removed under reduced pressure and the residue was purified by Combi-flash (PE/EA=0˜50%) to give the product tert-butyl 4-((3-(3-(4-chloropyridin-3-yl)phenyl)-2-oxotetrahydropyrimidin-1(2H)-yl)methyl)piperidine-1-carboxylate as yellow oil (90 mg, 39%). Mass (m/z): 485.1 [M+H]⁺.

Step 2. Preparation of 5-(4-((3-(3-(4-chloropyridin-3-yl)phenyl)-2-oxotetrahydropyrimidin-1(2H)-yl)methyl)piperidin-1-yl)-2-(2,6-dioxopiperidin-3-yl)isoindoline-1,3-dione: Following step 3 and 4 of general synthesis procedure I, compound 5-(4-((3-(3-(4-chloropyridin-3-yl)phenyl)-2-oxotetrahydropyrimidin-1(2H)-yl)methyl)piperidin-1-yl)-2-(2,6-dioxopiperidin-3-yl)isoindoline-1,3-dione was obtained as a yellow solid (14 mg, 15%). Mass (m/z): 641.3 [M+H]⁺. ¹H NMR (400 MHz, DMSO-d₆) δ 11.08 (s, 1H), 8.58 (s, 1H), 8.54 (d, J=5.2 Hz, 1H), 7.66 (dd, J=13.4, 6.8 Hz, 2H), 7.46-7.36 (m, 3H), 7.31 (s, 1H), 7.28-7.19 (m, 2H), 5.06 (dd, J=12.8, 5.4 Hz, 1H), 4.07 (d, J=13.2 Hz, 2H), 3.77-3.69 (m, 2H), 3.41 (s, 2H), 3.20 (d, J=7.2 Hz, 2H), 3.06-2.79 (m, 4H), 2.69-2.53 (m, 3H), 2.06-1.93 (m, 4H), 1.71 (d, J=11.4 Hz, 2H).

### Compound 4: 2-(2,6-dioxopiperidin-3-yl)-5-(4-{[2-oxo-3-(pyridin-2-yl)-1,3-diazinan-1-yl]methyl}piperidin-1-yl)isoindole-1,3-dione

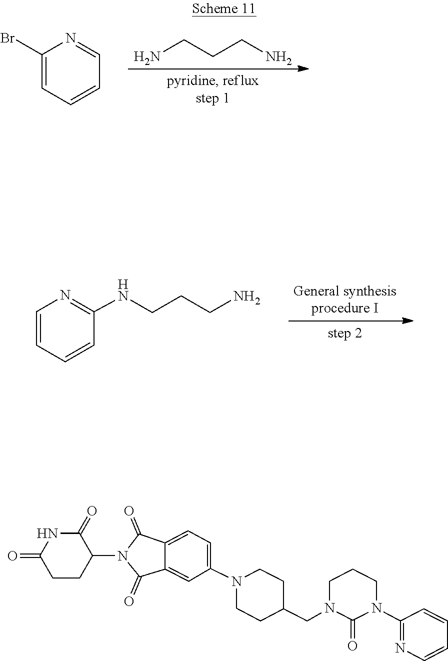 [SMILES: Brc1ccccn1 ; NCCCN ; NCCCNc1ccccn1 ; O=C1CCC(N2C(=O)c3ccc(N4CCC(CN5CCCN(c6ccccn6)C5=O)CC4)cc3C2=O)C(=O)N1]

Step 1. Preparation of N-(3-aminopropyl)pyridin-2-amine: To a solution of 2-bromopyridine (5 g, 31.6 mmol) in pyridine (30 mL) was added propane-1,3-diamine (37.6 mL, 450 mmol). The reaction mixture was stirred at 130° C. under N₂ for 16 hrs. After the reaction completed, H₂O (100 mL) was added to the reaction mixture, and then extracted with EA (100 mL×3). The combined organic layer was washed with brine (150 mL×2), then dried over anhydrous Na₂SO₄. After filtration, the solution was concentrated under vacuum. The residue was purified by Combi-flash (DCM/MeOH=0˜5%) to give the desired product (3.5 g, 66%) as yellow oil. Mass (m/z): 152.2 [M+H]⁺.

Step 2. Preparation of 2-(2,6-dioxopiperidin-3-yl)-5-(4-{[2-oxo-3-(pyridin-2-yl)-1,3-diazinan-1-yl]methyl}piperidin-1-yl)isoindole-1,3-dione: Following general synthesis procedure I, from N-(3-aminopropyl)pyridin-2-amine and tert-butyl 4-formylpiperidine-1-carboxylate, compound 2-(2,6-dioxopiperidin-3-yl)-5-(4-{[2-oxo-3-(pyridin-2-yl)-1,3-diazinan-1-yl]methyl}piperidin-1-yl)isoindole-1,3-dione was obtained (20 mg, 1.8%) as a yellow solid. Mass (m/z): 531.2 [M+H]⁺. ¹H NMR (400 MHz, DMSO-d₆) δ 11.08 (s, 1H), 8.31 (d, J=4.0 Hz, 1H), 7.73 (d, J=8.4 Hz, 1H), 7.66 (s, 1H), 7.63 (d, J=4.4 Hz, 1H), 7.31 (s, 1H), 7.24 (d, J=8.6 Hz, 1H), 7.05-6.98 (m, 1H), 5.06 (dd, J=12.8, 5.2 Hz, 1H), 4.07 (d, J=12.8 Hz, 2H), 3.94-3.82 (m, 2H), 3.42-3.36 (m, 2H), 3.24 (d, J=7.2 Hz, 2H), 3.11-2.80 (m, 4H), 2.33 (s, 1H), 2.07-1.91 (m, 4H), 1.71 (d, J=12.2 Hz, 2H), 1.22 (d, J=9.2 Hz, 2H).

### Compound 5: 5-(4-((3-cyclohexyl-2-oxotetrahydropyrimidin-1(2H)-yl) methyl) piperidin-1-yl)-2-(2,6-dioxopiperidin-3-yl) isoindoline-1,3-dione

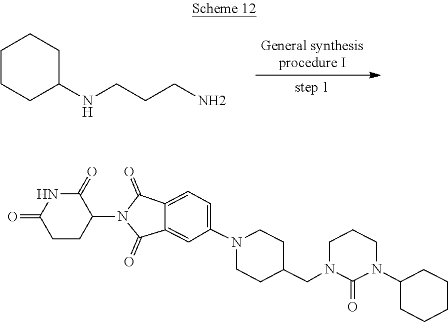 [SMILES: NCCCNC1CCCCC1 ; O=C1CCC(N2C(=O)c3ccc(N4CCC(CN5CCCN(C6CCCCC6)C5=O)CC4)cc3C2=O)C(=O)N1]

Step 1. Preparation of 5-(4-((3-cyclohexyl-2-oxotetrahydropyrimidin-1(2H)-yl) methyl) piperidin-1-yl)-2-(2,6-dioxopiperidin-3-yl) isoindoline-1,3-dione: Following general synthesis procedure I, from N-(3-aminopropyl) cyclohexan amine and tert-butyl 4-formylpiperidine-1-carboxylate, compound 5-(4-((3-cyclohexyl-2-oxotetrahydropyrimidin-1(2H)-yl) methyl) piperidin-1-yl)-2-(2,6-dioxopiperidin-3-yl) isoindoline-1,3-dione was obtained (13 mg, yield: 3.3%) as a yellow solid. Mass (m/z): 536[M+H]⁺. ¹H NMR (400 MHz, DMSO-dd) δ 11.05 (s, 1H), 7.61 (d, J=8.4 Hz, 1H), 7.27 (d, J=1.8 Hz, 1H), 7.19 (dd, J=8.7, 2.0 Hz, 1H), 5.02 (dd, J=128, 5.4 Hz, 1H), 4.02 (t, J=11.2 Hz, 3H), 3.15 (t, J=5.8 Hz, 2H), 3.11-3.00 (m, 4H), 2.91-2.81 (m, 3H), 2.64-2.50 (m, 2H), 2.01-1.93 (m, 1H), 1.86 (d, J=3.4 Hz, 1H), 1.82-1.74 (m, 2H), 1.69 (d, J=12.2 Hz, 2H), 1.63-1.49 (m, 3H), 1.44 (d, J=10.0 Hz, 2H), 1.32 (dt, J=12.2, 9.6 Hz, 2H), 1.26-1.07 (m, 4H), 1.00 (d, J=12.8 Hz, 1H).

### Compound 6: 2-(2,6-dioxopiperidin-3-yl)-5-(4-((2-oxo-3-phenyl-1,3-diazepan-1-yl)methyl) piperidin-1-yl)isoindoline-1,3-dione

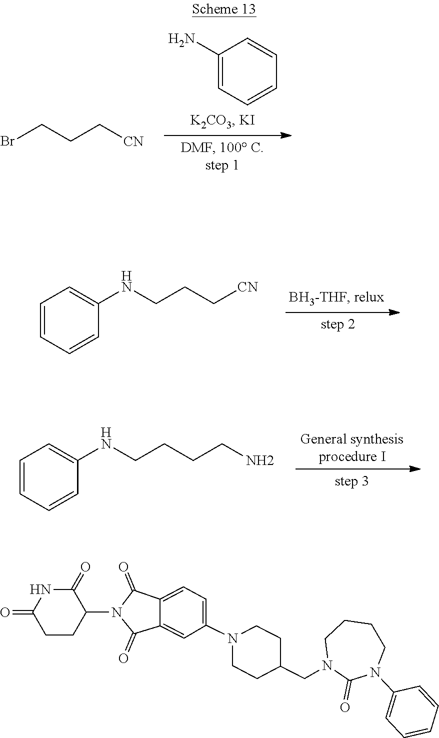 [SMILES: Nc1ccccc1 ; N#CCCCBr ; N#CCCCNc1ccccc1 ; NCCCCNc1ccccc1 ; O=C1CCC(N2C(=O)c3ccc(N4CCC(CN5CCCCN(c6ccccc6)C5=O)CC4)cc3C2=O)C(=O)N1]

Step 1. Preparation of 4-(phenylamino)butanenitrile: To a solution of 4-bromobutanenitrile (2 g, 13.51 mmol) and aniline (2.51 g, 27.02 mmol) in DME (14 mL) and DMF (3.5 mL) was added K₂CO₃ (1.86 g, 13.51 mmol) and KI (4.48 g, 27.02 mmol). The reaction mixture was stirred at 100° C. under N₂ for 16 hrs. Water (30 mL) was added and the mixture was extracted with EA (30 mL×3). The combined organic layers were washed with brine (20 mL×2), dried over Na₂SO₄. Then by filtration, the filtrate was concentrated to dryness to give the product as a brown oil (3 g, purity: 70%). Mass (m/z): 161.1[M+H]⁺.

Step 2. Preparation of N¹-phenylbutane-1,4-diamine: A mixture of 4-(phenylamino)butanenitrile (3000 mg, 18.72 mmol) in BH₃-THF (1M in THF) (37.4 mL, 37.44 mmol) was stirred at 65° C. under N₂ for 2 hrs. The reaction mixture was quenched by MeOH (100 mL) and evaporated. The residue was purified by silica gel column chromatography (DCM/MeOH/NH₃·H₂O=10:1:0.1) to give the product as colorless oil (900 mg, 42%). Mass (m/z): 165.2[M+H]⁺.

Step 3. Preparation of 2-(2,6-dioxopiperidin-3-yl)-5-(4-((2-oxo-3-phenyl-1,3-diazepan-1-yl)methyl) piperidin-1-yl)isoindoline-1,3-dione: Following general synthesis procedure I, from N¹-phenylbutane-1,4-diamine and tert-butyl 4-formylpiperidine-1-carboxylate, compound 2-(2,6-dioxopiperidin-3-yl)-5-(4-((2-oxo-3-phenyl-1,3-diazepan-1-yl)methyl) piperidin-1-yl)isoindoline-1,3-dione was obtained as a yellow solid (180 mg, 47%). Mass (m/z): 544.1[M+H]⁺. ¹H NMR (400 MHz, DMSO-d₆) δ 11.08 (s, 1H), 7.65 (d, J=8.6 Hz, 1H), 7.37-7.21 (m, 4H), 7.19-7.09 (m, 2H), 7.03 (t, J=7.4 Hz, 1H), 5.07 (dd, J=12.8, 5.4 Hz, 1H), 4.08 (d, J=13.2 Hz, 2H), 3.68-3.53 (m, 2H), 3.40-3.34 (m, 2H), 3.16 (d, J=7.2 Hz, 2H), 3.05-2.94 (m, 2H), 2.93-2.82 (m, 1H), 2.63-2.51 (m, 2H), 2.05-1.90 (m, 2H), 1.82-1.60 (m, 6H), 1.24-1.15 (m, 2H).

### Compound 7: 5-(4-((3-(tert-butyl)-2-oxotetrahydropyrimidin-1(2H)-yl)methyl)piperidin-1-yl)-2-(2,6-dioxopiperidin-3-yl)isoindoline-1,3-dione

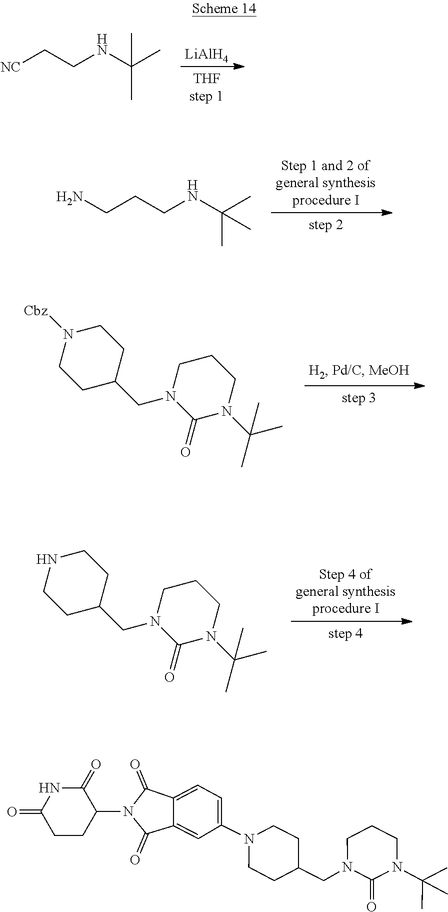

Step 1. Preparation of N¹-(tert-butyl)propane-1,3-diamine: To a solution of 3-(tert-butylamino)propanenitrile (1000 mg, 7.924 mmol) in ethylether (15 mL) was added LiAlH₄ (301 mg, 7.924 mmol). The reaction mixture was stirred at 30° C. under N₂ for 16 hrs. The reaction mixture was quenched by 15% aqueous NaOH (2 mL) and filtered. Filtrate was collected and evaporated to give the product as colorless oil (900 mg, 70%). Mass (m/z): 131.2[M+H]⁺.

Step 2. Preparation of benzyl 4-((3-(tert-butyl)-2-oxotetrahydropyrimidin-1(2H)-yl)methyl)piperidine-1-carboxylate: Following step 1 and 2 of general synthesis procedure I, from N¹-(tert-butyl)propane-1,3-diamine and tert-butyl 4-formylpiperidine-1-carboxylate, compound benzyl 4-((3-(tert-butyl)-2-oxotetrahydropyrimidin-1(2H)-yl)methyl)piperidine-1-carboxylate was obtained as colorless oil (800 mg, 39%). Mass (m/z): 388.2[M+H]⁺.

Step 3. Preparation of 1-(tert-butyl)-3-(piperidin-4-ylmethyl)tetrahydropyrimidin-2(1H)-one: To a solution of benzyl 4-((3-(tert-butyl)-2-oxotetrahydropyrimidin-1(2H)-yl)methyl)piperidine-1-carboxylate (400 mg, 1.03 mmol) in MeOH (5 mL) was added 10% Pd/C (40 mg, wt/wt 10%). The reaction mixture was stirred at 50° C. under H₂ (1 atm) for 16 hrs. The reaction mixture was filtered and the filtrate was evaporated to give the product as colorless oil (220 mg, 67%). Mass (m/z): 254.2[M+H]⁺.

Step 4. Preparation of 5-(4-((3-(tert-butyl)-2-oxotetrahydropyrimidin-1(2H)-yl)methyl)piperidin-1-yl)-2-(2,6-dioxopiperidin-3-yl)isoindoline-1,3-dione: Following step 4 of general synthesis procedure I, from 1-(tert-butyl)-3-(piperidin-4-ylmethyl)tetrahydropyrimidin-2(1H)-one and 2-(2,6-dioxopiperidin-3-yl)-5-fluoroisoindoline-1,3-dione, compound 5-(4-((3-(tert-butyl)-2-oxotetrahydropyrimidin-1(2H)-yl)methyl)piperidin-1-yl)-2-(2,6-dioxopiperidin-3-yl)isoindoline-1,3-dione was obtained as a yellow solid (190 mg, 45%). Mass (m/z): 510.1[M+H]⁺. ¹H NMR (400 MHz, DMSO-d₆) δ 11.08 (s, 1H), 7.65 (d, J=8.6 Hz, 1H), 7.31 (d, J=2.0 Hz, 1H), 7.23 (dd, J=8.6, 2.2 Hz, 1H), 5.06 (dd, J=12.8, 5.4 Hz, 1H), 4.05 (d, J=13.0 Hz, 2H), 3.22 (t, J=6.0 Hz, 2H), 3.17-3.07 (m, 4H), 3.00-2.92 (m, 2H), 2.92-2.81 (m, 1H), 2.66-2.51 (m, 2H), 2.04-1.98 (m, 1H), 1.92-1.77 (m, 3H), 1.68-1.61 (m, 2H), 1.33 (s, 9H), 1.22-1.12 (m, 2H).

### Compound 8: 5-(3-(2-(3-(3-(4-chloropyridin-3-yl)phenyl)-2-oxotetrahydropyrimidin-1(2H)-yl)ethyl)azetidin-1-yl)-2-(2,6-dioxopiperidin-3-yl)isoindoline-1,3-dione

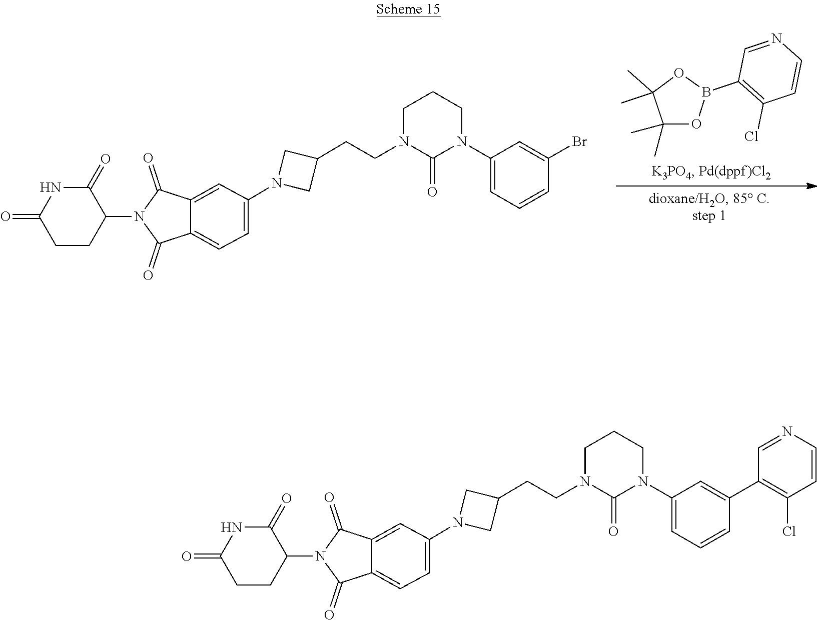 [SMILES: O=C1CCC(N2C(=O)c3ccc(N4CC(CCN5CCCN(c6cccc(Br)c6)C5=O)C4)cc3C2=O)C(=O)N1 ; CC1(C)OB(c2cnccc2Cl)OC1(C)C ; O=C1CCC(N2C(=O)c3ccc(N4CC(CCN5CCCN(c6cccc(-c7cnccc7Cl)c6)C5=O)C4)cc3C2=O)C(=O)N1]

Step 1. Preparation of 5-(3-(2-(3-(3-(4-chloropyridin-3-yl)phenyl)-2-oxotetrahydropyrimidin-1(2H)-yl)ethyl)azetidin-1-yl)-2-(2,6-dioxopiperidin-3-yl)isoindoline-1,3-dione: To a solution of 5-(3-(2-(3-(3-bromophenyl)-2-oxotetrahydropyrimidin-1(2H)-yl)ethyl)azetidin-1-yl)-2-(2,6-dioxopiperidin-3-yl)isoindoline-1,3-dione (80 mg, 0.13 mmol) in dioxane/H₂O (10:1, 10 mL) was added 4-chloro-3-(4,4,5,5-tetramethyl-1,3,2-dioxaborolan-2-yl)pyridine (39 mg, 0.16 mmol), K₃PO₄ (57 mg, 0.26 mmol) and Pd(dppf)Cl₂ (10 mg, 0.01 mmol). The reaction mixture was stirred at 85° C. under N₂ for 16 hrs. The solvent was removed under reduced pressure and the residue was purified by Combi-flash (PE/EA=0-50%) to give the product as a yellow solid (24 mg, 28%). Mass (m/z): 627.2 [M+H]⁺. ¹H NMR (400 MHz, DMSO-d₆) δ 11.08 (s, 1H), 8.58 (s, 1H), 8.53 (d, J=5.2 Hz, 1H), 7.67 (d, J=5.2 Hz, 1H), 7.62 (d, J=8.4 Hz, 1H), 7.46-7.37 (m, 3H), 7.26-7.21 (m, 1H), 6.76 (d, J=1.8 Hz, 1H), 6.62 (dd, J=8.4, 2.0 Hz, 1H), 5.05 (dd, J=12.8, 5.4 Hz, 1H), 4.14 (t, J=8.2 Hz, 2H), 3.75-3.67 (m, 4H), 2.95-2.66 (m, 3H), 2.63-2.51 (m, 4H), 2.10-1.94 (m, 4H), 1.93-1.84 (m, 2H).

### Compound 9: 5-(4-((3-(3-bromophenyl)-2-oxotetrahydropyrimidin-1(2H)-yl)methyl)piperidin-1-yl-2-(2 6-dioxopiperidin-3-yl)isoindoline-1,3-dione

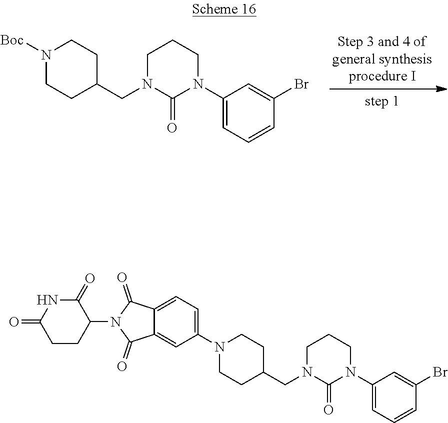 [SMILES: CC(C)(C)OC(=O)N1CCC(CN2CCCN(c3cccc(Br)c3)C2=O)CC1 ; O=C1CCC(N2C(=O)c3ccc(N4CCC(CN5CCCN(c6cccc(Br)c6)C5=O)CC4)cc3C2=O)C(=O)N1]

Step 1. Preparation of 5-(4-((3-(3-bromophenyl)-2-oxotetrahydropyrimidin-1(2H)-yl)methyl)piperidin-1-yl)-2-(2,6-dioxopiperidin-3-yl)isoindoline-1,3-dione: Following step 3 and 4 of general synthesis procedure I, from tert-butyl 4-((3-(3-bromophenyl)-2-oxotetrahydropyrimidin-1(2H)-yl)methyl)piperidine-1-carboxylate, compound 5-(4-((3-(3-bromophenyl)-2-oxotetrahydropyrimidin-1(2H)-yl)methyl)piperidin-1-yl)-2-(2,6-dioxopiperidin-3-yl)isoindoline-1,3-dione was obtained as a yellow solid (10 mg, 9%). Mass (m/z): 610.2 [M+H]⁺. ¹H NMR (400 MHz, DMSO-d₆) δ 11.08 (s, 1H), 7.65 (d, J=8.4 Hz, 1H), 7.52 (d, J=2.2 Hz, 1H), 7.32 (d, J=2.2 Hz, 1H), 7.29-7.21 (m, 4H), 5.06 (dd, J=12.8, 5.4 Hz, 1H), 4.07 (d, J=12.8 Hz, 2H), 3.69-3.64 (m, 2H), 3.37 (d, J=6.0 Hz, 3H), 3.19 (d, J=7.2 Hz, 2H), 3.03-2.82 (m, 4H), 2.62-2.53 (m, 2H), 2.06-1.96 (m, 4H), 1.70 (d, J=10.4 Hz, 2H).

### Compound 10: 5-(3-(2-(3-(3-bromophenyl)-2-oxotetrahydropyrimidin-1(2H)-yl)ethyl)azetidin-1-yl)-2-(2,6-dioxopiperidin-3-yl)isoindoline-1,3-dione

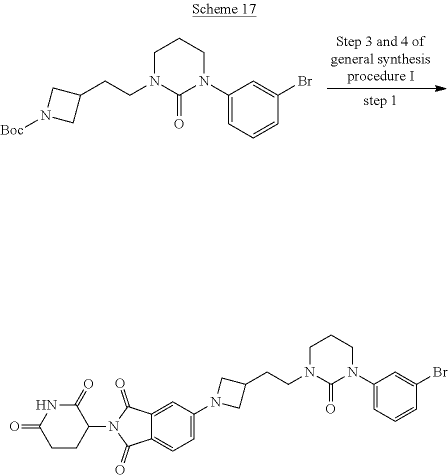 [SMILES: CC(C)(C)OC(=O)N1CC(CCN2CCCN(c3cccc(Br)c3)C2=O)C1 ; O=C1CCC(N2C(=O)c3ccc(N4CC(CCN5CCCN(c6cccc(Br)c6)C5=O)C4)cc3C2=O)C(=O)N1]

Step 1. Preparation of 5-(3-(2-(3-(3-bromophenyl)-2-oxotetrahydropyrimidin-1(2H)-yl)ethyl)azetidin-1-yl)-2-(2,6-dioxopiperidin-3-yl)isoindoline-1,3-dione: Following step 3 and 4 of general synthesis procedure I, from tert-butyl 3-(2-(3-(3-bromophenyl)-2-oxotetrahydropyrimidin-1(2H)-yl)ethyl)azetidine-1-carboxylate, compound 5-(3-(2-(3-(3-bromophenyl)-2-oxotetrahydropyrimidin-1(2H)-yl)ethyl)azetidin-1-yl)-2-(2,6-dioxopiperidin-3-yl)isoindoline-1,3-dione was obtained as a yellow solid (25 mg, 13%). Mass (m/z): 596.1 [M+H]⁺. ¹H NMR (400 MHz, DMSO-d₆) δ 11.08 (s, 1H), 7.63 (d, J=8.4 Hz, 1H), 7.52 (d, J=2.0 Hz, 1H), 7.31-7.22 (m, 3H), 6.77 (d, J=2.0 Hz, 1H), 6.63 (dd, J=8.4, 2.0 Hz, 1H), 5.05 (dd, J=12.8, 5.2 Hz, 1H), 4.14 (t, J=8.2 Hz, 2H), 3.73-3.63 (m, 4H), 3.30 (d, J=7.0 Hz, 3H), 2.92-2.75 (m, 2H), 2.56 (dd, J=15.2, 9.2 Hz, 2H), 2.09-1.94 (m, 4H), 1.88 (dd, J=14.2, 7.2 Hz, 2H).

### Compound 11: 2-(2,6-dioxopiperidin-3-yl)-5-(4-((2-oxo-3-(quinolin-6-yl)tetrahydropyrimidin-1(2H)-yl)methyl)piperidin-1-yl)isoindoline-1,3-dione

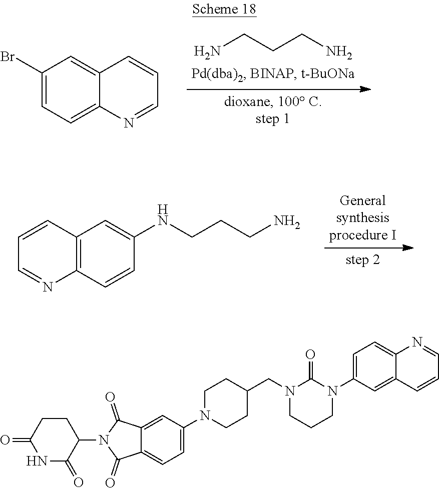 [SMILES: NCCCN ; Brc1ccc2ncccc2c1 ; NCCCNc1ccc2ncccc2c1 ; O=C1CCC(N2C(=O)c3ccc(N4CCC(CN5CCCN(c6ccc7ncccc7c6)C5=O)CC4)cc3C2=O)C(=O)N1]

Step 1. Preparation of N¹-(quinolin-6-yl)propane-1,3-diamine: To a solution of 6-bromoquinoline (2 g, 9.62 mmol), propane-1,3-diamine (2.14 g, 28.86 mmol), BINAP (270 mg, 0.028 mmol) and t-BuONa (1.4 g, 14.43 mmol) in 1, 4-dioxane (10 mL) was added Pd(dba)₂ (220 mg, 0.38 mmol). The resulting mixture was stirred at 100° C. under N₂ for 16 hrs. The solvent was removed under reduced pressure and the residue was purified by Combi-flash [DCM/MeOH (added 10% NH₃)=0˜10%] to give the product N¹-(quinolin-6-yl)propane-1,3-diamine as brown oil (1.69 g, 87%). Mass (m/z): 202.0 [M+H]⁺.

Step 2. Preparation of 2-(2,6-dioxopiperidin-3-yl)-5-(4-((2-oxo-3-(quinolin-6-yl)tetrahydropyrimidin-1(2H)-yl)methyl)piperidin-1-yl)isoindoline-1,3-dione: Following general synthesis procedure I, from N¹-(quinolin-6-yl)propane-1,3-diamine and tert-butyl 4-formylpiperidine-1-carboxylate, compound 2-(2,6-dioxopiperidin-3-yl)-5-(4-((2-oxo-3-(quinolin-6-yl)tetrahydropyrimidin-1(2H)-yl)methyl)piperidin-1-yl)isoindoline-1,3-dione was obtained as a yellow solid (35 mg, 19%). Mass (m/z): 581.1 [M+H]⁺. ¹H NMR (400 MHz, CDCl₃) δ 8.86 (dd, J=4.4, 1.6 Hz, 1H), 8.20 (s, 2H), 8.01 (s, 1H), 7.83 (dd, J=9.0, 2.2 Hz, 1H), 7.73 (d, J=2.0 Hz, 1H), 7.67 (d, J=8.6 Hz, 1H), 7.46 (s, 1H), 7.04 (dd, J=8.6, 2.4 Hz, 1H), 4.93 (dd, J=12.4, 5.4 Hz, 1H), 3.97 (d, J=13.2 Hz, 2H), 3.91-3.83 (m, 2H), 3.50 (t, J=5.8 Hz, 2H), 3.34 (d, J=7.2 Hz, 2H), 3.00 (t, J=11.4 Hz, 2H), 2.92-2.72 (m, 3H), 2.27-2.19 (m, 2H), 2.17-2.07 (m, 2H), 1.86 (d, J=11.2 Hz, 2H), 1.39 (dd, J=9.4, 6.0 Hz, 2H).

### Compound 12: 2-(2,6-dioxopiperidin-3-yl)-5-(3-(2-(2-oxo-3-phenylimidazolidin-1-yl)ethyl) azetidin-1-yl)isoindoline-1,3-dione

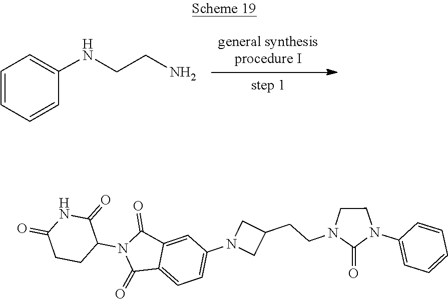 [SMILES: NCCNc1ccccc1 ; O=C1CCC(N2C(=O)c3ccc(N4CC(CCN5CCN(c6ccccc6)C5=O)C4)cc3C2=O)C(=O)N1]

Step 1. Preparation of 2-(2,6-dioxopiperidin-3-yl)-5-(3-(2-(2-oxo-3-phenylimidazolidin-1-yl)ethyl) azetidin-1-yl)isoindoline-1,3-dione: Following general synthesis procedure I, from N¹-phenylethane-1,2-diamine and tert-butyl 3-(2-oxoethyl)azetidine-1-carboxylate, compound 2-(2,6-dioxopiperidin-3-yl)-5-(3-(2-(2-oxo-3-phenylimidazolidin-1-yl)ethyl) azetidin-1-yl)isoindoline-1,3-dione was obtained as a yellow solid (54 mg, 13%). Mass (m/z): 502.2 [M+H]⁺. ¹H NMR (400 MHz, CDCl₃) δ 8.03 (s, 1H), 7.63 (d, J=8.4 Hz, 1H), 7.55 (d, J=8.0 Hz, 2H), 7.34 (t, J=8.0 Hz, 2H), 7.05 (t, J=7.4 Hz, 1H), 6.76 (d, J=2.0 Hz, 1H), 6.49 (dd, J=8.4, 2.0 Hz, 1H), 4.93 (dd, J=12.2, 5.2 Hz, 1H), 4.18 (t, J=8.0 Hz, 2H), 3.91-3.81 (m, 2H), 3.72 (dd, J=7.8, 5.6 Hz, 2H), 3.55-3.46 (m, 2H), 3.36 (t, J=6.8 Hz, 2H), 2.92-2.70 (m, 4H), 2.12 (dd, J=7.8, 5.4 Hz, 1H), 2.03-1.91 (m, 2H).

### Compound 13: 3-(4-{4-[(2-oxo-3-phenyl-1,3-diazinan-1-yl)methyl]piperidin-1-yl}phenyl) piperidine-2,6-dione

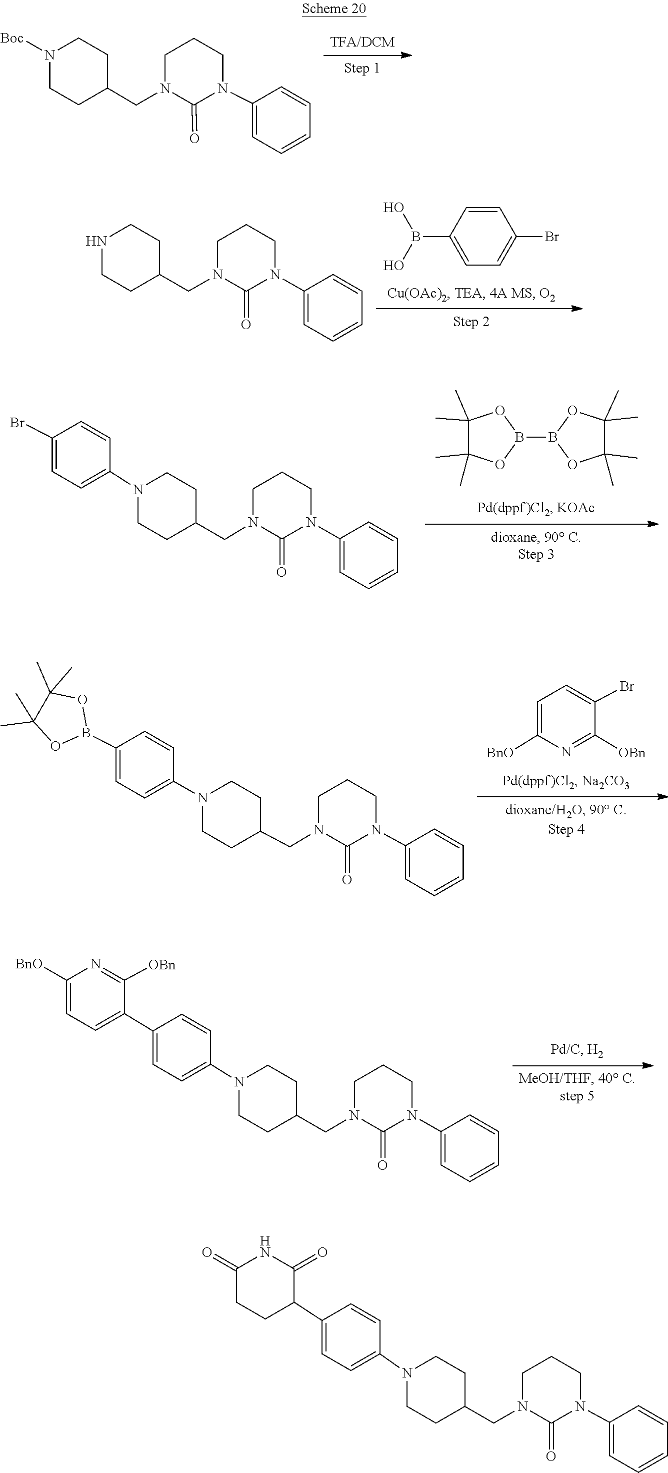

Step 1. Preparation of 1-phenyl-3-(piperidin-4-ylmethyl)-1,3-diazinan-2-one: To a solution of tert-butyl 4-((2-oxo-3-phenyltetrahydropyrimidin-1(2H)-yl)methyl)piperidine-1-carboxylate (230 mg, 0.61 mmol) in DCM (5 mL) was added TFA (2.5 mL). The reaction mixture was stirred at 25° C. under N₂ for 2 hrs. The solution was concentrated under vacuum to give the desired product (1.97 g, 80%) as yellow oil. Mass (m/z): 274.3 [M+H]⁺.

Step 2. Preparation of 1-{[1-(4-bromophenyl)piperidin-4-yl]methyl}-3-phenyl-1,3-diazinan-2-one: To a solution of 1-phenyl-3-(piperidin-4-ylmethyl)-1,3-diazinan-2-one (170 mg, 0.62 mmol) in DCM (10 mL) was added (4-bromophenyl)boranediol (150 mg, 0.75 mmol), Cu(OAc)₂ (169 mg, 0.93 mmol), TEA (252 mg, 2.49 mmol) and 4A Molecular sieves (100 mg). The reaction mixture was stirred at 25° C. under 02 for 16 hrs. The solvent was removed under reduced pressure and the residue was purified by Combi-flash (PE/EA=1:1) to give the product (150 mg, 51%) as a light yellow solid. Mass (m/z): 427.8 [M+H]⁺.

Step 3. Preparation of 1-phenyl-3-({1-[4-(4,4,5,5-tetramethyl-1,3,2-dioxaborolan-2-yl)phenyl]piperidin-4-yl}methyl)-1,3-diazinan-2-one: To a solution of 1-{[1-(4-bromophenyl)piperidin-4-yl]methyl}-3-phenyl-1,3-diazinan-2-one (150 mg, 0.35 mmol) and 4,4,4′,4′,5,5,5′,5′-octamethyl-2,2′-bi(1,3,2-dioxaborolane) (249 mg, 0.98 mmol) in 1, 4-dioxane (10 mL) was added Pd(dppf)Cl₂ (26 mg, 0.035 mmol) and KOAc (103 mg, 1.05 mmol) at 25° C. The reaction mixture was stirred at 90° C. under N₂ for 4 hrs. After the reaction completed, H₂O (10 mL) was added to the reaction mixture, and then extracted with EA (20 mL×3). The combined organic layer was washed with brine (50 mL×2), then dried over anhydrous Na₂SO₄. After filtration, the solution was concentrated under vacuum, and the residue was purified by Combi-flash (PE/EA=1:1) to give the product (130 mg, 62%) as yellow oil. Mass (m/z): 476.0 [M+H]⁺.

Step 4. Preparation of 1-[(1-{4-[2,6-bis(benzyloxy)pyridin-3-yl]phenyl}piperidin-4-yl)methyl]-3-phenyl-1,3-diazinan-2-one: To a solution of 1-phenyl-3-({1-[4-(4,4,5,5-tetramethyl-1,3,2-dioxaborolan-2-yl)phenyl]piperidin-4-yl}methyl)-1,3-diazinan-2-one (150 mg, 0.31 mmol) in 1,4-dioxane/H₂O (10/1, 11 mL) was added 2,6-bis(benzyloxy)-3-bromopyridine (116 mg, 0.31 mmol), Na₂CO₃ (100 mg, 0.95 mmol) and Pd(dppf)Cl₂ (23 mg, 0.031 mmol). The reaction mixture was stirred at 90° C. under N₂ for 4 hrs. The resulting mixture was concentrated and purified by flash chromatography (PE/EA=1:1) to give the product (120 mg, 49%) as yellow oil. Mass (m/z): 638.9 [M+H]⁺.

Step 5. Preparation of 3-(4-{4-[(2-oxo-3-phenyl-1,3-diazinan-1-yl)methyl]piperidin-1-yl}phenyl)piperidine-2,6-dione: To a solution of 1-[(1-{4-[2,6-bis(benzyloxy)pyridin-3-yl]phenyl}piperidin-4-yl)methyl]-3-phenyl-1,3-diazinan-2-one (120 mg, 0.19 mmol) in MeOH (5 mL) and THF (5 mL) was added 10% Pd/C (60 mg, 50% wt/wt). The reaction mixture was stirred at 40° C. under 0.4 MPa of H₂ atmosphere for 16 hrs. After filtration, the solution was concentrated under vacuum. The residue was purified by flash chromatography (DCM/MeOH=0˜5%) to give the desired product (45 mg, 48%) as a yellow solid. Mass (m/z): 461.3 [M+H]⁺. ¹H NMR (400 MHz, DMSO-d₆) δ 10.77 (s, 1H), 7.33-7.23 (m, 4H), 7.13-6.99 (m, 3H), 6.88 (d, J=8.2 Hz, 2H), 3.68 (dt, J=11.0, 5.2 Hz, 5H), 3.38 (s, 2H), 3.20 (d, J=7.2 Hz, 2H), 2.63 (t, J=11.8 Hz, 3H), 2.16-1.96 (m, 4H), 1.69 (d, J=11.8 Hz, 3H), 1.25 (d, J=12.2 Hz, 3H).

### Compound 14: 5-(4-((3-(3-chlorophenyl)-2-oxotetrahydropyrimidin-1(2H)-yl)methyl)piperidin-1-yl)-2-(2,6-dioxopiperidin-3-yl)isoindoline-1,3-dione

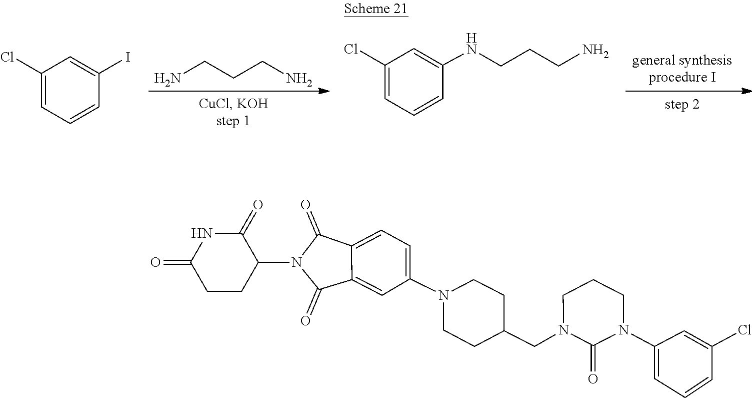 [SMILES: Clc1cccc(I)c1 ; NCCCN ; NCCCNc1cccc(Cl)c1 ; O=C1CCC(N2C(=O)c3ccc(N4CCC(CN5CCCN(c6cccc(Cl)c6)C5=O)CC4)cc3C2=O)C(=O)N1]

Step 1. Preparation of N¹-(3-chlorophenyl)propane-1,3-diamine: A mixture of 1-chloro-3-iodobenzene (2 g, 8.4 mmol), propane-1,3-diamine (1.86 g, 25.21 mmol), KOH (941 mg, 16.8 mmol) and CuCl (83 mg, 0.84 mmol) was stirred at 0° C. under N₂ for 16 hrs. After the reaction completed, H₂O (200 mL) was added to the reaction mixture, and then extracted with DCM (200 mL×3). The combined organic layer was washed with brine (300 mL×2), then dried over anhydrous Na₂SO₄. After filtration, the solution was concentrated under vacuum, and the residue was purified by Combi-flash [DCM/MeOH (10% NH₃·H₂O)=0˜10%] to give the product N¹-(3-chlorophenyl)propane-1,3-diamine as yellow oil (1.34 g, 85%). Mass (m/z): 185.2 [M+H]⁺.

Step 2. Preparation of 5-(4-((3-(3-chlorophenyl)-2-oxotetrahydropyrimidin-1(2H)-yl)methyl)piperidin-1-yl)-2-(2,6-dioxopiperidin-3-yl)isoindoline-1,3-dione: Following general synthesis procedure I, from N¹-(3-chlorophenyl)propane-1,3-diamine, compound 5-(4-((3-(3-chlorophenyl)-2-oxotetrahydropyrimidin-1(2H)-yl)methyl)piperidin-1-yl)-2-(2,6-dioxopiperidin-3-yl)isoindoline-1,3-dione was obtained as a yellow solid (55 mg, 10%). Mass (m/z): 563.9 [M+H]⁺. ¹H NMR (400 MHz, CDCl₃) δ 8.00 (s, 1H), 7.71 (d, J=8.4 Hz, 1H), 7.37 (s, 1H), 7.28 (dd, J=3.8, 2.0 Hz, 1H), 7.24 (d, J=6.4 Hz, 1H), 7.19-7.14 (m, 2H), 4.95 (dd, J=12.4, 5.2 Hz, 1H), 3.94 (d, J=13.2 Hz, 2H), 3.75-3.67 (m, 2H), 3.47 (t, J=5.8 Hz, 2H), 3.31 (d, J=7.2 Hz, 2H), 3.04 (t, J=12.0 Hz, 2H), 2.92-2.72 (m, 4H), 2.19-2.11 (m, 3H), 1.88 (d, J=12.4 Hz, 2H), 1.48 (d, J=10.8 Hz, 2H).

### Compound 15: 5-(4-((3-(3-cyclopropylphenyl)-2-oxotetrahydropyrimidin-1(2H)-yl)methyl) piperidin-1-yl)-2-(2,6-dioxopiperidin-3-yl)isoindoline-1,3-dione

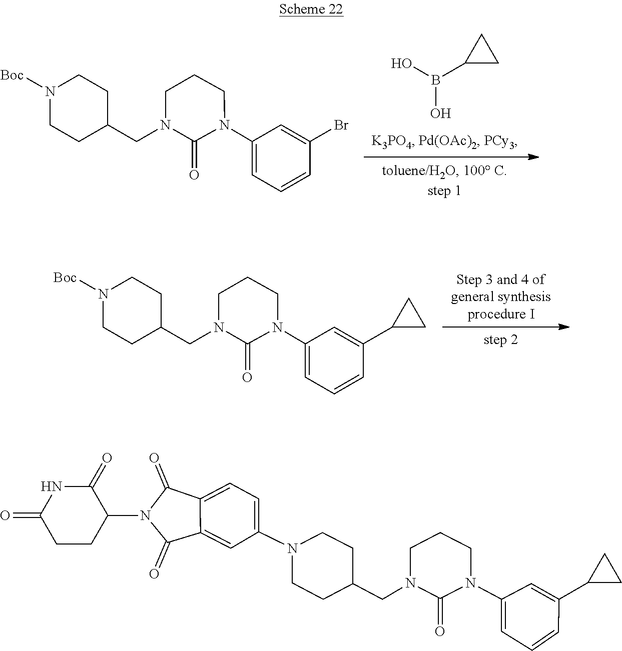 [SMILES: CC(C)(C)OC(=O)N1CCC(CN2CCCN(c3cccc(Br)c3)C2=O)CC1 ; OB(O)C1CC1 ; CC(C)(C)OC(=O)N1CCC(CN2CCCN(c3cccc(C4CC4)c3)C2=O)CC1 ; O=C1CCC(N2C(=O)c3ccc(N4CCC(CN5CCCN(c6cccc(C7CC7)c6)C5=O)CC4)cc3C2=O)C(=O)N1]

Step 1. Preparation of tert-butyl 4-((3-(3-cyclopropylphenyl)-2-oxotetrahydro pyrimidin-1(2H)-yl)methyl)piperidine-1-carboxylate: To a solution of tert-butyl 4-((3-(3-bromophenyl)-2-oxotetrahydropyrimidin-1(2H)-yl)methyl)piperidine-1-carboxylate (500 mg, 1.11 mmol) in Toluene/H₂O (20/1, 20 mL) was added cyclopropylboronic acid (124 mg, 1.44 mmol), K₃PO₄ (705 mg, 3.33 mmol), PCy₃ (31 mg, 0.11 mmol) and Pd(OAc)₂ (12 mg, 0.06 mmol). The reaction mixture was stirred at 100° C. under N₂ for 3 hrs. The solvent was removed under reduced pressure and the residue was purified by Combi-flash (PE/EA=0˜30%) to give the product tert-butyl 4-((3-(3-cyclopropylphenyl)-2-oxotetrahydropyrimidin-1(2H)-yl)methyl)piperidine-1-carboxylate as yellow oil (260 mg, 56%). Mass (m/z): 436.3 [M+Na]⁺.

Step 2. Preparation of 5-(4-((3-(3-cyclopropylphenyl)-2-oxotetrahydropyrimidin-1(2H)-yl)methyl)piperidin-1-yl)-2-(2,6-dioxopiperidin-3-yl)isoindoline-1,3-dione: Following step 3 and step 4 of general synthesis procedure I, from tert-butyl 4-((3-(3-cyclopropylphenyl)-2-oxotetrahydro pyrimidin-1(2H)-yl)methyl)piperidine-1-carboxylate, compound 5-(4-((3-(3-cyclopropylphenyl)-2-oxotetrahydropyrimidin-1(2H)-yl)methyl)piperidin-1-yl)-2-(2,6-dioxopiperidin-3-yl)isoindoline-1,3-dione was obtained as a yellow solid (90 mg, 20%). Mass (m/z): 569.8 [M+H]⁺. ¹H NMR (400 MHz, CDCl₃) δ 8.06 (s, 1H), 7.69 (d, J=8.4 Hz, 1H), 7.34 (s, 1H), 7.23-7.13 (m, 2H), 7.04 (d, J=8.4 Hz, 2H), 6.84 (d, J=7.6 Hz, 1H), 4.98-4.90 (m, 1H), 3.93 (d, J=13.0 Hz, 2H), 3.73-3.66 (m, 2H), 3.44 (t, J=5.8 Hz, 2H), 3.30 (d, J=7.2 Hz, 2H), 3.00 (d, J=10.8 Hz, 2H), 2.92-2.70 (m, 3H), 2.12 (dd, J=12.2, 5.8 Hz, 4H), 1.92-1.83 (m, 3H), 1.44 (d, J=9.6 Hz, 2H), 0.97-0.89 (m, 2H), 0.68 (dd, J=5.0, 1.6 Hz, 2H).

### Compound 16: 2-(2,6-dioxopiperidin-3-yl)-5-(4-((3-(4-fluorobenzyl)-2-oxotetrahydropyrimidin-1(2H)-yl)methyl)piperidin-1-yl)isoindoline-1,3-dione

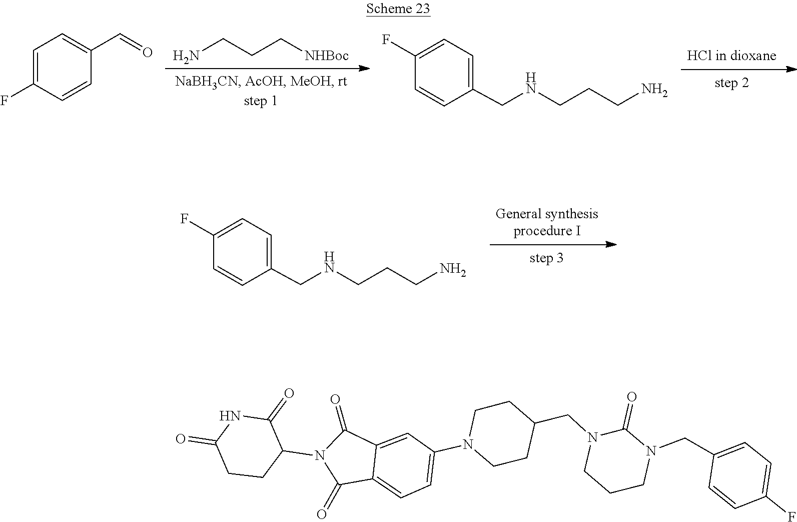

Step 1. Preparation of tert-butyl (3-((4-fluorobenzyl)amino)propyl)carbamate: To a solution of 4-fluorobenzaldehyde (1 g, 8.06 mmol) MeOH (30 mL) was added tert-butyl (3-aminopropyl)carbamate (1.4 g, 8.06 mmol), AcOH (0.05 mL) and NaBH₃CN (1.52 g, 25.19 mmol) at 0° C. The solvent was removed under reduced pressure and the residue was purified by Combi-flash [DCM/MeOH (10% NH₃·H₂O)=0˜10%] to give the product tert-butyl (3-((4-fluorobenzyl)amino)propyl)carbamate as colorless oil (2 g, 88%). Mass (m/z): 283.3 [M+H]⁺.

Step 2. Preparation of N¹-(4-fluorobenzyl)propane-1,3-diamine: A solution of tert-butyl (3-((4-fluorobenzyl)amino)propyl)carbamate (2 g, 7.09 mmol) in HCl/dioxane (4.0 M, 40 mL) was stirred at rt for 2 hrs. The solvent was removed under reduced pressure to give the product N¹-(4-fluorobenzyl) propane-1,3-diamine as a white solid (1.5 g, 81%). Mass (m/z): 183.0 [M+H]⁺.

Step 3. Preparation of 2-(2,6-dioxopiperidin-3-yl)-5-(4-((3-(4-fluorobenzyl)-2-oxotetrahydropyrimidin-1(2H)-yl)methyl)piperidin-1-yl)isoindoline-1,3-dione: Following general synthesis procedure I, from N¹-(4-fluorobenzyl)propane-1,3-diamine, compound 2-(2,6-dioxopiperidin-3-yl)-5-(4-((3-(4-fluorobenzyl)-2-oxotetrahydropyrimidin-1(2H)-yl)methyl)piperidin-1-yl)isoindoline-1,3-dione was obtained as a yellow solid (45 mg, 12%). Mass (m/z): 562.3 [M+H]⁺. ¹H NMR (400 MHz, CDCl₃) δ 8.15 (s, 1H), 7.67 (d, J=8.4 Hz, 1H), 7.24 (dd, J=8.4, 3.0 Hz, 2H), 7.07-6.97 (m, 3H), 4.94 (dd, J=12.4, 5.2 Hz, 1H), 4.52 (s, 2H), 3.96 (d, J=13.2 Hz, 2H), 3.34-3.25 (m, 4H), 3.19 (t, J=5.8 Hz, 2H), 3.04-2.95 (m, 2H), 2.87-2.72 (m, 3H), 2.15-2.09 (m, 1H), 2.05-1.98 (m, 1H), 1.94 (dt, J=11.6, 5.8 Hz, 2H), 1.81 (d, J=10.8 Hz, 2H), 1.42-1.32 (m, 2H).

### Compound 17: 2-(2,6-dioxopiperidin-3-yl)-5-(4-((3-(4-fluorobenzyl)-2-oxoimidazolidin-1-yl)methyl)piperidin-1-yl)isoindoline-1,3-dione

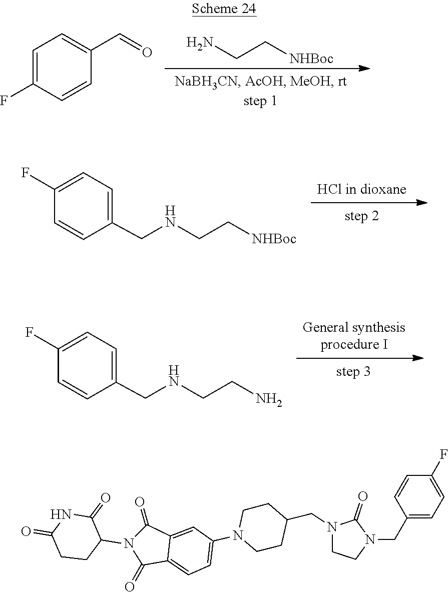 [SMILES: O=Cc1ccc(F)cc1 ; CC(C)(C)OC(=O)NCCN ; CC(C)(C)OC(=O)NCCNCc1ccc(F)cc1 ; NCCNCc1ccc(F)cc1 ; O=C1CCC(N2C(=O)c3ccc(N4CCC(CN5CCN(Cc6ccc(F)cc6)C5=O)CC4)cc3C2=O)C(=O)N1]

Step 1. Preparation of tert-butyl (2-((4-fluorobenzyl)amino)ethyl)carbamate: To a solution of 4-fluorobenzaldehyde (1 g, 8.06 mmol) in MeOH (30 mL) was added tert-butyl (2-aminoethyl)carbamate (1.29 g, 8.06 mmol), AcOH (0.05 mL) and NaBH₃CN (1.52 g, 25.19 mmol) at 0° C. The solvent was removed under reduced pressure and the residue was purified by Combi-flash [DCM/MeOH (10% NH₃·H₂O)=0˜10%] to give the product tert-butyl (3-((4-fluorobenzyl) amino) propyl) carbamate as colorless oil (1.46 g, 67%). Mass (m/z): 269.2 [M+H]⁺.

Step 2. Preparation of N¹-(4-fluorobenzyl)ethane-1,2-diamine: A solution of tert-butyl (3-((4-fluorobenzyl)amino)propyl)carbamate (1.46 g, 5.45 mmol) in HCl/dioxane (4.0 M, 40 mL) was stirred at rt for 2 hrs. The solvent was removed under reduced pressure to give the product N¹-(4-fluorobenzyl)ethane-1,2-diamine as a white solid (1.3 g, 64%). Mass (m/z): 169.0 [M+H]⁺.

Step 3. Preparation of 2-(2,6-dioxopiperidin-3-yl)-5-(4-((3-(4-fluorobenzyl)-2-oxoimidazolidin-1-yl)methyl)piperidin-1-yl)isoindoline-1,3-dione: Following general synthesis procedure I, from N¹-(4-fluorobenzyl)ethane-1,2-diamine, the product 2-(2,6-dioxopiperidin-3-yl)-5-(4-((3-(4-fluorobenzyl)-2-oxoimidazolidin-1-yl)methyl)piperidin-1-yl)isoindoline-1,3-dione was obtained as a yellow solid (60 mg, 16%). Mass (m/z): 548.0 [M+H]⁺. ¹H NMR (400 MHz, CDCl₃) δ 8.16 (s, 1H), 7.68 (d, J=8.4 Hz, 1H), 7.30 (s, 1H), 7.25-7.22 (m, 1H), 7.12-6.97 (m, 3H), 4.94 (dd, J=12.0, 5.2 Hz, 1H), 4.34 (s, 2H), 3.95 (d, J=12.8 Hz, 2H), 3.34 (t, J=7.6 Hz, 2H), 3.24-3.11 (m, 4H), 2.99 (t, J=11.8 Hz, 2H), 2.92-2.72 (m, 3H), 2.19-2.08 (m, 1H), 1.92-1.80 (m, 3H), 1.41 (dd, J=22.4, 9.4 Hz, 2H).

### Compound 18: 2-(2,6-dioxopiperidin-3-yl)-5-(4-{[3-(4-methyl-1,3-thiazol-2-yl)-2-oxo-1,3-diazinan-1-yl]methyl}piperidin-1-yl)isoindole-1,3-dione

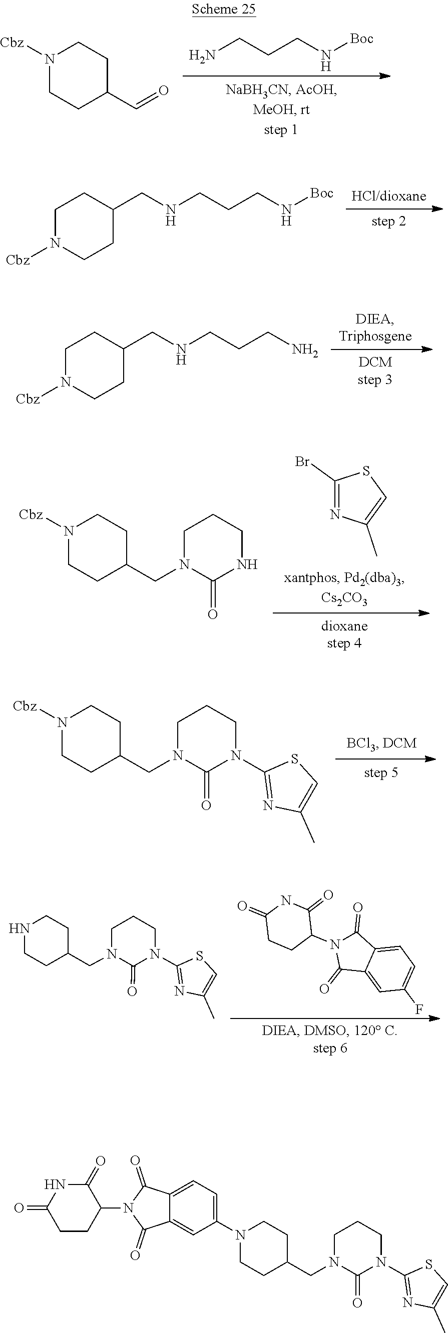

Step 1. Preparation of benzyl 4-(((3-((tert-butoxycarbonyl)amino)propyl)amino)methyl)piperidine-1-carboxylate: Following step 1 of general synthesis procedure I, the product was obtained as yellow oil (5 g, 66%). Mass (m/z): 406.3 [M+H]⁺.

Step 2. Preparation of benzyl 4-(((3-aminopropyl)amino)methyl)piperidine-1-carboxylate: To a solution of benzyl 4-(((3-((tert-butoxycarbonyl)amino)propyl)amino)methyl)piperidine-1-carboxylate (5 g, 12 mmol) in DCM (25 mL) and HCl/dioxane (4.0 M, 25 mL). The reaction mixture was stirred at 25° C. under N₂ for 3 hrs. The solution was concentrated under vacuum to give the desired product (1.97 g, 80%) as a white solid. Mass (m/z): 306.2 [M+H]⁺.

Step 3. Preparation of benzyl 4-((2-oxotetrahydropyrimidin-1(2H)-yl)methyl)piperidine-1-carboxylate: Following step 2 of general synthesis procedure I, the product was obtained as a yellow solid (1.16 g, 46%). Mass (m/z): 332.2 [M+H]⁺.

Step 4. Preparation of benzyl 4-((3-(4-methylthiazol-2-yl)-2-oxotetrahydropyrimidin-1(2H)-yl)methyl)piperidine-1-carboxylate: To a solution of benzyl 4-((2-oxotetrahydropyrimidin-1(2H)-yl)methyl)piperidine-1-carboxylate (500 mg, 1.51 mmol) in 1.4-dioxane (30 mL) was added 2-bromo-4-methyl-1,3-thiazole (268 mg, 1.51 mmol), xantphos (349 mg, 0.60 mmol), Pd₂(dba)₃ (165 mg, 0.18 mmol) and Cs₂CO₃ (1.77 g, 5.43 mmol) at 25° C. The reaction mixture was stirred at 100° C. under N₂ for 4 hrs. After the reaction completed, H₂O (50 mL) was added to the reaction mixture, and then extracted with EA (50 mL×3). The combined organic layer was washed with brine (50 mL×2), then dried over anhydrous Na₂SO₄. After filtration, the solution was concentrated under vacuum, and the residue was purified by Combi-flash (DCM/MeOH=20:1) to give the product as a brown solid (280 mg, 26%). Mass (m/z): 429.2 [M+H]⁺.

Step 5. Preparation of 1-(4-methyl-1,3-thiazol-2-yl)-3-(piperidin-4-ylmethyl)-1,3-diazinan-2-one: To a solution of benzyl 4-((3-(4-methylthiazol-2-yl)-2-oxotetrahydropyrimidin-1(2H)-yl)methyl)piperidine-1-carboxylate (220 mg, 0.51 mmol) in DCM (5 mL) was added BCl₃ (1.0 M in DCM, 4 mL, 3.95 mmol) at −78° C. under N₂. The reaction mixture was stirred at 25° C. under N₂ for 16 hrs. After the reaction completed, H₂O (20 mL) was added to the reaction mixture at 0° C., and then extracted with DCM (20 mL×3). The combined organic layer was washed with brine (20 mL×2), then dried over anhydrous Na₂SO₄. After filtration, the solution was concentrated under vacuum, and the residue was purified by prep-TLC (PE/EA=1:1) to give the product as a yellow solid (122 mg, 63%). Mass (m/z): 295.0 [M+H]⁺.

Step 6. Preparation of 2-(2,6-dioxopiperidin-3-yl)-5-(4-{[3-(4-methyl-1,3-thiazol-2-yl)-2-oxo-1,3-diazinan-1-yl]methyl}piperidin-1-yl)isoindole-1,3-dione: Following step 4 of general synthesis procedure I, the desired product was obtained (43 mg, 18%) as a yellow solid. Mass (m/z): 551.2 [M+H]⁺. ¹H NMR (400 MHz, DMSO-d₆) δ 11.08 (s, 1H), 7.65 (d, J=8.4 Hz, 1H), 7.31 (s, 1H), 7.24 (d, J=8.4 Hz, 1H), 6.62 (s, 1H), 5.06 (dd, J=12.8, 5.2 Hz, 1H), 4.04 (dd, J=17.6, 11.6 Hz, 4H), 3.40 (d, J=4.8 Hz, 2H), 3.25 (d, J=7.2 Hz, 2H), 2.94 (dd, J=29.6, 17.4 Hz, 4H), 2.67 (s, 1H), 2.22 (s, 3H), 2.03 (s, 4H), 1.70 (d, J=11.8 Hz, 2H), 1.22 (d, J=11.6 Hz, 2H).

### Compound 19: 3-(1-oxo-5-(4-((2-oxo-3-phenyltetrahydropyrimidin-1(2H)-yl)methyl)piperidin-1-yl)isoindolin-2-yl)piperidine-2,6-dione

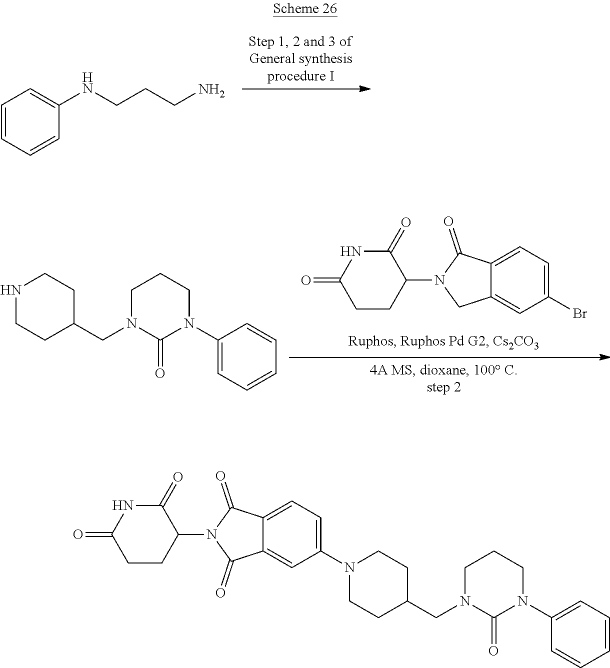 [SMILES: NCCCNc1ccccc1 ; O=C1N(CC2CCNCC2)CCCN1c1ccccc1 ; O=C1CCC(N2Cc3cc(Br)ccc3C2=O)C(=O)N1 ; O=C1CCC(N2C(=O)c3ccc(N4CCC(CN5CCCN(c6ccccc6)C5=O)CC4)cc3C2=O)C(=O)N1]

Step 1. Preparation of 1-phenyl-3-(piperidin-4-ylmethyl)tetrahydropyrimidin-2(1H)-one: Following step 1, 2 and 3 of general synthesis procedure I, the desired product 1-phenyl-3-(piperidin-4-ylmethyl)tetrahydropyrimidin-2(1H)-one was obtained as yellow oil (1800 mg, purity: 50%). Mass (m/z): 296.1 [M+H]⁺.

Step 2. Preparation of 3-(1-oxo-5-(4-((2-oxo-3-phenyltetrahydropyrimidin-1(2H)-yl)methyl)piperidin-1-yl)isoindolin-2-yl)piperidine-2,6-dione: To a solution of 3-(5-bromo-1-oxoisoindolin-2-yl)piperidine-2,6-dione (94 mg, 0.29 mmol) in dioxane (20 mL) was added 1-phenyl-3-(piperidin-4-ylmethyl)-1,3-diazinan-2-one (200 mg, 0.73 mmol), Cs₂CO₃ (284 mg, 0.87 mmol), Ruphos (27 mg, 0.06 mmol), RuPhos Pd G2 (45 mg, 0.06 mmol) and 4A molecular sieves (4 mg, 0.008 mmol). The reaction mixture was stirred at 100° C. under N₂ for 16 hrs. After the reaction completed, H₂O (20 mL) was added to the reaction mixture, and then extracted with EA (20 mL×3). The combined organic layer was washed with brine (30 mL×2), then dried over anhydrous Na₂SO₄. After filtration, the solution was concentrated under vacuum, and the residue was purified by Combi-flash (DCM/MeOH=0˜10%) to give the product as a white solid (10 mg, 6%). Mass (m/z): 516.3 [M+H]⁺. ¹H (400 MHz, CDCl₃) δ 8.00 (s, 1H), 7.84 (d, J=8.4 Hz, 1H), 7.38-7.27 (m, 4H), 7.21 (dd, J=16.6, 9.2 Hz, 2H), 5.20 (dd, J=13.4, 5.0 Hz, 1H), 4.46 (d, J=15.8 Hz, 1H), 4.34 (s, 1H), 3.81 (d, J=11.8 Hz, 2H), 3.74-3.70 (m, 2H), 3.48 (s, 2H), 3.36 (d, J=6.6 Hz, 2H), 3.10 (s, 2H), 2.95-2.78 (m, 3H), 2.17 (d, J=5.6 Hz, 4H), 2.00 (d, J=12.0 Hz, 2H), 1.72 (s, 2H).

### Compound 20: 3-(4-(4-(2-(2-oxo-3-phenyltetrahydropyrimidin-1(2H)-yl)ethyl)piperidin-1-yl)phenyl)piperidine-2,6-dione

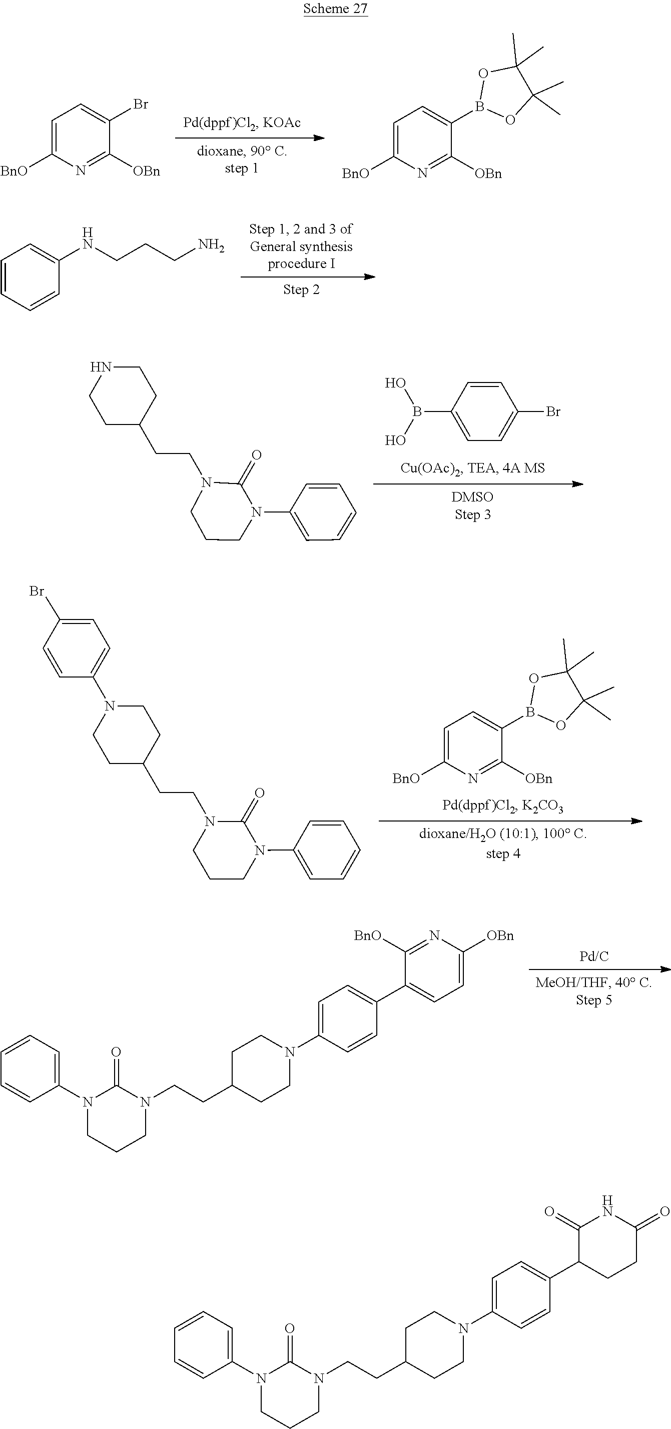

Step 1. Preparation of 2,6-bis(benzyloxy)-3-(4,4,5,5-tetramethyl-1,3,2-dioxaborolan-2-yl)pyridine: To a solution of 2,6-bis(benzyloxy)-3-bromopyridine (2 g, 5.4 mmol), 4,4,4′,4′,5,5, 5′,5′-octamethyl-2,2′-bi(1,3,2-dioxaborolane) (2.06 g, 8.1 mmol) and KOAc (1.06 g, 10.8 mmol) in dioxane (30 mL) was added Pd(dppf)Cl₂ (0.4 g, 0.54 mmol). The reaction mixture was stirred at 90° C. under N₂ for 16 hrs. The mixture was concentrated and the residue was purified by flash column (PE/EA=10:1) to give the product as a brown solid (1.5 g, 70%). Mass (m/z): 418.1 [M+H]⁺.

Step 2. Preparation of 1-phenyl-3-[2-(piperidin-4-yl)ethyl]-1,3-diazinan-2-one: Following step 1, 2 and 3 of general synthesis procedure I, the desired product was obtained as a brown solid (650 mg, purity: ˜50%). Mass (m/z): 288.0 [M+H]⁺.

Step 3. Preparation of 1-{2-[1-(4-bromophenyl)piperidin-4-yl]ethyl}-3-phenyl-1,3-diazinan-2-one: A solution of 1-phenyl-3-[2-(piperidin-4-yl)ethyl]-1,3-diazinan-2-one (500 mg, 1.739 mmol), (4-bromophenyl)boranediol (1.04 g, 5.21 mmol), Cu(OAc)₂ (474 mg, 2.609 mmol), TEA (702 mg, 6.958 mmol) and 4A MS (250 mg) in DCM (20 mL) was stirred at rt under 02 for 16 hrs. The mixture was concentrated and the residue was purified by flash column (DCM:MeOH=20:1) to give the product as a brown solid (280 mg, 36%). Mass (m/z): 442.9 [M+H]⁺.

Step 4. Preparation of 1-(2-(1-(4-(2,6-bis(benzyloxy)pyridin-3-yl)phenyl)piperidin-4-yl)ethyl)-3-phenyltetrahydropyrimidin-2(1H)-one: To a solution of 1-{2-[1-(4-bromophenyl)piperidin-4-yl]ethyl}-3-phenyl-1,3-diazinan-2-one (260 mg, 0.587 mmol), 2,6-bis(benzyloxy)-3-(4,4,5,5-tetramethyl-1,3,2-dioxaborolan-2-yl)pyridine (294 mg, 0.705 mmol) and K₂CO₃ (162 mg, 1.175 mmol) in dioxane/H₂O (10/1, 10 mL) was added Pd(dppf)Cl₂ (43 mg, 0.026 mmol). The reaction mixture was stirred at 100° C. under N₂ for 16 hrs. The mixture was concentrated and the residue was purified by flash column (DCM/MeOH=20:1) to give the product as a yellow solid (110 mg, 28%) Mass (m/z): 652.8 [M+H]⁺.

Step 5. 3-(4-(4-(2-(2-oxo-3-phenyltetrahydropyrimidin-1(2H)-yl)ethyl)piperidin-1-yl)phenyl)piperidine-2,6-dione: To a solution of 1-(2-(1-(4-(2,6-bis(benzyloxy)pyridin-3-yl)phenyl)piperidin-4-yl)ethyl)-3-phenyltetrahydropyrimidin-2(1H)-one (80 mg, 0.122 mmol) in MeOH (4 mL) and THF (4 mL) was added 10% Pd/C (40 mg, 50% wt/wt). The reaction mixture was stirred at 40° C. under H₂ at 0.4 MPa for 16 hrs. The mixture was filtered and the filtrate was concentrated and the residue was purified by Prep-HPLC [Gemini-C18, 150×21.2 mm, 5 um; ACN—H₂O (0.1% FA), 30-50] to give the product as a white solid (17 mg, 29%). Mass (m/z): 476.0 [M+H]⁺. ¹H NMR (400 MHz, DMSO-d₆) δ 10.74 (s, 1H), 7.27-7.19 (m, 4H), 7.11-6.93 (m, 3H), 6.84 (d, J=8.6 Hz, 2H), 3.69-3.57 (m, 6H), 2.61-2.53 (m, 6H), 2.15-1.92 (m, 4H), 1.75 (d, J=12.2 Hz, 2H), 1.40 (dd, J=22.8, 16.0 Hz, 2H), 1.33-1.09 (m, 4H).

### Compound 21: 2-(2,6-dioxopiperidin-3-yl)-5-(3-(2-(3-(3-(imidazo[1,2-a]pyridin-7-yl)phenyl)-2-oxotetrahydropyrimidin-1(2H)-yl)ethyl)azetidin-1-yl)isoindoline-1,3-dione

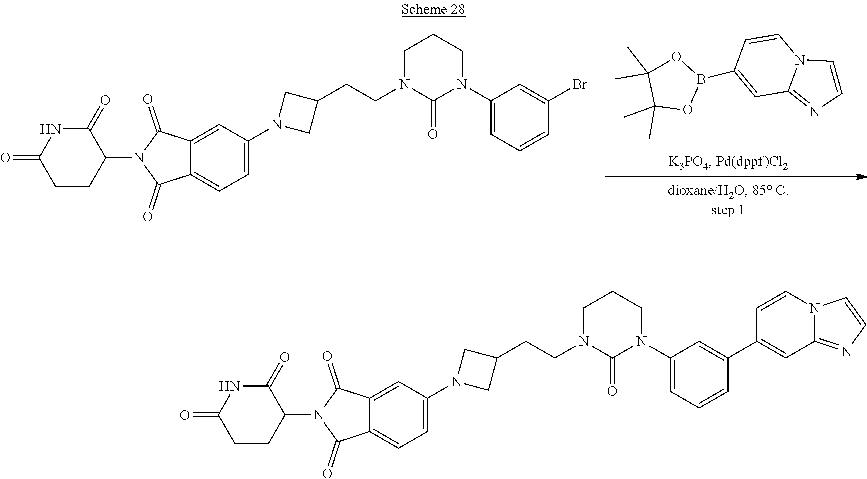

Step 1. Preparation of 2-(2,6-dioxopiperidin-3-yl)-5-(3-(2-(3-(3-(imidazo[1,2-a]pyridin-7-yl)phenyl)-2-oxotetrahydropyrimidin-1(2H)-yl)ethyl)azetidin-1-yl)isoindoline-1,3-dione: To a solution of 5-(3-{2-[3-(3-bromophenyl)-2-oxo-1,3-diazinan-1-yl]ethyl}azetidin-1-yl)-2-(2,6-dioxopiperidin-3-yl)isoindole-1,3-dione (150 mg, 0.25 mmol) and 7-(4,4,5,5-tetramethyl-1,3,2-dioxaborolan-2-yl)imidazo[1,2-a]pyridine (74 mg, 0.30 mmol) in dioxane (5 mL) and water (0.5 mL) was added K₃PO₄ (107 mg, 0.50 mmol) and Pd(dppf)Cl₂ (18 mg, 0.025 mmol). The reaction mixture was stirred at 85° C. under N₂ for 16 hrs. The solvent was removed under reduced pressure and the residue was purified by Pre-HPLC [chromatographic column:—Gemini-C18 150×21.2 mm, Sum; mobile phase: ACN—H₂O (0.1% FA), gradient: 20-50] to give the product as a yellow solid (50 mg, 30%). Mass (m/z): 632.0 [M+H]⁺. ¹H NMR (400 MHz, DMSO-d₆) δ 11.04 (s, 1H), 8.57 (d, J=7.2 Hz, 1H), 7.92 (s, 1H), 7.82 (s, 1H), 7.66 (t, J=1.8 Hz, 1H), 7.58 (d, J=8.2 Hz, 2H), 7.51 (d, J=8.2 Hz, 1H), 7.37 (t, J=7.8 Hz, 1H), 7.29-7.16 (m, 2H), 6.72 (d, J=2.0 Hz, 1H), 6.59 (dd, J=8.4, 2.0 Hz, 1H), 5.01 (dd, J=12.8, 5.4 Hz, 1H), 4.10 (t, J=8.2 Hz, 2H), 3.74-3.64 (m, 4H), 3.36 (t, J=5.9 Hz, 4H), 2.89-2.70 (m, 2H), 2.58-2.47 (m, 2H), 2.06-2.00 (m, 2H), 1.98-1.92 (m, 1H), 1.89-1.81 (m, 2H).

### Compound 22: 5-(3-(2-(3-(3-(1-cyclopropyl-1H-pyrazolo[3,4-b]pyridin-5-yl)phenyl)-2-oxotetrahydropyrimidin-1(2H)-yl)ethyl)azetidin-1-yl)-2-(2,6-dioxopiperidin-3-yl)isoindoline-1,3-dione

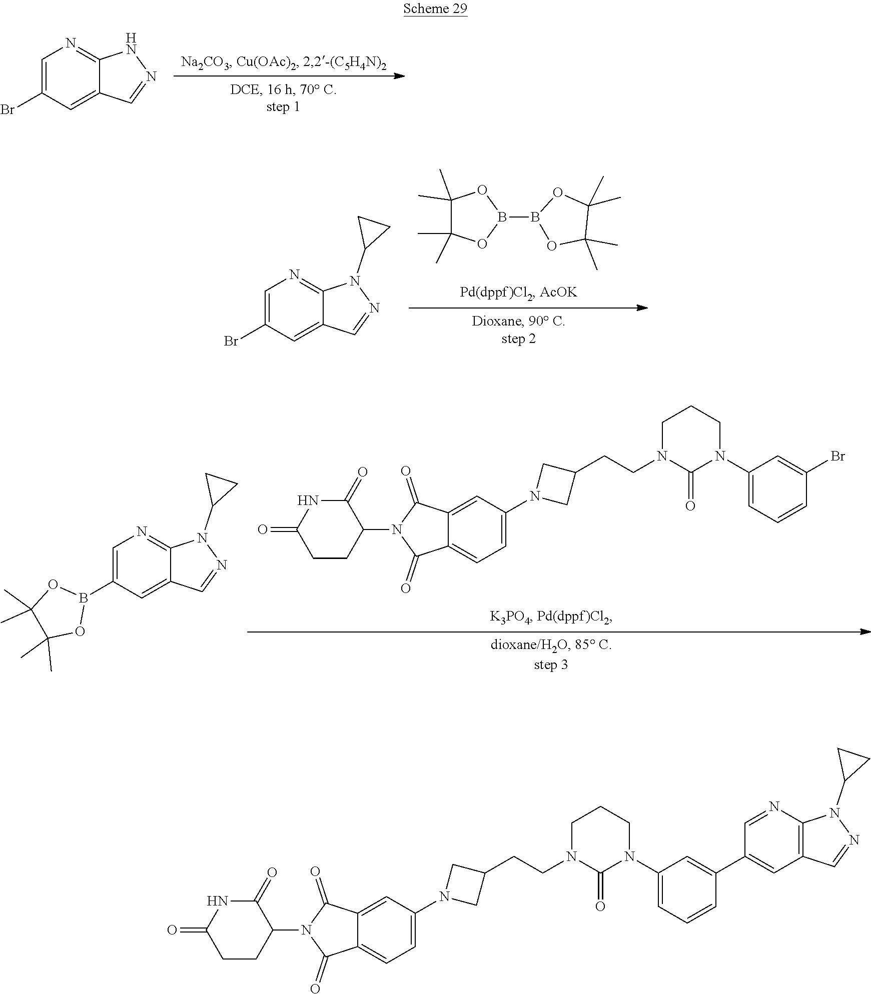

Step 1. Preparation of 5-bromo-1-cyclopropyl-1H-pyrazolo[3,4-b]pyridine: To a solution of 5-bromo-1H-pyrazolo[3,4-b]pyridine (1 g, 5.05 mmol) and cyclopropylboranediol (867 mg, 10.1 mmol) in DCE (30 mL) was added Na₂CO₃ (1.07 g, 10.1 mmol), Cu(OAc)₂ (917 mg, 5.05 mmol) and 2,2′-Bipyridine (789 mg, 5.05 mmol). The reaction mixture was stirred at 70° C. under 02 for 16 hrs. The mixture was filtered and the filtrate was concentrated. The residue was washed with saturated copper sulfate aqueous solution (20 mL) and extracted with EA (20 mL). The organic phase was evaporated to give the product as a yellow solid (800 mg, 53%). Mass (m/z): 238.0 240.0[M+H]⁺.

Step 2. Preparation of 1-cyclopropyl-5-(4,4,5,5-tetramethyl-1,3,2-dioxaborolan-2-yl)-1H-pyrazolo[3,4-b]pyridine: To a solution of 5-bromo-1-cyclopropylpyrazolo[3,4-b]pyridine (700 mg, 2.94 mmol) and B₂(Pin)₂ (896 mg, 3.53 mmol) in dioxane (10 mL) was added KOAc (866 mg, 8.82 mmol) and Pd(dppf)Cl₂ (215 mg, 0.29 mmol). The reaction mixture was stirred at 90° C. under N₂ for 16 hrs. The reaction mixture was concentrated and purified by silica gel column chromatography (PE:EA=2:1) to give the product as a yellow solid (450 mg, 43%). Mass (m/z): 286.1[M+H]⁺.

Step 3. Preparation of 5-(3-(2-(3-(3-(1-cyclopropyl-1H-pyrazolo[3,4-b]pyridin-5-yl)phenyl)-2-oxotetrahydropyrimidin-1(2H)-yl)ethyl)azetidin-1-yl)-2-(2,6-dioxo piperidin-3-yl)isoindoline-1,3-dione: To a solution of 5-(3-{2-[3-(3-bromophenyl)-2-oxo-1,3-diazinan-1-yl]ethyl}azetidin-1-yl)-2-(2,6-dioxopiperidin-3-yl)isoindole-1,3-dione (100 mg, 0.17 mmol) and 1-cyclopropyl-5-(4,4,5,5-tetramethyl-1,3,2-dioxaborolan-2-yl)-1H-pyrazolo[3,4-b]pyridine (72 mg, 0.25 mmol) in dioxane (3 mL) and water (0.3 mL) was added K₃PO₄ (71 mg, 0.34 mmol) and Pd(dppf)Cl₂ (12 mg, 0.017 mmol). The reaction mixture was stirred at 85° C. under N₂ for 16 hrs. The solvent was removed under reduced pressure and the residue was purified by Pre-HPLC [chromatographic column:—Gemini-C18 150×21.2 mm, 5 um; mobile phase: ACN—H₂O (0.1% FA), gradient: 40-70] to give the product as a yellow solid (60 mg, 52%). Mass (m/z): 673.0[M+H]⁺. ¹H NMR (400 MHz, DMSO-d₆) δ 11.07 (s, 1H), 8.87 (d, J=2.0 Hz, 1H), 8.45 (d, J=2.0 Hz, 1H), 8.14 (s, 1H), 7.67-7.58 (m, 2H), 7.51-7.40 (m, 2H), 7.32 (d, J=8.2 Hz, 1H), 6.76 (d, J=2.0 Hz, 1H), 6.63 (dd, J=8.4, 2.0 Hz, 1H), 5.05 (dd, J=12.8, 5.4 Hz, 1H), 4.14 (t, J=8.2 Hz, 2H), 4.00-3.93 (m, 1H), 3.82-3.63 (m, 4H), 3.41 (t, J=5.8 Hz, 2H), 3.32-3.30 (m, 1H), 2.94-2.75 (m, 2H), 2.63-2.51 (m, 3H), 2.12-2.04 (m, 2H), 2.03-1.97 (m, 1H), 1.90 (dd, J=14.0, 7.0 Hz, 2H), 1.24-1.20 (m, 2H), 1.16-1.11 (m, 2H).

### Compound 23: 2-(2,6-dioxopiperidin-3-yl)-5-(3-(2-(2-oxo-3-(3-(quinolin-3-yl)phenyl) tetrahydropyrimidin-1(2H)-yl)ethyl)azetidin-1-yl)isoindoline-1,3-dione

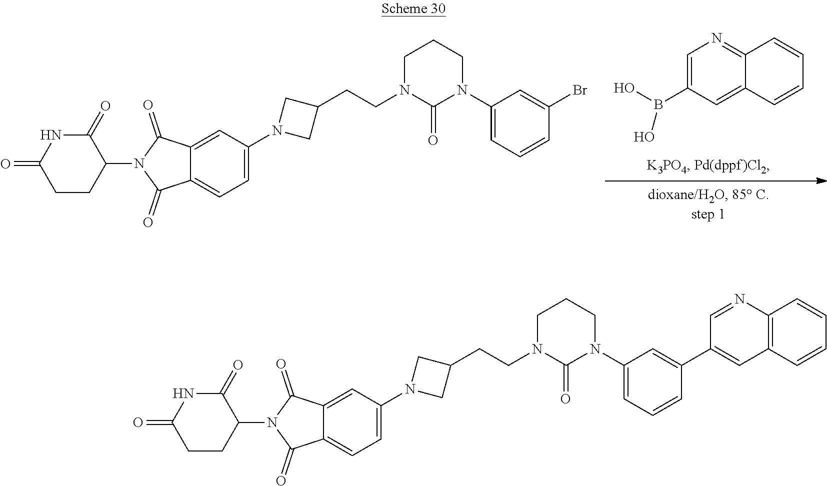 [SMILES: O=C1CCC(N2C(=O)c3ccc(N4CC(CCN5CCCN(c6cccc(Br)c6)C5=O)C4)cc3C2=O)C(=O)N1 ; OB(O)c1cnc2ccccc2c1 ; O=C1CCC(N2C(=O)c3ccc(N4CC(CCN5CCCN(c6cccc(-c7cnc8ccccc8c7)c6)C5=O)C4)cc3C2=O)C(=O)N1]

Step 1. Preparation of 2-(2,6-dioxopiperidin-3-yl)-5-(3-(2-(2-oxo-3-(3-(quinolin-3-yl)phenyl) tetrahydropyrimidin-1(2H)-yl)ethyl)azetidin-1-yl)isoindoline-1,3-dione: To a solution of 5-(3-{2-[3-(3-bromophenyl)-2-oxo-1,3-diazinan-1-yl]ethyl}azetidin-1-yl)-2-(2,6-dioxopiperidin-3-yl)isoindole-1,3-dione (100 mg, 0.17 mmol) and quinolin-3-ylboranediol (44 mg, 0.25 mmol) in dioxane (3 mL) and water (0.3 mL) was added K₃PO₄ (71 mg, 0.34 mmol) and Pd(dppf)Cl₂ (12 mg, 0.017 mmol). The reaction mixture was stirred at 85° C. under N₂ for 16 hrs. The solvent was removed under reduced pressure and the residue was purified by Pre-HPLC [chromatographic column:—Gemini-C18 150×21.2 mm, 5 um; mobile phase: ACN—H₂O (0.1% FA), gradient: 40-70] to give the product as a yellow solid (17 mg, 14%). Mass (m/z): 643.0 [M+H]⁺. ¹H NMR (400 MHz, DMSO-d₆) δ 11.07 (s, 1H), 9.32 (d, J=2.2 Hz, 1H), 8.80 (s, 1H), 8.11 (t, J=7.8 Hz, 2H), 7.87-7.79 (m, 2H), 7.73-7.60 (m, 3H), 7.50 (t, J=7.8 Hz, 1H), 7.39 (d, J=8.0 Hz, 1H), 6.77 (d, J=1.8 Hz, 1H), 6.63 (dd, J=8.4, 2.0 Hz, 1H), 5.05 (dd, J=12.8, 5.2 Hz, 1H), 4.15 (t, J=8.2 Hz, 2H), 3.79-3.77 (m, 2H), 3.71 (dd, J=8.2, 5.8 Hz, 2H), 3.38 (dt, J=13.6, 6.2 Hz, 4H), 2.90-2.75 (m, 2H), 2.64-2.52 (m, 2H), 2.14-2.05 (m, 2H), 2.02-1.97 (m, 1H), 1.90 (dd, J=14.0, 6.8 Hz, 2H).

### Compound 24: 2-(2,6-dioxopiperidin-3-yl)-5-(3-(2-(3-(3-(2-methyloxazolo[4,5-b]pyridin-6-yl)phenyl)-2-oxotetrahydropyrimidin-1(2H)-yl)ethyl)azetidin-1-yl)isoindoline-1,3-dione

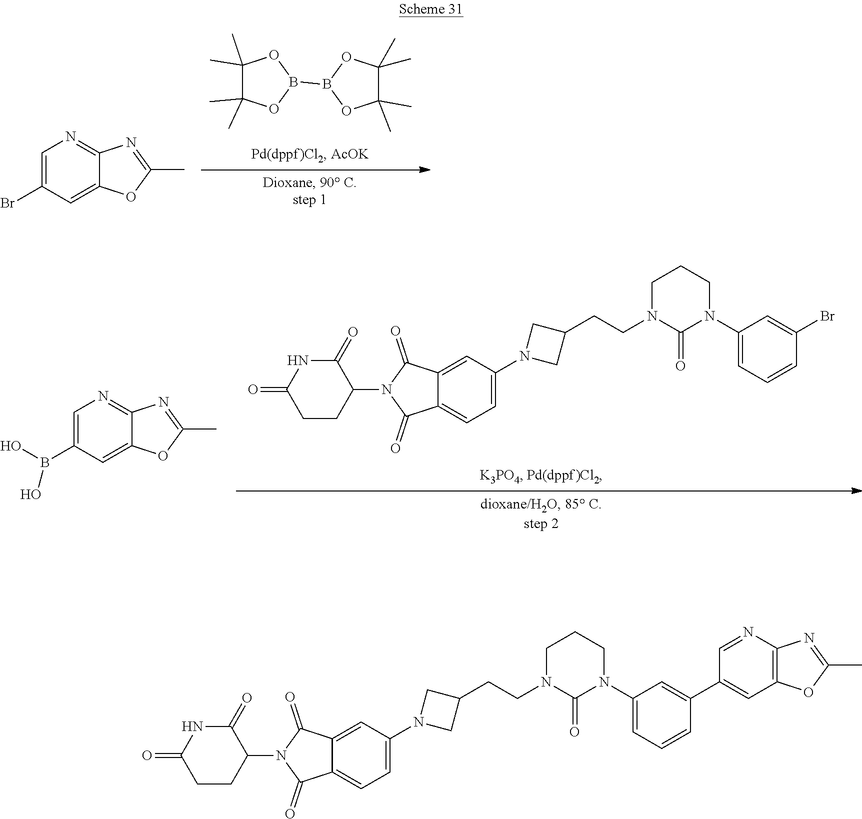

Step 1. Preparation of (2-methyloxazolo[4,5-b]pyridin-6-yl)boronic acid: To a solution of 6-bromo-2-methyl-[1,3]oxazolo[4,5-b]pyridine (1 g, 4.69 mmol) and B₂(Pin)₂ (1.43 g, 5.63 mmol) in dioxane (20 mL) was added KOAc (1.38 g, 14.08 mmol) and Pd(dppf)Cl₂ (343 mg, 0.469 mmol). The reaction mixture was stirred at 90° C. under N₂ for 16 hrs. The reaction mixture was evaporated and washed with n-hexane (20 mL). The mixture was filtered and evaporated to dryness. The residue was purified by Flash Chromatography (ACN/H₂O (0.1% FA)=20%-40%) to give the product as a yellow solid (500 mg, 47%). Mass (m/z): 179.1[M+H]⁺.

Step 2. Preparation of 2-(2,6-dioxopiperidin-3-yl)-5-(3-(2-(3-(3-(2-methyloxazolo[4,5-b]pyridin-6-yl)phenyl)-2-oxotetrahydropyrimidin-1(2H)-yl)ethyl)azetidin-1-yl)isoindoline-1,3-dione: To a solution of 5-(3-{2-[3-(3-bromophenyl)-2-oxo-1,3-diazinan-1-yl]ethyl}azetidin-1-yl)-2-(2,6-dioxopiperidin-3-yl)isoindole-1,3-dione (100 mg, 0.17 mmol) and (2-methyloxazolo[4,5-b]pyridin-6-yl)boronic acid (45 mg, 0.25 mmol) in dioxane (3 mL) and water (0.3 mL) was added K₃PO₄ (71 mg, 0.34 mmol) and Pd(dppf)Cl₂ (12 mg, 0.017 mmol). The reaction mixture was stirred at 85° C. under N₂ for 16 hrs. The solvent was removed under reduced pressure and the residue was purified by Prep-HPLC [chromatographic column:—Gemini-C18 150×21.2 mm, Sum; mobile phase: ACN—H₂O (0.1% FA), gradient: 50-80] to give the product as a yellow solid (40 mg, 34%). Mass (m/z): 648.0[M+H]⁺. ¹H NMR (400 MHz, DMSO-d₆) δ 11.07 (s, 1H), 8.76 (d, J=2.0 Hz, 1H), 8.41 (d, J=2.0 Hz, 1H), 7.68-7.60 (m, 2H), 7.52 (d, J=7.8 Hz, 1H), 7.44 (t, J=7.8 Hz, 1H), 7.35 (d, J=8.0 Hz, 1H), 6.76 (d, J=2.0 Hz, 1H), 6.63 (dd, J=8.4, 2.1 Hz, 1H), 5.05 (dd, J=12.8, 5.4 Hz, 1H), 4.14 (t, J=8.2 Hz, 2H), 3.81-3.66 (m, 4H), 3.41 (t, J=5.8 Hz, 2H), 3.31 (s, 2H), 2.94-2.76 (m, 2H), 2.70 (s, 3H), 2.62-2.51 (m, 2H), 2.11-1.97 (m, 3H), 1.89 (dd, J=14.0, 7.2 Hz, 2H).

### Compound 25: 2-(2,6-dioxopiperidin-3-yl)-5-(3-{2-[2-oxo-3-(3-{1H-pyrazolo[3,4-b]pyridin-5-yl}phenyl)-1,3-diazinan-1-yl]ethyl}azetidin-1-yl)isoindole-1,3-dione

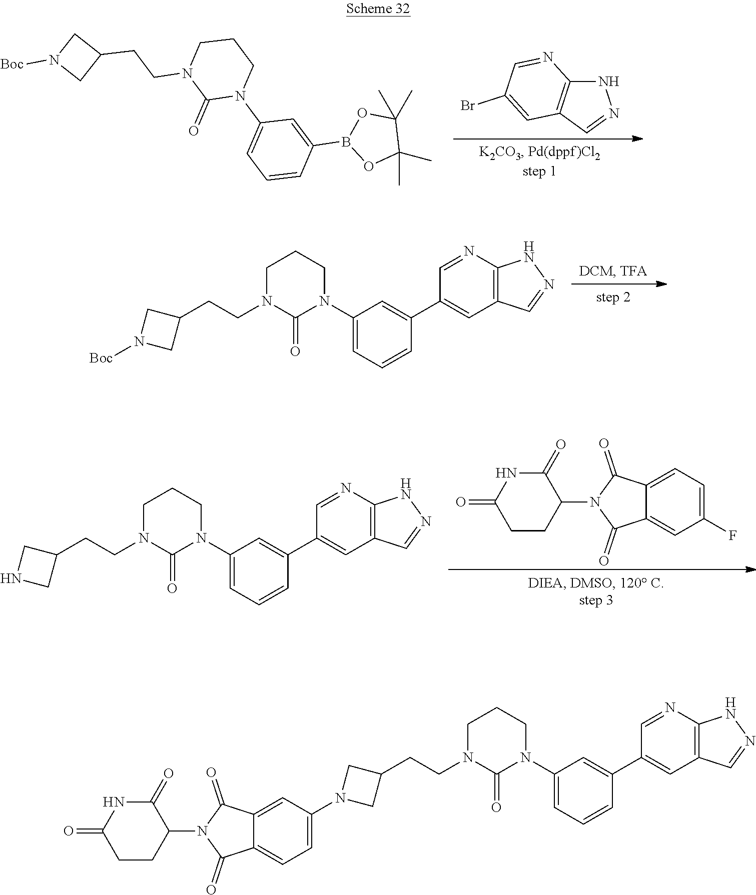

Step 1. Preparation of tert-butyl 3-(2-(3-(3-(1H-pyrazolo[3,4-b]pyridin-5-yl)phenyl)-2-oxotetrahydropyrimidin-1(2H)-yl)ethyl)azetidine-1-carboxylate: To a mixture of tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate (80 mg, 0.164 mmol) in dioxane/H₂O (10:1, 5.5 mL) was added K₂CO₃ (68 mg, 0.493 mmol), 5-bromo-1H-pyrazolo[3,4-b]pyridine (39 mg, 0.197 mmol) and Pd(dppf)Cl₂ (13 mg, 0.016 mmol). The reaction was degassed with N₂ for 3 times and stirred at 90° C. for 16 hrs. The reaction mixture was poured into water (10 mL), extracted with EtOAc (10 mL×3). The combined organic layers were washed with brine (10 mL×3), dried over Na₂SO₄ and concentrated under reduced pressure. The residue was purified by flash column (PE:EA=0˜100%) to give the desired product tert-butyl 3-(2-(3-(3-(1H-pyrazolo[3,4-b]pyridin-5-yl)phenyl)-2-oxotetrahydropyrimidin-1(2H)-yl)ethyl)azetidine-1-carboxylate as white solid (80 mg, 92%). Mass (m/z): 477.2 [M+H]⁺.

Step 2. Preparation of 1-[2-(azetidin-3-yl)ethyl]-3-(3-{1H-pyrazolo[3,4-b]pyridin-5-yl}phenyl)-1,3-diazinan-2-one: A mixture of tert-butyl 3-(2-(3-(3-(1H-pyrazolo[3,4-b]pyridin-5-yl)phenyl)-2-oxotetrahydropyrimidin-1(2H)-yl)ethyl)azetidine-1-carboxylate (80.0 mg, 0.167 mmol) in DCM/TFA (3:1, 4.0 mL) was stirred at 25° C. for 1 hour. The reaction mixture was concentrated under reduced pressure to give the product 1-[2-(azetidin-3-yl)ethyl]-3-(3-{1H-pyrazolo[3,4-b]pyridin-5-yl}phenyl)-1,3-diazinan-2-one as yellow solid (40 m g, 57%). Mass (m/z): 377.2 [M+H]⁺.

Step 3. Preparation of 2-(2,6-dioxopiperidin-3-yl)-5-(3-{2-[2-oxo-3-(3-{1H-pyrazolo[3,4-b]pyridin-5-yl}phenyl)-1,3-diazinan-1-yl]ethyl}azetidin-1-yl)isoindole-1,3-dione: To a mixture of 1-[2-(azetidin-3-yl)ethyl]-3-(3-{1H-pyrazolo[3,4-b]pyridin-5-yl}phenyl)-1,3-diazinan-2-one (40 mg, 0.106 mmol) in DMSO (3 mL) was added DIEA (27.5 mg, 0.212 mmol) and 2-(2,6-dioxopiperidin-3-yl)-5-fluoroisoindole-1,3-dione (29.4 mg, 0.106 mmol). The reaction was stirred at 120° C. for 1 hour. The reaction mixture was extracted with EtOAc (50 mL×3). The combined organic layers were washed with brine (100 mL×3), dried over Na₂SO₄ and concentrated under reduced pressure. The residue was purified by Prep-TLC (DCM/MeOH=10:1) to give the product 2-(2,6-dioxopiperidin-3-yl)-5-(3-{2-[2-oxo-3-(3-{1H-pyrazolo[3,4-b]pyridin-5-yl}phenyl)-1,3-diazinan-1-yl]ethyl}azetidin-1-yl)isoindole-1,3-dione as a yellow solid (10 mg, 14%). Mass (m/z): 633.2 [M+H]⁺. ¹H NMR (400 MHz, CDCl₃) δ 8.57 (s, 1H), 8.38 (s, 1H), 8.24 (s, 1H), 8.14 (s, 1H), 7.72 (s, 1H), 7.54 (d, J=8.4 Hz, 1H), 7.46 (t, J=7.8 Hz, 1H), 7.31 (dd, J=14.8, 7.6 Hz, 3H), 6.64 (s, 1H), 6.40 (d, J=7.8 Hz, 1H), 4.90 (dd, J=12.2, 5.2 Hz, 1H), 4.15 (t, J=7.8 Hz, 2H), 3.82 (d, J=5.4 Hz, 2H), 3.72 (d, J=5.8 Hz, 2H), 3.51-3.47 (m, 4H), 2.89-2.71 (m, 4H), 2.24-2.20 (m, 2H), 2.09-2.01 (m, 3H).

### Compound 26: 5-(3-(2-(3-(3-(4-chloro-3-ethyl-1H-indol-5-yl)phenyl)-2-oxotetrahydropyrimidin-1(2H)-yl)ethyl)azetidin-1-yl)-2-(2,6-dioxopiperidin-3-yl)isoindoline-1,3-dione

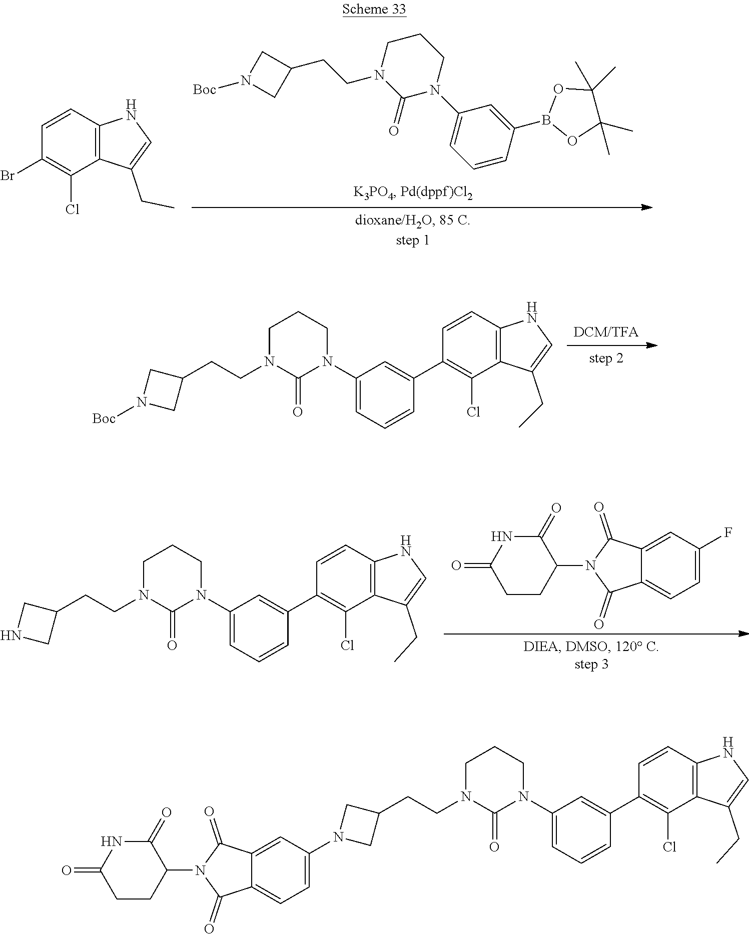

Step 1. Preparation of tert-butyl 3-(2-(3-(3-(4-chloro-3-ethyl-1H-indol-5-yl)phenyl)-2-oxotetrahydropyrimidin-1(2H)-yl)ethyl)azetidine-1-carboxylate: To a solution of 5-bromo-4-chloro-3-ethyl-1H-indole (100 mg, 0.39 mmol) in dioxane/H₂O (10:1, 10 mL) was added tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate (282 mg, 0.58 mmol), K₂CO₃ (107 mg, 0.77 mmol) and Pd(dppf)Cl₂ (28 mg, 0.04 mmol). The reaction mixture was stirred at 90° C. under N₂ for 16 hrs. After the reaction completed, H₂O (100 mL) was added to the reaction mixture, and then extracted with DCM (50 mL×3). The combined organic layer was washed with brine (30 mL×2), then dried over with anhydrous Na₂SO₄. After filtration, the solution was concentrated under vacuum, and the residue was purified by Combiflash (PE/EA=0˜10%) to give the product tert-butyl 3-(2-(3-(3-(4-chloro-3-ethyl-1H-indol-5-yl)phenyl)-2-oxotetrahydropyrimidin-1(2H)-yl)ethyl)azetidine-1-carboxylate as yellow oil (100 mg, 48%). Mass (m/z): 537.2 [M+H]⁺.

Step 2. Preparation of 1-(2-(azetidin-3-yl)ethyl)-3-(3-(4-chloro-3-ethyl-1H-indol-5-yl)phenyl)tetrahydropyrimidin-2(1H)-one: A solution of tert-butyl 3-(2-(3-(3-(4-chloro-3-ethyl-1H-indol-5-yl)phenyl)-2-oxotetrahydropyrimidin-1(2H)-yl)ethyl)azetidine-1-carboxylate (100 mg, 0.19 mmol) in TFA/DCM (3:1, 5 mL) was stirred at rt for 1 hour. The resulting mixture was concentrated to give the product 1-(2-(azetidin-3-yl)ethyl)-3-(3-(4-chloro-3-ethyl-1H-indol-5-yl)phenyl)tetrahydropyrimidin-2(1H)-one as yellow oil (80 mg, purity: 60%). Mass (m/z): 437.2 [M+H]⁺.

Step 3. Preparation of 5-(3-(2-(3-(3-(4-chloro-3-ethyl-1H-indol-5-yl)phenyl)-2-oxotetrahydropyrimidin-1(2H)-yl)ethyl)azetidin-1-yl)-2-(2,6-dioxopiperidin-3-yl)isoindoline-1,3-dione: To a solution of 1-(2-(azetidin-3-yl)ethyl)-3-(3-(4-chloro-3-ethyl-1H-indol-5-yl)phenyl)tetrahydropyrimidin-2(1H)-one (80 mg, 0.18 mmol) in DMSO (5 mL) was added 2-(2,6-dioxopiperidin-3-yl)-5-fluoroisoindole-1,3-dione (50 mg, 0.18 mmol) and DIEA (71 mg, 0.55 mmol). The reaction mixture was stirred at 120° C. under N₂ for 2 hrs. After the reaction completed, H₂O (30 mL) was added to the reaction mixture, and then extracted with EA (20 mL×3). The combined organic layer was washed with brine (30 mL×2), then dried over with anhydrous Na₂SO₄. After filtration, the solution was concentrated under vacuum, and the residue was purified by Combiflash (DCM/MeOH=0˜10%) to give the product 5-(3-(2-(3-(3-(4-chloro-3-ethyl-1H-indol-5-yl)phenyl)-2-oxotetrahydropyrimidin-1(2H)-yl)ethyl)azetidin-1-yl)-2-(2,6-dioxopiperidin-3-yl)isoindoline-1,3-dione as a yellow solid (15 mg, 6%). Mass (m/z): 693.1 [M+H]⁺. ¹H NMR (400 MHz, MeOD) δ 7.56 (d, J=8.4 Hz, 1H), 7.39 (d, J=7.8 Hz, 1H), 7.35 (t, J=1.6 Hz, 1H), 7.29 (d, J=8.4 Hz, 2H), 7.25 (s, 1H), 7.09-7.04 (m, 2H), 6.76 (s, 1H), 6.59 (s, 1H), 5.04 (dd, J=12.4, 5.4 Hz, 1H), 4.17 (t, J=7.8 Hz, 2H), 3.80-3.71 (m, 4H), 3.47 (dt, J=13.8, 6.4 Hz, 4H), 3.00 (d, J=7.4 Hz, 2H), 2.87-2.80 (m, 2H), 2.76-2.68 (m, 2H), 2.20-2.13 (m, 2H), 2.02-1.93 (m, 3H), 1.30 (t, J=7.4 Hz, 3H).

### Compound 27: 2-(2,6-dioxopiperidin-3-yl)-5-(3-{2-[3-(3-{3-ethyl-1H-pyrrolo[3,2-b]pyridin-5-yl}phenyl)-2-oxo-1,3-diazinan-1-yl]ethyl}azetidin-1-yl)isoindole-1,3-dione

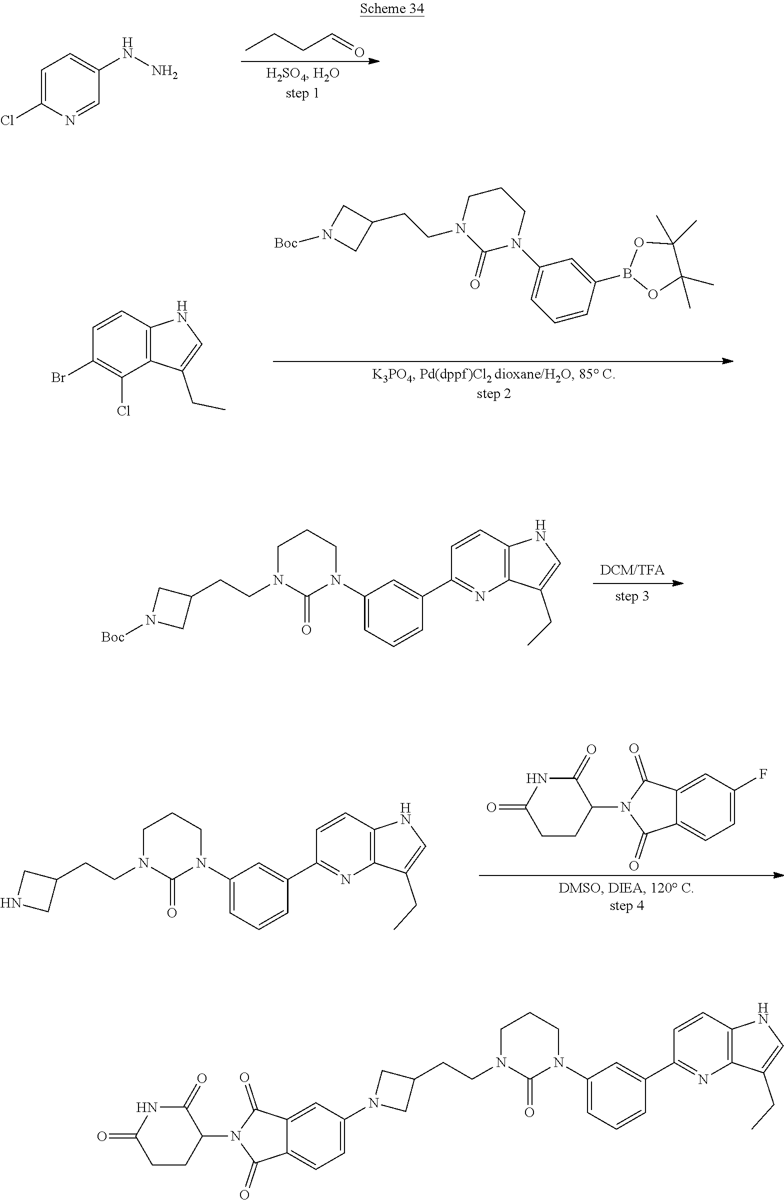

Step 1. Preparation of 5-chloro-3-ethyl-1H-pyrrolo[3,2-b]pyridine: To a solution of 2-chloro-5-hydrazinylpyridine (500 mg, 3.48 mmol) in 5% H₂SO₄ solution (20 mL) was added butanal (276 mg, 3.83 mmol). The reaction mixture was stirred at 110° C. under N₂ for 16 hrs. The reaction solution was adjusted pH to 8-9 by using 40% aqueous KOH solution, then extracted with EA (100 mL×3). The combined organic layer was washed with brine (30 mL×2), then dried over with anhydrous Na₂SO₄. After filtration, the solution was concentrated under vacuum, and the residue was purified by Combiflash (PE/EA=0˜50%) to give the product 5-chloro-3-ethyl-1H-pyrrolo[3,2-b]pyridine as a yellow solid (236 mg, 52%). Mass (m/z): 181.0 [M+H]⁺.

Step 2. Preparation of tert-butyl 3-(2-(3-(3-(3-ethyl-1H-pyrrolo[3,2-b]pyridin-5-yl)phenyl)-2-oxotetrahydropyrimidin-1(2H)-yl)ethyl)azetidine-1-carboxylate: To a solution of 5-chloro-3-ethyl-1H-pyrrolo[3,2-b]pyridine (55 mg, 0.30 mmol) in dioxane/H₂O (10/1, 5 mL) was added tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate (163 mg, 0.33 mmol), K₂CO₃ (84 mg, 0.61 mmol) and Pd(dppf)Cl₂ (22 mg, 0.03 mmol). The reaction mixture was stirred at 90° C. under N₂ for 16 hrs. The solvent was removed under reduced pressure and the residue was purified by Combiflash (PE/EA=0˜50%) to give the product tert-butyl 3-(2-(3-(3-(3-ethyl-1H-pyrrolo[3,2-b]pyridin-5-yl)phenyl)-2-oxotetrahydropyrimidin-1(2H)-yl)ethyl)azetidine-1-carboxylate as yellow oil (80 mg, 52%). Mass (m/z): 504.2 [M+H]f.

Step 3. Preparation of 1-(2-(azetidin-3-yl)ethyl)-3-(3-(3-ethyl-1H-pyrrolo[3,2-b]pyridin-5-yl)phenyl)tetrahydropyrimidin-2(1H)-one: To a solution of tert-butyl 3-(2-(3-(3-(3-ethyl-1H-pyrrolo[3,2-b]pyridin-5-yl)phenyl)-2-oxotetrahydropyrimidin-1(2H)-yl)ethyl)azetidine-1-carboxylate (80 mg, 0.16 mmol) in DCM (3 mL) was added TFA (1 mL). The reaction mixture was stirred at rt for 1 hour. The resulting mixture was concentrated to give the product 1-(2-(azetidin-3-yl)ethyl)-3-(3-(3-ethyl-1H-pyrrolo[3,2-b]pyridin-5-yl)phenyl)tetrahydropyrimidin-2(1H)-one as brown oil (80 mg, purity: 60%). Mass (m/z): 404.2 [M+H]⁺.

Step 4. Preparation of 2-(2,6-dioxopiperidin-3-yl)-5-(3-{2-[3-(3-{3-ethyl-1H-pyrrolo[3,2-b]pyridin-5-yl}phenyl)-2-oxo-1,3-diazinan-1-yl]ethyl}azetidin-1-yl)isoindole-1,3-dione: To a solution of 1-[2-(azetidin-3-yl)ethyl]-3-(3-{3-ethyl-1H-pyrrolo[3,2-b]pyridin-5-yl}phenyl)-1,3-diazinan-2-one (80 mg, 0.20 mmol) in DMSO (5 mL) was added 2-(2,6-dioxopiperidin-3-yl)-5-fluoroisoindole-1,3-dione (55 mg, 0.20 mmol) and DIEA (77 mg, 0.59 mmol). The reaction mixture was stirred at 120° C. under N₂ for 2 hrs. After the reaction completed, H₂O (50 mL) was added to the reaction mixture, and then extracted with EA (30 mL×3). The combined organic layer was washed with brine (30 mL×2), then dried over with anhydrous Na₂SO₄. After filtration, the solution was concentrated under vacuum, and the residue was purified by Combiflash (DCM/MeOH=0˜10%) to give the product 2-(2,6-dioxopiperidin-3-yl)-5-(3-{2-[3-(3-{3-ethyl-1H-pyrrolo[3,2-b]pyridin-5-yl}phenyl)-2-oxo-1,3-diazinan-1-yl]ethyl}azetidin-1-yl)isoindole-1,3-dione as a yellow solid (5 mg, 3%). Mass (m/z): 660.2 [M+H]⁺. ¹H NMR (400 MHz, MeOD) δ 7.89 (s, 1H), 7.81 (d, J=8.6 Hz, 2H), 7.59 (dd, J=8.4, 5.6 Hz, 2H), 7.48 (t, J=7.8 Hz, 1H), 7.36 (s, 1H), 7.31 (d, J=8.0 Hz, 1H), 6.78 (d, J=1.8 Hz, 1H), 6.61 (dd, J=8.4, 2.2 Hz, 1H), 5.04 (dd, J=12.4, 5.4 Hz, 1H), 4.61 (s, 1H), 4.20 (t, J=8.2 Hz, 2H), 3.86-3.72 (m, 4H), 3.50 (dt, J=13.8, 6.4 Hz, 4H), 2.95-2.80 (m, 4H), 2.73 (d, J=16.0 Hz, 2H), 2.20 (d, J=5.7 Hz, 2H), 2.02-1.98 (m, 2H), 1.36 (t, J=7.6 Hz, 3H).

### Compound 28: 5-[4-({3-[3-(6-amino-2-chloropyridin-3-yl)phenyl]-2-oxo-1,3-diazinan-1-yl}methyl)piperidin-1-yl]-2-(2,6-dioxopiperidin-3-yl)isoindole-1,3-dione

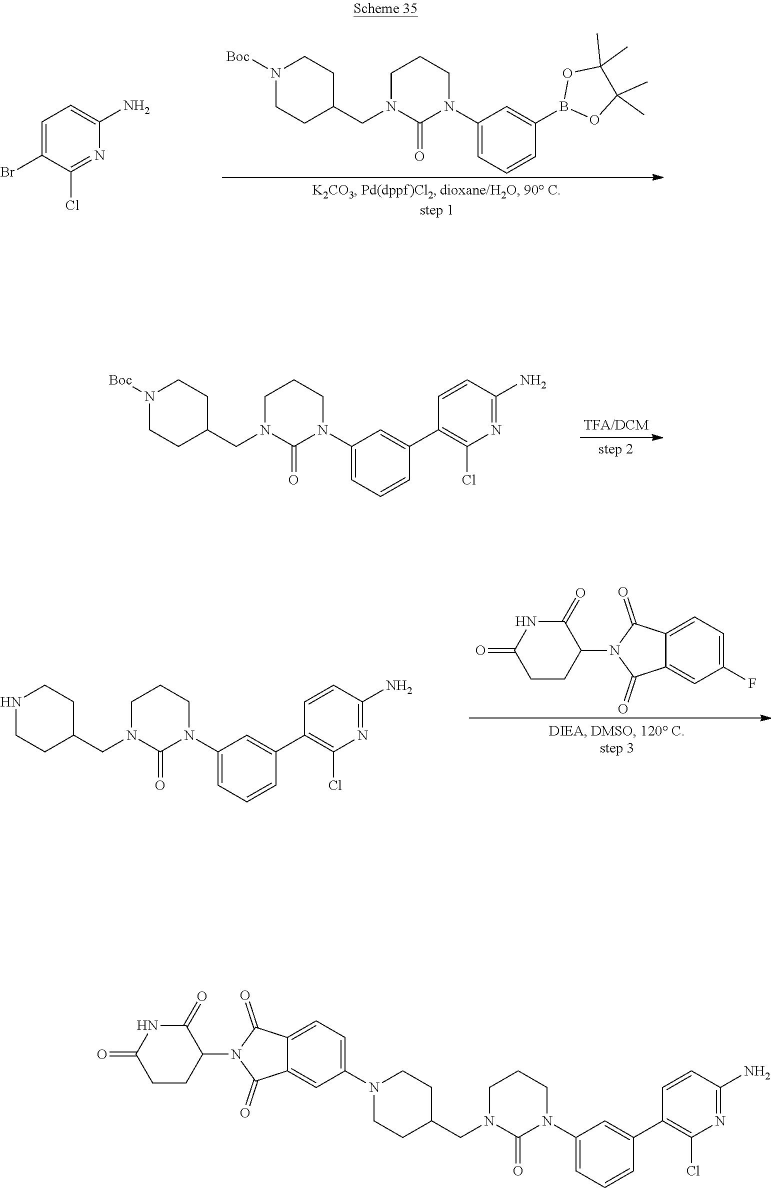 [SMILES: Nc1ccc(Br)c(Cl)n1 ; CC(C)(C)OC(=O)N1CCC(CN2CCCN(c3cccc(B4OC(C)(C)C(C)(C)O4)c3)C2=O)CC1 ; CC(C)(C)OC(=O)N1CCC(CN2CCCN(c3cccc(-c4ccc(N)nc4Cl)c3)C2=O)CC1 ; Nc1ccc(-c2cccc(N3CCCN(CC4CCNCC4)C3=O)c2)c(Cl)n1 ; O=C1CCC(N2C(=O)c3ccc(F)cc3C2=O)C(=O)N1 ; Nc1ccc(-c2cccc(N3CCCN(CC4CCN(c5ccc6c(c5)C(=O)N(C5CCC(=O)NC5=O)C6=O)CC4)C3=O)c2)c(Cl)n1]

Step 1. Preparation of tert-butyl 4-((3-(3-(6-amino-2-chloropyridin-3-yl)phenyl)-2-oxotetrahydropyrimidin-1(2H)-yl)methyl)piperidine-1-carboxylate: To a solution of 5-bromo-6-chloropyridin-2-amine (200 mg, 0.96 mmol) in dioxane/H₂O (10:1, 10 mL) was added tert-butyl 4-((2-oxo-3-(3-(4,4,5,5-tetramethyl-1,3,2-dioxaborolan-2-yl)phenyl)tetrahydropyrimidin-1(2H)-yl)methyl)piperidine-1-carboxylate (482 mg, 0.96 mmol), K₂CO₃ (267 mg, 1.93 mmol) and Pd(dppf)Cl₂ (70 mg, 0.1 mmol). The reaction mixture was stirred at 90° C. under N₂ for 16 hrs. The solvent was removed under reduced pressure and the residue was purified by Combiflash (DCM/MeOH=0˜3%) to give the product tert-butyl 4-((3-(3-(6-amino-2-chloropyridin-3-yl)phenyl)-2-oxotetrahydropyrimidin-1(2H)-yl)methyl)piperidine-1-carboxylate as yellow oil (322 mg, 66%). Mass (m/z): 522.3 [M+Na]⁺.

Step 2. Preparation of 1-(3-(6-amino-2-chloropyridin-3-yl)phenyl)-3-(piperidin-4-ylmethyl)tetrahydropyrimidin-2(1H)-one: To a solution of tert-butyl 4-((3-(3-(6-amino-2-chloropyridin-3-yl)phenyl)-2-oxotetrahydropyrimidin-1(2H)-yl)methyl)piperidine-1-carboxylate (322 mg, 0.81 mmol) in DCM (3 mL) was added TFA (1 mL). The reaction mixture was stirred at rt for 1 hour. The resulting mixture was concentrated to give the product 1-(3-(6-amino-2-chloropyridin-3-yl)phenyl)-3-(piperidin-4-ylmethyl)tetrahydropyrimidin-2(1H)-one as brown oil (370 mg, purity: 60%). Mass (m/z): 422.3 [M+Na]⁺.

Step 3. Preparation of 5-[4-({3-[3-(6-amino-2-chloropyridin-3-yl)phenyl]-2-oxo-1,3-diazinan-1-yl}methyl)piperidin-1-yl]-2-(2,6-dioxopiperidin-3-yl)isoindole-1,3-dione: To a solution of 1-(3-(6-amino-2-chloropyridin-3-yl)phenyl)-3-(piperidin-4-ylmethyl)tetrahydropyrimidin-2(1H)-one (200 mg, 0.5 mmol) in DMSO (10 mL) was added 2-(2,6-dioxopiperidin-3-yl)-5-fluoroisoindole-1,3-dione (138 mg, 0.5 mmol) and DIEA (323 mg, 2.5 mmol). The reaction mixture was stirred at 120° C. under N₂ for 2 hrs. After the reaction completed, H₂O (100 mL) was added to the reaction mixture, and then extracted with EA (50 mL×3). The combined organic layer was washed with brine (100 mL×3), then dried over with anhydrous Na₂SO₄. After filtration, the solution was concentrated under vacuum, and the residue was purified by Combiflash (DCM/MeOH=0˜10%) to give the product 5-[4-({3-[3-(6-amino-2-chloropyridin-3-yl)phenyl]-2-oxo-1,3-diazinan-1-yl}methyl)piperidin-1-yl]-2-(2,6-dioxopiperidin-3-yl)isoindole-1,3-dione as a yellow solid (80 mg, 24%). Mass (m/z): 656.2 [M+H]⁺. ¹H NMR (400 MHz, CDCl₃) δ 7.98 (s, 1H), 7.66 (d, J=8.6 Hz, 1H), 7.45 (d, J=8.2 Hz, 1H), 7.36 (dd, J=14.6, 6.8 Hz, 2H), 7.30 (s, 1H), 7.19 (d, J=7.6 Hz, 1H), 7.03 (dd, J=8.6, 2.2 Hz, 1H), 6.47 (d, J=8.2 Hz, 1H), 4.93 (dd, J=12.2, 5.4 Hz, 1H), 4.61 (s, 2H), 3.96 (d, J=13.0 Hz, 2H), 3.81-3.74 (m, 2H), 3.45 (t, J=5.8 Hz, 2H), 3.30 (d, J=7.2 Hz, 2H), 2.99 (t, J=11.6 Hz, 2H), 2.86 (dd, J=26.8, 14.6 Hz, 2H), 2.74 (d, J=15.2 Hz, 1H), 2.18-2.13 (m, 2H), 1.83 (d, J=10.6 Hz, 2H), 1.37 (dd, J=24.4, 12.2 Hz, 4H).

### Compound 29: 5-(4-((3-(4′-amino-2′-chloro-3′-ethyl-[1,1′-biphenyl]-3-yl)-2-oxotetrahydropyrimidin-1(2H)-yl)methyl)piperidin-1-yl)-2-(2,6-dioxopiperidin-3-yl)isoindoline-1,3-dione

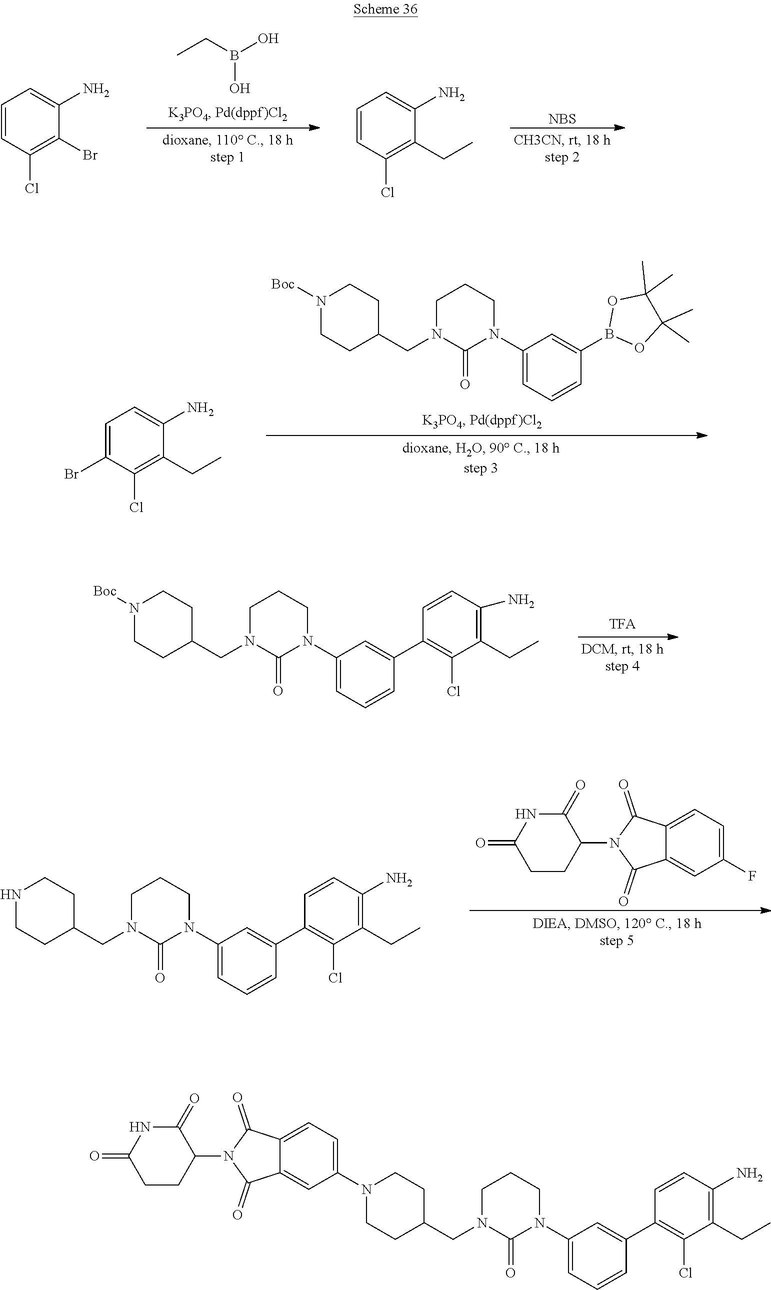

Step 1. Preparation of 3-chloro-2-ethylaniline: To a mixture of 2-bromo-3-chloroaniline (1000 mg, 4.84 mmol), ethylboronic acid (1.78 g, 24.217 mmol), and K₃PO₄ (3.08 g, 14.530 mmol) in dioxane/H₂O (10:1, 20 mL) was added Pd (dppf) Cl₂·CH₂Cl₂ (359 mg, 0.484 mmol). The reaction mixture was stirred at 1 10° C. under N₂ for 18 hrs. The reaction mixture was concentrated and the residue was purified by column chromatography (EA/PE=1:6) to give the desired product (300 mg, yield: 37%) as a yellow solid. Mass (m/z): 156 [M+H]⁺.

Step 2. Preparation of 4-bromo-3-chloro-2-ethylaniline: To a solution of 3-chloro-2-ethylaniline (200 mg, 1.927 mmol) in ACN (6 mL) was added NBS (377 mg, 2.12 mmol). The reaction mixture was stirred at rt under N₂ for 18 hrs. The reaction mixture was concentrated, and the residue was purified by column chromatography (EA/PE=1:1) to give the desired product (200 mg, yield: 42%) as a yellow solid. Mass (m/z): 234 [M+H]⁺.

Step 3. Preparation of tert-butyl 4-((3-(4′-amino-2′-chloro-3′-ethyl-[1,1′-biphenyl]-3-yl)-2-oxotetrahy dropyrimidin-1(2H)-yl) methyl) piperidine-1-carboxylate: To a mixture of 4-bromo-3-chloro-2-ethylaniline (220 mg, 0.938 mmol), tert-butyl 4-((2-oxo-3-(3-(4,4,5,5-tetramethyl-1,3,2-dioxaborolan-2-yl)phenyl)tetrahydropyrimidin-1(2H)-yl)methyl)piperidine-1-carboxylate (704 mg, 1.407 mmol) and Potassium carbonate (259 mg, 1.876 mmol) in 1,4-dioxane/H₂O (10:1, 5 mL) was added Pd(dppf)Cl₂—CH₂Cl₂ (76 mg, 0.093 mmol). The reaction mixture was stirred at 90° C. under N₂ for 18 hrs. The reaction was filtered, and the filtrate was concentrated. The residue was purified by combi-flash with MeOH/DCM (1:10) to afford target compound (100 mg, yield: 19%) as a white solid. Mass (m/z): 527 [M+H]⁺.

Step 4. Preparation of 1-(4′-amino-2′-chloro-3′-ethyl-[1,1′-biphenyl]-3-yl)-3-(piperidin-4-ylmethyl) tetrahydropyrimidin-2(1H)-one: To a solution of tert-butyl 4-((3-(4′-amino-2′-chloro-3′-ethyl-[1,1′-biphenyl]-3-yl)-2-oxotetrahy dropyrimidin-1(2H)-yl) methyl) piperidine-1-carboxylate (150 mg, 0.284 mmol) in DCM (2 mL) was added TFA (0.4 mL). The reaction mixture was stirred at rt under N₂ for 18 h. The solution was concentrated under reduced pressure. to give the product (125 mg, 97%) as a brown solid. Mass (m/z): 426.9 [M+H]⁺.

Step 5. Preparation of 5-(4-((3-(4′-amino-2′-chloro-3′-ethyl-[1,1′-biphenyl]-3-yl)-2-oxotetrahydropyrimidin-1(2H)-yl) methyl) piperidin-1-yl)-2-(2,6-dioxopiperidin-3-yl) isoindoline-1,3-dione: A solution of 2-(2,6-dioxopiperidin-3-yl)-5-fluoroisoindole-1,3-dione (75 mg, 0.271 mmol), 1-[3-(4-amino-2-chloro-3-ethylphenyl)phenyl]-3-(piperidin-4-ylmethyl)-1,3-diazinan-2-one (127 mg, 0.298 mmol) and DIEA (350 mg, 2.715 mmol) in DMSO (5 mL) was stirred at 120° C. under N₂ for 18 hrs. After the reaction completed, H₂O (10 mL) was added to the reaction mixture, and then extracted with EA (10 mL×3). The combined organic layer was washed with brine (20 mL×3), then dried over with anhydrous Na₂SO₄. After filtration, the solution was concentrated under vacuum, and the residue was purified by prep-HPLC [(Gemini-C18 150×21.2 mm, 5 um; ACN—H₂O (0.1% FA), 50-70] to give the desired product (10 mg, yield: 5%) as a white solid. Mass (m/z): 683[M+H]⁺. ¹H NMR (400 MHz, CDCl₃) δ 7.95 (s, 1H), 7.66 (d, J=8.6 Hz, 1H), 7.38-7.31 (m, 1H), 7.30-7.26 (m, 2H), 7.20-7.15 (m, 1H), 7.02 (dd, J=12.0, 5.2 Hz, 2H), 6.63 (d, J=8.2 Hz, 1H), 4.93 (dd, J=12.2, 5.2 Hz, 1H), 3.95 (d, J=13.0 Hz, 2H), 3.81-3.72 (m, 2H), 3.44 (t, J=5.9 Hz, 2H), 3.30 (d, J=7.2 Hz, 2H), 2.99 (t, J=11.4 Hz, 2H), 2.92-2.66 (m, 5H), 2.19-2.03 (m, 4H), 1.83 (d, J=10.8 Hz, 2H), 1.43-1.30 (m, 2H), 1.20 (t, J=7.6 Hz, 3H).

### Compound 30: 5-{4-[(3-{3-[2-chloro-4-(ethylamino)phenyl]phenyl}-2-oxo-1,3-diazinan-1-yl)methyl]piperidin-1-yl}-2-(2,6-dioxopiperidin-3-yl)isoindole-1,3-dione

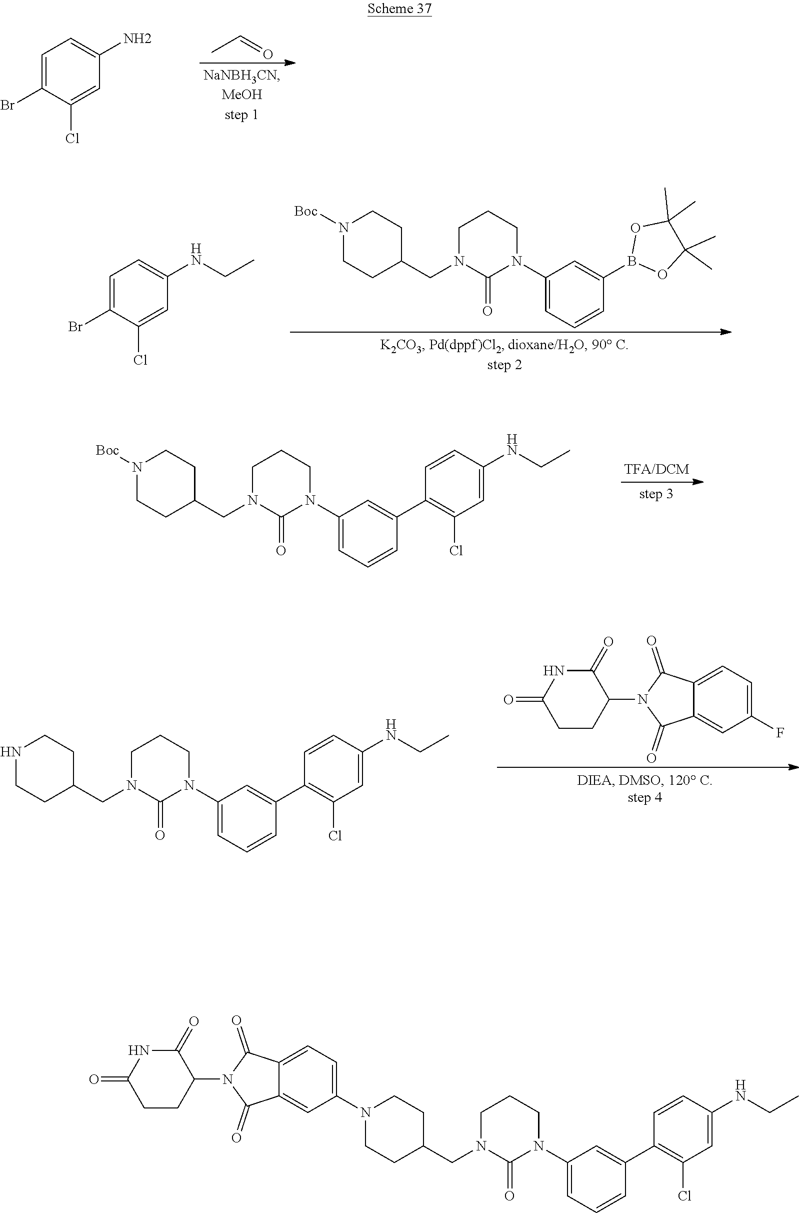

Step 1. Preparation of 4-bromo-3-chloro-N-ethylaniline: To a solution of 4-bromo-3-chloroaniline (1200 mg, 5.81 mmol) in MeOH (30 mL) was added acetaldehyde (256 mg, 75.8 mmol) and NaBH₃CN (1096 mg, 17.44 mmol) and AcOH (0.02 mL). The reaction mixture was stirred at 25° C. under N₂ for 2 hrs. The solvent was removed under reduced pressure and the residue was purified by Combiflash (PE/EA=0-2%) to give the product 4-bromo-3-chloro-N-ethylaniline as yellow oil (300 mg, 22%). Mass (m/z): 235.9 [M+H]⁺.

Step 2. Preparation of tert-butyl 4-((3-(2′-chloro-4′-(ethylamino)-[1,1′-biphenyl]-3-yl)-2-oxotetrahydropyrimidin-1(2H)-yl)methyl)piperidine-1-carboxylate: To a solution of 4-bromo-3-chloro-N-ethylaniline (300 mg, 1.28 mmol) in dioxane/H₂O (10/1, 10 mL) was added tert-butyl 4-((2-oxo-3-(3-(4,4,5,5-tetramethyl-1,3,2-dioxaborolan-2-yl)phenyl)tetrahydropyrimidin-1(2H)-yl)methyl)piperidine-1-carboxylate (960 mg, 1.92 mmol), K₂CO₃ (354 mg, 2.56 mmol) and Pd(dppf)Cl₂ (94 mg, 0.13 mmol). The reaction mixture was stirred at 90° C. under N₂ for 16 hrs. The solvent was removed under reduced pressure and the residue was purified by Combiflash (PE/EA=0˜50%) to give the product tert-butyl 4-((3-(2′-chloro-4′-(ethylamino)-[1,1′-biphenyl]-3-yl)-2-oxotetrahydropyrimidin-1(2H)-yl)methyl)piperidine-1-carboxylate as brown oil (370 mg, 54%). Mass (m/z): 549.2 [M+Na]⁺.

Step 3. Preparation of 1-(2′-chloro-4′-(ethylamino)-[1,1′-biphenyl]-3-yl)-3-(piperidin-4-ylmethyl)tetrahydropyrimidin-2(1H)-one: To a solution of tert-butyl 4-((3-(2′-chloro-4′-(ethylamino)-[1,1′-biphenyl]-3-yl)-2-oxotetrahydropyrimidin-1(2H)-yl)methyl)piperidine-1-carboxylate (270 mg, 0.51 mmol) in DCM (6 mL) was added TFA (2 mL). The reaction mixture was stirred at rt for 1 hour. The resulting mixture was concentrated to give the product 1-(2′-chloro-4′-(ethylamino)-[1,1′-biphenyl]-3-yl)-3-(piperidin-4-ylmethyl)tetrahydropyrimidin-2(1H)-one as yellow oil (300 mg, purity: 60%). Mass (m/z): 427.2 [M+H]⁺.

Step 4. Preparation of 5-{4-[(3-{3-[2-chloro-4-(ethylamino)phenyl]phenyl}-2-oxo-1,3-diazinan-1-yl)methyl]piperidin-1-yl}-2-(2,6-dioxopiperidin-3-yl)isoindole-1,3-dione: To a solution of 1-(2′-chloro-4′-(ethylamino)-[1,1′-biphenyl]-3-yl)-3-(piperidin-4-ylmethyl)tetrahydropyrimidin-2(1R)-one (300 mg, 0.70 mmol) in DMSO (10 mL) was added 2-(2,6-dioxopiperidin-3-yl)-5-fluoroisoindole-1,3-dione (157 mg, 0.70 mmol) and DIEA (366 mg, 3.5 mmol). The reaction mixture was stirred at 120° C. under N₂ for 2 hrs. After the reaction completed, H₂O (100 mL) was added to the reaction mixture, and then extracted with EA (50 mL×3). The combined organic layer was washed with brine (50 mL×3), then dried over with anhydrous Na₂SO₄. After filtration, the solution was concentrated under vacuum, and the residue was purified by Combiflash (DCM/MeOH=0˜10%) to give the product 5-{4-[(3-{3-[2-chloro-4-(ethylamino)phenyl]phenyl}-2-oxo-1,3-diazinan-1-yl)methyl]piperidin-1-yl}-2-(2,6-dioxopiperidin-3-yl)isoindole-1,3-dione as a yellow solid (58 mg, 14%). Mass (m/z): 683.1 [M+H]⁺. ¹H NMR (400 MHz, CDCl₃) δ 8.00 (s, 1H), 7.66 (d, J=8.4 Hz, 1H), 7.33 (dd, J=14.8, 7.0 Hz, 3H), 7.21 (d, J=7.4 Hz, 1H), 7.16 (d, J=8.4 Hz, 1H), 7.04 (s, 1H), 6.69 (s, 1H), 6.56 (s, 1H), 4.93 (dd, J=12.2, 5.4 Hz, 1H), 3.95 (d, J=12.6 Hz, 2H), 3.78 (d, J=5.4 Hz, 2H), 3.45 (d, J=5.4 Hz, 2H), 3.30 (d, J=6.8 Hz, 2H), 3.22-3.13 (m, 2H), 3.00 (d, J=12.4 Hz, 2H), 2.87 (s, 3H), 2.13 (d, J=12.4 Hz, 4H), 1.84 (d, J=12.6 Hz, 2H), 1.43-1.35 (m, 2H), 1.28 (t, J=7.0 Hz, 3H).

### Compound 31: 3-chloro-4-{3-[3-({1-[2-(2,6-dioxopiperidin-3-yl)-1,3-dioxoisoindol-5-yl]piperidin-4-yl}methyl)-2-oxo-1,3-diazinan-1-yl]phenyl}-N-methylbenzamide

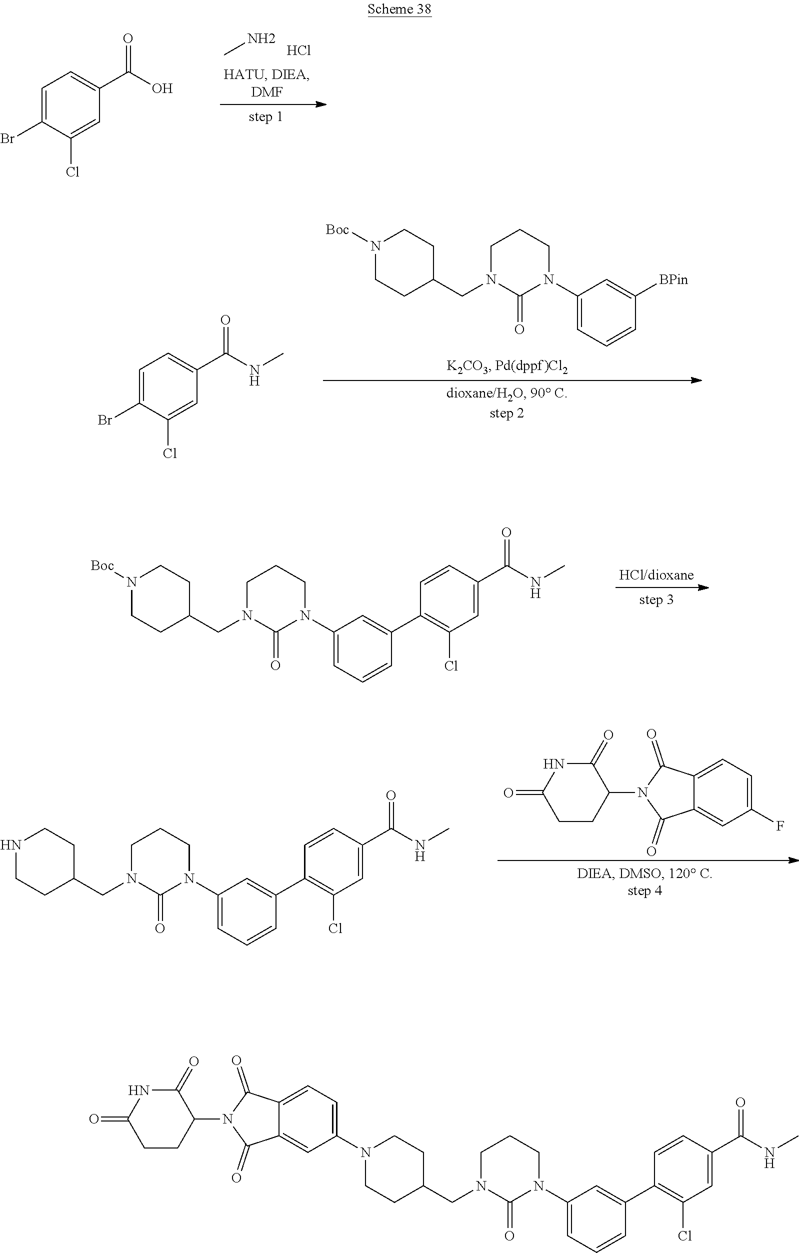

Step 1. Preparation of 4-bromo-3-chloro-N-methylbenzamide: To a solution of 4-bromo-3-chlorobenzoic acid (500 mg, 2.12 mmol) in DCM (20 mL) was added methanamine hydrochloride (143 mg, 2.12 mmol) and DIEA (1372 mg, 10.62 mmol) and HATU (2423 mg, 6.37 mmol). The reaction mixture was stirred at 25° C. under N₂ for 16 hrs. The solvent was removed under reduced pressure and the residue was purified by Combiflash (PE/EA=0-50%) to give the product 4-bromo-3-chloro-N-methylbenzamide as colorless oil (610 mg, 92%). Mass (m/z): 249.9 [M+H]⁺.

Step 2. Preparation of tert-butyl 4-((3-(2′-chloro-4′-(methylcarbamoyl)-[1,1′-biphenyl]-3-yl)-2-oxotetrahydropyrimidin-1(2H)-yl)methyl)piperidine-1-carboxylate: To a solution of 4-bromo-3-chloro-N-methylbenzamide (200 mg, 0.8 mmol) in dioxane/H₂O (10/1, 10 mL) was added tert-butyl 4-({2-oxo-3-[3-(4,4,5,5-tetramethyl-1,3,2-dioxaborolan-2-yl)phenyl]-1,3-diazinan-1-yl}methyl)piperidine-1-carboxylate (402 mg, 0.80 mmol) and K₂CO₃ (222 mg, 1.61 mmol) and Pd(dppf)Cl₂ (59 mg 0.08 mmol). The reaction mixture was stirred at 90° C. under N₂ for 16 hrs. The solvent was removed under reduced pressure and the residue was purified by Combiflash (PE/EA=0˜100%) to give the product tert-butyl 4-((3-(2′-chloro-4′-(methylcarbamoyl)-[1,1′-biphenyl]-3-yl)-2-oxotetrahydropyrimidin-1(2H)-yl)methyl)piperidine-1-carboxylate as a yellow solid (276 mg, 63%). Mass (m/z): 563.1 [M+Na]⁺.

Step 3. Preparation of 3-chloro-N-methyl-4-{3-[2-oxo-3-(piperidin-4-ylmethyl)-1,3-diazinan-1-yl]phenyl}benzamide: A solution of tert-butyl 4-((3-(2′-chloro-4′-(methylcarbamoyl)-[1,1′-biphenyl]-3-yl)-2-oxotetrahydropyrimidin-1(2H)-yl)methyl)piperidine-1-carboxylate (270 mg, 0.5 mmol) in HCl/dioxane (4.0 M, 10 mL) was stirred at rt for 1 hour. The resulting mixture was concentrated to give the product 3-chloro-N-methyl-4-{3-[2-oxo-3-(piperidin-4-ylmethyl)-1,3-diazinan-1-yl]phenyl}benzamide as yellow solid (210 mg, 95%). Mass (m/z): 441.0 [M+H]⁺.

Step 4. Preparation of 3-chloro-4-{3-[3-({1-[2-(2,6-dioxopiperidin-3-yl)-1,3-dioxoisoindol-5-yl]piperidin-4-yl}methyl)-2-oxo-1,3-diazinan-1-yl]phenyl}-N-methylbenzamide: To a solution of 3-chloro-N-methyl-4-{3-[2-oxo-3-(piperidin-4-ylmethyl)-1,3-diazinan-1-yl]phenyl}benzamide (210 mg, 0.48 mmol) in DMSO (10 mL) was added 2-(2,6-dioxopiperidin-3-yl)-5-fluoroisoindole-1,3-dione (132 mg, 0.48 mmol) and DIEA (307 mg, 2.38 mmol). The reaction mixture was stirred at 120° C. under N₂ for 2 hrs. After the reaction completed, H₂O (100 mL) was added to the reaction mixture, and then extracted with EA (50 mL×3). The combined organic layer was washed with brine (50 mL×3), then dried over with anhydrous Na₂SO₄. After filtration, the solution was concentrated under vacuum, and the residue was purified by Combiflash (DCM/MeOH=0˜6%) to give the product as a yellow solid (40 mg, 12%). Mass (m/z): 697.2 [M+H]⁺. ¹H NMR (400 MHz, CDCl₃) δ 8.07 (s, 1H), 7.88 (d, J=1.4 Hz, 1H), 7.69-7.62 (m, 2H), 7.40 (t, J=7.4 Hz, 2H), 7.37-7.32 (m, 2H), 7.26 (s, 1H), 7.22 (d, J=7.4 Hz, 1H), 7.04 (dd, J=8.6, 1.8 Hz, 1H), 6.28 (d, J=4.6 Hz, 1H), 4.93 (dd, J=12.2, 5.2 Hz, 1H), 3.95 (d, J=12.6 Hz, 2H), 3.81-3.76 (m, 2H), 3.45 (t, J=5.8 Hz, 2H), 3.30 (d, J=7.2 Hz, 2H), 3.02 (d, J=4.8 Hz, 3H), 2.97 (d, J=11.6 Hz, 2H), 2.91-2.71 (m, 3H), 2.20-2.08 (m, 4H), 1.84 (d, J=11.6 Hz, 2H), 1.38 (dd, J=21.2, 11.8 Hz, 2H).

### Compound 32: 3-chloro-4-{3-[3-({1-[2-(2,6-dioxopiperidin-3-yl)-1,3-dioxoisoindol-5-yl]piperidin-4-yl}methyl)-2-oxo-1,3-diazinan-1-yl]phenyl}-N,N-dimethylbenzamide

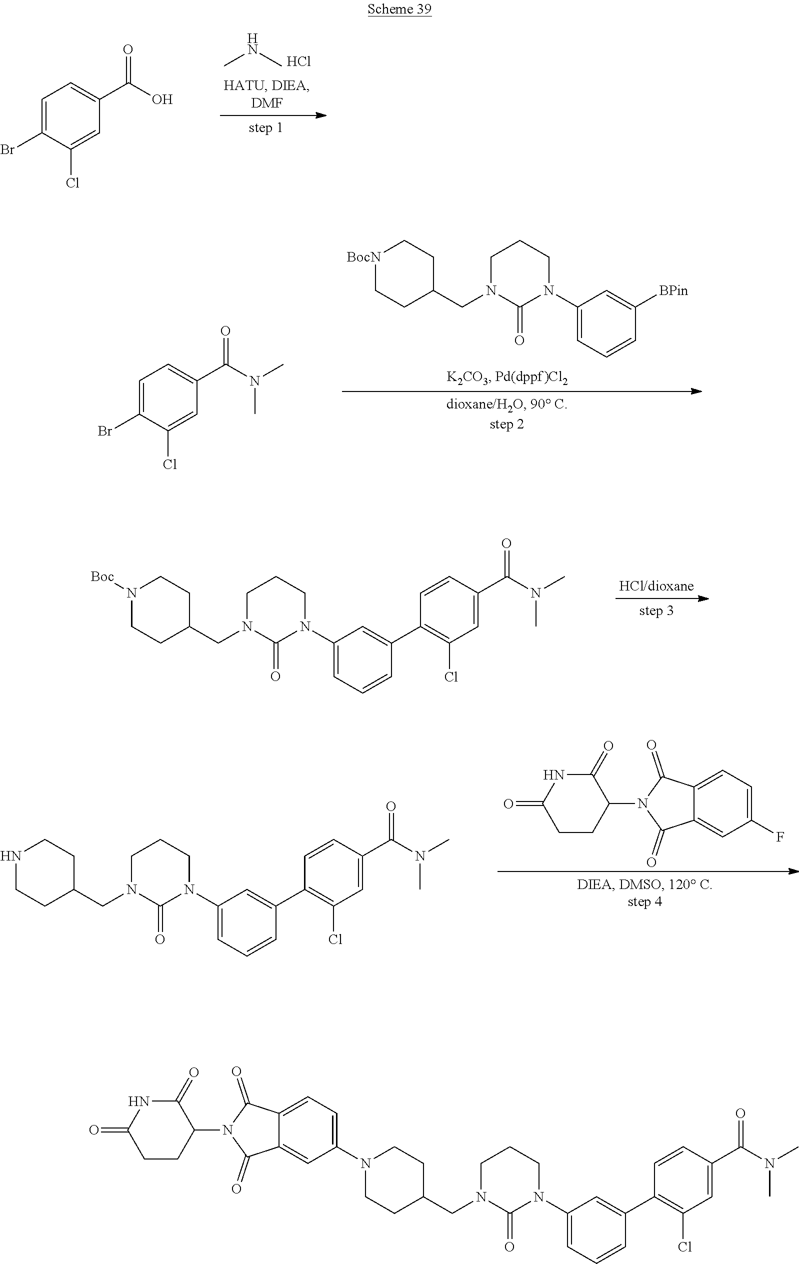

Step 1. Preparation of 4-bromo-3-chloro-N,N-dimethylbenzamide: To a solution of 4-bromo-3-chlorobenzoic acid (500 mg, 2.12 mmol) in DCM (20 mL) was added dimethylamine hydrochloride (173 mg, 2.12 mmol) and DIEA (1372 mg, 10.62 mmol) and HATU (2423 mg, 6.37 mmol). The reaction mixture was stirred at 25° C. under N₂ for 16 hrs. The solvent was removed under reduced pressure and the residue was purified by Combiflash (PE/EA=0˜50%) to give the product 4-bromo-3-chloro-N,N-dimethylbenzamide as colorless oil (690 mg, 74%). Mass (m/z): 263.9 [M+H]⁺.

Step 2. Preparation of tert-butyl 4-((3-(2′-chloro-4′-(dimethylcarbamoyl)-[1,1′-biphenyl]-3-yl)-2-oxotetrahydropyrimidin-1(2H)-yl)methyl)piperidine-1-carboxylate: To a solution of 4-bromo-3-chloro-N,N-dimethylbenzamide (158 mg, 0.6 mmol) in dioxane/H₂O (10/1, 15 mL) was added tert-butyl 4-({2-oxo-3-[3-(4,4,5,5-tetramethyl-1,3,2-dioxaborolan-2-yl)phenyl]-1,3-diazinan-1-yl}methyl)piperidine-1-carboxylate (300 mg, 0.6 mmol), K₂CO₃ (166 mg, 1.2 mmol) and Pd(dppf)Cl₂ (44 mg 0.06 mmol). The reaction mixture was stirred at 90° C. under N₂ for 16 hrs. The solvent was removed under reduced pressure and the residue was purified by Combiflash (PE/EA=0˜100%) to give the product tert-butyl 4-((3-(2′-chloro-4′-(dimethylcarbamoyl)-[1,1′-biphenyl]-3-yl)-2-oxotetrahydropyrimidin-1(2H)-yl)methyl)piperidine-1-carboxylate as yellow oil (240 mg, 71%). Mass (m/z): 577.0 [M+Na]⁺.

Step 3. Preparation of 3-chloro-N,N-dimethyl-4-{3-[2-oxo-3-(piperidin-4-ylmethyl)-1,3-diazinan-1-yl]phenyl}benzamide: A solution of tert-butyl 4-((3-(2′-chloro-4′-(dimethylcarbamoyl)-[1,1′-biphenyl]-3-yl)-2-oxotetrahydropyrimidin-1(2H)-yl)methyl)piperidine-1-carboxylate (240 mg, 0.43 mmol) in HCl/dioxane (4.0 M, 10 mL) was stirred at rt for 1 hour. The resulting mixture was concentrated to give the product 3-chloro-N,N-dimethyl-4-{3-[2-oxo-3-(piperidin-4-ylmethyl)-1,3-diazinan-1-yl]phenyl}benzamide as yellow solid (260 mg, 92%). Mass (m/z): 455.0 [M+H]⁺.

Step 4. Preparation of 3-chloro-4-{3-[3-({1-[2-(2,6-dioxopiperidin-3-yl)-1,3-dioxoisoindol-5-yl]piperidin-4-yl}methyl)-2-oxo-1,3-diazinan-1-yl]phenyl}-N,N-dimethylbenzamide: To a solution of 3-chloro-N,N-dimethyl-4-{3-[2-oxo-3-(piperidin-4-ylmethyl)-1,3-diazinan-1-yl]phenyl}benzamide (260 mg, 0.57 mmol) in DMSO (10 mL) was added 2-(2,6-dioxopiperidin-3-yl)-5-fluoroisoindole-1,3-dione (158 mg, 0.57 mmol) and DIEA (369 mg, 2.86 mmol). The reaction mixture was stirred at 120° C. under N₂ for 2 hrs. After the reaction completed, H₂O (100 mL) was added to the reaction mixture, and then extracted with EA (50 mL×3). The combined organic layer was washed with brine (50 mL×3), then dried over with anhydrous Na₂SO₄. After filtration, the solution was concentrated under vacuum, and the residue was purified by Combiflash (DCM/MeOH=0˜10%) to give the product 3-chloro-4-{3-[3-({1-[2-(2,6-dioxopiperidin-3-yl)-1,3-dioxoisoindol-5-yl]piperidin-4-yl}methyl)-2-oxo-1,3-diazinan-1-yl]phenyl}-N,N-dimethylbenzamide as a yellow solid (120 mg, 29%). Mass (m/z): 711.1 [M+H]⁺. ¹H NMR (400 MHz, CDCl₃) δ 8.00 (s, 1H), 7.66 (d, J=8.6 Hz, 1H), 7.53 (d, J=1.4 Hz, 1H), 7.40 (dd, J=7.6, 5.0 Hz, 2H), 7.37-7.32 (m, 3H), 7.27 (d, J=2.2 Hz, 1H), 7.24-7.21 (m, 1H), 7.06 (d, J=2.2 Hz, 1H), 4.96-4.90 (m, 1H), 3.95 (d, J=13.2 Hz, 2H), 3.82-3.75 (m, 2H), 3.45 (t, J=5.8 Hz, 2H), 3.30 (d, J=7.2 Hz, 2H), 3.13 (s, 3H), 3.04 (s, 3H), 2.99 (s, 2H), 2.87 (s, 3H), 2.21-2.07 (m, 4H), 1.84 (d, J=11.2 Hz, 2H), 1.39 (d, J=9.2 Hz, 2H).

### Compound 33: 5-[4-({3-[3-(2-chloro-4-methanesulfonylphenyl)phenyl]-2-oxo-1,3-diazinan-1-yl}methyl)piperidin-1-yl]-2-(2,6-dioxopiperidin-3-yl)isoindole-1,3-dione

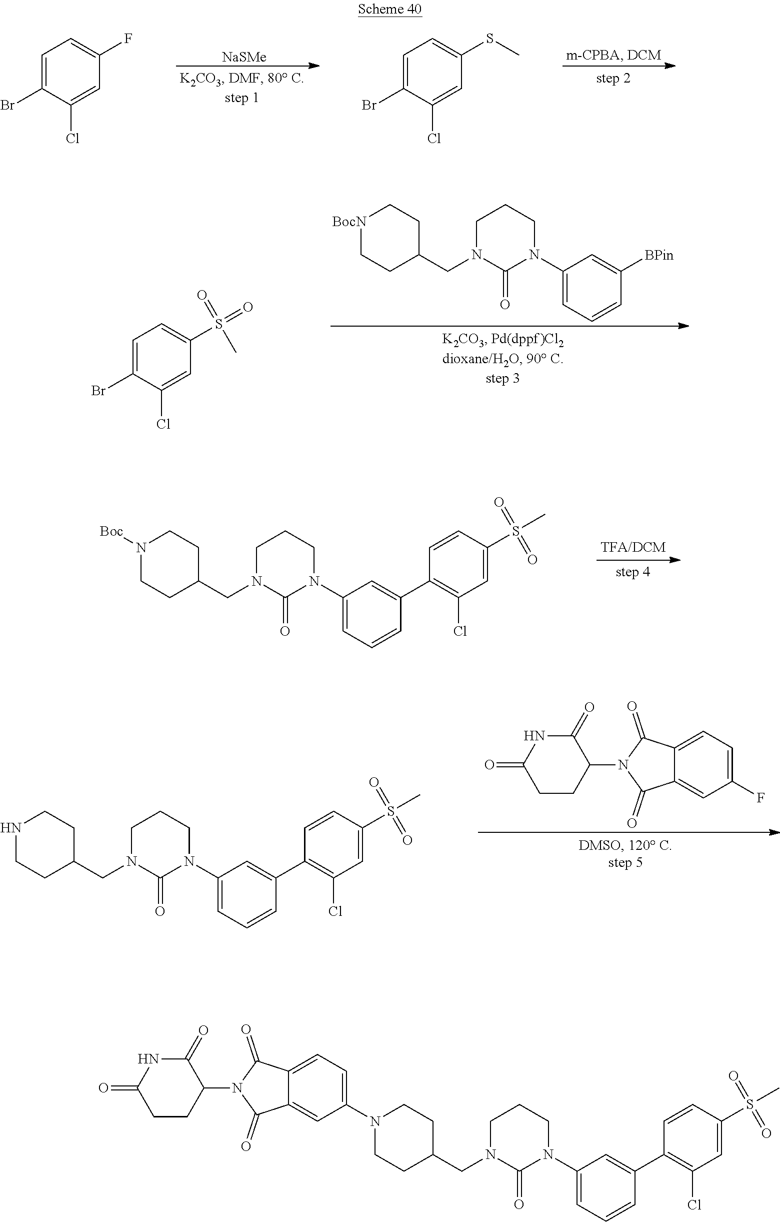

Step 1. Preparation of 4-bromo-2-iodo-1-(methylsulfanyl)benzene: To a solution of 4-bromo-1-fluoro-2-iodobenzene (1000 mg, 3.32 mmol) in DMF (20 mL) was added MeSNa (1396 mg, 19.94 mmol) and K₂CO₃ (919 mg, 6.65 mmol). The reaction mixture was stirred at 80° C. under N₂ for 16 hrs. After the reaction completed, H₂O (200 mL) was added to the reaction mixture, and then extracted with EA (100 mL×3), The combined organic layer was washed with brine (50 mL×3), then dried over with anhydrous Na₂SO₄. After filtration, the filtrate was concentrated to give the product 4-bromo-2-iodo-1-(methylsulfanyl)benzene as colorless oil (1300 mg, 71%). Mass (m/z): 352.0 [M+Na]⁺.

Step 2. Preparation of 1-bromo-2-chloro-4-methanesulfonylbenzene: To a solution of 1-bromo-2-chloro-4-(methylsulfanyl)benzene (700 mg, 2.95 mmol) in DCM (20 mL) was added 3-Chloroperoxybenzoic acid (1526 mg, 8.84 mmol). The reaction mixture was stirred at 25° C. under N₂ for 16 hrs. After the reaction completed, H₂O (100 mL) was added to the reaction mixture, and then extracted with DCM (100 mL×3). The combined organic layer was washed with brine (50 mL×3), then dried over with anhydrous Na₂SO₄. After filtration, the solution was concentrated under vacuum, and the residue was purified by Combiflash (PE/EA=0˜100%) to give the product 1-bromo-2-chloro-4-methanesulfonylbenzene as a white solid (500 mg, 62%). Mass (m/z): 267.2 [M+H]⁺.

Step 3. Preparation of tert-butyl 4-((3-(2′-chloro-4′-(methylsulfonyl)-[1,1′-biphenyl]-3-yl)-2-oxotetrahydropyrimidin-1(2H)-yl)methyl)piperidine-1-carboxylate: To a solution of 1-bromo-2-chloro-4-methanesulfonylbenzene (300 mg, 1.11 mmol) in dioxane/H₂O (15 mL) was added tert-butyl 4-({2-oxo-3-[3-(4,4,5,5-tetramethyl-1,3,2-dioxaborolan-2-yl)phenyl]-1,3-diazinan-1-yl}methyl)piperidine-1-carboxylate (1112 mg, 2.23 mmol), K₂CO₃ (308 mg, 2.23 mmol) and Pd(dppf)Cl₂ (81 mg 0.11 mmol). The reaction mixture was stirred at 80° C. under N₂ for 16 hrs. The solvent was removed under reduced pressure and the residue was purified by Combiflash (PE/EA=0˜100%) to give the product tert-butyl 4-((3-(2′-chloro-4′-(methylsulfonyl)-[1,1′-biphenyl]-3-yl)-2-oxotetrahydropyrimidin-1(2H)-yl)methyl)piperidine-1-carboxylate as a yellow solid (150 mg, 23%). Mass (m/z): 584.0 [M+Na]⁺.

Step 4. Preparation of 1-[3-(2-chloro-4-methanesulfonylphenyl)phenyl]-3-(piperidin-4-ylmethyl)-1,3-diazinan-2-one: To a solution of tert-butyl 4-((3-(2′-chloro-4′-(methylsulfonyl)-[1,1′-biphenyl]-3-yl)-2-oxotetrahydropyrimidin-1(2H)-yl)methyl)piperidine-1-carboxylate (150 mg, 0.27 mmol] in DCM (6 mL) was added TFA (2 mL). The reaction mixture was stirred at rt for 1 hour. The resulting mixture was concentrated to give the product 1-[3-(2-chloro-4-methanesulfonylphenyl)phenyl]-3-(piperidin-4-ylmethyl)-1,3-diazinan-2-one as a yellow solid (200 mg, putity: 60%). Mass (m/z): 462.0 [M+H]⁺.

Step 5. Preparation of 5-[4-({3-[3-(2-chloro-4-methanesulfonylphenyl)phenyl]-2-oxo-1,3-diazinan-1-yl}methyl)piperidin-1-yl]-2-(2,6-dioxopiperidin-3-yl)isoindole-1,3-dione: To a solution of 1-[3-(2-chloro-4-methanesulfonylphenyl)phenyl]-3-(piperidin-4-ylmethyl)-1,3-diazinan-2-one (200 mg, 0.43 mmol) in DMSO (10 mL) was added 2-(2,6-dioxopiperidin-3-yl)-5-fluoroisoindole-1,3-dione (120 mg, 0.43 mmol) and DIEA (279 mg, 2.16 mmol). The reaction mixture was stirred at 80° C. under N₂ for 2 hrs. After the reaction completed, H₂O (100 mL) was added to the reaction mixture, and then extracted with EA (50 mL×3). The combined organic layer was washed with brine (50 mL×3), then dried over with anhydrous Na₂SO₄. After filtration, the solution was concentrated under vacuum, and the residue was purified by Combiflash (DCM/MeOH=0˜10%) to give the product 5-[4-({3-[3-(2-chloro-4-methanesulfonyl phenyl)phenyl]-2-oxo-1,3-diazinan-1-yl}methyl)piperidin-1-yl]-2-(2,6-dioxopiperidin-3-yl)isoindole-1,3-dione as a yellow solid (15 mg, 4%). Mass (m/z): 718.1 [M+H]⁺. ¹H NMR (400 MHz, CDCl₃) δ 8.03 (s, 1H), 7.63 (d, J=8.4 Hz, 1H), 7.55 (d, J=8.0 Hz, 2H), 7.34 (t, J=8.0 Hz, 2H), 7.05 (t, J=7.4 Hz, 1H), 6.76 (d, J=1.8 Hz, 1H), 6.49 (dd, J=8.4, 2.0 Hz, 1H), 4.93 (dd, J=12.2, 5.2 Hz, 1H), 4.18 (t, J=8.0 Hz, 2H), 3.90-3.80 (m, 2H), 3.72 (dd, J=7.8, 5.6 Hz, 2H), 3.55-3.47 (m, 2H), 3.36 (t, J=6.8 Hz, 2H), 2.93-2.69 (m, 4H), 2.12 (dd, J=7.8, 5.4 Hz, 1H), 2.03-1.92 (m, 2H).

### Compound 34: 5-(4-((3-(3-(7-chloro-1-ethyl-1H-indazol-6-yl)phenyl)-2-oxotetrahydropyrimidin-1(2H)-yl)methyl)piperidin-1-yl)-2-(2,6-dioxopiperidin-3-yl)isoindoline-1,3-dione

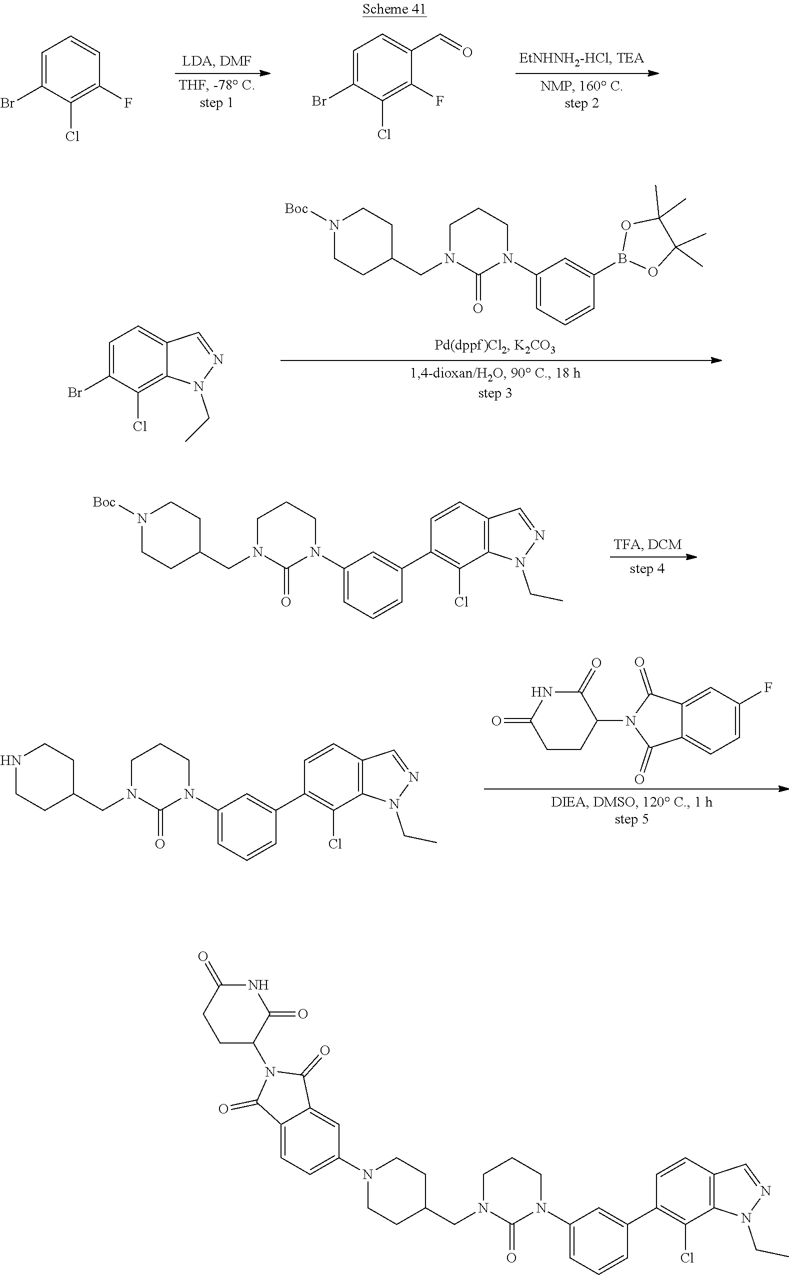

Step 1. Preparation of 4-bromo-3-chloro-2-fluorobenzaldehyde: To a solution of 1-bromo-2-chloro-3-fluorobenzene (2000 mg, 9.549 mmol) in THF (20 mL) was added dropwise LDA (2 M in THF, 7.2 mL, 14.32 mmol) at −78° C. under N₂. The reaction mixture was stirred at −78° C. under N₂ for 1 hour. DMF (1046 mg, 14.323 mmol) was added dropwise at −78° C. under N₂. The reaction mixture was stirred at RT under N₂ for 3 hrs. The solution was quenched with saturated ammonium chloride solution (50 mL) and extracted with ethyl acetate (20 mL×3). The combined organic layers were washed with brine (30 mL×2), dried over Na₂SO₄. Then by filtration, the filtrate was concentrated. The residue was purified by column chromatography (DCM/PE=1:10) to give the target compound (700 mg, 52%) as brown oil.

Step 2. Preparation of 6-bromo-7-chloro-1-ethyl-1H-indazole: To a solution of 4-bromo-3-chloro-2-fluorobenzaldehyde (600 mg, 2.562 mmol) in NMP (12 mL) was added ethylhydrazine dihydrochloride (1219 mg, 12.634 mmol), Potassium carbonate (1746 mg, 12.634 mmol) and TEA (1276 mg, 12.634 mmol). The reaction mixture was stirred 160° C. under N₂ for 18 hrs. Water (30 mL) was added and the mixture was extracted with EA (30 mL×3). The combined organic layers were washed with brine (20 mL×2) dried over Na₂SO₄. Then by filtration, the filtrate was concentrated. The residue was purified by column chromatography (EA/PE=1:2) to give the target product (300 mg, 43.46%) as a white solid. Mass (m/z): 259 [M+H]⁺.

Step 3. Preparation of tert-butyl 4-((3-(3-(7-chloro-1-ethyl-1H-indazol-6-yl) phenyl)-2 oxotetrahydro pyrimidin-1(2H)-yl) methyl) piperidine-1-carboxylate: To a mixture of 6-bromo-7-chloro-1-ethylindazole (300 mg, 1.155 mmol), tert-butyl [4-({2-oxo-3-[3-(4,4,5,5-tetramethyl-1,3,2-dioxaborolan-2-yl)phenyl]-1,3-diazinan-1-yl}methyl) piperidin-1-yl]formate (867 mg, 1.733 mmol) and Potassium carbonate (319 mg, 2.311 mmol) in 1,4-dioxane/H₂O (10:1, 6 mL) under N₂ was added Pd(dppf)Cl₂ (94 mg, 0.115 mmol). The reaction mixture was stirred at 90° C. for 18 hrs. The mixture was concentrated and the residue was purified by combi-flash (MeOH/DCM=1:10) to afford target compound (300 mg, yield: 44.58%) as a white solid. Mass (m/z): 474[M+Na]⁺.

Step 4. Preparation of 1-(3-(7-chloro-1-ethyl-1H-indazol-6-yl)phenyl)-3-(piperidin-4-ylmethyl) tetrahydropyrimidin-2(1H)-one: To a solution of tert-butyl [4-({3-[3-(7-chloro-1-ethylindazol-6-yl) phenyl]-2-oxo-1,3-diazinan-1-yl}methyl) piperidin-1-yl]formate (300 mg, 0.542 mmol) in DCM (3 mL) was added TFA (0.6 mL). The reaction mixture was stirred at rt under N₂ for 18 h. The solution was concentrated by reduced pressure to give the target compound (200 mg, 73%) as brown oil. Mass (m/z): 452 [M+H]⁺.

Step 5. Preparation of 5-(4-((3-(3-(7-chloro-1-ethyl-1H-indazol-6-yl) phenyl)-2-oxotetrahydropy rimidin-1(2H)-yl) methyl) piperidin-1-yl)-2-(2,6-dioxopiperidin-3-yl) isoindoline-1,3-dione: A solution of 1-[3-(7-chloro-1-ethylindazol-6-yl)phenyl]-3-(piperidin-4-ylmethyl)-1,3-diazinan-2-one (100 mg, 0.221 mmol), 2-(2,6-dioxopiperidin-3-yl)-5-fluoroisoindole-1,3-dione (73 mg, 0.265 mmol) and DIEA (285 mg, 2.212 mmol) in DMSO (5 mL) was stirred at 120° C. under N₂ for 1 hour. Water (20 mL) was added, and the mixture was extracted with EA (20 mL×3). The combined organic layers were washed with brine (20 mL×3), dried over Na₂SO₄. Then by filtration, the filtrate was concentrated. The residue was purified by prep-HPLC [Gemini-C18, 150×21.2 mm, 5 um; ACN—H₂O (0.1% FA), 20-50] to give the desired product (7 mg, 4.39%) as a yellow solid. Mass (m/z): 708 [M+H]⁺. ¹H NMR (400 MHz, DMSO-d₆) δ 11.08 (s, 1H), 8.21 (s, 1H), 7.80 (d, J=8.2 Hz, 1H), 7.64 (d, J=8.5 Hz, 1H), 7.47-7.28 (m, 4H), 7.28-7.18 (m, 2H), 7.13 (d, J=8.2 Hz, 1H), 5.06 (dd, J=12.7, 5.4 Hz, 1H), 4.84-4.75 (m, 2H), 4.06 (d, J=12.6 Hz, 2H), 3.73 (d, J=5.9 Hz, 2H), 3.38 (d, J=5.9 Hz, 2H), 3.20 (d, J=7.0 Hz, 2H), 2.97 (t, J=11.7 Hz, 2H), 2.86 (d, J=13.5 Hz, 1H), 2.60 (s, 2H), 2.05 (s, 4H), 1.71 (d, J=11.7 Hz, 2H), 1.41 (t, J=7.1 Hz, 3H), 1.21 (d, J=19.9 Hz, 2H).

### Compound 35: 5-(4-((3-(2-(4-chloro-3-ethyl-1H-indol-5-yl)pyridin-4-yl)-2-oxotetrahydropyrimidin-1(2H)-yl)methyl)piperidin-1-yl)-2-(2,6-dioxopiperidin-3-yl)isoindoline-1,3-dione

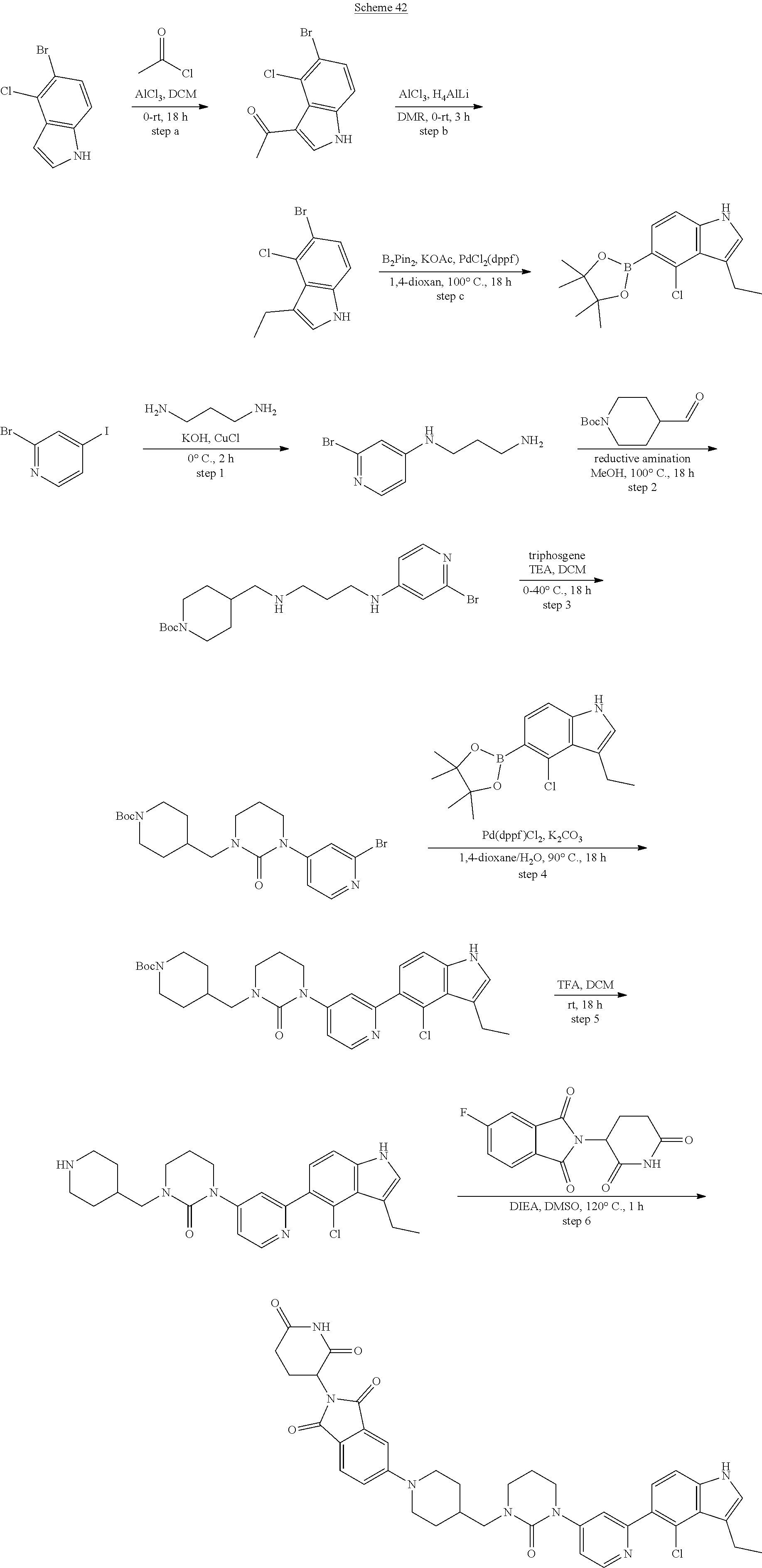

Step a. Preparation of 1-(5-bromo-4-chloro-1H-indol-3-yl) ethan-1-one: To a solution of AlCl₃ (28.91 g, 216.93 mmol) in DCM (100 mL) at 0° C. was added dropwise acetyl chloride (8.51 g, 108.46 mmol). Then 5-bromo-4-chloro-1H-indole (5 g, 21.69 mmol) was added at 0° C. The reaction mixture was stirred at rt under N₂ for 18 hrs. The mixture was poured into ice water and there was brown solid precipitated. The mixture was filtered, and the cake was dried to give the target compound (5.5 g, 88.38%) as a brown solid. Mass (m/z): 272 [M+H]⁺.

Step b. Preparation of 5-bromo-4-chloro-3-ethyl-1H-indole: To a solution of AlCl₃ (26.9 g, 201.81 mmol) in DME (500 mL) was added LiAlH₄ (3.83 g, 100.91 mmol) at 0° C. Then 1-(5-bromo-4-chloro-1H-indol-3-yl)ethenone (5.5 g, 20.181 mmol) was added at 0° C. The reaction mixture was stirred at rt under N₂ for 3 hrs. The mixture was poured into ice water and the mixture was extracted with EA (200 mL×3). The combined organic layers were washed with brine (200 mL×3), dried over Na₂SO₄ and concentrated to give the target compound (2.5 g, 45.52%) as a gray solid. Mass (m/z): 258 [M+H]⁺.

Step c. Preparation of 4-chloro-3-ethyl-5-(4,4,5,5-tetramethyl-1,3,2-dioxaborolan-2-yl)-1H-indole: To a mixture of 5-bromo-4-chloro-3-ethyl-1H-indole (500 mg, 1.933 mmol), B₂Pin₂ (736 mg, 2.9 mmol) and Potassium Acetate (379 mg, 3.867 mmol) in 1,4-dioxane (10 mL) under N₂ was added Pd(dppf)Cl₂ (157 mg, 0.193 mmol). The reaction mixture was stirred at 90° C. under N₂ for 18 hrs. The mixture was concentrated under reduced pressure. The residue was purified by column chromatography (EA/PE=1:6) to give the target compound (250 mg, 38.07%) as a white solid. Mass (m/z): 306 [M+H]⁺.

Step 1. Preparation of N¹-(2-bromopyridin-4-yl) propane-1,3-diamine: To a mixture of CuCl (17 mg, 0.176 mmol) in propane-1,3-diamine (2 mL) was added 2-bromo-4-iodopyridine (500 mg, 1.761 mmol) at 0° C. The reaction mixture was stirred at 0° C. under N₂ for 2 hrs. Water (20 mL) was added and extracted with DCM (20 mL×2). The DCM layer was washed with brine (20 mL×2) dried over Na₂SO₄ and concentrated. The residue was purified by column chromatography (MeOH/DCM=1:1) to give the target compound (200 mg, 44.42%) as a brown solid. Mass (m/z): 230 [M+H]⁺.

Step 2. Preparation of tert-butyl 4-(((3-((2-bromopyridin-4-yl) amino) propyl) amino) methyl) piperidine-1-carboxylate: To a solution of N-(3-aminopropyl)-2-bromopyridin-4-amine (200 mg, 0.869 mmol) and tert-butyl 4-formylpiperidine-1-carboxylate (185 mg, 0.869 mmol) in MeOH (2 mL) was added NaBH₃CN (163 mg, 2.607 mmol) at 0° C. The reaction mixture was stirred at rt under N₂ for 18 hrs. The mixture was concentrated under reduced pressure. The residue was purified by column chromatography (DCM/MeOH=10:1) to give the target compound (260 mg, yield: 66.49%) as brown oil. Mass (m/z): 427 [M+H]⁺.

Step 3. Preparation of tert-butyl 4-((3-(2-bromopyridin-4-yl)-2-oxotetrahydropyrimidin-1(2H)-yl) methyl) piperidine-1-carboxylate: To a solution of tert-butyl 4-[({3-[(2-bromopyridin-4-yl) amino]propyl}amino) methyl]piperidine-1-carboxylate (260 mg, 0.608 mmol) and DIEA (156 mg, 1.2168 mmol) in DCM (10 mL) was added a solution of Triphosgene (90 mg, 0.304 mmol) in DCM (2 mL) at 0° C. The reaction mixture was stirred at 40° C. under N₂ for 18 hrs. The mixture was concentrated under reduced pressure. The residue was purified by column chromatography (EA/PE=1:1) to give the target compound (150 mg, yield: 51.66%) as a brown solid. Mass (m/z): 453 [M+H]+.

Step 4. Preparation of tert-butyl 4-((3-(2-(4-chloro-3-ethyl-1H-indol-5-yl) pyridin-4-yl)-2-oxotetrahydropyrimidin-1(2H)-yl) methyl) piperidine-1-carboxylate: To a mixture of tert-butyl 4-{[3-(2-bromopyridin-4-yl)-2-oxo-1,3-diazinan-1-yl]methyl}piperidine-1-carboxylate (200 mg, 0.441 mmol), 4-chloro-3-ethyl-5-(4,4,5,5-tetramethyl-1,3,2-dioxaborolan-2-yl)-1H-indole (202 mg, 0.661 mmol) and Potassium carbonate (121 mg, 0.852 mmol) in 1,4-dioxane/H₂O (10:1, 2 mL) under N₂ was added Pd(dppf)Cl₂ (35 mg, 0.044 mmol). The reaction mixture was stirred at 90° C. under N₂ for 18 hrs. The reaction mixture was filtered and the filtrate was concentrated. The residue was purified by combi-flash (MeOH/DCM=1:10) to afford target compound (200 mg, yield: 73.91%) as a white solid. Mass (m/z): 552[M+H]⁺.

Step 5. Preparation of 1-(2-(4-chloro-3-ethyl-1H-indol-5-yl) pyridin-4-yl)-3-(piperidin-4-ylmethyl) tetrahydropyrimidin-2(1H)-one: To a solution of tert-butyl 4-({3-[2-(4-chloro-3-ethyl-1H-indol-5-yl) pyridin-4-yl]-2-oxo-1,3-diazinan-1-yl}methyl) piperidine-1-carboxylate (300 mg, 0.542 mmol) in DCM (3 mL) was added TFA (0.6 mL). The reaction mixture was stirred at RT under N₂ for 18 hrs. The solution was concentrated under reduced pressure to give the target compound (150 mg, 48.86%) as brown oil. Mass (m/z): 452 [M+H]⁺.

Step 6. Preparation of 5-(4-((3-(2-(4-chloro-3-ethyl-1H-indol-5-yl)pyridin-4-yl)-2-oxotetrahy dropyrimidin-1(2H)-yl)methyl)piperidin-1-yl)-2-(2,6-dioxopiperidin-3-yl) isoindoline-1,3-dione: A solution of 1-[2-(4-chloro-3-ethyl-1H-indol-5-yl)pyridin-4-yl]-3-(piperidin-4-ylmethyl)-1,3-diazinan-2-one (100 mg, 0.221 mmol), 2-(2,6-dioxopiperidin-3-yl)-5-fluoroisoindole-1,3-dione (73 mg, 0.265 mmol) and DIEA (285 mg, 2.212 mmol) in DMSO (5 mL). The reaction mixture was stirred at 120° C. under N₂ for 1 hour. Water (20 mL) was added, and the mixture was extracted with EA (20 mL×3). The combined organic layers were washed with brine (20 mL×3), dried over Na₂SO₄. Then by filtration, the filtrate was concentrated. The residue was purified by prep-HPLC [Gemini-C18, 150×21.2 mm, 5 um; ACN—H₂O (0.1% FA), 20-50] to give the desired product (7.5 mg, 4.66%) as a yellow solid. Mass (m/z): 708 [M+H]⁺.

¹H NMR (400 MHz, DMSO-d₆) δ 11.21 (s, 1H), 11.08 (s, 1H), 8.46 (d, J=5.8 Hz, 1H), 7.64 (d, J=8.6 Hz, 1H), 7.58 (d, J=2.0 Hz, 1H), 7.30 (d, J=43.1 Hz, 4H), 7.17 (d, J=8.3 Hz, 1H), 5.06 (dd, J=12.9, 5.3 Hz, 1H), 4.07 (d, J=12.6 Hz, 2H), 3.83-3.74 (m, 2H), 3.39 (s, 4H), 3.23 (d, J=7.2 Hz, 2H), 2.97 (d, J=7.0 Hz, 3H), 2.54 (s, 3H), 2.06-1.98 (m, 4H), 1.71 (d, J=10.9 Hz, 2H), 1.29-1.21 (m, 5H).

### Compound A: 5-(4-((3-(3-(4-chloro-3-ethyl-1H-pyrrolo[2,3-b]pyridin-5-yl)phenyl)-2-oxotetrahydropyrimidin-1(2H)-yl)methyl)piperidin-1-yl)-2-(2,6-dioxopiperidin-3-yl)isoindoline-1,3-dione

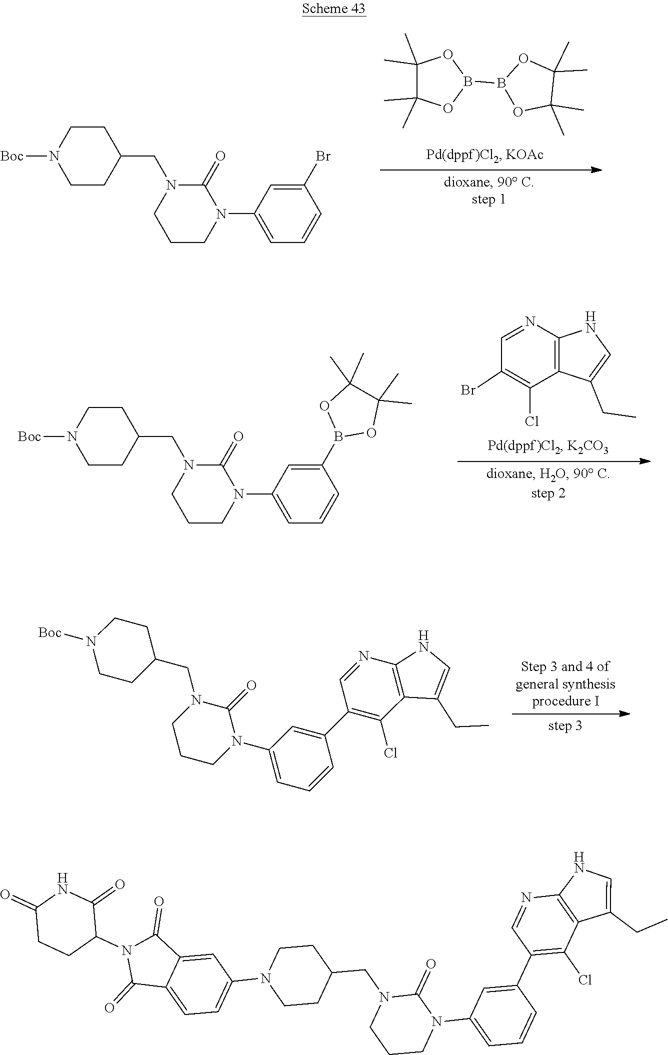

Step 1. Preparation of tert-butyl 4-((2-oxo-3-(3-(4,4,5,5-tetramethyl-1,3,2-dioxaborolan-2-yl)phenyl)tetrahydropyrimidin-1(2H)-yl)methyl)piperidine-1-carboxylate: To a solution of tert-butyl 4-((3-(3-bromophenyl)-2-oxotetrahydropyrimidin-1(2H)-yl)methyl)piperidine-1-carboxylate (11.1 g, 24.48 mmol) in dioxane (200 mL) was added 4,4,5,5-tetramethyl-2-(4,4,5,5-tetramethyl-1,3,2-dioxaborolan-2-yl)-1,3,2-dioxaborolane (9.3 g, 36.72 mmol), KOAc (7.2 g, 73.45 mmol) and Pd(dppf)Cl₂ (1.79 g, 2.45 mmol). The reaction mixture was stirred at 90° C. under N₂ for 2 hrs. The solvent was removed under reduced pressure and the residue was purified by Combi-flash (DCM/MeOH=0˜10%) to give the product as brown oil (12.3 g, 80%). Mass (m/z): 522.0 [M+H]⁺.

Step 2. Preparation of tert-butyl 4-((3-(3-(4-chloro-3-ethyl-1H-pyrrolo[2,3-b]pyridin-5-yl)phenyl)-2-oxotetrahydropyrimidin-1(2H)-yl)methyl)piperidine-1-carboxylate: To a solution of tert-butyl 4-((2-oxo-3-(3-(4,4,5,5-tetramethyl-1,3,2-dioxaborolan-2-yl)phenyl)tetrahydropyrimidin-1(2H)-yl)methyl)piperidine-1-carboxylate (12.3 g, 24.58 mmol) in dioxane/H₂O (10:1, 200 mL) was added 5-bromo-4-chloro-3-ethyl-1H-pyrrolo[2,3-b]pyridine (6.37 g, 24.58 mmol), K₂CO₃ (10.19 g, 73.73 mmol) and Pd(dppf)Cl₂ (1.79 g, 2.46 mmol). The reaction mixture was stirred at 90° C. under N₂ for 4 hrs. After the reaction completed, H₂O (300 mL) was added to the reaction mixture, and then extracted with DCM (200 mL×3). The combined organic layer was washed with brine (300 mL×2), then dried over anhydrous Na₂SO₄. After filtration, the solution was concentration under vacuum, and the residue was purified by Combi-flash (DCM/MeOH=0˜10%) to give the product as a brown solid (5.83 g, 39%). Mass (m/z): 552.0 [M+H]⁺.

Step 3. Preparation of 5-(4-((3-(3-(4-chloro-3-ethyl-1H-pyrrolo[2,3-b]pyridin-5-yl)phenyl)-2-oxotetrahydropyrimidin-1(2H)-yl)methyl)piperidin-1-yl)-2-(2,6-dioxopiperidin-3-yl)isoindoline-1,3-dione: Following step 3 and 4 of general synthesis procedure I, from tert-butyl 4-((3-(3-(4-chloro-3-ethyl-1H-pyrrolo[2,3-b]pyridin-5-yl)phenyl)-2-oxotetrahydropyrimidin-1(2H)-yl)methyl)piperidine-1-carboxylate, compound 5-(4-((3-(3-(4-chloro-3-ethyl-1H-pyrrolo[2,3-b]pyridin-5-yl)phenyl)-2-oxotetrahydropyrimidin-1(2H)-yl)methyl)piperidin-1-yl)-2-(2,6-dioxopiperidin-3-yl)isoindoline-1,3-dione was obtained as a yellow solid (1.45 g, 21%). Mass (m/z): 707.7 [M+H]⁺. ¹H NMR (400 MHz, DMSO-d₆) (J 11.79 (s, 1H), 11.08 (s, 1H), 8.11 (s, 1H), 7.64 (d, J=8.6 Hz, 1H), 7.37 (ddd, J=25.2, 13.8, 8.2 Hz, 5H), 7.22 (t, J=9.8 Hz, 2H), 5.06 (dd, J=12.8, 5.4 Hz, 1H), 4.07 (d, J=12.8 Hz, 2H), 3.77-3.70 (m, 2H), 3.39 (t, J=5.8 Hz, 2H), 3.20 (d, J=7.2 Hz, 2H), 3.02-2.82 (m, 5H), 2.62-2.53 (m, 2H), 2.03 (dd, J=12.8, 7.2 Hz, 4H), 1.71 (d, J=11.8 Hz, 2H), 1.28 (t, J=7.4 Hz, 3H).

### Compound B: 5-(4-((3-(3-(4-chloro-3-ethyl-1H-pyrrolo[2,3-b]pyridin-5-yl)phenyl)-2-oxotetrahydropyrimidin-1(2H)-yl)methyl)piperidin-1-yl)-2-(2,6-dioxopiperidin-3-yl)isoindoline-1,3-dione

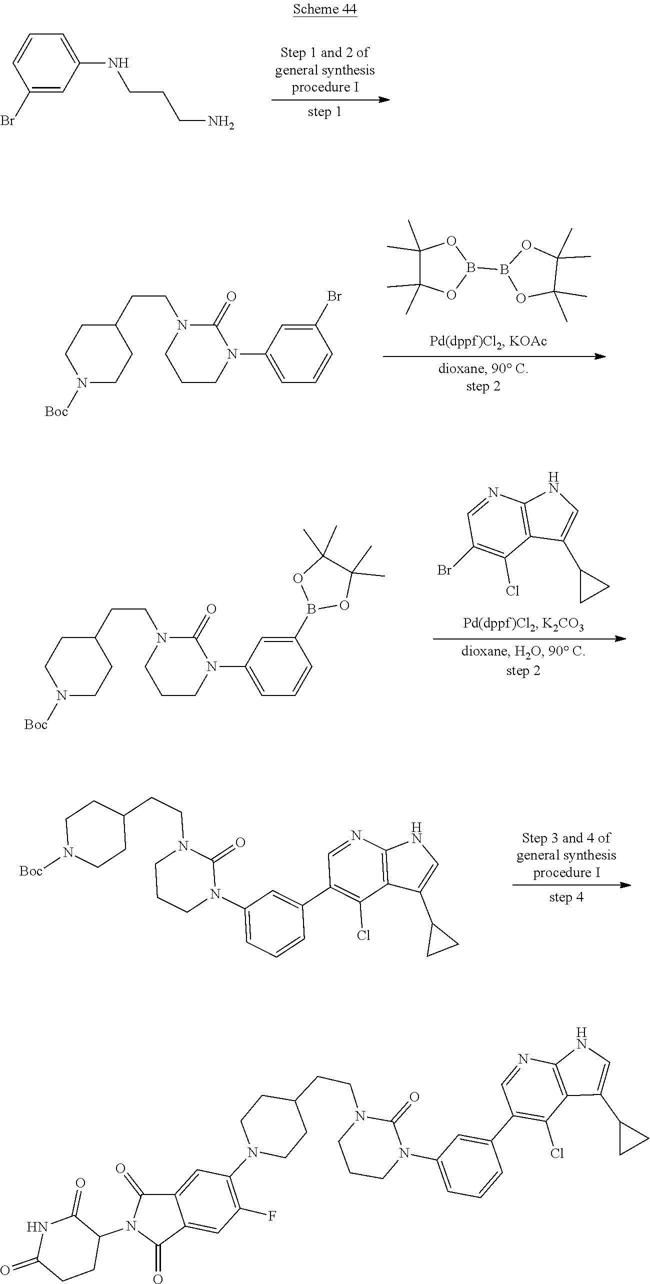

Step 1. Preparation of tert-butyl 4-(2-(3-(3-bromophenyl)-2-oxotetrahydropyrimidin-1(2H)-yl)ethyl)piperidine-1-carboxylate: Following step 1 and step 2 of general synthesis procedure I, from tert-butyl 4-(2-oxoethyl)piperidine-1-carboxylate, product tert-butyl 4-(2-(3-(3-bromophenyl)-2-oxotetrahydropyrimidin-1(2H)-yl)ethyl)piperidine-1-carboxylate was obtained (660 mg, 56%) as yellow oil. Mass (m/z): 488.2 [M+H]⁺.

Step 2. Preparation of tert-butyl 4-(2-(2-oxo-3-(3-(4,4,5,5-tetramethyl-1,3,2-dioxa borolan-2-yl)phenyl)tetrahydropyrimidin-1(2H)-yl)ethyl)piperidine-1-carboxylate: To a solution of tert-butyl 4-(2-(3-(3-bromophenyl)-2-oxotetrahydropyrimidin-1(2H)-yl)ethyl)piperidine-1-carboxylate (660 mg, 1.41 mmol) in dioxane (20 mL) was added 4,4,4′,4′,5,5,5′,5′-octamethyl-2,2′-bi(1,3,2-dioxaborolane) (538 mg, 2.12 mmol), KOAc (416 mg, 4.24 mmol) and Pd(dppf)Cl₂ (103 mg, 0.14 mmol). The reaction mixture was stirred at 90° C. under N₂ for 2 hrs. The solvent was removed under reduced pressure and the residue was purified by Combi-flash (DCM/MeOH=0˜10%) to give the product (650 mg, 89%) as brown oil. Mass (m/z): 536.0 [M+H]⁺.

Step 3. Preparation of tert-butyl 4-(2-(3-(3-(4-chloro-3-cyclopropyl-1H-pyrrolo[2,3-b]pyridin-5-yl)phenyl)-2-oxotetrahydropyrimidin-1(2H)-yl)ethyl)piperidine-1-carboxylate: To a solution of tert-butyl 4-(2-(2-oxo-3-(3-(4,4,5,5-tetramethyl-1,3,2-dioxaborolan-2-yl)phenyl)tetrahydropyrimidin-1(2H)-yl)ethyl)piperidine-1-carboxylate (650 mg, 1.26 mmol) in dioxane/H₂O (10:1, 15 mL) was added 5-bromo-4-chloro-3-cyclopropyl-1H-pyrrolo[2,3-b]pyridine (343 mg, 1.26 mmol), K₂CO₃ (523 mg, 3.79 mmol) and Pd(dppf)Cl₂ (92 mg, 0.13 mmol). The reaction mixture was stirred at 90° C. under N₂ for 4 hrs. After the reaction completed, H₂O (20 mL) was added to the reaction mixture, and then extracted with DCM (20 mL×3). The combined organic layer was washed with brine (30 mL×2), then dried over anhydrous Na₂SO₄. After filtration, the solution was concentration under vacuum, and the residue was purified by Combi-flash (DCM/MeOH=0˜10%) to give the product (250 mg, 34%) as yellow oil. Mass (m/z): 578.3 [M+H]⁺.

Step 4. Preparation of 5-(4-(2-(3-(3-(4-chloro-3-cyclopropyl-1H-pyrrolo[2,3-b]pyridin-5-yl)phenyl)-2-oxotetrahydropyrimidin-1(2H)-yl)ethyl)piperidin-1-yl)-2-(2,6-dioxopiperidin-3-yl)-6-fluoroisoindoline-1,3-dione: Following step 3 and 4 of general synthesis procedure I, from tert-butyl 4-(2-(3-(3-(4-chloro-3-cyclopropyl-1H-pyrrolo[2,3-b]pyridin-5-yl)phenyl)-2-oxotetrahydropyrimidin-1(2H)-yl)ethyl)piperidine-1-carboxylate, compound 5-(4-(2-(3-(3-(4-chloro-3-cyclopropyl-1H-pyrrolo[2,3-b]pyridin-5-yl)phenyl)-2-oxotetrahydropyrimidin-1(2H)-yl)ethyl)piperidin-1-yl)-2-(2,6-dioxopiperidin-3-yl)-6-fluoroisoindoline-1,3-dione was obtained as a yellow solid (85 mg, 23%). Mass (m/z): 751.7 [M+H]⁺. ¹H NMR (400 MHz, DMSO-d₆) δ 11.73 (d, J=2.4 Hz, 1H), 11.11 (s, 1H), 8.11 (s, 1H), 7.69 (d, J=11.6 Hz, 1H), 7.44-7.36 (m, 3H), 7.34-7.28 (m, 2H), 7.23-7.19 (m, 1H), 5.10 (dd, J=12.8, 5.4 Hz, 1H), 3.75-3.69 (m, 2H), 3.59 (d, J=12.4 Hz, 2H), 3.36 (d, J=10.2 Hz, 4H), 2.90-2.82 (m, 3H), 2.56 (dd, J=19.8, 10.6 Hz, 2H), 2.23-2.15 (m, 1H), 2.04 (dd, J=14.6, 9.2 Hz, 3H), 1.84 (d, J=11.8 Hz, 2H), 1.50 (d, J=6.8 Hz, 3H), 1.37-1.28 (m, 2H), 0.83 (ddd, J=8.2, 6.0, 4.0 Hz, 2H), 0.66-0.60 (m, 2H).

### Compound C: 3-(5-{4-[(3-{3-[4-chloro-5-(2,2-difluoroethyl)-7H-pyrrolo[2,3-b]pyridin-3-yl]phenyl}-2-oxo-1,3-diazinan-1-yl)methyl]piperidin-1-yl}-1-oxo-3H-isoindol-2-yl)piperidine-2,6-dione

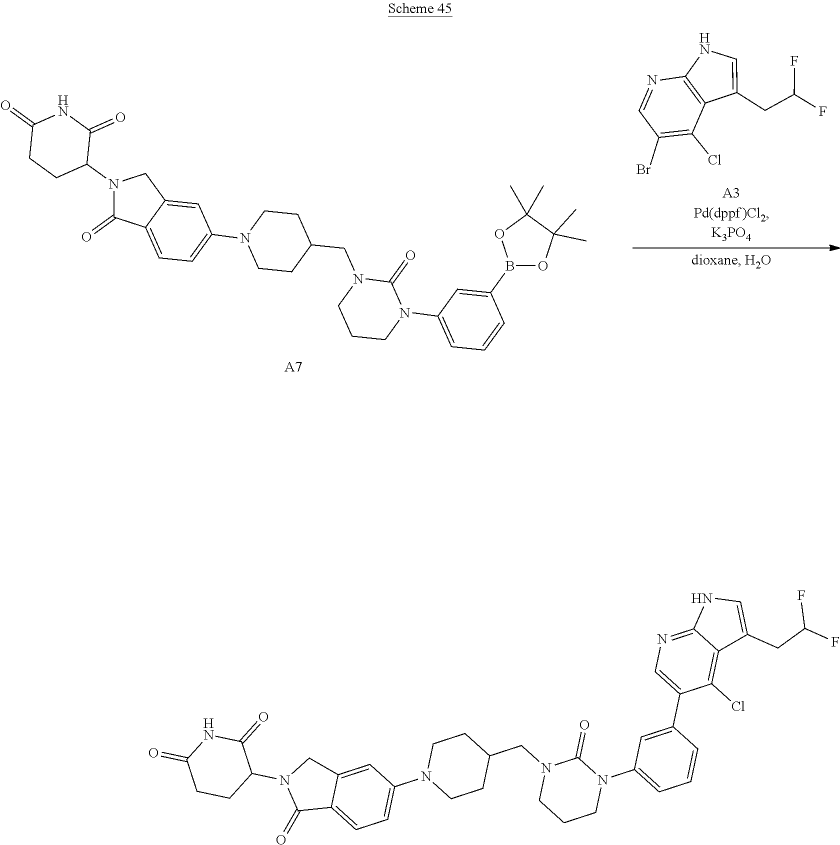 [SMILES: CC1(C)OB(c2cccc(N3CCCN(CC4CCN(c5ccc6c(c5)CN(C5CCC(=O)NC5=O)C6=O)CC4)C3=O)c2)OC1(C)C ; FC(F)Cc1c[nH]c2ncc(Br)c(Cl)c12 ; "A7" ; O=C1CCC(N2Cc3cc(N4CCC(CN5CCCN(c6cccc(-c7cnc8[nH]cc(CC(F)F)c8c7Cl)c6)C5=O)CC4)ccc3C2=O)C(=O)N1]

Step 1. Preparation of 3-(5-{4-[(3-{3-[4-chloro-5-(2,2-difluoroethyl)-7H-pyrrolo[2,3-b]pyridin-3-yl]phenyl}-2-oxo-1,3-diazinan-1-yl)methyl]piperidin-1-yl}-1-oxo-3H-isoindol-2-yl)piperidine-2,6-dione: To a solution of 3-{1-oxo-5-[4-({2-oxo-3-[3-(4,4,5,5-tetramethyl-1,3,2-dioxaborolan-2-yl)phenyl]-1,3-diazinan-1-yl}methyl)piperidin-1-yl]-3H-isoindol-2-yl}piperidine-2,6-dione (70 mg, 0.11 mmol), 5-bromo-4-chloro-3-(2,2-difluoroethyl)-1H-pyrrolo[2,3-b]pyridine (39 mg, 0.11 mmol) and K₃PO₄ (69 mg, 0.33 mmol) in 1.4-dioxane/H₂O (10:1, 3 mL) was added Pd(dppf)Cl₂ (8 mg, 0.011 mmol). The solution was stirred under nitrogen at 85° C. for 16 hrs. The reaction was cooled to room temperature. Water (15 mL) was added and the mixture was extracted with EA (10 mL×3). The combined organic layers were washed with brine (20 mL×2), dried over Na₂SO₄. Then by filtration, the filtrate was concentrated. The residue was purified by Prep-HPLC [Gemini-C18, 150×21.2 mm, Sum; ACN—H₂O (0.1% FA), 35-50] to give the product as a yellow solid (2 mg, 2.5%). Mass (m/z): 730.3 [M+H]⁺.

¹H NMR (400 MHz, DMSO-d₆) δ 12.09 (s, 1H), 10.93 (s, 1H), 8.40 (s, 1H), 8.16 (s, 1H), 7.58 (s, 1H), 7.49 (d, J=8.6 Hz, 1H), 7.43-7.27 (m, 3H), 7.21 (d, J=7.6 Hz, 1H), 7.03 (d, J=7.8 Hz, 1H), 6.30 (s, 1H), 5.03 (d, J=12.6 Hz, 1H), 4.31 (d, J=17.0 Hz, 1H), 4.18 (d, J=17.2 Hz, 1H), 3.89 (d, J=11.4 Hz, 2H), 3.74 (s, 2H), 3.54 (d, J=16.6 Hz, 2H), 3.40 (s, 2H), 3.20 (s, 2H), 2.85 (dd, J=25.6, 13.0 Hz, 2H), 2.63 (d, J=28.0 Hz, 2H), 2.33 (s, 1H), 2.06 (s, 2H), 1.93 (s, 2H), 1.70 (d, J=11.0 Hz, 2H), 1.24 (s, 2H).

### Compound D: 3-(6-{4-[(3-{3-[4-chloro-5-(2,2-difluoroethyl)-7H-pyrrolo[2,3-b]pyridin-3-yl]phenyl}-2-oxo-1,3-diazinan-1-yl)methyl]piperidin-1-yl}-1-oxo-3H-isoindol-2-yl)piperidine-2,6-dione

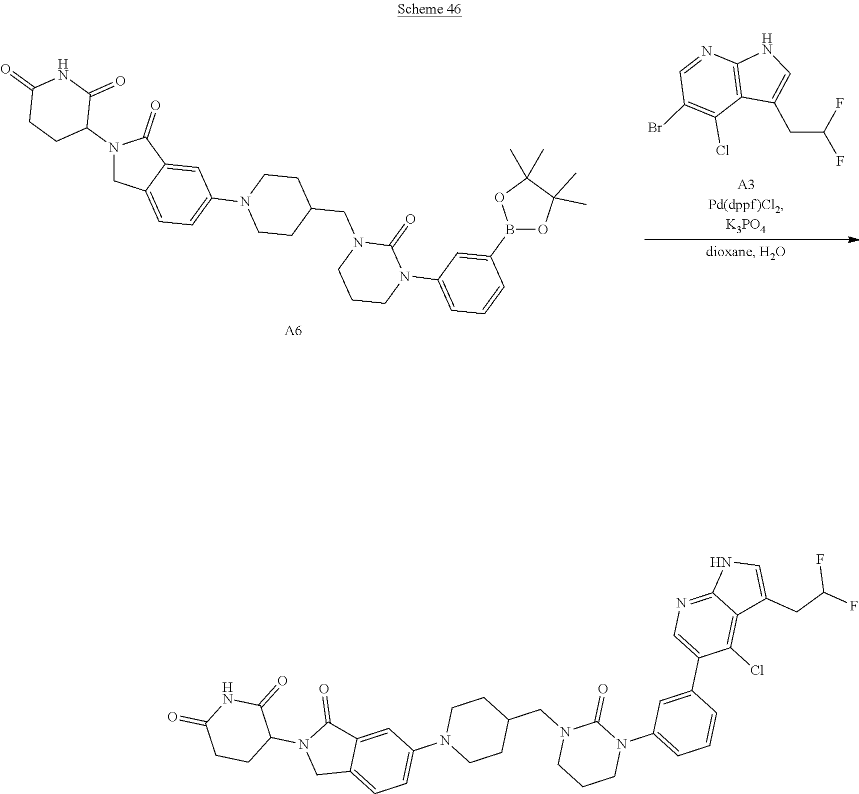 [SMILES: CC1(C)OB(c2cccc(N3CCCN(CC4CCN(c5ccc6c(c5)C(=O)N(C5CCC(=O)NC5=O)C6)CC4)C3=O)c2)OC1(C)C ; FC(F)Cc1c[nH]c2ncc(Br)c(Cl)c12 ; "A6" ; O=C1CCC(N2Cc3ccc(N4CCC(CN5CCCN(c6cccc(-c7cnc8[nH]cc(CC(F)F)c8c7Cl)c6)C5=O)CC4)cc3C2=O)C(=O)N1]

Step 1. Preparation of 3-(6-{4-[(3-{3-[4-chloro-5-(2,2-difluoroethyl)-7H-pyrrolo[2,3-b]pyridin-3-yl]phenyl}-2-oxo-1,3-diazinan-1-yl)methyl]piperidin-1-yl}-1-oxo-3H-isoindol-2-yl)piperidine-2,6-dione: To a solution of 3-{1-oxo-6-[4-({2-oxo-3-[3-(4,4,5,5-tetramethyl-1,3,2-dioxaborolan-2-yl)phenyl]-1,3-diazinan-1-yl}methyl)piperidin-1-yl]-3H-isoindol-2-yl}piperidine-2,6-dione (100 mg, 0.15 mmol), 3-bromo-4-chloro-5-(2,2-difluoroethyl)-7H-pyrrolo[2,3-b]pyridine (55 mg, 0.18 mmol and K₃PO₄ (99 mg, 0.46 mmol) in 1.4-dioxane/H₂O (5 mL) was added Pd(dppf)Cl₂ (11 mg, 0.015 mmol). The solution was stirred under nitrogen at 85° C. for 16 hrs. The reaction was cooled to room temperature. Water (15 mL) was added and the mixture was extracted with EA (10 mL×3). The combined organic layers were washed with brine (20 mL×2), dried over Na₂SO₄. Then by filtration, the filtrate was concentrated. The crude product was purified by Prep-HPLC [Gemini-C18, 150×21.2 mm, 5 um; ACN—H₂O (0.10% FA), 30-50] to give the product as a yellow solid (6 mg, 5.1%). Mass (m/z): 730.3 [M+H]⁺. ¹H NMR (400 MHz, DMSO-d₆) δ 12.10 (d, J=1.8 Hz, 1H), 10.97 (s, 1H), 8.15 (d, J=12.4 Hz, 1H), 7.58 (d, J=2.0 Hz, 1H), 7.38 (td, J=15.2, 7.8 Hz, 4H), 7.23 (dd, J=16.4, 8.0 Hz, 2H), 7.15 (s, 1H), 6.48-6.12 (m, 1H), 5.09 (dd, J=13.2, 5.0 Hz, 1H), 4.32 (d, J=16.8 Hz, 1H), 4.19 (d, J=16.8 Hz, 1H), 3.86-3.66 (m, 4H), 3.52 (td, J=17.2, 4.2 Hz, 2H), 3.44-3.37 (m, 2H), 3.22 (d, J=7.2 Hz, 2H), 2.96-2.85 (m, 1H), 2.71 (t, J=11.5 Hz, 2H), 2.58 (d, J=17.0 Hz, 1H), 2.42-2.29 (m, 1H), 2.10-2.00 (m, 2H), 1.98-1.85 (m, 2H), 1.71 (d, J=11.2 Hz, 2H), 1.32-1.23 (m, 2H).

### Compound E: 5-({2-[3-(3-{4-chloro-5-ethyl-7H-pyrrolo[2,3-b]pyridin-3-yl}phenyl)-2-oxo-1,3-diazinan-1-yl]ethyl}amino)-2-(2,6-dioxopiperidin-3-yl)isoindole-1,3-dione

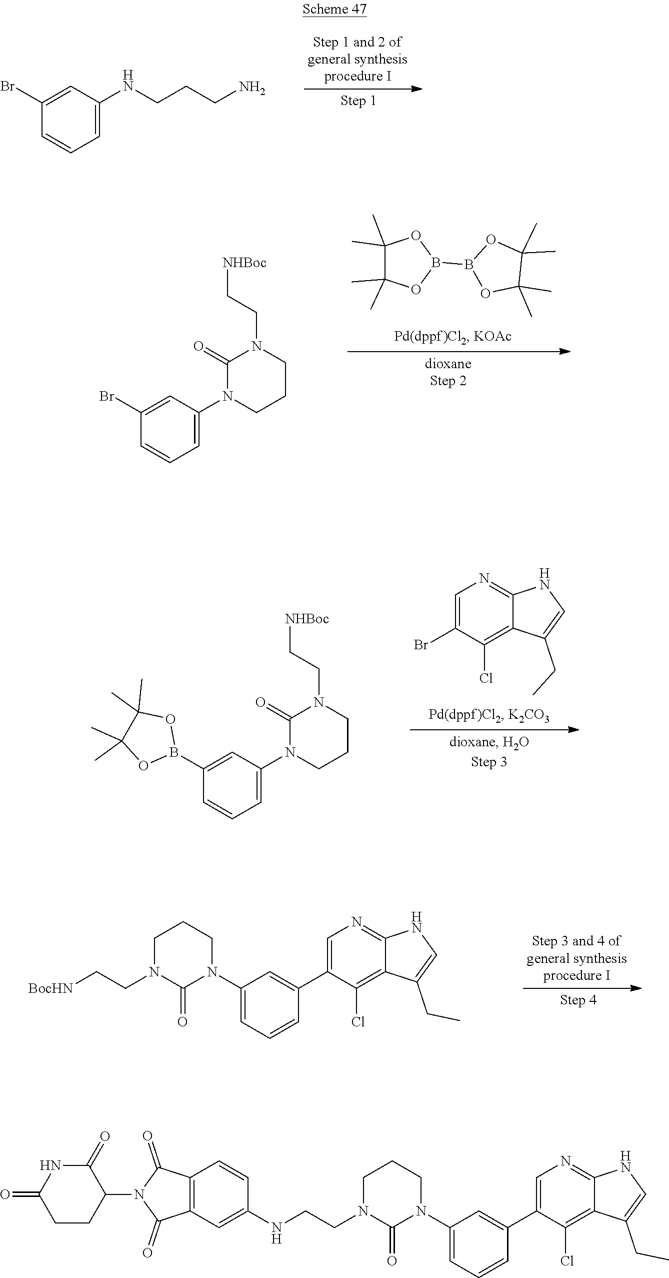

Step 1. Preparation of tert-butyl N-{2-[3-(3-bromophenyl)-2-oxo-1,3-diazinan-1-yl]ethyl}carbamate: Following step 1 and step 2 of general synthesis procedure I, from N-(3-aminopropyl)-3-bromoaniline, compound tert-butyl N-{2-[3-(3-bromophenyl)-2-oxo-1,3-diazinan-1-yl]ethyl}carbamate was obtained as a yellow solid (0.93 g, 85%). Mass (m/z): 420.0 [M+Na]⁺.

Step 2. Preparation of tert-butyl-N-(2-{2-oxo-3-[3-(4,4,5,5-tetramethyl-1,3,2-dioxaborolan-2-yl)phenyl]-1,3-diazinan-1-yl}ethyl)carbamate: To a mixture of tert-butyl-N-{2-[3-(3-bromophenyl)-2-oxo-1,3-diazinan-1-yl]ethyl}carbamate (930 mg, 2.33 mmol) in dioxane (20 mL) was added KOAc (687 mg, 7.01 mmol), 4,4,5,5-tetramethyl-2-(4,4,5,5-tetramethyl-1,3,2-dioxaborolan-2-yl)-1,3,2-dioxaborolane (1.18 g, 4.67 mmol) and Pd(dppf)Cl₂ (171 mg, 0.233 mmol). The reaction was degassed with N₂ and stirred at 90° C. for 16 hrs. The reaction mixture was filtered and the filtrate was concentrated under reduced pressure. The residue was diluted with water (50 mL), then extracted with EA (50 mL×3), washed with brine (100 mL), dried over Na₂SO₄ and concentrated under reduced pressure. The residue was purified by Flash Chromatography (PE/EA=0˜50%) to give the product tert-butyl-N-(2-{2-oxo-3-[3-(4,4,5,5-tetramethyl-1,3,2-dioxaborolan-2-yl)phenyl]-1,3-diazinan-1-yl}ethyl)carbamate as a yellow solid (0.53 g, 46%). Mass (m/z): 445.3 [M+H]⁺.

Step 3. Preparation of tert-butyl-N-{2-[3-(3-{4-chloro-5-ethyl-7H-pyrrolo[2,3-b]pyridin-3-yl}phenyl)-2-oxo-1,3-diazinan-1-yl]ethyl}carbamate: To a mixture of tert-butyl-N-(2-{2-oxo-3-[3-(4,4,5,5-tetramethyl-1,3,2-dioxaborolan-2-yl)phenyl]-1,3-diazinan-1-yl}ethyl)carbamate (500 mg, 1.12 mmol) in dioxane/H₂O (10:1, 10.0 mL) was added K₂CO₃ (465 mg, 3.37 mmol), 3-bromo-4-chloro-5-ethyl-7H-pyrrolo[2,3-b]pyridine (320 mg, 1.23 mmol) and Pd(dppf)Cl₂ (82 mg, 0.112 mmol). The reaction was degassed with N₂ and stirred at 100° C. for 16 hrs. The reaction mixture was filtered and the filtrate was concentrated under reduced pressure. The residue was diluted with water (50 mL), then extracted with EA (50 mL×3), washed with brine (100 mL), dried over Na₂SO₄ and concentrated under reduced pressure. The residue was purified by Flash Chromatography (PE/EA=0˜60%) to give the product tert-butyl-N-{2-[3-(3-{4-chloro-5-ethyl-7H-pyrrolo[2,3-b]pyridin-3-yl}phenyl)-2-oxo-1,3-diazinan-1-yl]ethyl}carbamate as a brown solid (430 mg, 69%). Mass (m/z): 498.2 [M+H]⁺.

Step 4. Preparation of 5-({2-[3-(3-{4-chloro-5-ethyl-7H-pyrrolo[2,3-b]pyridin-3-yl}phenyl)-2-oxo-1,3-diazinan-1-yl]ethyl}amino)-2-(2,6-dioxopiperidin-3-yl)isoindole-1,3-dione: Following step 3 and step 4 of general synthesis procedure I, from tert-butyl-N-{2-[3-(3-{4-chloro-5-ethyl-7H-pyrrolo[2,3-b]pyridin-3-yl}phenyl)-2-oxo-1,3-diazinan-1-yl]ethyl}carbamate, compound 5-({2-[3-(3-{4-chloro-5-ethyl-7H-pyrrolo[2,3-b]pyridin-3-yl}phenyl)-2-oxo-1,3-diazinan-1-yl]ethyl}amino)-2-(2,6-dioxopiperidin-3-yl)isoindole-1,3-dione was obtained as a yellow solid (15 mg, 5%). Mass (m/z): 654.0 [M+H]⁺. ¹H NMR (400 MHz, CDCl₃) δ 13.23 (s, 1H), 8.11 (d, J=8.8 Hz, 2H), 7.56 (d, J=7.8 Hz, 1H), 7.52-7.49 (m, 1H), 7.40-7.33 (m, 3H), 6.95 (s, 1H), 6.75 (d, J=7.8 Hz, 1H), 4.91 (dd, J=11.8, 5.0 Hz, 1H), 3.75 (s, 4H), 3.47 (d, J=28.4 Hz, 4H), 3.04 (dd, J=14.6, 7.2 Hz, 2H), 2.90-2.69 (m, 3H), 2.18-2.09 (m, 3H), 1.36 (t, J=7.2 Hz, 3H).

### Compound F: 5-(3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidin-1-yl)-2-(2,6-dioxopiperidin-3-yl)isoindoline-1,3-dione

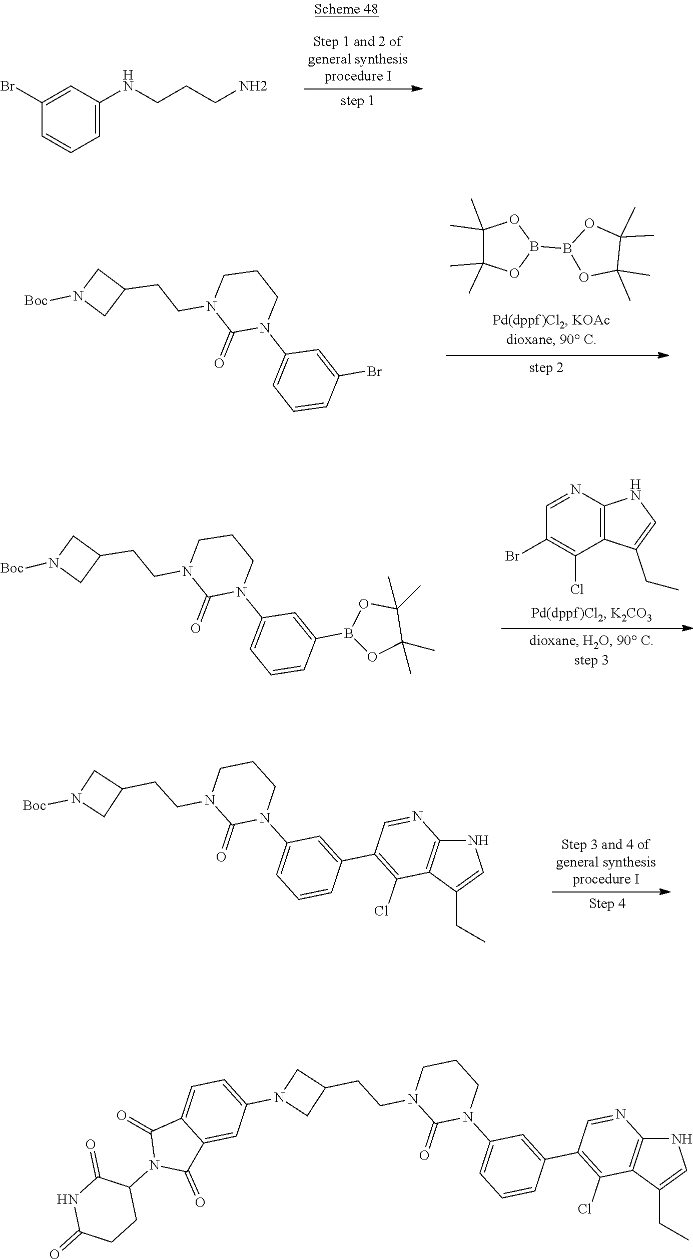

Step 1. Preparation of tert-butyl 3-(2-(3-(3-bromophenyl)-2-oxotetrahydropyrimidin-1(2H)-yl)ethyl)azetidine-1-carboxylate: Following step 1 and step 2 of general synthesis procedure I, from N¹-(3-bromophenyl)propane-1,3-diamine, compound tert-butyl 3-(2-(3-(3-bromophenyl)-2-oxotetrahydropyrimidin-1(2H)-yl)ethyl)azetidine-1-carboxylate was obtained as yellow oil (880 mg, 39%). Mass (m/z): 462.2 [M+H]⁺.

Step 2. Preparation of tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate: To a solution of tert-butyl 3-(2-(3-(3-bromophenyl)-2-oxotetrahydropyrimidin-1(2H)-yl)ethyl)azetidine-1-carboxylate (740 mg, 1.68 mmol) in dioxane (20 mL) was added Bis (pinacolato) diboron (642 mg, 2.53 mmol), KOAc (825 mg, 8.42 mmol) and Pd(dppf)Cl₂ (62 mg, 0.08 mmol). The reaction mixture was stirred at 90° C. under N₂ for 16 hrs. The solvent was removed under reduced pressure and the residue was purified by Combi-flash (PE/EA=0˜100%) to give the product tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate (450 mg, 54%). Mass (m/z): 486.2 [M+H]⁺.

Step 3. Preparation of tert-butyl 3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidine-1-carboxylate: To a solution of tert-butyl 3-(2-(2-oxo-3-(3-(4,4,5,5-tetramethyl-1,3,2-dioxaborolan-2-yl)phenyl)tetrahydropyrimidin-1(2H)-yl)ethyl)azetidine-1-carboxylate (450 mg, 0.9 mmol) in dioxane/H₂O (10:1, 15 mL) was added 5-bromo-4-chloro-3-ethyl-1H-pyrrolo[2,3-b]pyridine (240 mg, 0.93 mmol), K₂CO₃ (383 mg, 2.78 mmol) and Pd(dppf)Cl₂ (68 mg, 0.09 mmol). The reaction mixture was stirred at 90° C. under N₂ for 4 hrs. After the reaction completed, H₂O (20 mL) was added to the reaction mixture, and then extracted with DCM (20 mL×3). The combined organic layer was washed with brine (30 mL×2), then dried over anhydrous Na₂SO₄. After filtration, the solution was concentration under vacuum, and the residue was purified by Combi-flash (DCM/MeOH=0˜2%) to give the product tert-butyl 3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidine-1-carboxylate as a yellow oil (370 mg, 74%). Mass (m/z): 538.1 [M+H]⁺.

Step 4. Preparation of 5-(3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidin-1-yl)-2-(2,6-dioxopiperidin-3-yl)isoindoline-1,3-dione: Following step 3 and step 4 of general synthesis procedure I, from tert-butyl 3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidine-1-carboxylate, compound 5-(3-(2-(3-(3-(4-chloro-3-ethyl-1H-pyrrolo[2,3-b]pyridin-5-yl)phenyl)-2-oxotetrahydropyrimidin-1(2H)-yl)ethyl)azetidin-1-yl)-2-(2,6-dioxopiperidin-3-yl)isoindoline-1,3-dione was obtained as a yellow solid (136 mg, 21%). Mass (m/z): 694.1 [M+H]⁺. ¹H NMR (400 MHz, DMSO-d₆) δ 11.77 (d, J=2.4 Hz, 1H), 11.07 (s, 1H), 8.11 (s, 1H), 7.61 (d, J=8.2 Hz, 1H), 7.43-7.30 (m, 4H), 7.21 (d, J=7.6 Hz, 1H), 6.75 (d, J=2.0 Hz, 1H), 6.62 (dd, J=8.4, 2.0 Hz, 1H), 5.05 (dd, J=12.8, 5.4 Hz, 1H), 4.14 (t, J=8.2 Hz, 2H), 3.78-3.65 (m, 4H), 3.39 (t, J=5.8 Hz, 2H), 3.31 (s, 2H), 2.90 (dd, J=14.8, 7.4 Hz, 2H), 2.81 (dd, J=22.9, 6.8 Hz, 2H), 2.60 (s, 1H), 2.54 (s, 2H), 2.09-2.03 (m, 2H), 1.89 (d, J=6.8 Hz, 2H), 1.26 (t, J=7.4 Hz, 3H).

### General Assay Procedures:

### GSPT1 Degradation Assay

HL-60 cells (4×10\hat{ }⁶ cells/well) were seeded in 6-well culture plate (Costar, 3516) and treated with a variety of concentrations of test compound. After 2h incubation, cells were collected and lysed. The protein concentration was determined by BCA protein assay kit from Thermo (23227). The GSPT1 protein level was determined by western blots, using GSPT1 polyclonal antibody. Proteins were loaded into each well of the pre-casting gels and subjected to electrophoretic separation by SDS-PAGE. The protein resolved by SDS-PAGE were transferred to PVDF, blocked by 5% skim milk and probed with anti-GSPT1 antibody (Proteintech, 10763-1-AP) or anti-β-actin antibody (CST, 3700S), using following standard western blotting procedure. Blots intensities were quantified using ImageJ software and the intensity of GSPT1 bands were normalized to beta-Actin bands, respectively. The GSPT1 degradation results were then calculated. Reported compound C84971 (see WO2021126974A1 and WO2021126973A1) was used as a reference. The results of degradation assays are shown in the following Table 2.

TABLE 2
Results of compounds in GSPT1 protein degradation assays

| | % Degradation | % Degradation |
|---|---|---|
| Compound | (1 μM) | (0.1 μM) |
| C84971 | 0 | 0 |
| 1 | 51 | 45 |
| 2 | 0 | 0 |
| 3 | 48 | 30 |
| 4 | 0 | 0 |
| 5 | 7 | 17 |
| 6 | 52 | 46 |
| 7 | 10 | 0 |
| 8 | 63 | 70 |
| 9 | 15 | 0 |
| 10 | 45 | 40 |
| 11 | <5 | 14 |
| 12 | 0 | 0 |
| 13 | 0 | 0 |
| 14 | 0 | 0 |
| 15 | 57 | 30 |
| 16 | 32 | 26 |
| 17 | 22 | 36 |
| 18 | 36 | 32 |
| 19 | 38 | 15 |
| 20 | 0 | 0 |
| 21 | 40 | 35 |
| 22 | 29 | 29 |
| 23 | 45 | 33 |
| 24 | 29 | 26 |
| 25 | 57 | 55 |
| 26 | 85 | 63 |
| 27 | 70 | 45 |
| 28 | 78 | 77 |
| 29 | 47 | 45 |
| 30 | 45 | 37 |
| 31 | 55 | 45 |
| 32 | <10 | <10 |
| 33 | 15 | <10 |
| 34 | 53 | 58 |
| 35 | 0 | 0 |
| A | >95 | 85 |
| B | 75 | 45 |
| E | 90 | 63 |
| F | 95 | 86 |

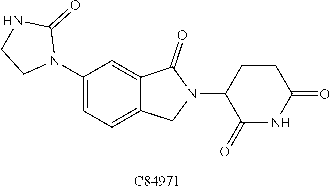 [SMILES: O=C1CCC(N2Cc3ccc(N4CCNC4=O)cc3C2=O)C(=O)N1]

The results above show that, most compounds set forth in Table 2 have much better GSPT1 degradation than previously reported compound C84971 (see WO2021126974A1 and WO2021126973A1).

### Cell Viability Assay

HL-60 cells were seeded at a density of 7000 cells per well in 96-well culture plates (Corning 3903) with IMDM and treated with test compounds following a 6-point serial dilution. RPMI 1640 (0.1% DMSO) was used as control for each well. After 72 h incubation, cell viability was determined using the CellTiter-Glo assay kit (Promega, G9242) according to the manufacturer's instructions. The dose-response curves were determined and IC₅₀ values were calculated using the GraphPad Prism software following a nonlinear regression method. The results of cell viability assays are shown in the following Table 3.

TABLE 3
Results of compounds in cell viability assays

| Compound | HL-60 IC₅₀ (nM) |
|---|---|
| C84971 | >5000 |
| 1 | 1464 |
| 3 | 234 |
| 6 | 400 |
| 8 | 196 |
| 9 | >1000 |
| 10 | 144 |
| 11 | >1000 |
| 12 | >1000 |
| 15 | 290 |
| 19 | >1000 |
| 22 | >1000 |
| 23 | >1000 |
| 25 | 137 |
| 26 | 78 |
| 27 | 397 |
| 28 | 51 |
| 29 | 222 |
| A | 10 |
| B | 122 |
| E | 23 |
| F | 9.2 |

### OTHER EMBODIMENTS

The present disclosure provides merely exemplary embodiments. One skilled in the art will readily recognize from the present disclosure and claims, that various changes, modifications and variations can be made therein without departing from the spirit and scope of the present disclosure as defined in the following claims.

1. A compound of Formula (I): 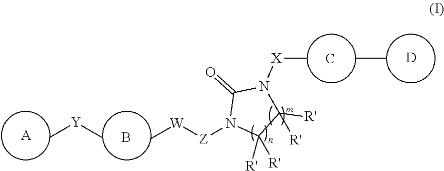
a tautomer thereof, a deuterated derivative of the compound or the tautomer, or a pharmaceutically acceptable salt of the foregoing, wherein:
(xvii) each R′ is independently chosen from hydrogen, halogen groups, linear, branched, and cyclic alkyl groups;
(xviii) m and n are independently chosen from 0, 1, and 2;
(xix) X and Z are independently absent or is chosen from linear, branched, cyclic alkylene groups, linear, branched, and cyclic heteroalkylene groups, and linear, branched, cyclic alkyl groups;
(xx) Y and W are independently absent or chosen from —O—, —C(O)—, —C(O)R^{x}—, —C(S)—, —C(S)R^{x}—, —[C(R^{x}R^{y})]ₚ—, —S—, —S(O)₂—, —S(O)₂R^{x}—, NR^{x}—, and —NR^{x}C(O)—; further wherein p is chosen from 1, 2, 3, 4, 5, and 6; and R^{x} is chosen from hydrogen, linear, branched, and cyclic alkyl groups, carbocyclic groups, heterocyclic groups, aryl groups, and heteroaryl groups;
(xxi) ring A is chosen from 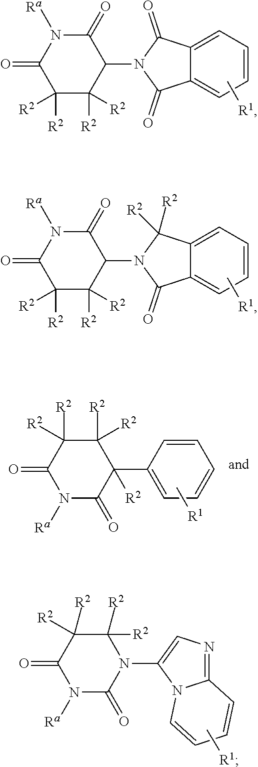
wherein R^{a} is chosen from hydrogen, linear, branched, and cyclic alkyl groups, and pro-drug groups; each R¹ and each R² are independently chosen from hydrogen, halogen groups, OR^{z}, and linear, branched, and cyclic alkyl groups; further wherein R^{z} is chosen from hydrogen, linear, branched, and cyclic alkyl groups, carbocyclic groups, heterocyclic groups, aryl groups, and heteroaryl groups;
(xxii) ring B is absent or is chosen from optionally substituted cycloalkyl groups and heterocycloalkyl groups;
(xxiii) ring C is absent or is chosen from optionally substituted aryl groups and heteroaryl groups,
(xxiv) ring D is absent or is chosen from optionally substituted cycloalkyl groups, heterocycloalkyl groups, and heteroaryl groups;
wherein the linear, branched, and cyclic alkyl groups, linear, branched, and cyclic alkenyl groups, the linear, branched, and cyclic alkylene groups, carbocyclic groups, linear and branched heteroalkenyl groups, linear, branched, and cyclic alkynyl groups, heterocyclic groups, aryl groups, and heteroaryl groups are optionally substituted with at least one group chosen from the following groups:
halogen groups,
hydroxy,
thiol,
amino,
cyano,
—OC(O)C₁-C₆ linear, branched, and cyclic alkyl groups,
—C(O)OC₁-C₆ linear, branched, and cyclic alkyl groups,
—NHC₁-C₆ linear, branched, and cyclic alkyl groups,
—N(C₁-C₆ linear, branched, and cyclic alkyl groups)₂,
—NHC(O)C₁-C₆ linear, branched, and cyclic alkyl groups,
—C(O)NHC₁-C₆ linear, branched, and cyclic alkyl groups,
—C(O)N(C₁-C₆)₂ linear, branched, and cyclic alkyl groups,
—NHaryl groups,
—N(aryl groups)₂,
—NHC(O)aryl groups,
—C(O)NHaryl groups,
—NHheteroaryl groups,
—N(heteroaryl groups)₂,
—NHC(O)heteroaryl groups,
—C(O)NHheteroaryl groups,
—S(O)₂C₁-C₆ linear, branched, and cyclic alkyl groups,
C₁-C₆ linear, branched, and cyclic alkyl groups,
C₂-C₆ linear, branched, and cyclic alkenyl groups,
C₁-C₆ linear, branched, and cyclic hydroxyalkyl groups,
C₁-C₆ linear, branched, and cyclic aminoalkyl groups,
C₁-C₆ linear, branched, and cyclic alkoxy groups,
C₁-C₆ linear, branched, and cyclic thioalkyl groups,
C₁-C₆ linear, branched, and cyclic haloalkyl groups,
C₁-C₆ linear, branched, and cyclic haloaminoalkyl groups,
C₁-C₆ linear, branched, and cyclic halothioalkyl groups,
C₁-C₆ linear, branched, and cyclic haloalkoxy groups,
benzyloxy, benzylamino, and benzylthio groups,
3 to 6-membered heterocycloalkenyl groups,
3 to 6-membered heterocyclic groups, and
5 and 6-membered heteroaryl groups.

2. The compound, tautomer, deuterated derivative, or pharmaceutically acceptable salt of any of claims 1-46, wherein ring A is

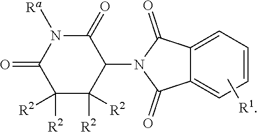

3. The compound, tautomer, deuterated derivative, or pharmaceutically acceptable salt of any of claims 1-46, wherein ring A is

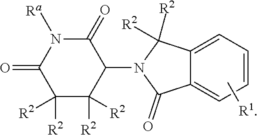

4. The compound, tautomer, deuterated derivative, or pharmaceutically acceptable salt of any of claims 1-46, wherein ring A is

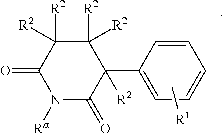

5. The compound, tautomer, deuterated derivative, or pharmaceutically acceptable salt of any of claims 1-46, wherein ring A is

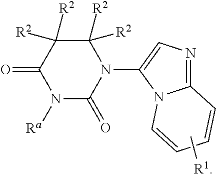

6. A compound chosen from 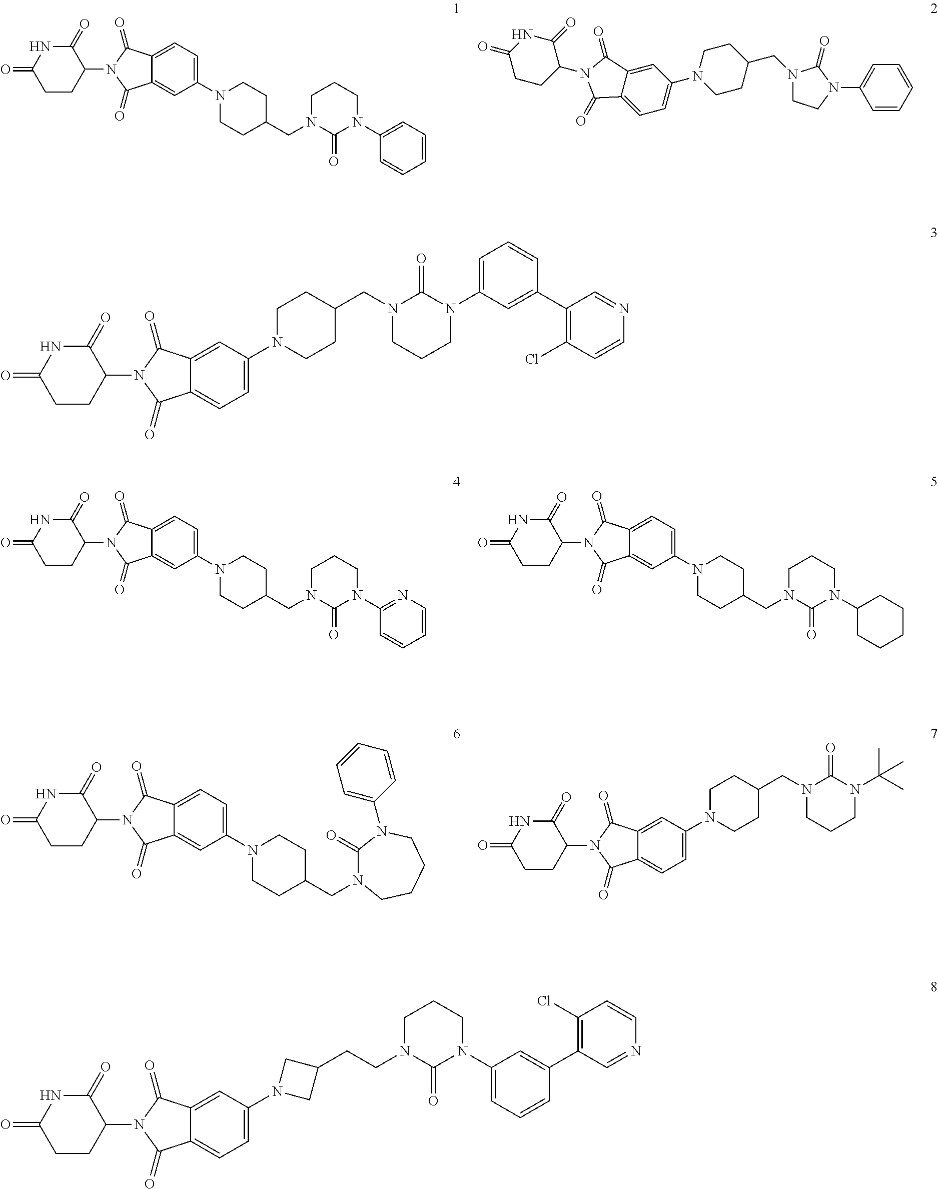 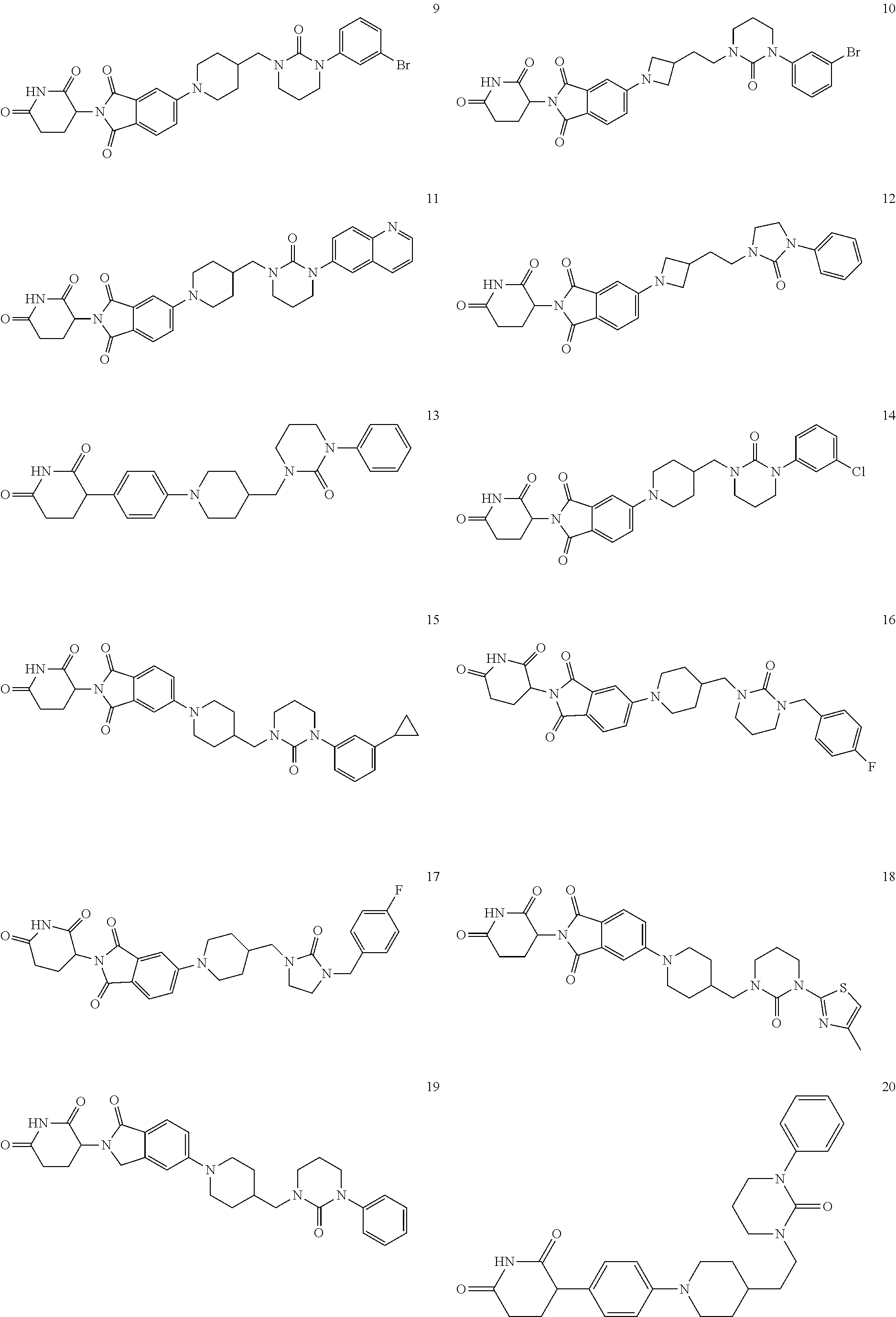 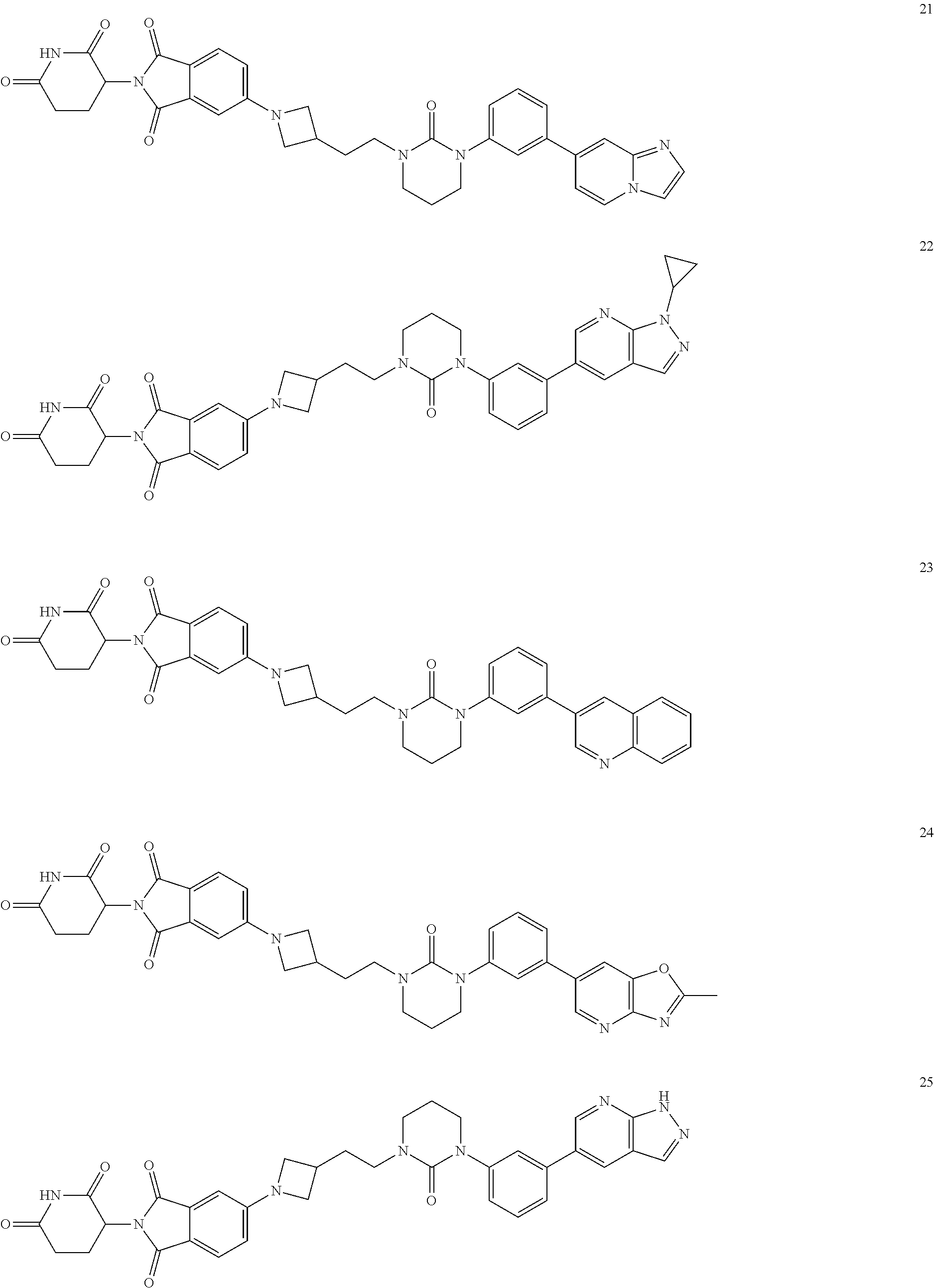 [SMILES: O=C1CCC(N2C(=O)c3ccc(N4CC(CCN5CCCN(c6cccc(-c7ccn8ccnc8c7)c6)C5=O)C4)cc3C2=O)C(=O)N1 ; O=C1CCC(N2C(=O)c3ccc(N4CC(CCN5CCCN(c6cccc(-c7cnc8c(cnn8C8CC8)c7)c6)C5=O)C4)cc3C2=O)C(=O)N1 ; O=C1CCC(N2C(=O)c3ccc(N4CC(CCN5CCCN(c6cccc(-c7cnc8ccccc8c7)c6)C5=O)C4)cc3C2=O)C(=O)N1 ; Cc1nc2ncc(-c3cccc(N4CCCN(CCC5CN(c6ccc7c(c6)C(=O)N(C6CCC(=O)NC6=O)C7=O)C5)C4=O)c3)cc2o1 ; O=C1CCC(N2C(=O)c3ccc(N4CC(CCN5CCCN(c6cccc(-c7cnc8[nH]ncc8c7)c6)C5=O)C4)cc3C2=O)C(=O)N1] 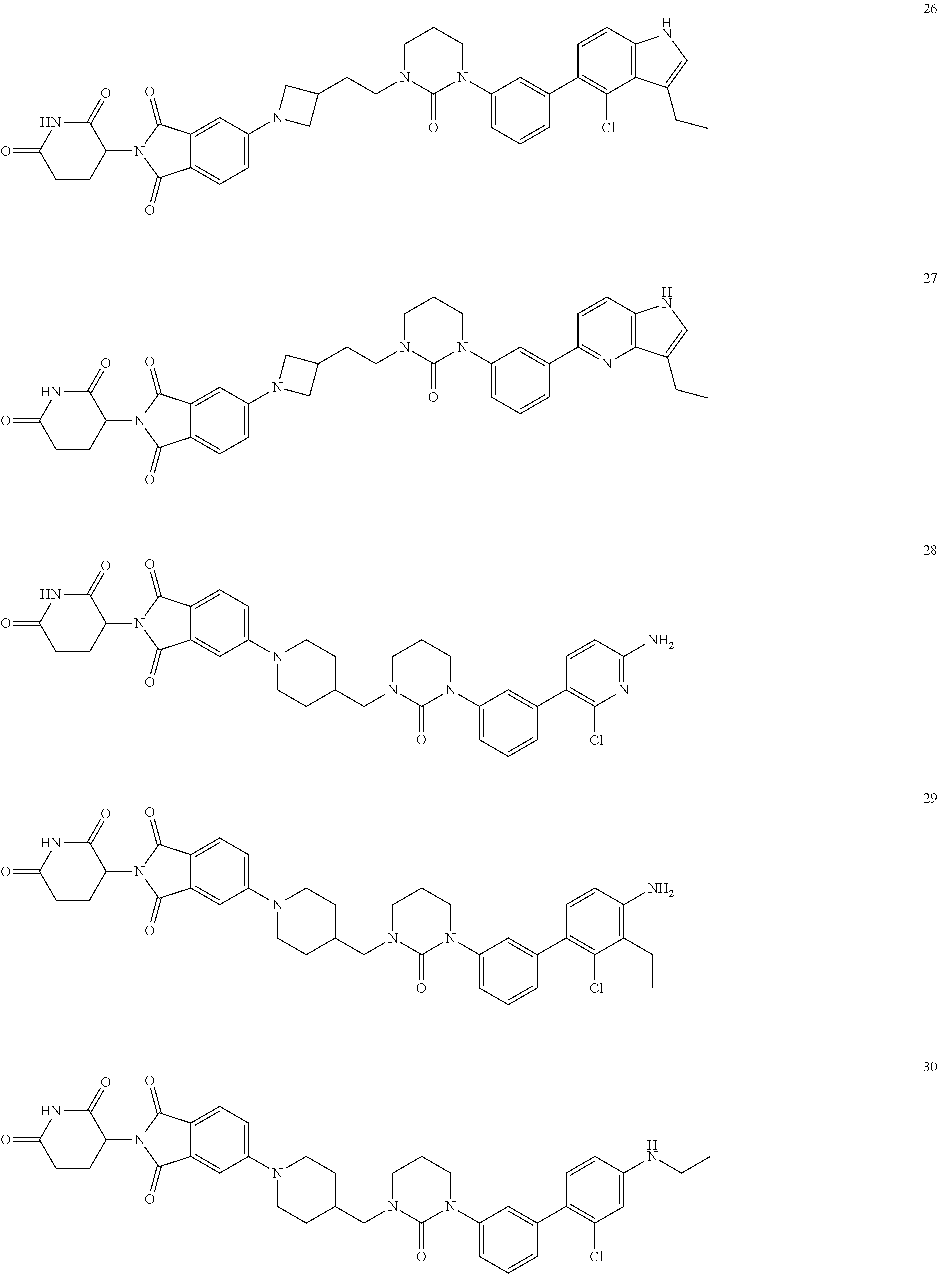 [SMILES: CCc1c[nH]c2ccc(-c3cccc(N4CCCN(CCC5CN(c6ccc7c(c6)C(=O)N(C6CCC(=O)NC6=O)C7=O)C5)C4=O)c3)c(Cl)c12 ; CCc1c[nH]c2ccc(-c3cccc(N4CCCN(CCC5CN(c6ccc7c(c6)C(=O)N(C6CCC(=O)NC6=O)C7=O)C5)C4=O)c3)nc12 ; Nc1ccc(-c2cccc(N3CCCN(CC4CCN(c5ccc6c(c5)C(=O)N(C5CCC(=O)NC5=O)C6=O)CC4)C3=O)c2)c(Cl)n1 ; CCc1c(N)ccc(-c2cccc(N3CCCN(CC4CCN(c5ccc6c(c5)C(=O)N(C5CCC(=O)NC5=O)C6=O)CC4)C3=O)c2)c1Cl ; CCNc1ccc(-c2cccc(N3CCCN(CC4CCN(c5ccc6c(c5)C(=O)N(C5CCC(=O)NC5=O)C6=O)CC4)C3=O)c2)c(Cl)c1] 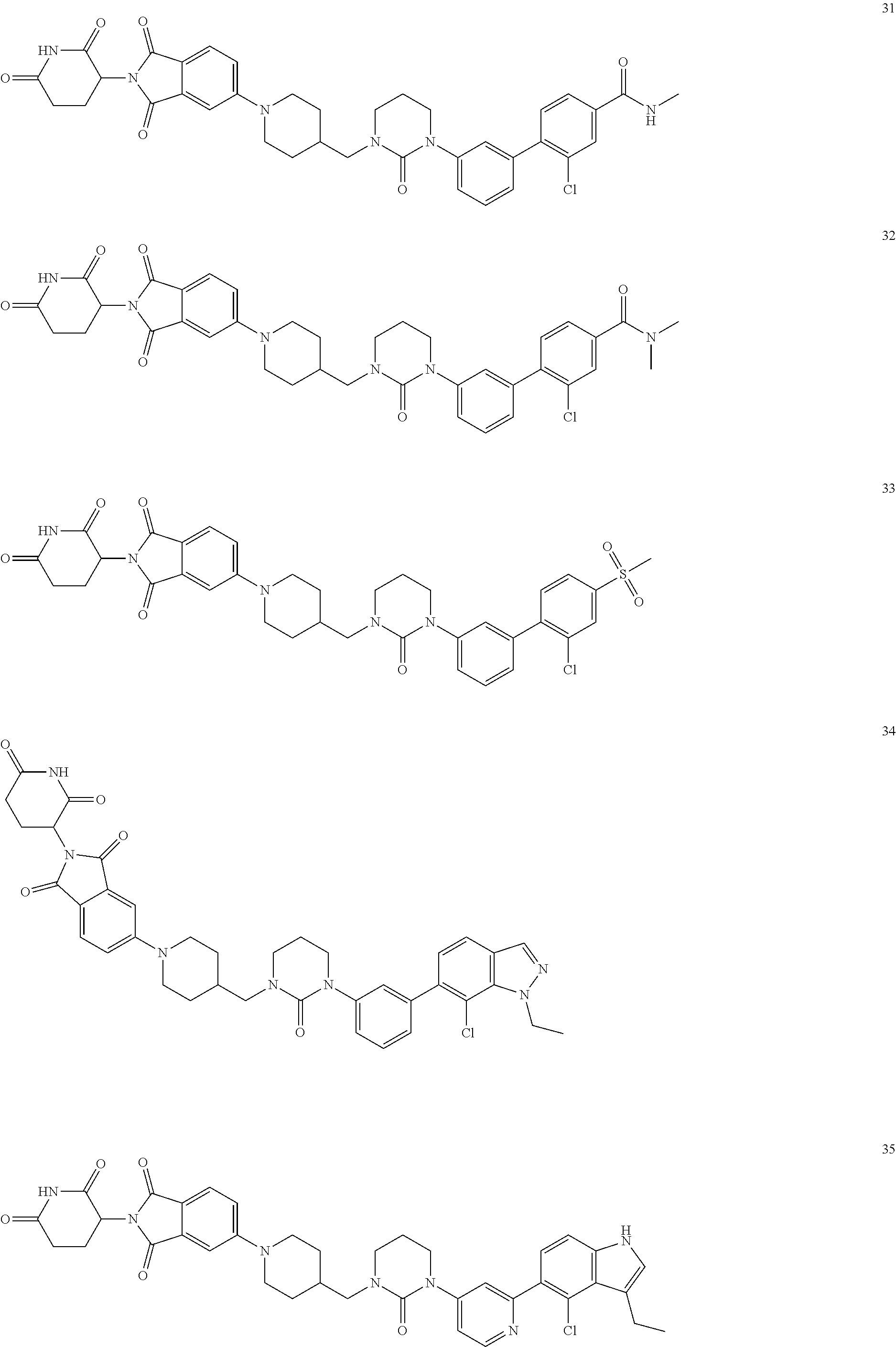 [SMILES: CNC(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CN(C)C(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CS(=O)(=O)c1ccc(-c2cccc(N3CCCN(CC4CCN(c5ccc6c(c5)C(=O)N(C5CCC(=O)NC5=O)C6=O)CC4)C3=O)c2)c(Cl)c1 ; CCn1ncc2ccc(-c3cccc(N4CCCN(CC5CCN(c6ccc7c(c6)C(=O)N(C6CCC(=O)NC6=O)C7=O)CC5)C4=O)c3)c(Cl)c21 ; CCc1c[nH]c2ccc(-c3cc(N4CCCN(CC5CCN(c6ccc7c(c6)C(=O)N(C6CCC(=O)NC6=O)C7=O)CC5)C4=O)ccn3)c(Cl)c12]
a tautomer thereof, a deuterated derivative of the compound or the tautomer, or a pharmaceutically acceptable salt of the foregoing.

7. A pharmaceutical composition comprising a compound, tautomer, deuterated derivative, and/or pharmaceutically acceptable salt according to any one of claims 1-6 and at least one pharmaceutically acceptable carrier.

8. A method for treating or alleviating a disease, a disorder or a condition mediated by the degradation of the GSPT1 protein, comprising administering to a subject in need thereof a therapeutically effective amount of a compound, tautomer, deuterated derivative, and/or pharmaceutically acceptable salt according to any one of the claims 1-6 or the pharmaceutical composition according to claim 7.

9. A method for decreasing GSPT1 protein activity in a disease, a disorder or a condition, comprising administering to a subject in need thereof a therapeutically effective amount of a compound, tautomer, deuterated derivative, and/or pharmaceutically acceptable salt according to any one of the claims 1-6 or the pharmaceutical composition according to claim 7.

10. A method for treating or alleviating a disease, a disorder or a condition mediated by the degradation of the GSPT1 protein, comprising administering to a subject in need thereof a therapeutically effective amount of a compound chosen from 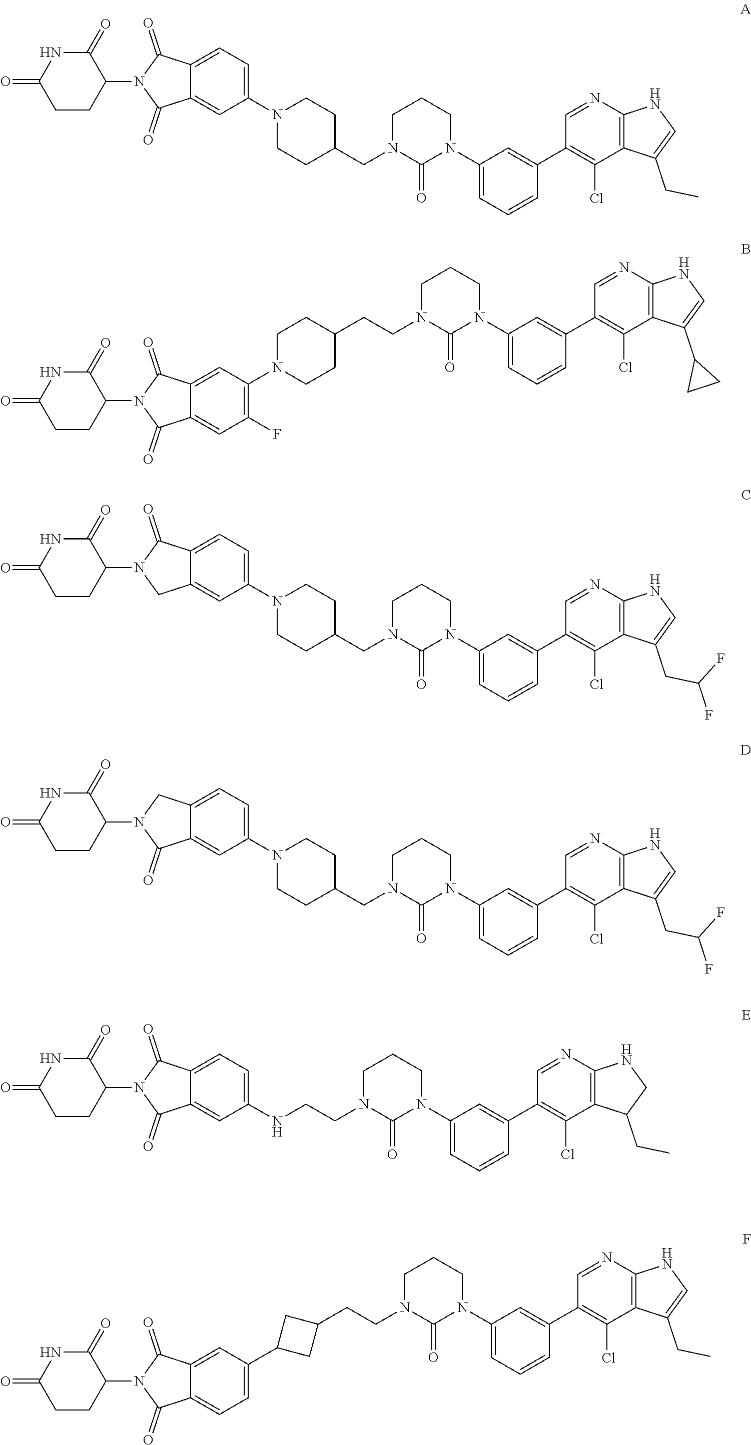
a tautomer thereof, a deuterated derivative of the compound or the tautomer, or a pharmaceutically acceptable salt of the foregoing.

11. A method for decreasing GSPT1 protein activity in a disease, a disorder or a condition, comprising administering to a subject in need thereof a therapeutically effective amount of a compound chosen from 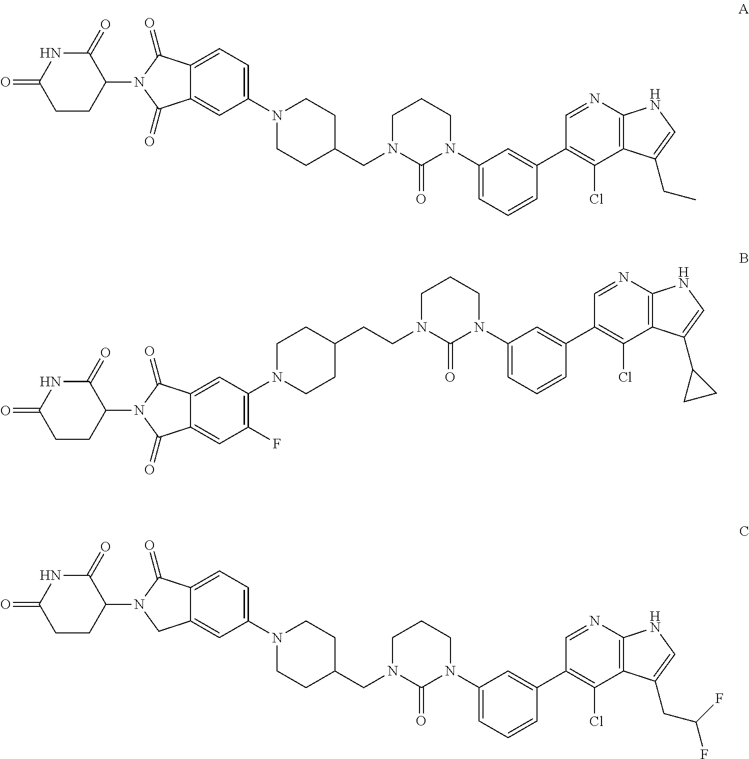 [SMILES: CCc1c[nH]c2ncc(-c3cccc(N4CCCN(CC5CCN(c6ccc7c(c6)C(=O)N(C6CCC(=O)NC6=O)C7=O)CC5)C4=O)c3)c(Cl)c12 ; O=C1CCC(N2C(=O)c3cc(F)c(N4CCC(CCN5CCCN(c6cccc(-c7cnc8[nH]cc(C9CC9)c8c7Cl)c6)C5=O)CC4)cc3C2=O)C(=O)N1 ; O=C1CCC(N2Cc3cc(N4CCC(CN5CCCN(c6cccc(-c7cnc8[nH]cc(CC(F)F)c8c7Cl)c6)C5=O)CC4)ccc3C2=O)C(=O)N1] 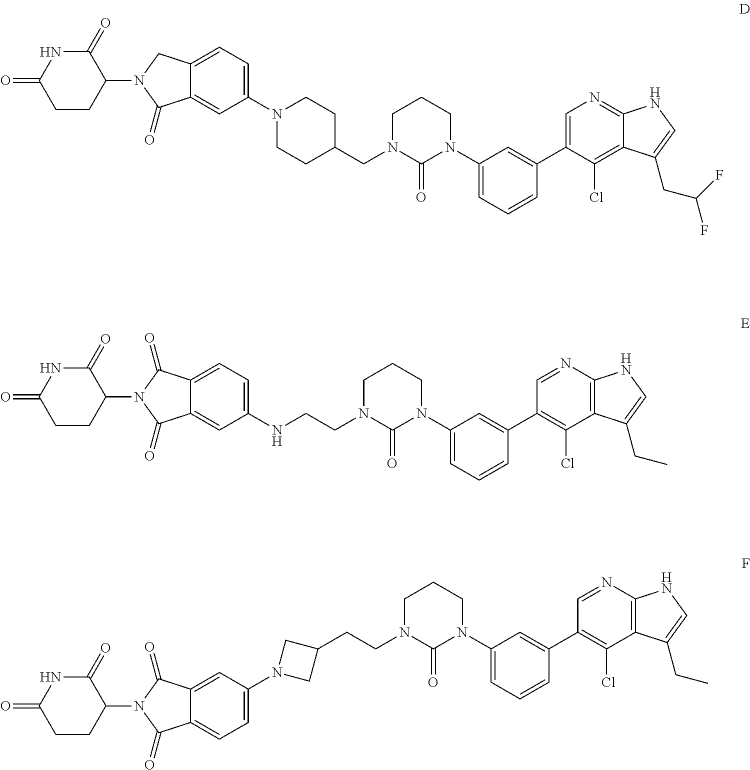 [SMILES: O=C1CCC(N2Cc3ccc(N4CCC(CN5CCCN(c6cccc(-c7cnc8[nH]cc(CC(F)F)c8c7Cl)c6)C5=O)CC4)cc3C2=O)C(=O)N1 ; CCc1c[nH]c2ncc(-c3cccc(N4CCCN(CCNc5ccc6c(c5)C(=O)N(C5CCC(=O)NC5=O)C6=O)C4=O)c3)c(Cl)c12 ; CCc1c[nH]c2ncc(-c3cccc(N4CCCN(CCC5CN(c6ccc7c(c6)C(=O)N(C6CCC(=O)NC6=O)C7=O)C5)C4=O)c3)c(Cl)c12]
a tautomer thereof, a deuterated derivative of the compound or the tautomer, or a pharmaceutically acceptable salt of the foregoing.

12. The method of any of claim 8-11, wherein the disease, the disorder, or the condition is cancer.
